(19) United States
(12) Patent Application Publication
(10) Pub. No.: US 2024/0190983 A1
(43) Pub. Date: Jun. 13, 2024

(54) Novel TNFR2 Binding Molecules

(71) Applicant: JULIUS-MAXIMILIANS-UNIVERSITÄT WÜRZBURG, Würzburg (DE)

(72) Inventors: Harald Wajant, Kist (DE); Andreas Beilhack, Würzburg (DE); Juliane Medler, Fulda (DE); Tim Steinfatt, Würzburg (DE); Luc Van Rompaey, Keerbergen (BE)

(21) Appl. No.: 18/553,282

(22) PCT Filed: Apr. 1, 2022
(86) PCT No.: PCT/EP2022/058788

§ 371 Date: Sep. 29, 2023

(30) Foreign Application Priority Data
Apr. 1, 2021 (EP) 21166756.3

(51) Int. Cl. C07K 16/28; A61K 39/00; A61K 49/00

(52) U.S. Cl. CPC C07K 16/2878; A61K 49/0002; A61K 2039/505; C07K 2317/35; C07K 2317/569; C07K 2317/75

(57) ABSTRACT

The present invention provides a binding molecule that specifically binds TNFR2, but not TNFR1. Preferably the binding molecule is an FcγR-independent agonist of TNFR2 and more preferably has a valency of at least two for binding TNFR2. The binding molecules have a variety of uses including diagnostic and therapeutic. The ability of the binding molecules to be able to modulate immune responses means that they are particularly suited for treating autoimmune diseases, or inflammatory conditions.

### FIELD OF INVENTION

The present invention relates to binding molecules specific for TNFR2 (Tumor Necrosis Factor Receptor 2, also known as TNFRSF1B, and CD120B). The binding molecules are typically able to bind to TNFR2 and trigger TNFR2 signalling independent from FcγR (Fc gamma receptor) binding. The binding molecules may be used to target cells expressing TNFR2. The present invention also relates to the use of the binding molecules in methods of treatment and diagnosis, particularly in treating conditions such as inflammatory disorders, and autoimmune disease.

### BACKGROUND OF INVENTION

TNFα is a cytokine which plays a wide range of roles in inflammation, differentiation and tissue homeostasis and occurs in soluble and transmembrane forms. It is a key inflammatory mediator which is targeted in the clinic with TNF blocking reagents to suppress unwanted immune responses in inflammatory and autoimmune conditions. TNFR1 and TNFR2 are distinct receptors for Tumor Necrosis Factor-alpha (TNFα) with signalling through each receptor eliciting different effects. Signalling via TNFR1 triggers strong and manifold pro-inflammatory effects, whilst signalling via TNFR2 has a wider range of effects ranging from immune stimulation, to suppression of immune responses, to tissue regeneration. The two receptors also differ from each other in terms of which cells they are expressed on. TNFR1 is ubiquitously expressed, whilst TNFR2 is found on a more restricted group of cells, in particular subsets of immune cells, endothelial cells, and neurons. Immune cells expressing TNFR2 include immune suppressive cell types such as T regulatory cells (Tregs), B regulatory cells (B regs), and MDSC (myeloid derived suppressor cells), as well as other cell types, such as CD8+ T cells.

TNFR1 and TNFR2 have a extracellular domain containing four characteristic cystein3=e rich domains (CRDs), a transmembrane segment, and an intracellular domain. Unlike TNFR1, TNFR2 does not include a cytoplasmic death domain, but can still play a role in apoptosis by controlling cell death regulatory factors such as TRAF2 and cIAPs. TNFR2 molecules are able to associate with each other, forming dimeric and trimeric complexes of TNFR2 which can bind TNFα with high affinity, resulting in the formation of oligomers of trimeric TNF-TNFR2 complexes capable of intracellular signalling. Though both TNFR1 and TNFR2 may be expressed on the surface of the same cell type, the two do not form complexes with each other. TNFR2 is able to bind both soluble and membrane bound TNFα. Binding of membrane TNFα is able to activate TNFR2 effectively, but with soluble TNFα the oligomerisation of liganded TNFR2 trimers to hexamers or higher complexes is inadequate and thus fails to trigger effective TNFR2 activation. As well as TNFα, lymphotoxin-alpha (LTα) is also thought to act as a ligand for TNFR2.

When TNFR2 is activated by membrane TNFα, the intracellular domains of the receptor are thought to recruit existing cytoplasmic TRAF2 trimers, resulting in recruitment of the E3 ligases cIAP1 and cIAP2 to TNFR2 complexes, which leads to classical NFκB signalling. TRAF2 recruitment is also associated with activating the alternative NFκB signalling pathway. Binding of membrane TNFα to TNFR2 is also thought to trigger signalling through the PI3K/Akt pathway, maintaining survival and enhancing proliferation, as well as promoting other functions such as cellular adhesion and migration. TNFR2 knockout mice are more susceptible to inflammatory disease and, whilst they have Treg cells, they are less responsive. Naturally occurring polymorphisms of TNFR2 also exist and are associated with a wide variety of autoimmune diseases.

The expression and function of TNFR2 on Treg cells make it a desirable target for modulating the immune system, especially as TNFR2 is found on a more limited subset of cells than TNFR1. The ability to stimulate Treg cells offers a way to control unwanted immune responses in a wide range of conditions where their ability to suppress immune responses may be useful including allergy, autoimmunity, transplantation, graft versus host disease (GVHD) and infectious disease. As well as Treg cells, other cell types expressing TNFR2 may be targeted to bring about desired functions. There is therefore an ongoing need for agents that target and activate TNFR2.

### SUMMARY OF THE INVENTION

The present invention provides binding molecules which are able to bind the extracellular domain of TNFR2. In a preferred embodiment, a binding molecule of the present invention is able to bind TNFR2 and trigger signalling through the receptor thus acting as an agonist. In a further particularly preferred embodiment the binding molecules may act as agonists of TNFR2 independent of FcγR binding. In a particularly preferred embodiment, the binding molecules of the present invention are antibodies or antibody fragments. In a further particularly preferred embodiment of the present invention the binding molecules are selective in that they specifically bind TNFR2, but not TNFR1.

The binding molecules can have a variety of uses from diagnostic applications to therapeutic uses. As the binding molecules have the ability to modulate the immune system they are particularly useful in treating autoimmune conditions, and inflammatory conditions. The binding molecules may be used to downregulate the immune system due to their action on Treg cells and so are especially useful in treating such conditions, including in particular Graft versus Host Disease (GvHD).

Accordingly, in one embodiment the present invention provides a binding molecule that specifically binds TNFR2, but not TNFR1, where the binding molecule is an FcγR independent agonist of TNFR2 and has a valency of at least two for binding TNFR2. In an especially preferred embodiment, a binding molecule has a valency of at least four for TNFR2. In a further especially preferred embodiment, a binding molecule has a valency of four for TNFR2. In an especially preferred embodiment, a binding molecule has a valency of at least six for TNFR2. In a further especially preferred embodiment, a binding molecule has a valency of six for TNFR2.

In another especially preferred embodiment, a binding molecule of the present invention comprises at least one binding domain for TNFR2 which is a VHH binding domain specific for TNFR2. In another especially preferred embodiment, all of the binding domains specific for TNFR2 are VHH binding domains specific for TNFR2. In a further especially preferred embodiment, the binding molecule is a hexavalent antibody comprising six VHH binding domains specific for TNFR2.

The present invention also provides:
The binding molecule of the invention for use as a medicament.
The binding molecule of the invention for use in a method of treating or preventing an autoimmune disorder, or an inflammatory disorder.
A method of treating or preventing an autoimmune disorder, or an inflammatory disorder comprising administering a binding molecule of the invention to a subject suffering from, or at risk of, the disorder.
Use of a binding molecule of the invention for manufacturing a medicament for use in a method of treating or preventing an autoimmune disorder, or an inflammatory disorder.
A method of stimulating cell proliferation comprising contacting a target cell expressing TNFR2 with a binding molecule of the invention.
A pharmaceutical composition comprising a binding molecule of the invention and a pharmaceutically acceptable carrier.
A method of detecting TNFR2 comprising contacting a test sample with a binding molecule of the invention and detecting binding of the binding molecule to TNFR2 if present.
A diagnostic method comprising administering a binding molecule of the invention to a subject and detecting binding of the binding molecule to TNFR2 if present.
A binding molecule of the invention for use in a method of diagnosis, the method comprising administering a binding molecule of the invention to a subject and detecting binding of the binding molecule to TNFR2.

### SEQUENCE LISTING

The present application includes a sequence listing in electronic form which forms part of the application as filed.

### BRIEF DESCRIPTION OF THE FIGURES

FIG. 1—Nomenclature and schemes of the domain architecture of exemplary anti-TNFR2 antibody variants with N-terminal organized TNFR2 binding sites (Fab or scFv domains). CI=clone identifier of parental anti-TNFR2 antibody, e.g. the mouse anti-human TNFR2 antibodies C4, C19, C9, C40 etc.; N297A refers to the corresponding mutation in the heavy chain of human IgG1 which reduces FcγR interaction; N-RGY refers to the N297A mutation plus mutations promoting IgG1 hexamerization.

FIG. 2—Western blot analysis of variants of the anti-TNFR2 antibody C4 with N-terminal unidirectionally orientated TNFR2 binding sites. Flag-tagged variants of the indicated C4 constructs (200 ng) were analyzed by Western blotting with anti-human IgG and anti-Flag antibodies.

FIG. 3—TNFR2-mediated IL8 production in HT1080-Bcl2-TNFR2 cells after treatment with C4 variants with unidirectionally organized N-terminal TNFR2 binding sites. HT1080-Bcl2-TNFR2 cells stably expressing human TNFR2 were stimulated with supernatants containing the indicated concentrations of the various C4 constructs. Next day, supernatants were harvested and analyzed by ELISA for their IL8 content. The data shown are derived from parallel processed experiments which for better resolution were shown in three diagrams. The maximum response induced with a highly active oligomeric form of the TNFR2-specific TNF mutant TNF(143N/145R) positive control is indicated by a dotted line.

FIG. 4. TNFR2-mediated cell death in Kym-1 cells after treatment with C4 variants with unidirectionally orientated N-terminal TNFR2 binding sites. Kym-1 cells were stimulated with supernatants containing the indicated concentrations of the various C4 constructs. Next day, cell viability was determined using crystal violet staining. A highly active oligomeric form of the TNFR2-specific TNF mutant TNF(143N/145R) (oligo. TNF80) served as a positive control for TNFR2 engagement.

FIG. 5—TNFR2-mediated p100 processing in Kym-1 cells after treatment with C4 variants with N-terminal unidirectionally orientated TNFR2 binding sites. Kym-1 cells were stimulated overnight with supernatants containing the indicated concentrations of the various C4 variants. Treatment was performed in the presence of 20 μM ZVAD to prevent TNFR2-induced cell death. A nonameric TNFR2-specific mutant of mouse TNF (oligo. TNF80), which potently activates human and murine TNFR2, was used as a positive control.

FIG. 6—TNFR2-mediated IL8 production in HT1080-Bcl2-TNFR2 cells after treatment with variants of C19, C27 and C40 with N-terminal unidirectionally orientated TNFR2 binding sites. HT1080-Bcl2-TNFR2 cells were stimulated with supernatants containing the indicated concentrations of the various constructs. Next day, supernatants were harvested and analyzed by ELISA for their IL8 content. The maximum response induced with a highly active oligomeric form of the TNFR2-specific TNF mutant TNF(143N/145R) positive control is indicated by a dotted line. Note: even constructs derived of the lower affinity antibody C27 showed significant agonistic activity (lower panel).

FIG. 7—Nomenclature and schemes of the domain architecture of anti-TNFR2 antibody variants with N-terminal and C-terminal organized TNFR2 binding sites (Fab or scFv domains). CI=clone identifier, e.g. C4, C19 etc.; N297A refers to the corresponding mutation in the heavy chain of human IgG1 reducing FcγR binding.

FIG. 8—Western blot analysis of C4 variants with N-terminal and C-terminally fused TNFR2 binding sites. Flag-tagged variants of the indicated C4 constructs (200 ng) were analyzed by Western blotting with anti-human IgG and anti-Flag antibodies. Note that the heavy chain of C4-IgG1(N297A)-HC:scFvC4 does not contain a Flag tag.

FIG. 9—TNFR2-mediated IL8 production in HT1080-Bcl2-TNFR2 cells after treatment with C4 constructs with N-terminal and C-terminal TNFR2 binding sites. HT1080-Bcl2-TNFR2 cells were stimulated with supernatants containing the indicated concentrations of the various C4 constructs. Next day, supernatants were harvested and analyzed by ELISA for their IL8 content. The data shown are derived from parallel processed experiments which for better resolution were shown in three diagrams. The maximum response induced with a positive control (oligo. TNF80; highly active oligomeric form of the TNFR2-specific TNF mutant TNF(143N/145R)) is indicated by a dotted line. Corresponding constructs with only N-terminal binding sites were analyzed in parallel for comparison.

FIG. 10—TNFR2-mediated cell death in Kym-1 cells after treatment with C4 constructs with N-terminal and C-terminal organized TNFR2 binding sites. Kym-1 cells were stimulated with supernatants from HEK293 transfected cells containing the indicated approximate concentrations of the various C4 constructs. Next day, cell viability was determined using the MTT assay. A highly active oligomeric form of the TNFR2-specific TNF mutant TNF(143N/145R) was used as a positive control.

FIG. 11—Binding properties of TNFR2-specific antibodies. (A) Equilibrium binding of the indicated anti-TNFR2 antibody GpL fusion proteins to cell expressed human TNFR2 at 37° C. (B) HeLa-TNFR2 cells were pretreated with the indicated amounts of anti-TNFR2 antibodies or remained untreated. 5 ng/ml GpL-TNF were added and binding was measured after 1 hour incubation at 37° C. Binding of the various constructs to untransfected HeLa cells was analyzed to obtain unspecific binding values.

FIG. 12—Competition of αTNFR2 antibodies. HeLa-TNFR2 cells were pretreated with 3-10 μg/ml of the indicated anti-TNFR2 antibodies or remained untreated. 50 ng/ml of the various αTNFR2-IgG1(N297A)-LC:GpL variants were added and binding was measured after 1 hour FIG. 13—Tetravalent mAb binding TNFR2 molecules trigger much stronger IL8 induction than TNFR2 molecules bound by the conventional bivalent mAb variant. (A,B) TNFR2-negative Hela cells and HeLa-TNFR2 cells were pairwise stimulated for 8 hours with the indicated concentrations of the GpL fusion proteins of the bi- and tetravalent version of the TNFR2-specific mAb C4 (see inset for scheme). Supernatants of HeLa-TNFR2 cells were then analyzed for TNFR2-induced IL8 production (B) and repeatedly washed cells (HeLa, and HeLa-TNFR2) were analyzed for cell-associated GpL activity. Specific binding to TNFR2, shown in (A) was calculated by subtraction of the unspecific binding values obtained from the HeLa cells from the total binding values derived of the HeLa-TNFR2 cells. Please note luciferase activity was normalized according to the ratio of the number of GpL reporter domains to the number of TNFR2-binding domains within the two mAb variants (1 versus 0.5). (C) IL8 production by the mAb variants were directly plotted as a function of their specific binding. The latter was transformed into “occupied receptors per cells” by help of the number of cells in the assay and the measured specific activity (RLU/molecule) of the GpL constructs. Maximum binding of the two constructs obtained from A is indicated by dashed vertical lines. The dotted lines indicate linear regression of the IL8 production as function of receptor occupancy.

FIG. 14—Binding of TNFR2-specific antibodies to human, murine and cynomolgus TNFR2. HEK293 cells were transiently transfected with human, murine and cynomologus TNFR2 or empty vector. Equilibrium binding of the indicated anti-TNFR2 antibody GpL fusion proteins were then determined at 37° C. Binding to empty vector transfected cells was determined to obtain unspecific binding values.

FIG. 15—Scheme of the domain architecture of TNFR2 and indication of the extracellular TNFR2 domains recognized by the anti-TNFR2 antibodies C4, C19, C27 and C40 and tetravalent CI-IgG1(N297A)-HC:scFvCI variants derived thereof. CI, anti-TNFR2 clone identifier; CRD, cysteine-rich domain.

FIG. 16—Western blot analysis of mono- and bispecific TNFR2-specific CI-IgG-HC:scFvCI variants. Flag-tagged variants of the indicated antibody constructs (200 ng) were analyzed by Western blotting with anti-human IgG (lower panel) and anti-Flag antibodies (upper panel). Conventional IgG variants were included for comparison. Please note: i) The heavy chains of C4-IgG1(N297A), C4-IgG1(N297A)-HC:scFvC4 and C4-IgG1(N297A)-HC:scFvC19 do not contain a Flag tag, ii) the conventional C19 variant used was in the murine IgG1(D265A) isoform.

FIG. 17—TNFR2-mediated IL8 production in HT1080-Bcl2-TNFR2 cells after treatment with mono- (upper level) and biparatopic (lower panel) TNFR2-specific CI-IgG-HC:scFvCI variants. The response induced by 200 ng/ml of a highly agonistic oligomerized TNFR2-specific TNF variant is indicated (dotted line). Please note, in the upper panel, the C27 antibody which has lower affinity still gave some agonistic activity in the tetravalent form.

FIG. 18—TNFR2-mediated cell death in Kym-1 cells after treatment with mono- (upper level) and bispecific (lower panel) TNFR2-specific CI-IgG-HC:scFvCI variants. Kym-1 Cells were stimulated with supernatants containing the indicated approximate concentrations of the various constructs. Next day, cell viability was determined using crystal violet staining. Conventional antibodies (upper panel) were analyzed in parallel for comparison.

FIG. 19—TNFR2-mediated p100 processing in Kym-1 cells after treatment with mono- (A) and bispecific (B) TNFR2-specific CI-IgG-HC:scFvCI variants. Cells were stimulated overnight with supernatants containing the indicated approximate concentrations of the various constructs. Treatment was performed in the presence of 20 μM ZVAD to prevent TNFR2-induced cell death.

FIG. 20—Purification and functional analysis of C4-IgG1(N297A)-HC:scFvC4, C4-IgG1(N297A)-LC:scFvC4 and C4-IgG1(N297A)-LC:scFvC4-HC:scFvC4. (A) Silver stained SDS-PAGE gel. (B) Gelfiltration analysis. (C) IL8 induction in HT1080-Bcl2-TNFR2 cells. (D) Cell death induction in Kym-1 cells. (E) Induction of p100 processing in Kym-1 cells. ZVAD was added to prevent cell death.

FIG. 21—Ex vivo assessment of Treg and Tcon cells after treatment with tetravalent TNFR2 agonists. PBMCs were collected from donors, cultured, harvested, then treated with tetravalent antibodies (mono- or bi-specific; Fab-HC-scFv format). Untreated negative control cells were also assessed as was a positive control with the Star2 (also named TNC-sc(mu)TNF(143N/145R) TNFR2 agonist (oligo TNF80), an oligomerized highly potent TNFR2-selective TNF variant (Chopra et al., 2016). The top panel shows percentage fold change in Tregs, the bottom in Tcon cells.

FIG. 22—Dose-dependent increase of Tregs in syngeneic FoxP3-Luciferase transgenic mice by the human-mouse crossreactive tetravalent TNFR2 agonist C19-IgG1(N297A)-HC:scFvC19. Transgenic FoxP3-Luci mice were administered 8 doses of a TNFR2 agonist (N=1) and then assessed daily by bioluminescence imaging. After four days the mice were sacrificed and cells from the spleen analysed. Panel (A) shows the time line for the experiment. Panel (B) shows the results of the FACS analysis of splenocytes.

FIG. 23—Dose-dependent increase of Tregs in syngeneic FoxP3− Luciferase transgenic mice by the human-mouse crossreactive tetravalent TNFR2 agonist C19-IgG1(N297A)-HC:scFvC19. Transgenic FoxP3-Luciferase mice were administered different doses of C19-IgG1(N297A)-HC;scFvC19 and then assessed daily with bioluminescence imaging. After four days the mice were euthanized and cells from the spleen analyzed with flow cytometry.

FIG. 24—Production and analysis of TNFR2-specific TNF constructs. Panel (A): Domain architecture of TNF-based molecules. TNF80 refers to a TNFR2-specific mutant of soluble TNF. Panel (B): murine soluble TNF80 based constructs were purified by anti-Flag affinity purification by help of a Flag tag contained in all constructs and analyzed by SDS-PAGE and silver staining. Panel (C): Gel filtration analysis of the purified protein shown in “B”.

FIG. 25—Agonistic activity of different formats of soluble murine TNF80. Panel (A): Kym-1 cells were stimulated with the indicated concentrations of the various muTNF80 variants. Next day, cell viability was determined using crystal violet staining. Panel (B): HT1080-Bcl2-TNFR2 cells were stimulated with the indicated concentrations of the various TNF80 variants shown in FIG. 24. Next day, supernatants were harvested and analyzed by ELISA for their IL8 content. The data shown are derived from four independent experiments.

FIG. 26—In vivo Treg expansion by Fc(DANA)-TNC-muTNF80. Mice with luciferase under the control of the Foxp3 promoter were injected i.v with a single injection of the indicated amount of Fc(DANA)-TNC-muTNF80 and were analyzed daily by bioluminescence imaging (BLI)(upper panel). Day 0 and day 4 BLI pictures are shown in the middle panel. The lower panel shows flow cytometry analysis based quantification of Treg frequency on day 4.

FIG. 27—Demonstration of selective binding of TNFR2, but not TNFR1, by binding molecules of the invention. HEK293 cells were transiently transfected with: (i) a TNFR2 expression plasmid; (ii) an expression plasmid encoding the extracellular domain of TNFR1 with a GPI anchor domain; or (iii) empty vector (ev). Next day, the indicated GpL tagged binding molecules were added at the indicated concentration to the cells. After incubation for 1 hour at 37° C., followed by two washes with PBS, the remaining cell bound GpL activity was determined. GpL-TNF served as positive control binding to both TN receptor types.

FIG. 28—IgG1(N297A)-HC:scFv, IgG1(N297A)-HC:TNC and IgG1(N-RGY) variants of 005-B08 from WO 2020/089474 are strong TNFR2 agonists as measured using IL-8 release. The maximum response induced with C4-IgG1(N297A)-HC:C4 as positive control is indicated by a dotted line.

FIG. 29—Linker length variation between Heavy and Light Chains in scFv, and scFv fused to Heavy or Light chains of C4-IgG1(Durv)-scFv:C4 variants do not affect TNFR2 agonism activity. (A) Domain architecture of constructs. “G4S” refers to the linker sequence GGGGS and “Durv” indicates constant region sequence of the antibody Durvalumab. (B) Gel filtration of purified C4-IgG1(Durv)-HC:scFvC4(G4S)4 and C4-IgG1(Durv)-LC:scFvC4(G4S)4. (C) IL-8 release assay. The maximum response induced with a highly active oligomeric form of the TNFR2-specific TNF mutant TNF(143N/145R) positive control was indicated by a dotted line.

FIG. 30—Sequence optimization does not affect the activity of C4-IgG1(Durv)-HC/LC:scFvC4 variants. (A) Gel filtration analysis of purified C4-IgG1(Durv)-HC/LC:scFvC4 variants. (B) IL8 release assay as a measure of TNFR2 activation. The maximum response induced with a highly active oligomeric form of the TNFR2-specific TNF mutant TNF(143N/145R) positive control is indicated by a dotted line.

FIG. 31—TNFR2 binding of TNFR2-specific sdAbs. Equilibrium binding of the indicated TNFR2-specific VHH-Fc-GpL VHH constructs to cell expressed human TNFR2 at 37° C. Specific binding was obtained by subtraction of the non-specific binding values (Hek293 cells) from the transfectants expressing human TNFR2 (total binding).

FIG. 32—Binding of TNFR2-specific hexavalent 3×VHH-Fc-GpL constructs to human and murine TNFR2. Equilibrium binding of the indicated TNFR2-specific 3×VHH-Fc-GpL VHH constructs to cell expressed human TNFR2 at 37° C. The GpL reporter domain has been cloned to the C-terminus of the Fc domain. Open circles=unspecific binding; Open squares=total binding; Filled circles=specific binding.

FIG. 33-3×VHH-Fc(DANA) variants of VHH:C18, VHH:C74, VHH:C188 and VHH:C238 block TNFα binding to TNFR2. HT1080-Bcl2-TNFR2 cells were pretreated with the indicated amounts of TNFR2-specific VHH constructs or with the CD40-specific construct 3×VHH:CD40(V12t)-Fc(DANA) as a negative control or remained untreated. 5 ng/ml GpL-TNF were added and binding was measured after 1 h incubation at 37° C.

FIG. 34—TNFR2-specific VHHs do not bind TNFR1. HEk293 cells were transiently transfected with expression vectors encoding TNFR1 and TNFR2 and were then analyzed for binding of the indicated proteins. Binding to empty vector transfected cells served as a negative control for non-specific binding. TNF-GpL served as a positive control for successful TNFR1 binding. Filled circles=total binding; x=unspecific binding; Open circles=specific binding.

FIG. 35 VHH:C18, VHH:C188 and VHH:C238 bind CRD4 of TNFR2 and VHH:C74 binds CRD3. (A) Black 96-well plates were coated with protein G and then loaded with the 3×VHH-Fc(DANA) versions of the indicated TNFR2-specific nanobodies. After removal of the unbound proteins, immobilized antibody constructs were analyzed for binding of the GpL-tagged deletion mutants of TNFR2(ed) shown in the western blot in the right panel. Finally, binding of these GpL fusion proteins were quantified. (B) Clones C188 and C4 compete with TNF for binding to human TNFR2. HT1080-Bcl2-TNFR2 cells were pretreated with the indicated amounts of TNFR2-specific VHH constructs or with the CD20-specific mAb Rituximab as a negative control or remained untreated. 5 ng/ml GpL-TNF were added and binding was measured after 1 h incubation at 37° C.

FIG. 36—TNFR2-mediated IL8 release after treatment with VHH-based constructs with four N-terminal-only TNFR2 binding sites. HT1080-Bcl2-TNFR2 cells were stimulated with supernatants containing the indicated constructs and the resulting supernatant analyzed the next day for IL-8 release. The maximum response induced with a highly active oligomeric form of the TNFR2-specific TNF mutant TNF(143N/145R) (upper panel) or with C4-IgG1(N297A)-HC:C4 (middle and lower panel) served as positive control and is indicated by a dotted line.

FIG. 37—TNFR2-mediated IL8 release and Kym-1 cell death after treatment with VHH-based constructs with six N-terminal-only TNFR2 binding sites. (A) HT1080-Bcl2-TNFR2 cells were stimulated with the indicated constructs and release of IL-8 measured in conditioned medium. The maximum response induced with a highly active oligomeric form of the TNFR2-specific TNF mutant TNF(143N/145R) positive control is indicated by a dotted line. (B) Kym-1 cells were stimulated with the various hexavalent variants and the next day, cell viability was determined using the MTT assay. Maximum killing induced by 200 ng/ml of a highly active oligomeric form of the TNFR2-specific TNF mutant TNF(143N/145R) is again indicated by dotted line.

FIG. 38—Side-by-side comparison of the agonistic activity of VHH:C188-Fc, 2×VHH:C188-Fc-GpL and 3×VHH:C188-Fc. (A,B) Purified proteins were analyzed by SDS-PAGE (A) and gel filtration (B). (C) Measurement of IL-8 release as an indicator of TNFR2 activation. The maximum response induced with a highly active oligomeric form of the TNFR2-specific TNF mutant TNF(143N/145R) positive control is indicated by a dotted line. (D) Cell viability assay after Kym-1 cells were stimulated with the various VHH:C188 variants. Maximum killing induced by C4-IgG1(N297A)-HC:scFvC4 is indicated by a dotted line.

FIG. 39—Side-by-side comparison of IL8 release and Kym-1 cell death-inducing activity of the VHH-Fc-GpL, 2×VHH-Fc-GpL and 3×VHH-Fc-GpL variants of C18 and C74. (A) IL-8 release assay. The maximum responses induced with a highly active oligomeric form of the TNFR2-specific TNF mutant TNF(143N/145R) is indicated.

FIG. 40—Linker length between VHH domains of 3×VHH:C188-IgG1(Durv) variants do not affect TNFR2 agonism. Linker length between VHH domains of 3×VHH:C188-IgG1(Durv) variants do not affect TNFR2 agonism. (A) Domain architecture of constructs. (B) SDS-PAGE analysis of purified constructs. (C) Gel filtration analysis. (D) HT1080-Bcl2-TNFR2 cells were stimulated with supernatants containing the indicated constructs. Next day, supernatants were analyzed by ELISA for their IL8 content. The maximum response induced with a highly active oligomeric form of the TNFR2-specific TNF mutant TNF(143N/145R) is indicated by a dotted line. The parental molecule 3×VHH:C188-Fc(DANA) was included for comparison.

FIG. 41—Constructs of different domain architecture with 4, 6 or 9 VHH:C188 domains elicit strong TNFR2 agonism. (A) Domain architecture of constructs. TNC refers to the trimerization domain of tenascin-C. (B) IL-8 release assay. The maximum response induced with a highly active oligomeric form of the TNFR2-specific TNF mutant TNF(143N/145R) is indicated by a dotted line.

FIG. 42—Protein G enhances the agonistic activity of sequence optimized variants of the anti-TNFR2 mAb C4. (A) Western blot analysis with anti-human IgG1 antibody of cell culture supernatants of HEK293 cells transiently transfected with expression plasmids encoding the light and heavy chain of the indicated antibody variants. (B) HT1080-TNFR2 cells were challenged with the indicated concentrations of the C4 variants in the presence and absence of 500 ng/ml protein G (PG). Next day, cell supernatants were analyzed for IL8 production by ELISA. The maximum response induced with a highly active oligomeric form of the TNFR2-specific TNF mutant TNF(143N/145R) is indicated by a dotted line. Enhanced activity in the presence of protein G indicates the presence of latent agonism that can be realized by generation of constructs with appropriate domain architecture.

FIG. 43—TNFR2 Agonism of sequence optimized variants of 3×VHH:C188-Fc(Durv). (A) Western blot analysis with anti-human IgG1 antibody of cell culture supernatants of HEK293 cells transiently transfected with expression plasmids encoding the indicated 3×VHH:C188-Fc(Durv) variants. (B) HT1080-TNFR2 cells were challenged with the indicated concentrations of the 3×VHH:C188-Fc(Durv) variants and the parental 3×VHH:C188-Fc(DANA) molecule. Next day, cell supernatants were analyzed for IL8 production by ELISA. The maximum response induced with a highly active oligomeric form of the TNFR2-specific TNF mutant TNF(143N/145R) is indicated by a dotted line.

FIG. 44—TNFR2-agonists increase Foxp3⁺ regulatory T cells (Tregs) in human peripheral blood mononuclear cells (PBMC)—Fold change of Treg frequency of human peripheral blood mononuclear cells (PBMCs) after 4 days of stimulation with indicated TNFR2-agonist relative to untreated control of the same donor. Samples were measured with flow cytometry and Tregs were defined as CD3⁺CD4⁺FoxP3⁺ T cells. Number of individual donors are indicated with n. Significance was tested with one-sample t test with a hypothetical value 1 and is indicated with *P=0.05, **P=0.01, ***P=0.001, ****P<0.0001.

FIG. 45—Activation of Tregs by C4-IgG1(N297A)Hc-scFvC4 antibody. Human PBMCs were stimulated for 4 days with 100 ng/ml C4-IgG1(N297A)Hc-scFvC4 and expression of markers related to Treg activity was measured with flow cytometry. Fold change expression relative to untreated control of the same donor is shown. Tregs were defined as CD3⁺CD4⁺FoxP3⁺ T cells. Mean±SD and each dot represents individual donor.

FIG. 46—Activation of Tregs by 3×VHHC188(G4S)1-Fc(Durv) antibodies. Human PBMCs were stimulated for 4 days with indicated concentration of 3×VHHC188(G4S)1-Fc(Durv) and expression of markers related to Treg activity was measured with flow cytometry. Fold change expression relative to untreated control of the same donor is shown. Tregs were defined as CD3⁺CD4⁺FoxP3⁺ T cells. Mean±SD and each dot represents individual donor.

FIG. 47—TNFR2 agonists expand human-TNFR2-expressing CD4 T cells in vitro. Enriched CD4 T cells obtained from human-TNFR2 knockin mice were stimulated with different concentrations of (A) C4-C4 (C4-IgG1(N297A)-HC:scFvC4) or (B) 3×VHH:C188-Fc(DANA) increased Treg frequencies after 4 days of culture with the respective agonists. Data represented as fold change increase of CD25^{high}FoxP3^{high} CD4 T cells over untreated (isotype control) samples. Data analyzed by one-way ANOVA.* p≤0.05, ** p≤0.005.

FIG. 48—Serum retention of Fc(DANA)-TNC-muTNF80. IL-8 concentrations measured by ELISA of supernatants of HT1080-Bcl2-TNFR2 cell cultures stimulated with different dilutions of serum (isolated from peripheral blood at the given time points) from wild type mice injected at 0 hours with 75 μg of Fc(DANA)-TNC-muTNF80 or isotype control antibody. The time points in the diagram refer to delta t after injection when the blood sample has been collected. The x-axis gives the dilution of these samples in the in vitro assay for the determination of TNFR2 agonistic activity.

FIG. 49—TNFR2 agonist expands Tregs in vivo and upregulates activation markers. (A) Treg frequency of viable cells in spleens isolated four days after stimulation with 75 g Fc(DANA)-TNC-muTNF80 in wild type mice. (B) Fold change of given extracellular markers defined as the mean fluorescence intensity of Tregs normalized against isotype control antibody samples.

FIG. 50—TNFR2 agonist increases splenic Tregs in vivo. (A) Fold change of Treg frequency of viable cells in spleens from huTNFR2-expressing mice isolated four days after stimulation with 300, 1000 μg 3×VHH:C188-Fc(DANA) or 1000 μg isotype control antibody. Fold change was calculated relative to isotype control. Comparison of means between treatment groups and isotype control done through unpaired t-Test.* p≤0.05, ns=not significant. (B) Fold change of Treg frequency (Foxp3+) of CD4+CD3+ T cells in spleens from huTNFR2-expressing mice isolated four days after stimulation with 40 μg, 100 μg, or 250 μg C4-IgG1(N297A)-HC:scFv:C4. Fold change was calculated relative to untreated control. Comparison of means between treatment groups and isotype control done through unpaired t-Test.* p≤0.05, ns=not significant.

FIG. 51—TNFR2 agonists increases survival after allogeneic hematopoietic cell transplantation (allo-HCT) induced acute graft-versus-host disease (GvHD). (A) Survival frequencies after allo-HCT induced experimental acute GvHD in mice (FVB/N H-2q→C57BL/6 H-2b, MHC major mismatch model). Statistical significance assessed with Log-Rank test between C19-IgG1(N297A)-HC;scFvC19 and isotype control antibody.* p≤0.05. (B) Pathology score of intestinal samples isolated on day 6 after allo-HCT based on immune cell infiltrates and intestinal crypt damage. (C) Day 6 ex vivo analysis of relative bioluminescence signal from allogeneic donor T cells in the indicated organs normalized against signal from mice treated with isotype control antibody.

FIG. 52—C4-IgG1(N297A)-HC:scFvC4 expands Treg from African Green monkey (vervet) peripheral blood mononuclear cells (PBMCs)—Fold change and total change of Treg frequency of vervet monkey PBMCs after 5 days of stimulation with indicated C4-IgG1(N297A)-HC:scFvC4 relative to untreated control of the same donor. Samples were measured with flow cytometry and Tregs were defined as CD3+CD4+ FoxP3+ T cells. Expansion was tested in n=1 vervet monkey sample.

FIG. 53: Amino acid sequence of the 3×VHH-Fc(durv) (hexavalent) format based on C188 parental and sequence optimized variant 14. Hence, the Figure illustrates polypeptides for preferred hexavalent binding molecules. The CDRs, overall sets of three CDRs, variable regions, and overall polypeptides shown represent preferred embodiments, as do variants of any thereof as discussed further herein.

FIG. 54: Amino acid sequence of the C4-IgG1(Durv)-HC:scFvC4(G4S)4 (tetravalent) format based on C4 parental. Hence, the Figure illustrates the polypeptides for preferred tetravalent binding molecules. The CDRs, sets of three light chain CDRs, sets of three heavy chain CDRs, overall sets of six CDRs, variable regions, pairs of variable regions, and overall polypeptides shown represent preferred embodiments, as do variants of any thereof as discussed further herein.

FIG. 55: Amino acid sequence of the tetravalent C4var7-IgG1(Durv)-HC:scFvC4var7(G4S)4 and C4var16-IgG1(Durv)-LC:scFvC4var16(G4S)4 based on the sequence optimized variant 7 and variant 16 respectively. Hence, the Figure illustrates the polypeptides for preferred tetravalent binding molecules. The CDRs, sets of three light chain CDRs, sets of three heavy chain CDRs, overall sets of six CDRs, variable regions, pairs of variable regions, and overall polypeptides shown represent preferred embodiments, as do variants of any thereof as discussed further herein.

### DETAILED DESCRIPTION OF THE INVENTION

### TNFR2 and TNFR2 Target Cells

The present invention provides binding molecules that bind TNFR2 and in particular which bind the extracellular domain of TNFR2. TNFR2 comprises a characteristic extracellular domain, a transmembrane segment, and an intracellular domain. The extracellular region contains four cysteine rich domains (CRD) that are repeated. The TNFR2 bound by a binding molecule of the present invention is typically a mammalian TNFR2. In a particularly preferred embodiment, the TNFR2 bound by a binding molecule of the present invention is human TNFR2. The amino acid sequence of human TNFR2 is provided as SEQ ID NO: 1. The human TNFR2 amino acid sequence provided in SEQ ID NO: 1 is subdivided into the signal peptide comprising amino acids 1 to 22; the extracellular domain of amino acids 23 to 257 and the transmembrane region of amino acids 258 to 287. A binding molecule of the present invention typically binds the extracellular domain of TNFR2.

In one embodiment, a binding molecule of the present invention may bind human TNFR2, but also TNFR2 of a different species such as a mouse TNFR2 and/or a monkey TNFR2. In one embodiment, it binds human, mouse, and a monkey TNFR2. In one embodiment, a binding molecule may bind human and cynomolgus monkey TNFR2. In one embodiment, a binding molecule may bind human and African green monkey TNFR2. In another embodiment, a binding molecule may binding human, cynomolgus monkey, and African green monkey TNFR2. In another embodiment, a binding molecule may bind human and mouse TNFR2. In another embodiment, a binding molecule may bind human, mouse, and African green monkey TNFR2. In another embodiment, a binding molecule may bind human, mouse, and cynomolgus monkey TNFR2. In another embodiment, a binding molecule may bind human, mouse, cynomolgus monkey and African green monkey TNFR2. In another embodiment, a biding molecule will bind at least those species of TNFR2.

A binding molecule of the present invention will typically be used to bind a target cell, in particular a target cell expressing TNFR2. The target cell may be any cell type expressing TNFR2. In one embodiment the target cell is an immune cell, endothelial cell, mesenchymal stem cell, or neuron. In a particularly preferred embodiment, the target cell is an immune cell. In an especially preferred embodiment, the target cell is a Treg cell. In one embodiment the immune cells expressing TNFR2 are selected from a T regulatory cells (Tregs), B regulatory cells (B regs), and MDSC (myeloid derived suppressor cells). In one embodiment, the target cell may be a Treg cell which is a Foxp3+CD25+ regulatory T cell.

### Binding Molecules

The present invention provides binding molecules that bind TNFR2. In one preferred embodiment, a binding molecule of the present invention will bind to TNFR2 and stimulate signalling through TNFR2. In one preferred embodiment, the binding molecules are agonistic antibodies or antibody fusion proteins or antibody fragments or antibody fragment fusion proteins against TNFR2 and so activate the receptor. In a preferred embodiment, the binding molecules are antibodies that specifically bind TNFR2, preferably they are antibodies that specifically bind TNFR2 and act as an agonist of it without FcgR binding. In one preferred embodiment, a binding molecule of the present invention will bind TNFR2 to a greater extent than to TNFR1. For instance, the strength of binding for TNFR2 compared to TNFR1 may be at least 10, 50, 100, 500, 1000 or more times higher. In one embodiment, the strength of binding for TNFR2 compared to TNFR1 may be at least 10,000, or at least 100,000 times greater. In one embodiment, the binding molecule does not bind TNFR1 at all. In one preferred embodiment, a binding molecule, and in particular of an antibody or antibody fragment or fusion proteins thereof of the present invention, is said to specifically bind TNFR2 when the KD for the binding has a value below 10⁻⁶M, 10⁻⁷M, 10⁻⁸M, 10⁻⁹M, or 10⁻¹⁰M. In one embodiment the KD value is measured using cells expressing TNFR2, for example HeLa transfectants expressing TNFR2. In one preferred embodiment, KD values are determined using the approach set out in Kums J, Nelke J, Ruth B, Schafer V, Siegmund D, Wajant H.MAbs. 2017 April; 9(3):506-520. Hence, in one embodiment, a fusion of the binding molecule with Gaussia princeps luciferase as a reporter domain is employed to measure the KD value of the binding molecule as described in Kums et al., supra. In one embodiment, cell lines expressing TNFR2 may be used in such measurement, for example HeLa or HEK293 cells that stably express TNFR2 on their surface. Typically, the binding molecules of the invention bind to TNFR2 and trigger TNFR2 signalling. In one embodiment, the binding molecules, and in particular antibodies, provided are agonistic for TNFR2, but are not agonistic for TNFR1. In one embodiment, a binding molecule of the present invention binds TNFR2, but does not bind any other TNFR super family members.

In a particularly preferred embodiment, the binding molecule of the present invention is an antibody. The term “antibody” as used herein typically refers to any functional antibody or antibody fusion protein or antibody fragment that is capable of specific binding to the antigen of interest, as generally outlined in chapter 7 of Paul, W. E. (Ed.).: Fundamental Immunology 2nd Ed. Raven Press, Ltd., New York 1989, which is incorporated herein by reference or shown in the “Periodic Table of Antibodies” (https://absoluteantibody.com/periodic-table-of-antibodies/). Reference to an antibody herein includes antibody fragments unless specifically stated. An antibody of the present invention typically specifically binds TNFR2. Reference to an antibody includes all of the various antibody formats discussed herein including antibodies that do not have the naturally occurring four chain structure of an antibody seen in humans, for example in one preferred embodiment an antibody of the present invention may be, or may comprise, a single chain antibody. An antibody of the present invention may also comprise non-antibody sequences such as a ligand based molecule that binds TNFR2. In one embodiment an antibody of the present invention may comprise polypeptide(s) which are a fusion of antibody sequences and non-antibody sequences, for example TNC peptide and TNF-alpha polypeptides (particularly TNFR2-specific mutants such as TNF80 (or TNF(143N/145R) as discussed herein. In one especially preferred embodiment the binding molecule is an antibody which does not comprise any non-antibody binding sites specific for TNFR2.

Any of the antibodies set out herein may further comprise one or more ligand based molecule and in particular such a ligand based molecule specific for TNFR2. For instance, the present application sets out various hexavalent antibodies where the valency of the molecule may be increased to more than six by including one or more ligand based binding molecule for TNFR2 such as any of those set out herein. In one embodiment, any of the antibodies set out herein may further comprise at least one ligand based binding molecule for TNFR2 which is either part of the polypeptide, linked to the polypeptide, or conjugated to it. In one embodiment, a hexavalent binding molecule comprising VHH domains may be further modified to include at least one ligand based binding molecule either as part of a polypeptide, linked to the polypeptide, or conjugated to it. In one embodiment, a binding molecule comprises two polypeptides, where each polypeptide comprises three VHH domains in series, a heavy chain constant region lacking a CH1 region, with a ligand based TNFR2 binding molecule either as part of a polypeptide, linked to the polypeptide, or conjugated to it. In one embodiment, a binding molecule of the present invention is modified so that each heavy chain has a ligand based TNFR2 binding molecule either as part of a polypeptide, linked to the polypeptide, or conjugated to it. In one embodiment, the light chains of the binding molecule may be so modified. In a preferred embodiment binding molecules may have the ligand based molecule ligand based TNFR2 as part of a polypeptide, linked to the polypeptide, or conjugated to it at the C-terminal half of the polypeptide. The present invention also provides various tetravalent binding molecules and in such binding molecules it may be that rather than scFv or other antibody based TNFR2 binding site in the “South” or “C-terminal” portion, the binding molecule has ligand based molecules. So, for instance, various C4 and C4 variant antigen-binding sites are set out herein and the present invention provides binding molecules where the “North” antigen-binding sites are C4 or C4 variant antigen-binding sites and the “South” antigen-binding sites are ligand-based TNFR2 antigen-binding molecules.

An antibody of the invention also includes a portion or fragment of an antibody. For example, an antibody of the present invention may comprise just an antibody Fc region modified so that it does not bind Fc receptors and ligand based binding molecules joined to the Fc region. In one embodiment, the ligand based molecules present in an antibody of the present invention is a TNF-based ligand for TNFR2, e.g. a TNF protomer or a mutant thereof. In one embodiment it is a single-chain version of a TNF trimer.

The “valency” of a binding molecule as used herein denotes the number of binding sites that a binding molecule has, with the valency for TNFR2 denoting the number of binding sites for TNFR2 that a binding molecule has. The “specificity” of a binding site denotes what it binds to, for instance it may denote what target molecule the binding molecule binds, but it may also denote where on the target it binds. In respect of an antibody, the specificity of an antibody denotes what target the antibody binds, it may also be used to denote where on the antigen it binds in terms of the epitope bound on that target molecule. Binding molecules may have different specificities in the sense that they bind epitopes on different target molecules (antigens). However, binding molecules may have different specificities in the sense that they bind different epitopes on the same target molecule (antigen). For example, a bispecific binding molecule may be bispecific in the sense it can bind two different target molecules, but alternatively it may be bispecific in the sense it can bind two different epitopes of the same target molecule and in particular of TNFR2.

In one embodiment, all of the binding sites of a binding molecule of the present invention will bind the same target. In one embodiment, where the binding molecule of the present invention is an antibody, it may be that all of the antigen-binding sites of the antibody bind the same epitope of TNFR2. In another embodiment, it may be that a binding molecule of the present invention will comprise at least two different target binding sites that each bind a different target site on TNFR2, for example which each recognise a different epitope of TNFR2. In one embodiment, a binding molecule of the present invention is an antibody which comprises at least two antigen-binding sites that bind a different epitope of TNFR2. In one embodiment, at least one of the binding sites of the molecule will bind a target other than TNFR2. For example, in one embodiment a binding molecule of the present invention may include a binding site with a different specificity that recognises a target of a different cell type. For example, the different specificity can be used as a homing entity to bring the target the cell expressing TNFR2, together with a different cell type. In one embodiment, a binding molecule of the present invention may include at least one binding site that binds a target that alters the serum half-life of the binding molecule, for example in one embodiment it may bind serum albumin. In one embodiment, only one of the binding sites of the molecule will target something other than TNFR2. In one embodiment, there may be two such sites.

A binding molecule of the present invention will have a valency of at least two for TNFR2. In one embodiment, a binding molecule of the present invention will have a valency of two, three, four, five, six, seven, eight, nine, ten, eleven or twelve, or at least those valencies for TNFR2. In one preferred embodiment, a binding molecule of the present invention will have a valency of two. In another preferred embodiment, it will have a valency of three. In one embodiment, a binding molecule of the present invention is trispecific. In another preferred embodiment, a binding molecule of the present invention will have a valency of at least four. Hence, in a preferred embodiment, a binding molecule of the present invention is tetravalent. In one preferred embodiment, a binding molecule of the present invention will have a valency of four. In one embodiment, a binding molecule of the present invention will have a valency of at least six. In a preferred embodiment, it will have a valency of six. In one preferred embodiment, a binding molecule of the present invention will have a valency of twelve. In one embodiment, a binding molecule of the present invention is multi-specific, for instance as it has a valency of at least two, and preferably at least four. A particularly preferred embodiment is a tetravalent antibody. A further particularly preferred embodiment is a hexavalent antibody. A preferred hexavalent antibody is one where the TNFR2 binding sites are single domain antibodies (sdAbs) specific for TNFR2. A particularly preferred hexavalent antibody is one where the TNFR2 binding sites are VHH binding domains specific for TNFR2. SdAbs and VHHs are discussed further below.

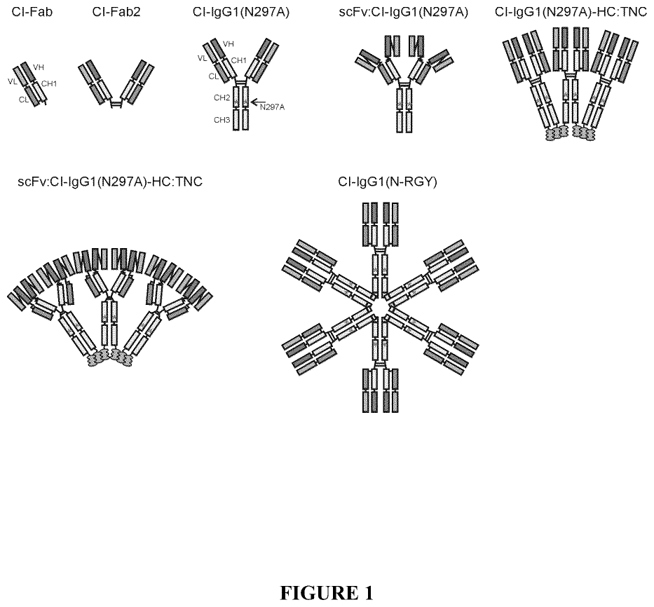

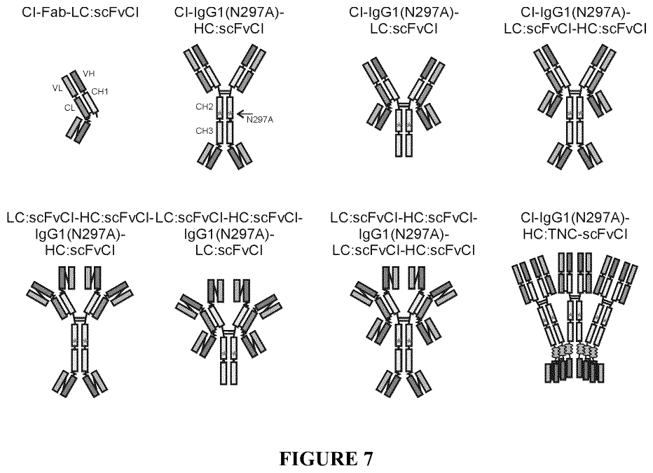

As well as the “valency” of a binding molecule of the present invention, a further point is the orientation/localization of the binding sites of the binding molecules. That may be best considered using the examples of the antibodies shown in FIGS. 1 and 7. FIG. 1 depicts antibodies with (N-terminal) unidirectional oriented antigen-binding sites, solely in what can be thought of as “North” orientation, for example in one embodiment such binding molecules may be thought of having the antigen-binding sites that bind TNFR2 at what would be considered the N-terminal portion of the heavy and light chains of a conventional four chain antibody and they may be located at the N-terminal portions of the polypeptides of the binding molecules. The orientation of the binding sites may also be defined by referring to binding sites in the same orientation as “parallel” binding sites, for example where all of the binding sites are at the N-terminal portions of the antibody chains or all at the C-terminal portion of the antibody chains, the binding sites may be considered as parallel. Such binding sites may also be considered as unidirectionally orientated. FIG. 7 of the present application depicts antibodies with dual N- and C-terminal antigen binding sites in what can be thought of as “North”+“South” orientations or “opposite” orientations. In one embodiment, such binding sites may be thought of as having antigen-binding sites in more than one plane or more than one direction. In one embodiment, a construct of the invention is one where the antigen binding sites are solely in the “North” orientation, so the antigen-binding sites are N-terminal. In one embodiment, a binding molecule of the present invention is a North construct with scFvs replacing the variable domains of the HC and LC, for example (scFv-IgG(N297A) and scFv-IgG(N297A)-HC:TNC). In another embodiment, a binding molecule of the present invention may have binding sites in both the “North” and “South” orientation, so effectively have antigen-binding sites at both the N-terminal and C-terminal locations.

In one embodiment, “North” or “N-terminal” facing antigen-binding sites may be thought of as in the same orientation or plane as the binding site of a conventional monomeric IgG molecule, in particular what would be normally considered the N-terminal portion of the light and heavy chain variable regions of such a conventional IgG molecule. In one embodiment, “South” of “C-terminal” facing antigen binding sites may be thought of those in a direction or a plane that a conventional monomeric IgG molecule does not have antigen binding sites. In one embodiment the orientation is the opposite from that of the Fab subdomains (VH+VL) domain in a conventional monomeric IgG molecule thus having the TNFR2 binding domain at the Fc domain C-terminus directing to neighboring cells of the antigen expressing cell. Such binding sites can be thought of as in the opposite to the “normal” orientation of a Fab domain in an antibody. Whilst the hexameric IgG molecule depicted in FIG. 1 has antigen-binding sites in the top and bottom of the hexamer it is not considered to have “North” and “South” or “opposite” orientated antigen-binding sites, because all of the antigen-binding sites are in the same format as a conventional monomeric IgG molecules, for example in the sense that it does not have antigen-binding sites at what would be considered the C-terminal portion of a conventional monomeric IgG molecule. In one preferred embodiment, an antibody of the present invention comprises antigen binding sites at the N-terminal ends of the polypeptides making up the antibody. In one embodiment, it may be that a binding molecule of the present invention is an antibody in the sense that it has antigen binding sites at both the N-terminus and C-terminus of polypeptide chains of the antibody. In one particularly preferred embodiment, a binding molecule is a tetravalent antibody with two N-terminal (“north”) and two C-terminal (“south”) binding sites for TNFR2. In a preferred embodiment, the antibody is an IgG with a scFv at the C terminus of each light chain. In a further preferred embodiment, the antibody is an IgG with a scFv at the C terminus of each heavy chain. In a further preferred embodiment, the antibody is an IgG with a scFv at the C terminus of each heavy chain and each light chain, with such an antibody then being hexavalent.

In one preferred embodiment, the binding molecule employed is a multimer of individual subunits. For example, in one preferred embodiment the binding molecule of the present invention is a dimer. In one embodiment of the present invention the dimer is form of two scFv-Fab molecules. In one preferred embodiment the binding molecule of the present invention is a trimer. In another preferred embodiment, a binding molecule of the present invention is a hexamer. In one embodiment, the binding molecule employed may be an antibody. In one embodiment the binding molecule employed may be an antibody which is an antibody fusion protein hexamer. In one preferred embodiment, the heavy chains of the individual IgG molecules making up the hexamers comprise an RGY peptide motif and in particular may do so at their C-terminus. In one particularly preferred embodiment an antibody of the present invention comprises the mutations of the RGY motif which are E345R/E430G/S440Y in the heavy chain and in particular in the Fc tail. The RGY motif is described, for instance, by de Jong et al (2016) PLOSBiology|DOI 10.1371/journal.pbio.1002344 which is incorporated by reference in its entirety, as well as specifically in relation to the RGF motif and its use in the formation of hexamers.

Typically, where a binding molecule of the invention comprises a multimer or assembly of smaller units it will still be considered a “binding molecule” or “antibody” of the present invention, for instance even though it may comprise several subunits that might individually be considered an antibody, such as an IgG and scFv. So, for instance, an “antibody” of the present invention may comprise an antibody fragment or fragments as part of the overall “antibody” of the present invention. As discussed below, it may comprise other entities such as ligands for TNFR2, such as TNF-alpha and in particular TNF80 and other TNFR2-specific TNF mutants. Any of the specific formats set out herein may be modified to further include a ligand for TNF2.

Antibody hexamers represent one embodiment of the present invention. In one embodiment, an antibody of the present invention will comprise at least a heavy chain modification at E345 and in particular E345R. In another embodiment, an antibody of the present invention may comprise at least a heavy chain modification at E430 and in particular E430G. In another embodiment, an antibody of the present invention may comprise at least a heavy chain modification at S440 and in particular S440Y. In another embodiment, an antibody of the present invention may comprise at least heavy chain modifications at E345 and E430 and in particular E345R and E430G. In a preferred embodiment, an antibody of the present invention may comprise at least heavy chain modifications at E345, E439, and S440, in particular E345R, E439G and S440Y. In a preferred embodiment, an antibody of the invention will comprise such modifications and comprise a hexamer of IgG units. In a further preferred embodiment, an antibody of the present invention will undergo on-target hexamerisation once it has bound to TNFR2.

In one preferred embodiment, a binding molecule of the present invention may comprise sequences that bring about trimerization. Examples of such sequences include trimerisation domains from collagen, leucine zipper, T4 fibritin, and trimerization domains of TRAF family members. A binding molecule of the present invention in some embodiments may comprise such trimerization sequences. In one embodiment, a binding molecule of the resent invention may comprise, or may be, a trimer due to the presence of such domains. In one preferred embodiment, the trimerization sequence may be that from tenascin-C(TNC). However, in any of the embodiments discussed herein where TNC is referred to a trimerization sequence in general may be employed, for example any of the specific ones referred to herein and not just TNC, though in a preferred embodiment TNC is used. In one preferred embodiment the TNC sequence used is from chicken TNC. In another preferred embodiment, it is from a mammalian TNC. In a further preferred embodiment, the TNC is human TNC. In one embodiment, a binding molecule of the present invention does not comprise TNC.

In another preferred embodiment, the tenascin-C(TNC) trimerization domain may be used to assemble individual antibody or other subunits into a multimer. Hence, a binding molecule, and in particular an antibody of the present invention, may comprise a TNC trimerization domain peptide such as that in SEQ ID NO: 2. It may comprise a variant TNC peptide, for example one with five, four, three, two, or one amino acid sequence change relative to SEQ ID NO:2, but which is still able to bring about the formation of multimers. In one preferred embodiment, the C-terminus of the heavy chains of the antibody comprise, or are linked to, the TNC peptide sequence. In one embodiment, such an approach is used to bring together a trimer of individual TNFR2 binding moieties such as antibody subunits antibodies, and/or ligands. Any of the binding molecules set out herein, unless otherwise stated, may comprise a TNC peptide as a way to allow association of individual units and in particular as a way to form multimers.

In one embodiment, a binding molecule of the present invention is an FcγR independent agonist of TNFR2. For example, in the case where the binding molecule is an antibody, it may be that the antibody lacks an Fc region and so does not bind FcγR for that region. Alternatively, in another embodiment, it may be that the antibody does have an Fc region, but that it has been modified so that it is unable to bind FcγR. Various Fc modifications are discussed herein that may be used to accomplish that.

In one embodiment, a binding molecule of the present invention will be an antibody selected from an IgG, IgM, IgA, IgE and IgD antibody or comprise such an antibody. It may comprise the Fc modifications discussed herein that render it Fc gamma receptor independent. In one particularly preferred embodiment, it will be, or comprise, an IgG antibody or comprise such an antibody. In one particularly preferred embodiment it will be an IgG1 antibody or comprise such an antibody. The antibody may be an IgG2, IgG3, or IgG4 antibody or comprise such an antibody. In a particularly preferred embodiment, an antibody of the present invention may comprise single domain antibodies, for example VHH single domain antibodies. In one embodiment, an antibody of the present invention will comprise at least two suchs dAbs, in particular VHHs.

In a preferred embodiment, the binding molecules provided specifically bind TNFR2, but not TNFR1. In a further preferred embodiment, the binding molecule provided is an FcγR independent agonist of TNFR2. The binding molecule has a valency of at least two for binding TNFR2.

In a preferred embodiment, the binding sites of a binding molecule of the present invention may be selected from antibody antigen-binding sites and ligands that bind to TNFR2. In an especially preferred embodiment, a binding molecule of the present invention is an antibody or comprises at least part of an antibody. As defined herein an antibody includes various formats that include antigen-binding sites, but are not in a naturally occurring antibody format. An antibody does not necessarily have to be a whole antibody, for example an antibody of the present invention may be, or comprise, an antibody fragment, such as Fab2 antibody fragment. Antibodies of the present invention may comprise antibody fragments, for example in one preferred embodiment an antibody of the present invention may comprise an antigen-binding site provided by a scFv fragment or Fab as part of the antibody. An antibody of the present invention may be, or comprise, a single chain antibody.

Reference to an antibody includes antibody molecules that comprise, are linked to, or conjugated to moieties that do not themselves originate from antibody based sequences. So, for instance, reference to an antibody herein includes an antibody which is linked or conjugated to a TNFR2 ligand or a modified version of a TNFR2 ligand. Examples of TNFR2 ligands that may be employed, or be modified to be employed, include TNFα and LTα, as well as variants thereof. In one embodiment, a binding molecule, and in particular an antibody, of the present invention may comprise sequences that bring about multimerisation. An example of one such sequence is that an antibody of the present invention may comprise a heavy chain with a sequence that brings about multimerisation, for example by including the RGY mutations that bring about multimerisation. In one preferred embodiment, it is the Fc tailpiece which comprises the RGY modifications to bring about hexamerisation. In another particularly preferred embodiment, an antibody of the present invention may comprise a tenascin-C trimerization domain peptide sequence (TNC) at the C-terminus of heavy chains. Hence, in one embodiment, a binding molecule of the present invention, particularly an antibody, may comprise a peptide sequence from Tenascin-C which is provided as SEQ ID NO: 2 or a variant TNC peptide which is still able to bring about multimerisation. In another embodiment, it may comprise the human TNC sequence which brings about trimerization. In another embodiment, it may comprise a trimerization domain, such as any of the specific ones mentioned herein.

In one embodiment all of the binding sites of a TNFR2 binding molecule of the present invention may be antibody antigen-binding sites. In another embodiment, a binding molecule of the present invention comprises at least one antibody antigen-binding site for TNFR2 and at least one ligand, or ligand derivative, that binds TNFR2, in other words it may comprise a mixture of such binding sites. In one embodiment, a binding molecule of the present invention comprises an antibody which further comprises ligand based binding sites for TNFR2. In one preferred embodiment, a binding molecule, and in particular an antibody, of the present invention does not comprise ligand based TNFR2 binding sites. In embodiments where the binding molecule is, or comprises, an antibody, an antigen-binding site will generally comprise six CDRs, three light chain variable region CDRs (LCDR1, LCDR2, and LCDR2) and three heavy chain CDRs (HCDR1, HCDR2, and HCDR3). However, as discussed further below the binding molecules of the present invention may comprise VHH binding domains, and other single domain binders, in particular for TNFR2. Such VHH domains comprise three CDRs, CDR1, CDR2, and CDR3. The present invention provides a binding molecule comprising a binding site with any of the specific sets of six or three CDRs set out herein in an antigen-binding site for TNFR2. In another embodiment, a binding molecule may comprise any pair of VH and VL regions set out herein. In a further embodiment, it may comprise a binding site comprising, or consisting of, any of the VHHs sets out here. Such specific sequences are set out in the Tables of the present application. Such CDR sets are also set out herein by reference to SEQ ID NOs. Such sets of CDRs, pairs of VL and VH, or VHH domains may be used in any of the structures sets out herein, including those shown in the Figures and employed in the Examples. Variant sequences of any of the specific sequences set out herein may also be employed.

In one preferred embodiment, the antigen binding sites of the antibody and the ligand based binding sites are in different orientations, so that the two may be in opposite orientations, for example the antigen-binding sites may be in “North” orientation and the ligand based binding sites in “South” or “opposite” orientation. In another embodiment, the binding molecule may have antibody antigen-binding sites in both “North” and “South” orientations, but ligand based binding sites in only one orientation, for example solely in “South” orientation. “North” and “South” denote whether the antigen binding sites are present in the N-terminal or C-terminal portion of polypeptides. So, for instance, in conventional antibodies the antigen binding sites provided by the variable regions of the antibody are in the N-terminal portion of the heavy and light chain polypeptides. In contrast, some of the binding molecules provided represent antibodies where the antigen binding sites are at, or towards the C-terminal end of the polypeptides, for instance where scFvs are either the C-terminal portion of the light and heavy chain polypeptides or joined to them. Some binding molecules comprise both N-terminal and C-terminal antigen binding sites.

In one embodiment of the invention an antibody with only “North” orientated binding sites is employed. FIG. 1 of the present application shows illustrative examples of preferred TNFR2 binding molecules, as well as Fab which has a valency of one and so on its own is not a binding molecule of the present invention.

In the formats shown by the Figures, in particular FIGS. 1, 7, 24, 29, 40, and 41, the structures are indicated with a “CI” or “Clone Identifier” where an parental TNFR2-specific IgG molecule is referred to, with the Clone Identifier indicating from which clone discussed in the Examples the CDRs of the IgG antigen-binding sites originate. Where reference is made herein to such structures, but without the Clone Identifier being used, the structure is then not limited to reference to a specific clone, so is not limited to the CDRs being from a particular clone from the Examples, and the antigen-binding sites may be from any suitable TNFR2 antibody. So, for instance, reference to scFv-C4-IgG1(N297A) includes the clone identifier (CI) for Clone 4, but where scFv-IgG1(N297A) is referred to it indicates such a format binding molecule but without the antigen-binding site being limited to sequences from a particular clone. For any of the formats set out herein in relation to a specific clone, one of the other antigen-binding sites provided herein may be employed instead and the format is also provided where it is not limited to a particular clone. For any binding molecule comprising antigen-binding sites comprising heavy and light chain variable regions, the C4 and C4 variant CDRs, CDR sets, variable regions, and pairs of variable regions (see Table 10) may be employed in a preferred embodiment.

In preferred embodiments, a binding molecule of the present invention is selected from the following:
i. A Fab2 antibody where both specificities are specific for TNFR2.
ii. An IgG antibody which comprises an Fc region that is not able to activate Fc receptors and in particular does not bind FcγR. Both valencies of the molecule are specific for TNFR2 and may be thought of as “North” orientated. In one embodiment, the Fc region of the antibody comprises a modification that means it does not bind to Fc receptors, such as any of the specific modifications set out herein. In one embodiment, the antibody is an IgG1 antibody. In a preferred embodiment it has the format IgG1(N297A) shown in FIG. 1, but not limited to a specific clone.
iii. A tetravalent antibody molecule, comprising four antigen-binding sites specific for TNFR2 which are all “North” orientated, where the antibody has the basic structure of an IgG molecule modified so that each light and heavy chain has at its N terminus an antigen-binding site linked to it. In one embodiment, each antigen-binding site comprises a pair of heavy and light chain variable regions (scFv domain) which are joined to a light or heavy chain of the basic four chain IgG structure. In one embodiment, the antibody comprises the heavy and light chain constant regions of a basic IgG molecule, where each N-terminus of an antibody chain has a scFv joined to it. The antibody typically comprises a Fc region that has been modified so that it does not bind FcγR. In one embodiment it comprises any of the modifications discussed herein that prevent or reduce FcγR binding. In one embodiment, the antibody has the format scFv-IgG1(N297A) as shown in FIG. 1 but is not limited to the sequences originating from a particular clone.
iv. An antibody with a valency of six which is a multimer of three IgG antibody subunits, the Fc regions of each individual IgG subunit being unable to bind FcγR. All six antigen-binding sites may be thought of as “North” orientated. In one embodiment, the Fc regions are unable to bind receptors of the Fcgamma-Receptor group because they comprise a modification, such as any of those disclosed herein. In another embodiment, the Fc regions comprise the N297A modification. Any suitable way may be used to multimerise the individual IgG subunits. In a particularly preferred embodiment, the subunits associate through the use of a tenascin-C peptide (TNC) and in particular the presence of a peptide derived from TNC such as the peptide sequence of SEQ ID NO: 2. For example, in one embodiment, the heavy chains of the IgG subunits comprise or are linked to a tenascin peptide, with the association of the tenascin peptides leading to assembly of the multimer. In a preferred embodiment the peptides have the sequence of SEQ ID NO: 2 or a variant sequence thereof still able to multimerise. In one embodiment, the antibody has the format IgG1(N297A)-HC:TNC as shown in FIG. 1, but is not limited to sequences from a particular clone.
v. An antibody with a valency of 12 representing a multimer of three subunits. The subunits in one embodiment comprising a IgG structure where the variable regions have each been replaced with a scFv specific for TNFR2, where all of the antigen binding sites may be thought of as “North” facing, e.g. scFv-IgG1(297A)-HC:TNC. The antibody is unable to bind Fc receptors, in one embodiment it does not bind FcγR because the Fc region has been modified to render it unable to do so, for example any of the modifications discussed herein have been employed. In a preferred embodiment the N297A modification is employed. Any suitable way may be used to bring together the three subunits and in one embodiment the TNC trimerization domain is employed. In a preferred embodiment, the C terminus of each heavy chain either comprises, or is linked to, a TNC trimerization domain to bring about multimerisation of several Fab2 or IgGs, such as that of SEQ ID NO: 2. In one embodiment the antibody has the format scFv:IgG1(N297A)-HC:TNC shown in FIG. 1, but is not limited to sequences from a particular clone.
vi. A hexameric IgG antibody. Typically, all the antigen binding sites of the antibody are specific for TNFR2 so that the antibody has a valency of twelve. The Fc regions of each individual IgG subunit have a modification preventing binding to FcγR. Any of the modifications discussed herein may be employed, in one preferred embodiment the modification being N297A. Any suitable way to achieve hexamerisation either on or off target may be employed. In one particularly preferred embodiment each heavy chain of the individual IgG subunit comprises the RGY mutations to allow for hexamerisation. In one embodiment, the antibody carries the RGY mutation plus the N297A mutation (IgG1(N-RGY) shown in FIG. 1) and so has both the mutation preventing Fc receptor binding and the mutations resulting in hexamerisation. Hence, in one embodiment the binding molecule has the format IgG1(N-RGY) as shown in FIG. 1 but is not limited to sequences from a particular clone.

In one particularly preferred embodiment, the binding molecule is one of those set out in (iii) to (vi) above. In another preferred embodiment, the binding molecule is a tetravalent binding molecule, for example that set out in (iv). In a further preferred embodiment, the binding molecule is one with a valency of 12, for example selected from those set out in (v) and (vi) above.

In one embodiment of the invention, an antibody with both N-terminal and C-terminal placed binding sites on an antibody scaffold with parallel oriented chains (Fc, IgG, Fc-TNC, TNC-Fc, IgG-TNC) is employed. Hence, an antibody with “opposite” orientated binding sites is employed. FIG. 7 of the present application shows illustrative examples of particularly preferred TNFR2 binding molecules which have binding sites which may be considered as in “opposing” orientations. In preferred embodiments, a binding molecule of the present invention is selected from the group set out below:
i. An antibody comprising a Fab region joined to a scFv, where the orientation of the binding sites of the Fab and the scFv for TNFR2 are in opposite orientation, thus the scFv domain is linked to the C-terminus of LC or the truncated HC. In a preferred embodiment, the scFv is joined to the C-terminus of the light chain of the Fab. Hence, in one embodiment an antibody of the present invention is in the format Fab-LC:scFv shown in FIG. 7, but not limited to sequences from a particular clone.
ii. An antibody which is an IgG molecule which has scFv attached to the C-terminus of each heavy chain and an Fc region modified so that it does not bind to Fc receptors and in particular FcγR. The Fc region may comprise any of the modifications discussed herein that eliminate Fc binding and in particular the N297A heavy chain modification. In one embodiment, the antibody is of the format IgG1(N297A)-HC:scFv shown in FIG. 7, but not limited to sequences from a particular clone.
iii. An antibody which is an IgG molecule which has scFv attached to the C-terminus of each light chain and an Fc region modified so that it does not bind to Fc receptors and in particular FcγR. The Fc region may comprise any of the modifications discussed herein that eliminate Fc binding and in particular the N297A heavy chain modification. In one embodiment, the antibody is of the format IgG1(N297A)-LC:scFv shown in FIG. 7, but which is not limited to sequences from a particular clone.
iv. An antibody which is an IgG molecule which has scFv attached to the C-terminus of each light and heavy chain and an Fc region modified so that it does not bind to Fc receptors and in particular FcγR. The Fc region may comprise any of the modifications discussed herein that eliminate Fc binding and in particular the N297A heavy chain modification. In one embodiment, the antibody is of the format IgG1(N297A)-LC:scFv-HC:scFv shown in FIG. 7, but not limited to sequences from a particular clone.
v. An antibody which is an IgG molecule which has been modified so that the heavy and light chain variable regions have each been replaced with a TNFR2-specific scFv and where the heavy chains of the IgG molecule each have a TNFR2-specific scFv joined to them. Hence, such an antibody has a valency of six for TNFR2. The Fc region of the antibody having been modified so that it does not bind to Fc receptors and in particular FcγR. The Fc region may comprise any of the modifications discussed herein that eliminate Fc binding and in particular the N297A heavy chain modification. In one embodiment, the antibody is of the format LC:scFv-HC:scFv-IgG1(N297A)-HC:scFv shown in FIG. 7, but not limited to sequences from a particular clone.
vi. An antibody which is an IgG molecule which has been modified so that the heavy and light chain variable regions have each been replaced with a scFv and where the light chains of the IgG molecule each have a scFv joined to them. Hence, such an antibody has a valency of six for TNFR2. The Fc region of the antibody having been modified so that it does not bind to Fc receptors and in particular FcγR. The Fc region may comprise any of the modifications discussed herein that eliminate Fc binding and in particular the N297A heavy chain modification. In one embodiment, the antibody is of the format LC:scFv-HC:scFv-IgG1(N297A)-LC:scFv shown in FIG. 7, but not limited to sequences from a particular clone.
vii. An antibody which is an IgG molecule which has been modified so that the heavy and light chain variable regions have each been replaced with a scFv and where the light and heavy chains of the IgG molecule each have a scFv joined to them. Hence, such an antibody has a valency of eight for TNFR2. The Fc region of the antibody having been modified so that it does not bind to Fc receptors and in particular FcγR receptors. The Fc region may comprise any of the modifications discussed herein that eliminate Fc binding and in particular the N297A heavy chain modification. In one embodiment, the antibody is of the format LC:scFv-HC:scFv-IgG1(N297A)-LC:scFv-HC:scFv shown in FIG. 7, but not limited to sequences from a particular clone.
viii. A multimer comprising three individual IgG antibody molecules that comprise at the C-terminus of the heavy chain a tenascin-C(TNC) trimerization peptide, with each tenascin peptide joined with its C-terminus to a scFv. The antibody molecule having a valency of twelve for TNFR2. The Fc region of the antibody having been modified so that it does not bind to Fc receptors and in particular FcγR. The Fc region may comprise any of the modifications discussed herein that eliminate Fc binding and in particular the N297A heavy chain modification. In one embodiment, the antibody is of the format IgG1(N297A)-HC:TNC-scFv shown in FIG. 7, but not limited to sequences from a particular clone.

As discussed above, the formats discussed above shown in FIG. 1 are provided where they are not limited to the sequences originating from a particular clone. In one embodiment, also provided are the specific formats where they do have the sequences from a specific clone provided in the Examples or variant sequences as discussed herein. In one embodiment for the formats with Fc regions, the heavy chains comprise heavy chain modifications as discussed herein, such as the DANA modifications discussed herein, and not just the N297A modification.

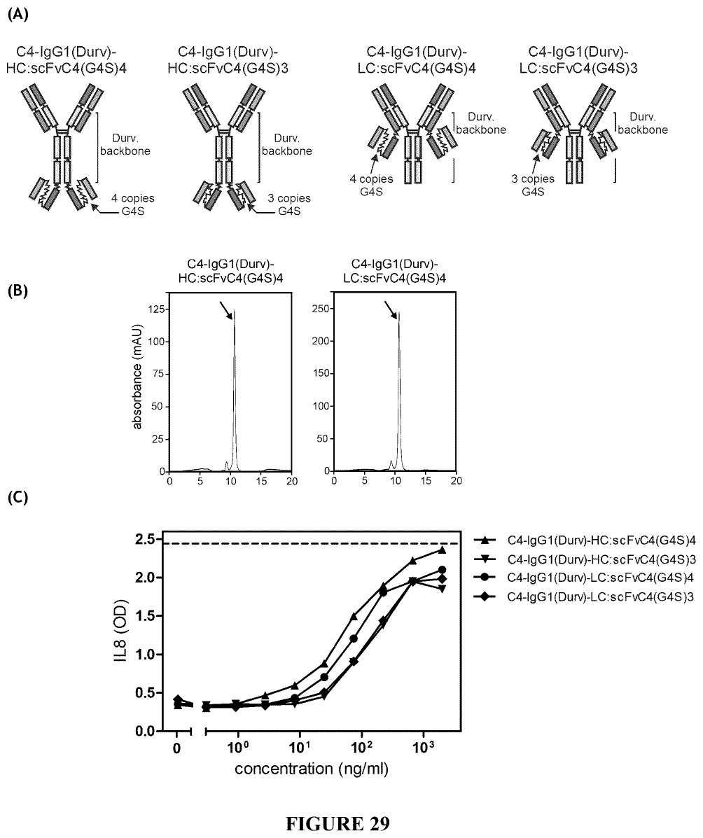

In one particularly preferred embodiment, the set of six CDRs, or VL and VH sequences, from the C4 clone or one of the specific variants of the C4 clone provided herein are employed in one of the formats shown in FIGS. 1 and 7. They may also be employed in the formats shown in FIG. 29. In one embodiment, any of the sets of six CDRs set out in Table 5 or pairs of variable regions set out in Table 6 may be employed in any of the formats set out herein, particular those formats shown in FIGS. 1 and 7. Variant sets of CDRs or pairs of variable regions may also be employed as discussed further herein. Particularly preferred CDRs, CDR sets, and variable regions to be employed are those set out for C4 and the C4 variants set out herein.

In one particularly preferred embodiment, the binding molecule of the present invention is an antibody selected from one of the following formats, with some of the advantages of the formats mentioned in brackets:
Fab1-scFv (high tissue penetrance, short-lived);
IgG-HC:scFv (tested for a variety of antibodies, easy generation of bispecific variants);
scFv-IgG (easy generation of bispecific variants);
IgG-TNC (potentially useful in pathway discrimination); and
scFv-IgG-TNC (highly active, easy generation of bispecific variants).

In one particularly preferred embodiment, where IgG is referred to above IgG1 is employed. Alternatively, in another embodiment, where IgG1 is referred to in a particular format, the same format is also provided where the IgG may be any IgG subtype.

In one embodiment, a binding molecule of the present invention is an antibody that:
(a) comprises a TNFR2 binding site at both the N-terminus and C-terminus of one or more heavy chain of the antibody; and/or
(b) comprises a TNFR2 binding site at both the N-terminus and C-terminus of one or more light chain of the antibody.

In one preferred embodiment, the binding molecule comprises a scFv at the C-terminal portion of heavy chains of the binding molecule, where the scFv binds TNFR2. In another embodiment, a binding molecule of the present invention comprises scFv at the C-terminal portion of the light chains in the molecule.

In one particularly preferred embodiment, a binding molecule of the present invention is an antibody that just comprises an Fc region which at the C-terminal portion of the heavy chains either has, or is linked to, or is conjugated to, ligands specific for TNFR2. In one embodiment, each heavy chain has three such ligands present at the C terminal portion of the heavy chains.

In one particularly preferred embodiment, a binding molecule of the present invention is an antibody or Fc region with a C-terminal TNC trimerization followed by a TNFR2-specific TNF mutant, in particular TNF80. In one embodiment, a polypeptide may comprise a single chain polypeptide comprising three TNF-alpha sequences that can tri-merise, such as three TNF80 units.

In one preferred embodiment, a binding molecule of the present invention is bivalent with spatial movable C- and N-terminal located antigen-binding sites.

In one embodiment, an antibody of the present invention is a bivalent antibody which comprises an Fab specific for TNFR2, where the heavy or light chain of the Fab portion of the antibody comprises an scFv specific for TNFR2. One advantage of such a small molecule is that it may show high tissue penetration. In one preferred embodiment, the antibody may comprise the scFv joined to the light chain of the Fab, for example with the two being part of the same polypeptide. In one preferred embodiment, the antibody is in the format of Fab-LC:scFv as shown by FIG. 7, but is not limited to sequences from the clones provided in the present Examples and may be any suitable sequences from an antibody for TNFR2. The Fab and scFv domains may, for instance, recognize in these embodiments the same or different TNFR2 epitopes.

In one preferred embodiment, the binding molecule, and in particular an antibody, of the present invention is tetravalent. In one embodiment, the binding molecule of the present invention is in the format Fab-HC-scFv, where the antibody comprises an Fc region modification eliminating or reducing Fc receptor binding, and where the antibody comprises a scFv at the C-terminal end of each heavy chain. In another preferred embodiment, the antibody is in the format Fab-HC-scFv where the antibody is bispecific in the sense that it has two different specificities for TNFR2. For example, in one preferred embodiment, the scFvs present bind a different epitope than the antigen-binding sites of the IgG. In an alternative embodiment, all of the TNFR2 binding sites recognize the same epitope of TNFR2. In a further preferred embodiment, an antibody of the present invention is in the format scFv:IgG, where the light and heavy chains of the antibody have been modified to include an scFv at the N-terminal portion, with the Fc region of the antibody modified so that it does not bind Fc receptors. Any of the modifications discussed herein that reduce or eliminate Fc binding may be employed. Such an antibody may be bispecific in the sense that the scFvs expressed on the heavy chain polypeptide may bind one epitope of TNFR2 and those of the light chains another specificity of TNFR2. In an alternative embodiment, all of the scFv regions may bind the same epitope of TNFR2.

In one embodiment, a binding molecule of the present invention is hexavalent. In one embodiment the antibody is in the format IgG-LC:scFv-HC:scFv and also has a modified Fc region so that it does not bind Fc receptors, for example having any of the Fc modifications discussed herein that eliminate or reduce binding to Fcγ receptors. In a preferred embodiment, the IgG is IgG1. In another preferred embodiment, the antibody is hexavalent, where it comprises an antibody Fc region, with each of the heavy chains linked to a trimer of TNF-α, or a variant thereof, at the C-terminal portion.

In another preferred embodiment, a binding molecule of the invention is 12-valent. In one preferred embodiment, a 12-valent binding molecule is a multimer of three individual antibody subunits where the heavy and light chain variable regions of the antibody are replaced with scFv molecules. In a preferred embodiment, the three antibody subunits are associated as the heavy chains for each antibody comprise a tenascin-C peptide at the C-terminus comprising the trimerization domain of TNC. The binding molecule will not bind Fc receptors and in a preferred embodiment comprises an Fc region with any of the modifications set out herein. In another embodiment, a binding molecule of the present invention may be a hexabody comprising six IgG molecules. For example, the binding molecule may be a hexabody of six IgG antibodies, where the Fc regions comprise RGY HexaBody mutations and also a modification that means they do not bind Fc receptors, such as any of the specific modifications set out herein. In a preferred embodiment the IgG is IgG1.

In one preferred embodiment, a binding molecule of the present invention is four or 12 valent for TNFR2. In one preferred embodiment a binding molecule of the present invention is an antibody selected from the format scFv-IgG(*)-HC-TNC and scFv-IgG(*), where * indicates an Fc region modification which eliminates or reduces Fc receptor binding. In a particularly preferred embodiment, the IgG portion of the binding molecule is IgG1. HC-TNC indicates that the heavy chains of the IgG comprise a tenascin peptide, for example that of SEQ ID NO:2 or a variant thereof. In another embodiment, a binding molecule of the present invention is an antibody selected from the format scFv-IgG(*)-HC-TNC, scFv-IgG(*), and IgG(*)-HC:RGY. For the IgG(*)-HC:RGY format again preferable the IgG portion of the molecule is an IgG1 antibody.

In an especially preferred embodiment, a binding molecule of the present invention will be able to bring about the formation of hexamers of TNFR2 molecules on the surface of the cell or at least hexamers. In one embodiment a binding molecule of the present invention may bind TNFR2 receptors in cis, that is bind a plurality of TNFR2 receptors on the surface of the same cell. In one embodiment, the binding molecule is able to at least bring about the formation of hexamers of TNFR2 on the surface of the same cell. In another embodiment, a binding molecule of the present invention is able to bind TNFR2 molecules in trans, that is the binding molecule binds at least one TNFR2 molecule on the surface of one cell and at least one TNFR2 molecule on the surface of another cell. In one embodiment, the binding molecule is able to bind cis and trans to TNFR2. For example, it may have the capability to do so and in another embodiment it may actually do so. In one embodiment a binding molecule of the present invention results in the formation of hexamers of TNFR2 on both cells when trans binding occurs. In one embodiment, the orientation of the binding sites may influence whether an antibody binds to TNFR2 in cis and/or trans fashion. In one embodiment, a binding molecule of the present invention is one that has at least two TNFR2 binding sites that have opposing orientation compared to each other preferably wherein the binding molecule has at least two such TNFR2 binding sites that are antibody antigen-binding sites. In one preferred embodiment, a binding molecule of the present invention is one that has at least two TNFR2 binding sites that are able to bind TNFR2 molecules on different cells allowing for trans-binding, preferably wherein the binding molecule has at least two such TNFR2 binding sites that are antibody antigen-binding sites. In another preferred embodiment a binding molecule of the present invention has a valency of at least four and is able to bind a plurality of different molecules of TNFR2 on the surface of the same cell, preferably where the binding molecule has a valency of at least six.

In another embodiment, a binding molecule of the present invention may be a diabody. A diabody is typically a small bivalent antigen-binding antibody portion, and there are a variety of diabody formats, any of which may be used, for example a diabody may comprise a heavy chain variable domain linked to a light chain variable domain on the same polypeptide chain linked by a peptide linker that is too short to allow pairing between the two domains on the same chain. This results in pairing with the complementary domains of another chain and in the assembly of a dimeric molecule with two antigen binding sites for TNFR2.

A wide variety of antibody formats exist and may be employed either as an antibody of the present invention or as part of it, for example Fab, Fv, scFv, (ScFv)2, (ScFv2), bispecific (scFv2), triabody, tetrabody, bispecific sc(Fv)2, tandem di-scFv, tandem tri-scFv, and tribodies all represent possible antibody formats of the present invention. As discussed above, a binding molecule of the present invention has a valency of at least two. In one preferred embodiment, a binding molecule of the present invention has a valency of at least two spatially movable potentially cell-cell connecting TNFR2 binding sites or at least six “parallel” or “unidirectionally” oriented cis-acting binding sites. In embodiments where formats mentioned herein have a valency of one they may be employed as part of an antibody of the present invention in such formats, rather than as the whole antibody.

In one embodiment, rather than comprising one of the specific binding sites for TNFR2 set out herein, a binding molecule of the present invention may comprise a variant binding site for TNFR2. In one preferred embodiment, a variant binding molecule for TNFR2 will be defined by its ability to cross-block one of the specific binding molecules for TNFR2 set out herein binding to TNFR2. The ability to cross-block may be assessed using a binding assay for measuring binding of a binding molecule to TNFR2 and the ability of a further binding molecule to cross-block such binding to TNFR2. In one embodiment, the ability to cross-block is studied using a monovalent molecule that just comprises one copy of the binding site for TNFR2. For example, the ability of a scFv comprising the binding site to cross-block a scFv that only differs in having a binding site for TNFR2 from one of the specific binding molecules set out herein is studied. In another embodiment, the ability of the actual binding molecules to cross-block each other is studied without converting them into scFv molecules. In a further embodiment, a binding molecule of the present invention will be able to block, or reduce, the binding of TNFα to TNFR2. In another embodiment, a binding molecule of the present invention will be able to reduce, or block the binding of lymphotoxin-alpha to TNFR2.

In one embodiment, the affinity of the binding domain for TNFR2 in a binding molecule, in particular an antibody, of the present invention, has a K_{D} which is about 400 nM or smaller, 200 nM or smaller such as about 100 nM, 50 nM, 20 nM, 10 nM, 1 nM, 500 μM, 250 μM, 200 μM, 100 μM or smaller. In one embodiment, the binding affinity is 50 μM or smaller. For example, in one embodiment an individual TNFR2 binding site in a binding molecule of the present invention will show such affinity for TNFR2, rather than that being the overall affinity for the binding molecule. In one embodiment, such affinity is the overall avidity of the antibody for TNFR2. In one embodiment, the affinity of an individual antigen-binding site of a binding molecule of the present invention may be less than 1 μM, less than 750 nM, less than 500 nM, less than 250 nM, less than 200 nM, less than 150 nM, less than 100 nM, less than 75 nM, less than 50 nM, less than 10 nM, less than 1 nM, less than 0.1 nM, less than 10 μM, less than 1 μM, or less than 0.1 μM. In some embodiments, the Kd is from about 0.1 μM to about 1 μM.

In one embodiment, an antibody of the present invention is a monoclonal antibody. In one embodiment, an antibody of the present invention is an IgG1, IgG2 or IgG4 antibody or comprises heavy chain constant region of that class of antibody. In one especially preferred embodiment, an antibody of the present invention is a IgG1 isotype antibody or comprises such an antibody. In one preferred embodiment, the constant region employed is a human IgG constant region. In a particularly preferred embodiment, a human IgG1 constant region is employed. It may have any of the modifications set out herein. Without particular limitation, the term “antibody” encompasses antibodies from any appropriate source species, including chicken, sharks and mammalian such as mouse, hamster, rabbit, goat, cow, llama, non-human primate and human. In one preferred embodiment, an antibody of the present invention is a humanised antibody. In another preferred embodiment, the binding molecule, and in particular antibody, of the present invention is wholly human. Non-limiting examples for methods to generate humanized antibodies are known in the art, e.g. from Riechmann et al. (Nature. 1988 Mar. 24; 332(6162):323-7) or Jones et al. (Nature. 1986 May 29-June 4; 321 (6069):522-5). In a further preferred embodiment, an antibody of the present invention is a human antibody. As discussed herein, a binding molecule of the present invention, and in particular an antibody, may comprise non-human sequences such as a tenascin-c peptide, but in one preferred embodiment all of the antibody based sequences of the antibody are human notwithstanding the presence of the tenascin peptide sequence. The term “antibody” encompasses monomeric antibodies (such as IgD, IgE, IgG) or oligomeric antibodies (such as IgA or IgM). The term “antibody” also encompasses—without particular limitations—isolated antibodies and modified antibodies such as genetically engineered antibodies, e.g. chimeric or humanized antibodies.

In one preferred embodiment a binding molecule of the present invention has only one specificity for TNFR2. Hence, in one preferred embodiment all of the antigen-binding sites of an antibody of the present invention which are specific for TNFR2 will be specific for the same epitope of TNFR2. In another embodiment, a binding molecule of the present invention will comprise more than one specificity for TNFR2. For example, that may be because the binding molecule that comprises both antibody antigen-binding sites, but also ligand based TNFR2 binding sites. In one preferred embodiment though, a binding molecule of the present invention is an antibody that comprises antigen-binding sites that recognise more than one epitope of TNFR2. For example, in one preferred embodiment a binding molecule of the present invention, and in particular an antibody, is bispecific for TNFR2. In one preferred embodiment, a binding molecule of the present invention one specificity of the binding molecule for TNFR2 is provided by a pair of light and heavy chain variable regions whilst the other is provided by scFv fragments present as part of the overall binding molecule. In another embodiment, all of the TNFR2 binding sites of a binding molecule, and in particular antibody, are provided by scFvs with the binding molecule formed from two different polypeptides, each of which comprise a scFv with a different specificity.

In one embodiment, a binding molecule of the present invention may comprise an Fc region or an antibody molecule which has irrelevant specificity and the ability to bind to TNFR2 is provided by other portions of the binding molecule. For example, in one embodiment a binding molecule of the present invention comprises an Fc region where at the C-terminal portion of the heavy chains are TNF-alpha molecules and in particular mutant TNF-alpha molecules that are selective for TNFR2, such as the TNF80 mutant. In one embodiment, a binding molecule of the present invention may comprise an immunoglobulin of irrelevant specificity and at the C-terminal portion of the heavy chains may be TNF-alpha molecules, and in particular mutant TNF-alpha molecules such as TNF80. In one embodiment, the heavy chains of the Fe or irrelevant antibody may comprise as part of the same polypeptide a TNF-alpha and in particular a TNF80 mutant. In one embodiment, they may comprise a single chain polypeptide comprising three TNF-alpha polypeptides and in particular three TNF80 polypeptides. In another embodiment, before the TNF-alpha portion of the polypeptides the heavy chains may comprise a TNC peptide or variant thereof. In a further preferred embodiment, a binding molecule of the present invention may comprise an Fe region that, rather than have variable regions has the two heavy chain portions of the Fc region as part of a single polypeptide.

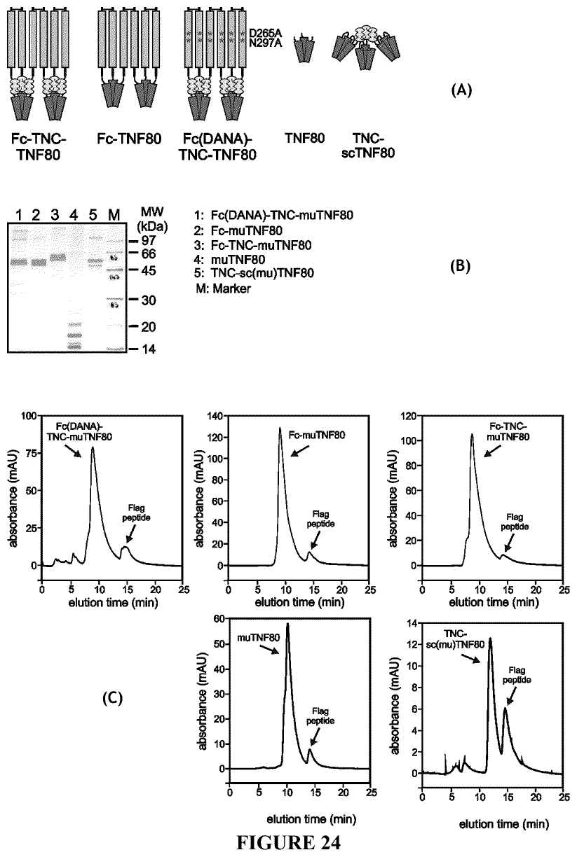

An example of a preferred binding molecule comprising a TNF ligand is Fc(DANA)-TNC-TNF80 as depicted in FIG. 24 where DANA indicates the heavy chains of the Fe regions comprising D265A and N297A mutations and TNC indicates the tenascin trimerization domain peptide sequence. Hence, a preferred binding molecule of the present invention comprises three Fe regions where each heavy chain has the DANA modifications of D265A and N297A. In a further preferred embodiment, each of those heavy chain polypeptides comprises a TNC peptide. In a further preferred embodiment, each heavy chain polypeptide of each Fe region comprises a TNC peptide sequence followed by a TNF-alpha sequence and in particular a TNF80 sequence.

A further preferred binding molecule of the present invention is Fc(DANA)-TNF80 which comprises three Fe regions where each heavy chain polypeptide comprises the DANA modifications of D265A and N297A and a TNF-alpha, in particular a TNF80 sequence. The present invention also provides as binding molecules the Fc-TNC-TNF80 and Fc-TNF80 format molecules shown in FIG. 24.

In another embodiment, a binding molecule is provided that specifically binds TNFR2, but not TNFR1, where the binding molecule is an FcγR independent agonist of TNFR2, has a valency of at least two for binding TNFR2, and comprises polypeptides comprising mutant TNF-alpha that bind TNFR2, but not TNFR1, and a tenascin peptide sequence that oligomerises the polypeptides. In one embodiment, such a binding molecule does not include antibody sequences. In one embodiment, the molecule comprises polypeptides that each comprise a mutant TNF-alpha sequence with a tenascin peptide, for example any of the tenascin polypeptides disclosed herein. In one preferred embodiment, the tenascin peptide sequences results in oligomerisation of such polypeptides. In one embodiment, the mutant TNF-alpha has 143N/145R modifications resulting in selective binding for TNFR2 over TNFR1. Such a TNF mutant may also be referred to as TNF80.

Binding Molecules with More than One Specificity for TNFR2

In a preferred embodiment, all of the antigen-binding binding sites of the binding molecule specific for TNFR2 have the same specificity for TNFR2. In particular, they may all have the same amino acid sequence and hence bind the same epitope. In one embodiment, all of the antigen-binding sites are specific for the same epitope of TNFR2. In another embodiment, all of the TNFR2 antigen-binding sites are capable of cross-blocking the binding of the other TNFR2 antigen-binding sites to TNFR2.

However, in a further preferred embodiment, a binding molecule may have antigen-binding sites that bind TNFR2, but at least two of those antigen-binding sites have different specificities for TNFR2. In one embodiment, the binding molecule comprises at least two antigen-binding sites for TNFR2 that bind a different epitope of the same TNFR2 molecule. In another embodiment, the at least two antigen-binding sites do not cross-block the binding of each other to TNFR2. In one embodiment, a binding molecule is biparatopic for TNFR2 in the sense that it comprises antigen-binding sites recognising at least two different epitopes of TNFR2. In another embodiment, at least two, three, four, five, or six different specificities for TNFR2 are conferred by the antigen binding sites. In one embodiment, all of the antigen-binding sites of the binding molecule specific for TNFR2 have a different specificity for TNFR2.

In one embodiment, the binding molecule is a hexavalent binding molecule comprising two polypeptides where each individual polypeptide comprises at least two different antigen-binding sites with a different specificity for TNFR2. In one embodiment, each polypeptide comprises three such specificities. In one embodiment, though the two polypeptides comprises different specificities on the same polypeptide, the two polypeptides are identical. In another embodiment, the two polypeptides are not identical, with each polypeptide comprising three different specificities for TNFR2 and in total six different specificities for TNFR2 are present.

In another embodiment where the antibody comprises “North” and “South” orientated antigen-binding sites the “North” and “South” orientated antigen-binding sites confer a different specificity to each other for TNFR2. For instance, in one embodiment, any of the tetravalet antigen-binding molecules set out herein may comprise two antigen-binding sites of one specificity and two of another specificity for TNFR2. In one embodiment, the two “South” orientated scFvs may have a different specificity for TNFR2 than the “North” orientated antigen-binding sites.

sdBs, SdAbs and VHH Binding Domains

In one particularly preferred embodiment, a binding molecule of the present invention comprises a single domain binders (sdB). Single domain binders include, for instance, non-Ig engineered protein scaffolds such as darpins, affibodies, adnectins, anticalin proteins, or peptides and the like. So wherever reference is made to sdB, sdAb, HCAb, and VHH, it may be possible to also employ a darpin, affibody, adnectin, anticalin, or peptide that is able to bind TNFR2 and the term sdB encompasses such binding entities being employed.

Examples of single binding domain binders (sdBs) include in particular single domain antibodies (sdAb), for example, heavy chain only antibodies (HCAb), particularly VHH domain antibodies. Employing VHH antigen-binding domains is an especially preferred embodiment. A single-domain antibody (sdAb) is an antibody fragment consisting of a single monomeric variable antibody domain. Like a whole antibody, sdAb is able to bind selectively to a specific antigen. sdAb may be antibody fragments that can be engineered from single monomeric variable domains of either camelids' heavy-chain antibody (VHH) or cartilaginous fishes' IgNAR (VNAR), or be developed from camelized human antibodies. Any such sdAbs may be employed. Especially preferred sdAbs are VHH domains. In one embodiment, a binding molecule of the present invention may comprise at least two sdAb domains. In one embodiment, the binding molecule may comprise at least two sdAb domains on the same polypeptide. In a preferred embodiment, an antibody of the present invention may be a molecule that comprises a single domain antibody or single domain antibodies where the overall valency of the binding molecule is at least two. For example, an antibody of the present invention may comprise two such single domain antibodies joined together as part of the overall antibody.

In another preferred embodiment, a binding molecule of the present may comprise VHH binding domains as sdAbs. SdAbs from organisms such as Camelids, sharks, and other cartilaginous fish that produce heavy chain-only antibodies may be employed. The single-domain variable fragments of these heavy chain-only antibodies are termed VHHs or nanobodies or sdAb. VHHs retain the immunoglobulin fold shared by antibodies, using three hypervariable loops, CDR1, CDR2 and CDR3, to bind to their targets. A VHH fragment (e.g., NANOBODY®) is a recombinant, antigen-specific, single-domain, variable fragment derived from camelid heavy chain antibodies.

As a binding molecule of the present invention preferably has a valency of at least two for TNFR2, in one embodiment where the binding molecule comprises VHH fragments, it will comprise at least two such VHH fragments. The individual VHH fragments may be joined together, for example through the use of a linker and hence, in one embodiment, may be expressed as a single polypeptide which overall comprises at least two VHH fragments which are each able to specifically bind TNFR2. VHH fragments do not have a Fc region and in one embodiment where VHH fragments are present as part of a binding molecule of the present invention, the binding molecule will lack an Fc region and so not bind Fc receptors for that reason. In one embodiment, a binding molecule of the present invention will comprise, or at least comprise, three, four, five, six or more VHH fragments specific for TNFR2. In another embodiment, a binding molecule of the present invention will comprise a single VHH domain antibody. In a particularly preferred embodiment, an antibody will comprise at least four sdAbs. In a particularly preferred embodiment, an antibody will comprise four sdAbs. In a particularly preferred embodiment, an antibody will comprise at least six sdAbs. In a particularly preferred embodiment, an antibody will comprise six sdAbs. In a further preferred embodiment, the sdAbs are VHHs. In one embodiment, a binding molecule will comprise that number of sdBrs.

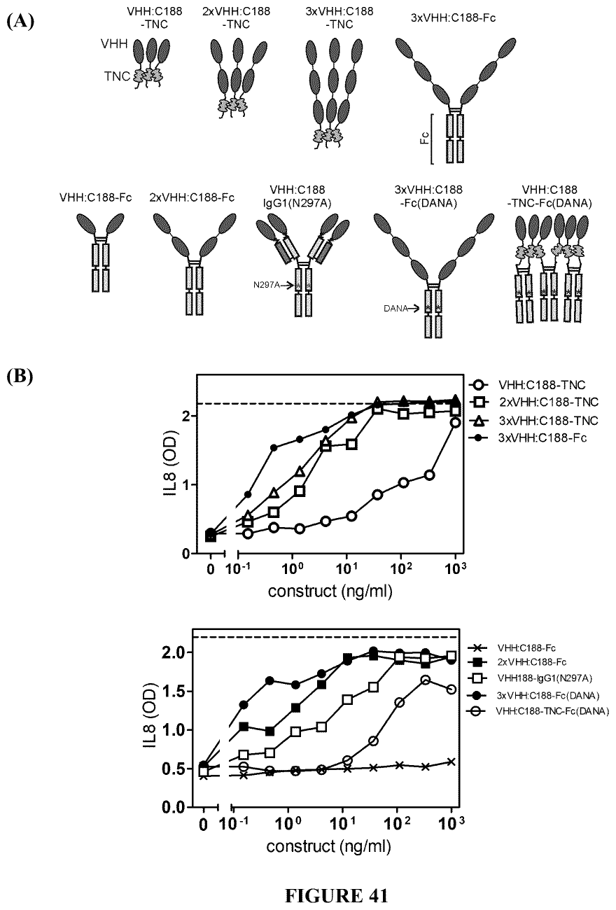

In one embodiment, a binding molecule of the present invention will comprise a Fc region, but no CH1 region and be able to bind TNFR2 without the participation of a light chain. In one preferred embodiment, a binding molecule of the present invention will comprise one or more VHH, comprise an Fc region and be able to bind TNFR2 without the participation of a light chain. In another embodiment, a binding molecule of the present invention will comprise one or more VHH and comprise TNC trimerization domains or Fc region, or both, as depicted in some of the structures shown in FIG. 41.

### Antibody Constant Regions and Fc Region Functions

In a preferred embodiment, a binding molecule of the present invention does not bind Fc receptors and in particular does not bind to FcγR receptors. In one preferred embodiment, the binding molecule of the present invention is an antibody and it does not bind to Fc receptors, either because it does not comprise an Fc region or alternatively as it is has an Fc region modified so that it does not bind Fc receptors. Fc domain as employed herein generally refers to —(CH₂CH₃)2, unless the context clearly indicates otherwise, where CH2 is the heavy chain CH2 domain, CH3 is the heavy chain CH3 domain, and there are two CH₂CH₃ with one from each heavy chain. In one embodiment, an antibody of the present invention does not comprise a —CH₂CH₃ fragment. In one embodiment, an antibody of the present invention does not comprise a CH₂ domain. In one embodiment, an antibody of the present invention does not comprise a CH₃ domain.

In one embodiment, a binding molecule of the present invention binds to an Fc gamma receptor but to a substantially decreased extent relative to binding of an identical antibody comprising an unmodified Fc region to the FcgR (e.g., a decrease in binding to a FcγR by at least 70%, at least 80%, at least 90%, at least 95%, at least 98%, at least 99%, or 100% relative to binding of the identical antibody comprising an unmodified Fc region to the FcγR as measured). In a particularly preferred embodiment though the binding molecule has no detectable binding to an FcγR at all. Binding, including the presence or absence of binding, can be determined using a variety of techniques known in the art, for example but not limited to, equilibrium methods (e.g., enzyme-linked immunoabsorbent assay (ELISA); KinExA, Rathanaswami et al. Analytical Biochemistry, Vol. 373:52-60, 2008; or radioimmunoassay (RIA)), or by a surface plasmon resonance assay or other mechanism of kinetics-based assay (e.g., BIACORE™ analysis or Octet™ analysis (forteBIO)), and other methods such as indirect binding assays, competitive binding assays fluorescence resonance energy transfer (FRET), gel electrophoresis and chromatography (e.g. gel filtration).

In one embodiment, where an Fc region is present, the Fc region employed is mutated, in particular comprising a mutation described herein. In one embodiment the mutation is to remove binding to Fc receptors and in particular FcγR. In one preferred embodiment the antibody of the invention has been mutated so that it does not bind Fc receptors.

In one embodiment, an antibody of the present invention may comprise an Fc region, but not the variable regions that would normally be found in a naturally occurring four chain antibody. In one preferred embodiment, such Fc regions may though comprise other sequences as part of the polypeptides comprising the heavy chains of the Fc region, in particular TNC peptide and/or TNF-alpha (particularly TNF80) and preferably both, in particular in the C-terminal portion of the polypeptides.

In one embodiment, an antibody of the present invention may comprise an aglycosyl IgG, for example to bring about reduced Fc function and in particular a nearly Fc-null phenotype. In one embodiment, an antibody of the invention has a modification at N297 and in particular N297A. In one embodiment an antibody of the invention has modifications at F243 and/or F244, in particular ones that mean that the antibody comprises an aglycosyl IgG. In one embodiment, an antibody of the present invention may comprise the F243A and/or F244A heavy chain modifications. In another embodiment, one or more of F241, F243, V262 and V264 may be modified and particularly to amino acids that influence glycosylation. In one embodiment, an antibody of the present invention may have modifications at F241A, F243A, V262E and V264E. Such modifications are discussed in Yu et al (2013) 135(26): 9723-9732, which is incorporated by reference in its entirety, particularly in relation to the modifications discussed therein. Such modifications provide a way to modulate, for example, Fc receptor binding. A modification which influences the glycosylation of the antibody may be present. Further, an antibody of the invention may be produced in a cell type that influences glycosylation as a further approach for sugar engineering. In one embodiment, the fucosylation, sialylation, galactosylation, and/or mannosylation of an antibody of the present invention may be altered either by sequence modifications and/or via the type of cell used to produce the antibody.

In one embodiment, an antibody of the present invention has modifications at position 297 and/or 299. For example, in one embodiment, an antibody of the present invention comprises a N297A modification in its heavy chains, preferably N297Q or mutation of Ser or Thr at 299 to other residues. In one embodiment it has both those modifications.

In one embodiment, a binding molecule of the present invention, and in particular an antibody, comprises two different heavy chains where the heavy chains comprise modifications that allow the different heavy chains to preferentially associate compared to heavy chains associating with identical heavy chains. Such an approach may be in particular employed where the antibody has more than one specificity for TNFR2, for instance where the antibody binds two different epitopes of TNFR2. In one embodiment, the two different heavy chains comprise knob-in-hole mutations. In certain aspects, the knob-in hole mutations are a T366W mutation in one heavy chain constant region and a T366S, L368A, and a Y407V mutation in the other domain. In certain aspects, the modifications comprise charge-pair mutations. In certain aspects, the charge-pair mutations are a T366K mutation in one of the heavy chain constant regions and a corresponding L351D mutation in the other domain. In an alternative embodiment, rather than have modifications that result in preferential pairing of different heavy chains, the heavy chains comprise modifications that mean a heterodimer comprising the two heavy chains can be purified preferentially from the homodimers only comprising one type of heavy chain. For example, the modifications may alter affinity for Protein A, with one heavy chain still able to bind Protein A, whilst the modified heavy chain does not do so, meaning that heterodimers of the two different heavy chains can be purified based on their affinity for Protein A.

In other embodiments, heavy and light chains may comprise modifications that change whether or not a disulphide bridge is formed between them. In a variety of embodiments, the modifications comprise mutations that generate engineered disulfide bridges between light and heavy chains. As described herein, “engineered disulfide bridges” are mutations that provide non-endogenous cysteine amino acids in two or more polypeptides such that a non-native disulfide bond forms when the two or more domains associate. Engineered disulfide bridges are described in greater detail in Merchant et al. (Nature Biotech (1998) 16:677-681), the entirety of which is hereby incorporated by reference. In a particular embodiment, the mutations that generate engineered disulfide bridges are a K392C mutation in one of a first or second CH3 domains, and a D399C in the other CH3 domain. In a preferred embodiment, the mutations that generate engineered disulfide bridges are a S354C mutation in one of a first or second CH3 domains, and a Y349C in the other CH3 domain. In another preferred embodiment, the mutations that generate engineered disulfide bridges are a 447C mutation in both the first and second CH3 domains that are provided by extension of the C-terminus of a CH3 domain incorporating a KSC tripeptide sequence.

In one embodiment, binding molecules of the present invention may comprise modifications that alter serum half-life of the binding molecule. Hence, in another embodiment, an antibody of the present invention has Fc region modification(s) that alter the half-life of the antibody. Such modifications may be present as well as those that alter Fc functions. In one preferred embodiment, an antibody of the present invention has modification(s) that alter the serum half-life of the antibody. In one particularly preferred embodiment, an antibody of the present invention has modification(s) that alter serum half-life of the antibody compared to an antibody lacking such modifications. In one embodiment, the modifications result in increased serum half-life. In another embodiment, they result in decreased serum half-life. In another preferred embodiment, an antibody of the present invention comprises one or more modifications that collectively both silence the Fc region and decrease the serum half-life of the antibody compared to an antibody lacking such modifications.

In an especially preferred embodiment, the antibody has a constant region with minor to no effector functions, such as an antibody derived from the FDA-approved antibody Durvalumab with Fc modifications L234F/L235E/P33IS. The Fc modifications in Durvalumab help eliminate Fc functions and so the use of the light and heavy chain constant regions or Durvalumab is a particularly effective way to provide a constant region with the desired lack of Fc functions. As Durvalumab has gained clinical approval that further represents a reason why the use of its constant regions represents a particularly preferred embodiment. Hence, in one preferred embodiment, the binding molecule comprises the Fc region of Durvalumab.

In one preferred embodiment, where the binding molecule comprises VHH binding domains, the polypeptides comprising the VHH domains further comprise the CH2 and CH2 domains of Durvalumab. In a further preferred embodiment, a binding molecule comprises heavy and light chains, where the antibody comprises light and heavy chain constant regions of Durvalumab. In any of the embodiments, where the binding molecule comprises constant region sequences derived from Durvalumab, they may comprise any of the constant region modifications discussed herein. Durvalumab has a human IgG1 backbone. Hence, in a further preferred embodiment, where the binding molecule comprises a constant region, it may comprise a human IgG region. In a preferred embodiment, it may comprise a human IgG1 region. Such regions may be modified to eliminate Fc function. In one embodiment, the constant regions may be modified to delete the CH1 region, particularly where the antigen-binding domains are sdBrs.

The heavy and light chain constant region sequences of Durvalumab are provided respectively as SEQ ID NOs 135 and 136. In one preferred embodiment a binding molecule of the present invention comprises such light and heavy chain variable sequences or a variant of such a sequence. In one preferred embodiment, the variant sequence or sequences have at least 90% sequence identity to the relevant specific sequence. In another embodiment, the variant has at least 95% sequence identity. In one embodiment, a binding molecule of the present invention comprises the heavy and light chain constant region sequences of SEQ ID Nos: 135 and 136, but with one or more of the constant region sequence modifications discussed herein. In one preferred embodiment, such heavy and light chain constant regions are employed in tetravalent binding molecules provided herein.

A variant of the heavy chain constant region of Durvalumab sequence with the CH1 region deleted is provided as SEQ ID NO: 316. In one preferred embodiment, such a constant region is employed where the binding molecule comprises sdAb based antigen binding sites, preferably with VHH antigen-binding sites. Such a sequence, but with one or more of the other constant region modifications discussed herein may be employed. In one embodiment, a binding molecule of the present invention may comprise the sequence of SEQ ID NO: 316 or a variant thereof with at least 90% sequence identity. In another embodiment, the variant may have at least 95% sequence identity. A variant in such embodiments will still be CH1 deleted. Such heavy chain constant regions with a CH1 deletion is preferably employed in one embodiment where at least one of the antigen-binding sites is a VHH antigen-binding domain specific for TNFR2.

### Illustrative Preferred High Valency TNFR2 Binding Molecules

In a preferred embodiment, a binding molecule of the present invention has a valency for TNFR2 of at least four. In one embodiment, the valency of the binding molecule is from four to nine for TNFR2. Whilst not wishing to be bound to a particular theory, it is thought that a higher density of TNFR2 binding sites within a limited space may help promote TNFR2 agonist activity. Surprisingly, the present inventors have found that it is possible to convert known TNFR2 antagonist antibodies to TNFR2 agonist molecules through reformatting the antibodies into the formats described herein, particularly with those with a valency of four or higher.

In one particularly preferred embodiment, the binding molecule provided is tetravalent for TNFR2. Examples of tetravalent formats are provided in the Examples of the present application. The present invention provides any of those formats comprising one of the specific binding domains disclosed herein. Examples of preferred formats include: scFv-IgG (see FIG. 1); IgG-HC:scFv (see FIG. 7); IgG-LC:scFv (see FIG. 7). IgG-HC:scFv represents a particularly preferred format. IgG-LC:scFv also represents a particularly preferred embodiment. In one embodiment, the scFv groups are joined to the heavy or light chain in such formats by a linker. Hence, in one preferred embodiment, the G4S linker is employed to join the scFv. In another embodiment, it may be that the antibody comprises a linker separating at least two of the binding domains. Examples of preferred tetravalent formats are illustrated in FIG. 29. The constructs shown in FIG. 29 comprise the C4 binding domain and that represents a preferred binding domain. However, the formats depicted in FIG. 29 are also provided where the VH and VL regions of each binding sites are any of the variant C4 sequences set out herein. The formats shown in FIG. 29 may also be employed where the binding molecule is one where the binding sites comprise any of the sets of six CDRs set out herein.

A further particularly preferred binding molecule format of the present invention is a hexavalent binding molecule, in particular one having six specific TNFR2 binding sites. The inventors have unexpectedly found that binding molecules in the hexameric format shows less variation in TNFR2 agonist activity where different TNFR2 antigen-binding domains are used to generate an otherwise identical hexavalentc molecule.

Illustrative hexavalent formats include any of those employed in the Examples or Figures of the present application. In one preferred embodiment, the hexavalent antibody comprises at least two binding sites specific for TNFR2 on the same polypeptide. In one particularly preferred embodiment, the hexavalent antibody comprises two polypeptides where each polypeptide has three binding sites for TNFR2, particularly three VHHs, particularly the particular VHHs set out herein.

In one embodiment, all of the VHH antigen-binding sites in a hexavalent binding molecule are the same. In another embodiment, a binding molecule may have at least two antigen-binding sites that have a different specificity for TNFR2, for instance which binding a different epitope. In another embodiment, all of the antigen-binding sites of the binding molecule that are specific for TNFR2 may have a different specificity for TNFR2 i.e. bind a different epitope. In another embodiment, the antigen-binding sites may be different in the sense that they do not cross-block each other when binding to TNFR2.

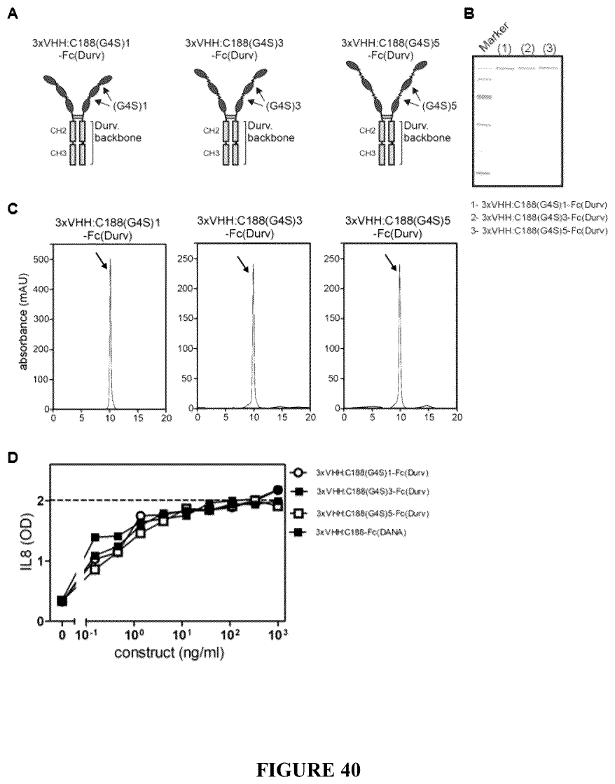

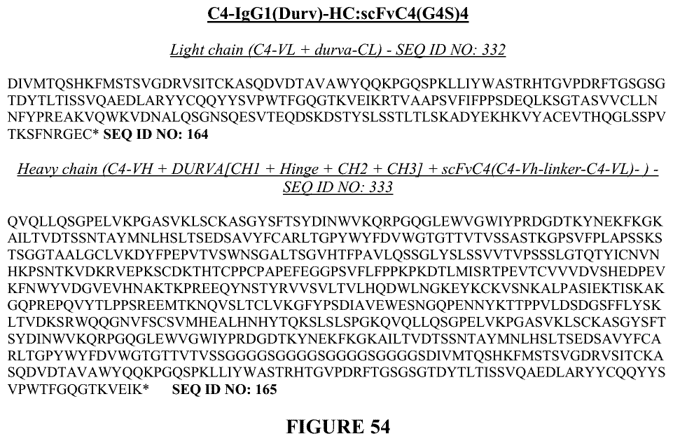

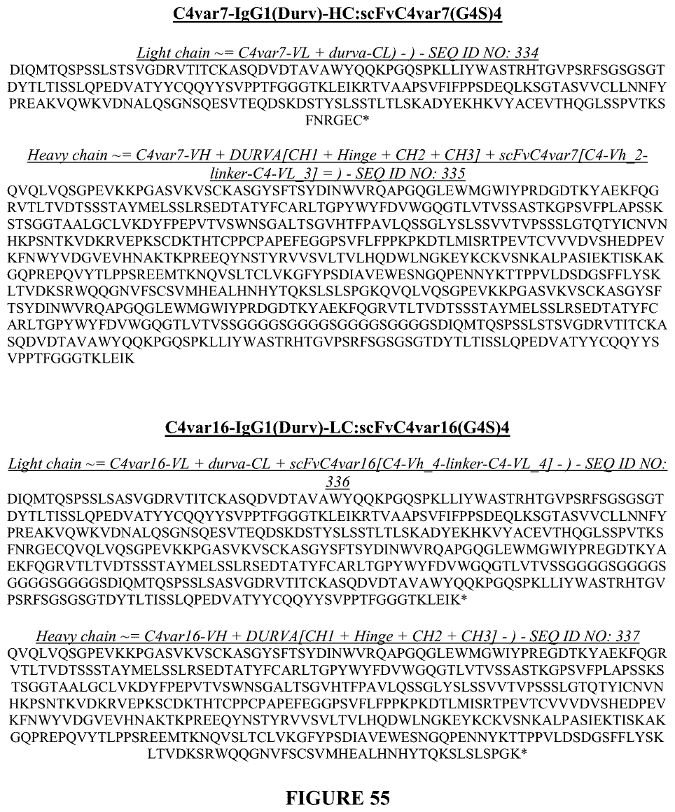

Examples of preferred hexavalent binding molecule formats include those described in the Examples and Figures of the present application. In instances where a Figure or Example describes an antibody format with a particular TNFR2 binding domain, the present invention also provides the same format antibody but with one of the other TNFR2 binding domains described herein. In a particularly preferred embodiment, the TNFR2 binding domains are VHH domains. In a preferred embodiment, the VHH domain is employed is one of the specific VHH domains described herein or a variant thereof. In one preferred embodiment, the binding molecule is in one of the hexavalent formats depicted in FIG. 40. Further preferred formats and specific sequences are provided in FIGS. 54 and 55. An especially preferred format comprises two polypeptides, each with three VHH domains present. In a further especially preferred format, the binding molecule is in the format 3×VHH-Fc. For instance, a particularly preferred format is 3×VHH-Fc. A further preferred format is 3×VHH-Fc(DANA). A particularly preferred VHH domain to be employed in a binding molecule of the present invention is C188. A further preferred VHH domain is one of the specific variants of C188 described herein. Hence, 3×VHH:C188-Fc is a particularly preferred binding molecule. A further preferred binding molecule is 3×VHH:C188-Fc(DANA). Alternatively, in either of those formats C188 may be substituted for one of the other VHH domains described herein.

In a further particularly preferred embodiment, where the hexavalent binding molecule comprises an Fc region it will comprise the Fc region for the antibody Durvalumab. In an alternative embodiment, the binding molecule will comprise the Fc region for Durvalumab, but with any of the Fc region set out herein.

### Binding Molecule Generation and Screening

In one embodiment, the binding molecules, in particular antibodies, of the present invention are mutated to provide improved affinity for a target antigen or antigens and in particular for TNFR2. Such variants can be obtained by a number of affinity maturation protocols including mutating the CDRs (Yang et al., J. Mol. Biol., 254, 392-403, 1995), chain shuffling (Marks et al., Bio/Technology, 10, 779-783, 1992), use of mutator strains of E. coli (Low et al J. Mol. Biol., 250, 359-368, 1996), DNA shuffling (Patten et al Curr. Opin. Biotechnol., 8, 724-733, 1997), phage display (Thompson et al., J. Mol. Biol., 256, 77-88, 1996) and sexual PCR (Crameri et al Nature, 391, 288-291, 1998). Vaughan et al (supra) discusses these methods of affinity maturation. Binding domains for use in the present invention may be generated by any suitable method known in the art, for example CDRs may be taken from non-human antibodies including commercially available antibodies and grafted into human frameworks or alternatively chimeric antibodies can be prepared with non-human variable regions and human constant regions etc. As the antibodies of the present invention may not be in a naturally occurring format, it may be that such screening is used to identify antibody antigen-binding sites with desirable properties for binding TNFR2 and then they are reformatted so that they are in one of the formats set out herein.

The skilled person may generate antibodies for use in the antibodies of the invention using any suitable method known in the art. Antigen polypeptides, for use in generating antibodies for example for use to immunize a host or for use in panning, such as in phage display, may be prepared by processes well known in the art from genetically engineered host cells comprising expression systems or they may be recovered from natural biological sources. In the present application, the term “polypeptides” includes peptides, polypeptides and proteins. These are used interchangeably unless otherwise specified. The antigen polypeptide may in some instances be part of a larger protein such as a fusion protein for example fused to an affinity tag or similar. In one embodiment, the host may be immunised with a cell transfected with TNFR2, for instance expressing TNFR2 on its surface.

Antibodies generated against an antigen polypeptide may be obtained, where immunisation of an animal is necessary, by administering the polypeptides to an animal, preferably a non-human animal, using well-known and routine protocols, see for example Handbook of Experimental Immunology, D. M. Weir (ed.), Vol 4, Blackwell Scientific Publishers, Oxford, England, 1986). Many warm-blooded animals, such as rabbits, mice, rats, sheep, cows, camels or pigs may be immunized. However, mice, rabbits, pigs and rats are generally most suitable. Monoclonal antibodies may be prepared by any method known in the art such as the hybridoma technique (Kohler & Milstein, 1975, Nature, 256:495-497), the trioma technique, the human B-cell hybridoma technique (Kozbor et al 1983, Immunology Today, 4:72) and the EBV-hybridoma technique (Cole et al Monoclonal Antibodies and Cancer Therapy, pp77-96, Alan R Liss, Inc., 1985). Antibodies may also be generated using single lymphocyte antibody methods by cloning and expressing immunoglobulin variable region cDNAs generated from single lymphocytes selected for the production of specific antibodies by, for example, the methods described by Babcook, J. et al 1996, Proc. Natl. Acad. Sci. USA 93(15):7843-78481; WO92/02551; WO2004/051268 and WO2004/106377. The antibodies for use in the present invention can also be generated using various phage display methods known in the art and include those disclosed by Brinkman et al. (in J. Immunol. Methods, 1995, 182: 41-50), Ames et al. (J. Immunol. Methods, 1995, 184:177-186), Kettleborough et al. (Eur. J. Immunol. 1994, 24:952-958), Persic et al. (Gene, 1997 187 9-18), Burton et al. (Advances in Immunology, 1994, 57:191-280) and WO90/02809; WO91/10737; WO92/01047; WO92/18619; WO93/11236; WO95/15982; WO95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743; 5,969,108, and WO20011/30305.

In one example an antibody of the present invention is fully human, in particular one or more of the variable domains are fully human. Fully human molecules are those in which the variable regions and the constant regions (where present) of both the heavy and the light chains are all of human origin, or substantially identical to sequences of human origin, not necessarily from the same antibody. Examples of fully human antibodies may include antibodies produced, for example by the phage display methods described above and antibodies produced by mice in which the murine immunoglobulin variable and optionally the constant region genes have been replaced by their human counterparts e.g. as described in general terms in EP0546073, U.S. Pat. Nos. 5,545,806, 5,569,825, 5,625,126, 5,633,425, 5,661,016, 5,770,429, EP 0438474 and EP0463151 which are each incorporated by reference.

In one example, the antigen-binding sites, and in particular the variable regions, of the antibodies according to the invention are humanised. Humanised (which include CDR-grafted antibodies) as employed herein refers to molecules having one or more complementarity determining regions (CDRs) from a non-human species and a framework region from a human immunoglobulin molecule (see, e.g. U.S. Pat. No. 5,585,089; WO91/09967 which are incorporated by reference). It will be appreciated that it may only be necessary to transfer the specificity determining residues of the CDRs rather than the entire CDR (see for example, Kashmiri et al., 2005, Methods, 36, 25-34). In a preferred embodiment though, the whole CDR or CDRs is/are transplanted. Humanised antibodies may optionally further comprise one or more framework residues derived from the non-human species from which the CDRs were derived. As used herein, the term “humanised antibody molecule” refers to an antibody molecule wherein the heavy and/or light chain contains one or more CDRs (including, if desired, one or more modified CDRs) from a donor antibody (e.g. a murine monoclonal antibody) grafted into a heavy and/or light chain variable region framework of an acceptor antibody (e.g. a human antibody). For a review, see Vaughan et al, Nature Biotechnology, 16, 535-539, 1998. In one embodiment, rather than the entire CDR being transferred, only one or more of the specificity determining residues from any one of the CDRs described herein above are transferred to the human antibody framework (see for example, Kashmiri et al., 2005, Methods, 36, 25-34). In one embodiment only the specificity determining residues from one or more of the CDRs described herein above are transferred to the human antibody framework. In another embodiment, only the specificity determining residues from each of the CDRs described herein above are transferred to the human antibody framework.

When the CDRs or specificity determining residues are grafted, any appropriate acceptor variable region framework sequence may be used having regard to the class/type of the donor antibody from which the CDRs are derived, including mouse, primate and human framework regions. Suitably, the humanised antibody according to the present invention has a variable domain comprising human acceptor framework regions as well as one or more of the CDRs provided herein. Examples of human frameworks which can be used in the present invention are KOL, NEWM, REI, EU, TUR, TEI, LAY and POM (Kabat et al supra). For example, KOL and NEWM can be used for the heavy chain, REI can be used for the light chain and EU, LAY and POM can be used for both the heavy chain and the light chain. Alternatively, human germline sequences may be used; these are available at:
http://www2.mrc-lmv.cam.ac.uk/vbase/list2.php.

In a humanised antibody molecule of the present invention, the acceptor heavy and light chains do not necessarily need to be derived from the same antibody and may, if desired, comprise composite chains having framework regions derived from different chains. The framework regions need not have exactly the same sequence as those of the acceptor antibody. For instance, unusual residues may be changed to more frequently-occurring residues for that acceptor chain class or type. Alternatively, selected residues in the acceptor framework regions may be changed so that they correspond to the residue found at the same position in the donor antibody (see Reichmann et al 1998, Nature, 332, 323-324). Such changes should be kept to the minimum necessary to recover the affinity of the donor antibody. A protocol for selecting residues in the acceptor framework regions which may need to be changed is set forth in WO 91/09967. Derivatives of frameworks may have 1, 2, 3, 4, 5, 6, 7, 8, 9, 10 or more amino acids replaced with an alternative amino acid, for example with a donor residue. Donor residues are residues from the donor antibody, i.e. the antibody from which the CDRs were originally derived, in particular the residue in a corresponding location from the donor sequence is adopted. Donor residues may be replaced by a suitable residue derived from a human receptor framework (acceptor residues).

The residues in antibody variable domains are conventionally numbered according to a system devised by Kabat et al. This system is set forth in Kabat et al., 1987, in Sequences of Proteins of Immunological Interest, US Department of Health and Human Services, NIH, USA (hereafter “Kabat et al. (supra)”). This numbering system is used in the present specification except where otherwise indicated. The Kabat residue designations do not always correspond directly with the linear numbering of the amino acid residues. The actual linear amino acid sequence may contain fewer or additional amino acids than in the strict Kabat numbering corresponding to a shortening of, or insertion into, a structural component, whether framework or complementarity determining region (CDR), of the basic variable domain structure. The correct Kabat numbering of residues may be determined for a given antibody by alignment of residues of homology in the sequence of the antibody with a “standard” Kabat numbered sequence. The CDRs of the heavy chain variable domain are located at residues 31-35 (CDR-H1), residues 50-65 (CDR-H2) and residues 95-102 (CDR-H3) according to the Kabat numbering system. However, according to Chothia (Chothia, C. and Lesk, A.M. J. Mol. Biol., 196, 901-917 (1987)), the loop equivalent to CDR-H1 extends from residue 26 to residue 32. Thus unless indicated otherwise ‘CDR-H1’ as employed herein is intended to refer to residues 26 to 35, as described by a combination of the Kabat numbering system and Chothia's topological loop definition. The CDRs of the light chain variable domain are located at residues 24-34 (CDR-L1), residues 50-56 (CDR-L2) and residues 89-97 (CDR-L3) according to the Kabat numbering system.

In one embodiment, the invention extends to an antibody sequence disclosed herein. In another it extends to humanized versions of such antibodies.

The skilled person is able to test variants of CDRs or humanised sequences in any suitable assay such as those described herein to confirm activity is maintained.

In one embodiment, variant binding molecules may be identified by identifying such binding molecules that are able to cross-block specific binding molecules set out herein. Cross-blocking antibodies can be identified using any suitable method in the art, for example by using competition ELISA or BIAcore assays where binding of the cross blocking antibody to antigen (TNFR2) prevents the binding of an antibody of the present invention or vice versa. Such cross blocking assays may use cells expressing TNFR2 as a target. In one embodiment, flow cytometry is used to assess binding to cells expressing TNFR2.

Variant binding molecules may be employed where they still retain the desired properties of binding molecules of the present invention. Hence, binding molecules and in particular antibodies with degrees of sequence identity to specific ones set out herein are also provided. The sequence identity may be over the entire length of a polypeptide or it may be in relation to specific portions such as just over the variable regions or just over the CDR sequences. Degrees of identity and similarity can be readily calculated (Computational Molecular Biology, Lesk, A.M., ed., Oxford University Press, New York, 1988; Biocomputing. Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987, Sequence Analysis Primer, Gribskov, M. and Devereux, J., eds., M Stockton Press, New York, 1991, the BLAST™ software available from NCBI (Altschul, S. F. et al., 1990, J. Mol. Biol. 215:403-410; Gish, W. & States, D. J. 1993, Nature Genet. 3:266-272. Madden, T. L. et al., 1996, Meth. Enzymol. 266:131-141; Altschul, S. F. et al., 1997, Nucleic Acids Res. 25:3389-3402; Zhang, J. & Madden, T. L. 1997, Genome Res. 7:649-656). The present invention also extends to polypeptide sequences disclosed herein and sequences at least 80% similar or identical thereto, for example 85% or greater, such 90% or greater, in particular 95%, 96%, 97%, 98% or 99% or greater similarity or identity. In one embodiment, a variant has at least 80% sequence identity. In another embodiment, a variant has at least 90% sequence identity. In another embodiment, a variant has at least 95% sequence identity. In one embodiment a sequence may have at least 99% sequence identity to at least one of the specific sequences provided herein. Anywhere a variant is referred to herein it may have such a degree of sequence identity/“Identity”, as used herein, indicates that at any particular position in the aligned sequences, the amino acid residue is identical between the sequences. “Similarity”, as used herein, indicates that, at any particular position in the aligned sequences, the amino acid residue is of a similar type between the sequences. For example, leucine may be substituted for isoleucine or valine. Other amino acids which can often be substituted for one another include but are not limited to:
phenylalanine, tyrosine and tryptophan (amino acids having aromatic side chains);
lysine, arginine and histidine (amino acids having basic side chains);
aspartate and glutamate (amino acids having acidic side chains);
asparagine and glutamine (amino acids having amide side chains); and
cysteine and methionine (amino acids having sulphur-containing side chains).

In one embodiment, a variant may have from one to ten, such as one, two, three, four, five or up to those values of amino acid sequence changes or at least those values, or up to those values, so long as the variant is still able to specifically bind TNFR2 and preferably act as an agonist of it in the formats described previously. In another embodiment, an antibody of the present invention may have at least five, six, seven, eight, nine, ten, eleven or twelve amino acid sequence changes compared to the CDRs of one of the specific antibodies set out herein, for example is may have that number of sequence changes in the six CDRs making up the antigen-binding site. In one embodiment, where a binding molecule comprises a VHH domain, the VHH domain may comprise a set of three CDRs which have the above mentioned numbers of amino acid sequence changes compared to a specific set of three CDRs for a VHH provided herein. An antibody of the present invention may have that number of sequence changes in the CDRs compared to the specific antibody set out herein. It may have up to that number of sequence changes. It may have at least that number of amino acid sequence changes. Such variant antibody molecules will typically retain the ability to specifically bind TNFR2. They may also retain out of the other functions set out herein.

In one preferred embodiment, where a variant pair of variable regions is employed they will have at least 90% sequence identity to the specific ones set out herein. In another they will have at least 90% sequence identity. In another they will have at least 95% sequence identity. In one embodiment, they will have no more than ten amino acid sequence changes. In another embodiment, they will have no more than five amino acid sequence changes compared to the specific sequences. In one embodiment, they will have one, two, or three sequences changes compared to the specific sequences. Variants will retain the ability to bind TNFR2. For instance, variants may have at least 50% of the binding activity of the specific sequence. In another embodiment, they will have at least 75% of the binding activity. In one embodiment, they will have at least the same binding activity. In one embodiment, the sequence changes will only be in the framework regions. In another embodiment, the sequence changes will be conservative amino acid changes.

In embodiments, where a set of CDRs is identified, variant sets may be employed. For instance, sets with up to a total of ten amino acid changes. For instance, variants with up to five amino acid sequence changes. In one embodiment, one, two, or three amino acid changes may be present. The amino acid sequence changes may all be conservative in one embodiment, The variant CDRs will still allow the antigen-binding site to bind TNFR2. For instance, variants may have at least 50% of the binding activity of the specific sequence. In another embodiment, they will have at least 75% of the binding activity. In one embodiment, they will have at least the same binding activity.

In one embodiment, variant sequences will have the ability to block the binding of a binding site comprising the specific sequences. In one embodiment, a variant sequence will compete for binding. The Examples of the present application include illustrative assays which may be used to measure binding and hence blocking/competition.

It will be appreciated that this aspect of the invention also extends to variants of the specific binding molecules, and in particular antibodies, including humanised versions and modified versions, including those in which amino acids have been mutated in the CDRs to remove one or more isomerisation, deamidation, glycosylation site or cysteine residue as described herein above.

In a preferred embodiment where sequence identity is referred to it is in respect of the full length of the two sequences being compared. For instance, the level of sequence identity specified may be over the length of a CDR. It may be over the length of a variable region. It may be over the entire length of the sequence, for instance over the entire length of the shorter of the two sequences.

Anywhere a specific sequence is indicated a variant may be employed, for instance one as defined above or below. Variant CDRs may be employed, as may be variant sets of CDRs. Variant variable regions may be employed, as may be pairs of such variable regions. Variant hinge regions may be employed. Variant constant regions may be employed. In one embodiment, a variant may be of the overall polypeptide. In one embodiment, such variants have at least 90% sequence identity compared to the specific sequence. In one embodiment, they have at least 95% sequence identity. A variant antigen-binding site will still retain TNFR2 binding activity. A variant binding molecule will typically retain TNFR2 agonist activity. A variant may retain any of the functions set out here. Preferred variants have one to ten amino acid changes. Further variants have one to five amino acid changes. Preferred variants have one, two or three amino acid changes. Other variants have only one amino acid change compared to the specific sequence. In one embodiment, a variant may be a biosimilar of a binding molecule as set out herein.

### Further Illustrative Antibody and CDR Sequences

The Examples of the present application include various specific antibody molecules, including molecules with TNF ligands as part of their structure to help illustrate the invention and its advantages. The invention is not limited to those sequences. In one embodiment a binding molecule, and in particular an antibody, of the present invention may comprise specific sequences from those illustrative antibodies, or variants of them. Variant antibodies are also provided, including variants as defined here, particularly those that specifically bind TNFR2, but not TNFR1, in an FcγR independent manner.

Table 3 of the present invention sets out the various pairs of expression constructs that were used to express a particular antibody. The Table also sets the polypeptides that are expressed by the pairs of vectors. In one embodiment, the present invention provides a binding molecule that comprises one of the pairwise combinations of polypeptides set out by Table 3. The original antibody clones used to generate the non-VHH based antibodies in the Examples are mouse antibodies. In one embodiment, the present invention provides a binding molecule comprising one of the pairwise combinations of polypeptides set out in Table 3 where the mouse CDR sequences are retained but the rest of the antibody has been humanised. In another embodiment, a binding molecule of the present invention comprises one of the pairwise combinations of polypeptides set out in the Table except the whole of the mouse sequences have been swapped for those of a human antibody that is able to specifically bind TNFR2. In one embodiment, a binding molecule that cross-blocks the binding molecule that results from the pairwise combinations of polypeptides set out in Table 3 is employed. In another embodiment, a binding molecule that cross-reacts with a binding molecule that results from the pairwise combinations of polypeptides set out in Table 3 is employed. In one embodiment, a binding molecule of the present invention, and in particular an antibody, will comprise an antigen-binding site comprising a set of six CDRs from those of the pairwise combinations of heavy and light chains set out in Table 3 of the present application. In another embodiment, a binding molecule and in particular an antibody of the present invention will comprise a pair of light and heavy chain variable regions pair from those that are set out in Table 3.

Table 4 of the present applications sets out the SEQ ID NOs for the various polypeptides expressed by the expression vectors used in the Examples of the present application. In one embodiment, a binding molecule of the present invention comprises at least one of those polypeptides. In another embodiment, a binding molecule of the present invention comprises at least one of those polypeptides, where the polypeptide has been humanised, with the mouse CDR sequences retained, but the rest of the mouse sequences replaced by human ones. In a preferred embodiment, the binding molecule may comprise two of those polypeptides, or humanized versions where all but the mouse CDRs have been replaced by the corresponding sequences. In one embodiment, a binding molecule of the present invention, and in particular an antibody, will comprise an antigen-binding site comprising a set of six CDRs from those of the pairwise combinations of heavy and light chain SEQ ID Nos set out in Table 4 of the present application. In another embodiment, a binding molecule and in particular an antibody of the present invention will comprise a pair of light and heavy chain variable regions pair from the variable regions in the SEQ ID Nos that are set out in Table 4.

Table 5 of the present application sets out the CDR sequences from the specific antibodies used as a starting point for the antibody formats set out in the present application. The three CDRs from a light chain are set out grouped together, as are the three CDRs from the heavy chains, with the CDRs from a light and heavy chains also grouped together. In one embodiment, a binding molecule of the present invention will comprise a set of three light chain CDRs as set out in Table 5. In another embodiment, it will comprise a set of three heavy chain CDRs as set out in Table 6, so both the light and heavy chain CDR sequences from one of the clones or functional variants of such sequences. In one embodiment, a binding molecule of the present invention comprises the CDRs for more than one specificity, so the binding molecule may, for instance, comprise two polypeptides that collectively comprise the six CDRs from one of the antibodies as set out in Table 5, but the binding molecule may comprise a further set of six CDRs for another of the antibodies, for example present as a scFv which is part of the binding molecule. In one embodiment, a binding molecule of the present invention is triparatopic for TNFR2 and so has three different specificities for TNFR2. In one embodiment, at least one of the specificities is conferred by a set of six CDRs from Table 5 or variants of those CDRs. In another embodiment, at least two of the specificities are conferred by sets of six CDRs from Table or variants thereof. In a further embodiment, all three of the specificities are. In another embodiment a binding molecule of the present invention may be tetraparatopic, for example with at least one, two, or three of the specificities being conferred by sets of six CDR set out in Table 5 or variants thereof. In one embodiment all four specificities may be conferred by sets of six CDRs from Table 5 or variants thereof.

In one preferred embodiment, a binding molecule of the present invention comprises an antigen binding site comprising a set of six CDRs from the C40 clone. In another preferred embodiment the binding molecule comprises an antigen binding site comprising a set of six CDRs that are from the C19 clone. In a further preferred embodiment an antibody has both an antigen binding site comprising a set of six CDRs from the C40 clone and also a further antigen-binding site comprising a set of six CDRs from the C19 clone. In one preferred embodiment, the CDRs are from the C4 clone. In one embodiment, all of the antigen-binding sites of an antibody of the present invention comprise the same set of six CDRs. In another embodiment, different antigen-binding sites of the antibody comprise a different set of six CDRs. In one embodiment, rather than the six CDRs from C40, C19, or C4 a variant set of six CDRs may be employed, such as any of the variants discussed herein.

In one embodiment any of the antibody formats discussed herein may be provided where the six CDRs of the antigen binding sites are those of C40. In another embodiment, they are those of C19. In another embodiment, they are those of C4. In another embodiment the CDRs are variants of those from C40, C19 or C4 which still retain the ability to bind to TNFR2.

The present invention also provides variants of the above binding molecules and specific binding molecules employed in the Example. For example, in one embodiment a variant may have from one, two, three, four, five, six or more amino acid sequence changes in the CDR sequences of an antigen-binding site compared to the six CDRs set out in Table 5 as a set, but still be able to act as an agonist of TNFR2. In another embodiment, it may have six or more such amino acid changes, for example it may have from six to fifteen such changes, for instance six, seven, eight, nine, ten or more such changes. In one embodiment, an antibody of the present invention may have at least the number of amino acid sequence changes specified. In another embodiment, it may have up to the value recited. In a further embodiment, it may have the recited number of amino acid sequence changes. In one embodiment, the sequence changes may be conservative amino acid sequence changes. In another embodiment, the sequence changes may include non-conservative sequence changes. Various specific modifications are discussed herein and they may be present in such variant molecules. In any of the embodiments set out herein where a specific antibody format is referred to in a further embodiment a binding molecule may be provided, or employed, which is the antibody of that format from the Examples of the present application, but which has been modified to replace all but the mouse CDR sequences with the human CDR sequences.

### Preferred C4 and C4 Variant Antigen-Binding Sites

In one particularly preferred embodiment, a binding molecule of the present invention comprises an antigen-binding site comprising sequences of the C4 clone or variant sequences thereof. For instance, a binding molecule may comprise an antigen-binding site comprising the light and heavy chain variable regions from the C4 clone. It may comprise the six CDRs of the C4 clone. Variant C4 variable region and CDR sequences may be also employed, with the present application setting out preferred examples of those.

The light and heavy chain variable regions for the C4 clone are set out as SEQ ID Nos: 295 and 296 respectively and may be employed in an antigen-binding site. The light chain variable region CDR1, CDR2, and CDR3 sequences for the C4 clone are set out as SEQ ID Nos: 69/70/71 respectively and those three CDRs may be employed in an antigen binding site. The heavy chain variable region CDR1, CDR2, and CDR3 sequences for the C4 clone are provided as SEQ ID Nos: 102/103/104 respectively and those three CDRs may be employed in an antigen binding site. In a preferred embodiment, an antigen-binding site comprises all six CDRs from CDR, so light chain CDRs of SQ ID NOs: 69/70/71 and the heavy chain CDRs of SEQ ID Nos: 102/103/104. Variant C4 sequences may also be employed. In one embodiment, a variant set of those six CDRs may be employed which have a maximum of 10 amino acid sequence changes compared to the sequence of the specific CDRs. In one embodiment, the variant set will have a maximum of 5 amino acid sequence changes. In one embodiment, they may have one, two or three amino acid sequence changes compared to the sequences of the specific set of six CDRs.

Particularly preferred C4 variant light chain variable regions are provided as SEQ ID Nos: 262 to 265 and may be employed. The CDRs from those light chain variable regions are provided as SEQ ID Nos: 266 to 277 and may be employed. A set of variant CDRs which may have up to 10 amino acid sequence changes compared to the sequence of the specific light chain variable CDRs. For instance, a variant set may have up to five amino acid sequence changes compared to the specific Particularly preferred heavy chain variable regions are set out as SEQ ID NOs: 278 to 281 and may be employed. The CDRs from those heavy chain variable regions are set out as SEQ ID NOs: 282 to 293 and may be employed. Variants may be employed with up to ten amino acid sequence changes for the heavy chain CDRs. In one embodiment, a variant set with up to five amino acid sequence changes compared to the sequence of the set of heavy chain CDRs may be employed.

In one preferred embodiment, a binding molecule of the present invention comprises an antigen binding site comprising a light chain variable region selected from one of SEQ ID NOs: 295, 262 to 265, or a variant of any thereof and a heavy chain variable region selected from one of SEQ ID NOs: 306, 278 to 281, or a variant of any thereof. In one preferred embodiment, a binding molecule comprises an antigen binding site comprising a light chain variable region with the CDR1, CDR2, and CDR3 of SEQ ID NOs 69, 70, and 71 or variants thereof and a heavy chain variable region with the CDR1, CDR2, and CDR3 of SEQ ID NOs: 102, 103, and 104 or variants thereof. In a further preferred embodiment, a binding molecule comprises an antigen binding site comprising a set of three light chain CDRs for CDR1/CDR2/CDR3 selected from SEQ ID NOs: 266/267/268; 269/270/271; 272/273/274; 275/276/278; or variants thereof. In a further preferred embodiment, a binding molecule comprises an antigen-binding site comprising a set of three heavy chain CDRs for CDR1/CDR2/CDR3 selected from SEQ ID NOs 282/283/284; 285/286/287; 288/289/290; 291/292/293; or variants thereof. In one embodiment, the binding site comprises both such light and heavy chain CDRs. For instance, in one embodiment, a binding molecule comprises the CDR for the light chain, the heavy chain, or both from one of the specific C4 variants provided herein.

C4 and C4 variant based binding sites may be employed in any of the antibody formats discussed herein that comprise an antigen-binding site comprising a light and heavy chain variable region. In one preferred embodiment, such antigen-binding sites are employed in a tetravalent binding molecule of the present invention. In one particularly preferred embodiment, the set of six CDRs, or a pair of VL and VH sequences, from the C4 clone or one of the specific variants of the C4 clone provided herein are employed in one of the formats shown in the Figures, in particular those shown in FIGS. 1 and 7. They may also be employed in the format shown in FIG. 29A. Variants of the specific sequences may also be employed.

In an especially preferred embodiment of the present invention, a binding molecule of the present invention comprises a polypeptide having the sequence of SEQ ID No: 259 and a polypeptide having the sequence of SEQ ID NOs: 261. In one embodiment, a binding molecule consists of two of each such polypeptides. In a further preferred embodiment, the binding molecule comprises two such polypeptides, but where the light chain variable regions of the SEQ ID NO: 261 are swapped for the variable regions of one of SEQ ID Nos: 262 to 265 or a variant thereof. In a further preferred embodiment, the binding molecule comprises two such polypeptides, but where the heavy chain variable region of SEQ ID NO: 259 are swapped for the variable regions of one of SEQ ID Nos: 278 to 281 or a variant thereof. In one preferred embodiment, both the heavy and light chain variable regions are swapped in that way.

In a further preferred embodiment, a binding molecule comprises a polypeptide with the sequence set out in FIG. 54 or 55. In one embodiment, a binding molecule comprises:
(a) a polypeptide having the sequence of SEQ ID NO: 332 or a variant sequence with at least 90% sequence identity; and
(b) a polypeptide having the sequence of SEQ ID NO: 333 or a variant sequence with at least 90% sequence identity.

In one embodiment, a binding molecule comprises two polypeptides as defined in (a) and two polypeptides as defined in (b).

In one embodiment, a binding molecule is one comprising the light and heavy chain variable regions present in SEQ ID NOs: 332 and 333 or variant sequences thereof with at least 90% sequence identity where the binding molecule is able to act as a TNFR2 agonist.

In one embodiment, a binding molecule comprises:
(c) a polypeptide having the sequence of SEQ ID NO: 334 or a variant sequence with at least 90% sequence identity; and
(d) a polypeptide having the sequence of SEQ ID NO: 335 or a variant sequence with at least 90% sequence identity.

In one embodiment, a binding molecule comprises two polypeptides as defined in (a) and two polypeptides as defined in (b).

In one embodiment, a binding molecule is one comprising the light and heavy chain variable regions present in SEQ ID NOs: 334 and 335 or variant sequences thereof with at least 10% sequence identity where the binding molecule is able to act as a TNFR2 agonist.

In one embodiment, a binding molecule comprises:
(e) a polypeptide having the sequence of SEQ ID NO: 336 or a variant sequence with at least 90% sequence identity; and
(f) a polypeptide having the sequence of SEQ ID NO: 337 or a variant sequence with at least 90% sequence identity.

In one embodiment, a binding molecule comprises two polypeptides as defined in (a) and two polypeptides as defined in (b).

In one embodiment, a binding molecule is one comprising the light and heavy chain variable regions present in SEQ ID NOs: 336 and 337 or variant sequences thereof with at least 10% sequence identity where the binding molecule is able to act as a TNFR2 agonist.

In one preferred embodiment, all of the antigen-binding sites specific for TNFR2 in a binding molecule of the present invention are one of the C4 or C4 variant based binding sites discussed herein.

Table 10 sets out examples of preferred light and heavy chain pairings for C4 variant binding antigen-binding sites. In one preferred embodiment, a pair of sequences as indicated in table 10 may be employed as the antigen-binding site. In another embodiment, a pair of sequences with at least 90% sequence identity to one of those pairs of sequences may be employed. Variants with 95% sequence identity may be employed.

C4 and the specific C4 variants set out herein may be employed in any of the binding formats set out herein, as may variants of their CDRs, sets of light chain CDRs, sets of heavy chain CDRs, sets of six CDRs, light chain variable regions, heavy chain variable regions, and pairs of variable regions. Specific pairs of C4 variants that may be employed are set out in Table 10 and variant sequences of those specific pairs may also be employed. FIGS. 54 and 55 show particularly preferred C4 variant based polypeptides which may be employed, particularly the heavy and light chains indicated.

### Preferred VHH Sequences

In one particularly preferred embodiment, the TNFR2 binding domains of a binding molecule of the present invention are VHH domains. Hence, in one preferred embodiment, all of the TNFR2 binding domains in the binding molecule are VHH domains. The Examples of the present application describe the generation of 14 VHH regions specific for TNFR2, the sequences for which are provided as SEQ ID NOs: 140 to 153. Each of those specific VH regions represent a preferred VHH domain to be employed in a binding molecule of the present invention, including in any of the binding molecule formats described herein. Variant sequences of such specific VHH domains may also be employed provided that the variant is still able to bind to TNFR2 and the binding molecule acts as an agonist of TNFR2.

An especially preferred VHH domain to be employed in a binding molecule is the C188 VHH binding domain of SEQ ID NO: 140 or a variant thereof. The three CDRs of C188 are provided as SEQ ID NOs: 154/155/156. In one preferred embodiment, a binding molecule comprises an antigen binding site comprising those three CDRs. In one preferred embodiment, all of the antigen-binding sites specific for TNFR2 comprise those three CDRs. In addition, variant CDRs may be employed, provided that the binding site is still able to bind TNFR2.

As described in the Examples of the present application, specific variant sequences of C188 have been generated as C188-VHH_1 to C188-VHH_15, the sequence of which is provided as SEQ ID NOs: 198 to 212 respectively. Hence, in a one embodiment, a TNFR2 binding domain of a binding molecule of the present invention has as VHH domain of one of SEQ ID NOs: 198 to 212. Preferably all of the TNFR2 binding domains have that same sequence. In a particularly preferred embodiment, the variant has the sequence of one of SEQ ID NOs: 198 to 211. Again, preferably all of the binding domains have the same sequences. In one embodiment, variant VHH binding domains may be employed with at least 90% sequence identity to one of SEQ ID NOs: 198 to 211. In another embodiment, the level of sequence identity is at least 95%. Such variants will still be able to act as TNFR2 agonists.

In a further preferred embodiment, a binding molecule comprises an antigen binding site which is one of the specific C188 VHH variants provided as SEQ ID Nos: 198 to 211 or is a variant of such a sequence. In one embodiment, all of the TNFR2 specific binding sites are such a VHH variant binding domain. In one embodiment, a binding molecule comprises a set of three CDRs from one of those C188 variant VHH binding domains. For instance, in one embodiment, a binding molecule comprises an antigen-binding domain comprising a set of three CDRs selected from 213/214/215; 216/217/218; 219/220/221; 222/223/224; 225/226/227; 227/229/230; 231/232/233; 234/235/236; 237/238/239; 240/241/242; 243/244/245; 246/247/248; 249/250/251; 252/253/254; 255/256/257; or a variant set thereof. In a preferred embodiment, a binding molecule comprises an antigen-binding domain comprising a set of three CDRs selected from 213/214/215; 216/217/218; 219/220/221; 222/223/224; 225/226/227; 227/229/230; 231/232/233; 234/235/236; 237/238/239; 240/241/242; 243/244/245; 246/247/248; 249/250/251; 252/253/254; or a variant set thereof wherein the variant has a total of up to ten amino acid sequence changes but the VHH can still bind TNFR2. In one embodiment, the variant has up to five amino acid sequence changes but is still able to bind TNFR2.

In a particularly preferred embodiment, the binding molecule employing such VHH domains is tetravalent or hexavalent binding for such a VHH, such as one of the formats set out herein. The specific VHH regions of SEQ ID NOs: 140 to 153 and 198 to 212 may be employed in any of the binding molecule formats set out herein, including those shown in the Figures and Examples. In one embodiment, the specific VHH regions of SEQ ID NOs: 140, 142 to 145, 149, 151, and 153 may be employed in any of the binding molecule formats set out herein, including those shown in the Figures and Examples. In a particularly preferred embodiment, a binding molecule may comprise an antigen-binding site comprising a VHH having a variable region selected from one of those of SEQ ID NOs: 198 to 211, or a variant thereof with at least 90% sequence identity that is still able to bind TNFR2. In one embodiment, one of the binding molecule formats in the Examples or Figures may be modified to use such an antigen-binding site.

FIGS. 40 and 41 illustrate particularly preferred formats for VHH antibodies. The hexavalent formats shown are particularly preferred. Any of the VHH domains or set of three CDRs from those VHH domains set out herein may be employed in a VHH based binding molecule, particularly those of the C188 or specific C188 variants set out herein. A particularly preferred format is the 3×VHH-Fc format, optionally where linkers are present between the VHH binding domains. An especially preferred binding molecule comprises two polypeptides of SEQ ID NO: 197. Further particularly preferred binding molecules comprise two polypeptides of the sequence corresponding to SEQ ID NO: 197, except that the VHH domains are those of one of SEQ ID NOs: 198 to 212. Further particularly preferred binding molecules comprise two polypeptides of the sequence corresponding to SEQ ID NO: 197, except that the VHHs domains are different and include a set of three CDRs from one of SEQ ID NOs: 213 to 257.

In one embodiment, a binding molecule comprises a polypeptide having the amino acid sequence of SEQ ID NO: 331 or a variant thereof with at least 90% sequence identity. In another embodiment, the binding molecule comprises three such polypeptides. In one embodiment, the binding molecule comprises a polypeptide which is a variant of the amino acid sequence of SEQ ID NO: 331 with a different hinge region with the variant having at least 90% amino acid sequence identity. In another embodiment, it comprises three such polypeptides. In another embodiment, a binding molecule comprises an antigen-binding site that comprises the variable region present in SEQ ID NO: 331 or a variant with at least 90% sequence identity which is still able to act as an agonist. In one embodiment, the binding molecule, comprises an antigen binding site comprising the three CDRs present in the amino acid sequence of SEQ ID NO: 331.

In one embodiment, the binding molecule comprising a variant of a specific VHH region discussed herein, such as one of those set out above. For instance, a variant may be one with at least 80% amino acid sequence identity to one of the specific sequences. It may have at least 90% sequence identity. It may have at least 95% sequence identity. It may have at least 98% sequence identity. It may have at least 99% sequence identity. In one embodiment, a variant has from one to ten amino acid sequence changes. In one embodiment, it may have from one to five amino acid sequence changes. It may have one, two, three, four, or five amino acid sequence changes compared to the specific sequence. It may have one, two, or three amino acid sequence changes compared to the specific sequences. Possible variants of a given sequence are discussed elsewhere herein and any such types of variants may be employed. A variant will preferably retain at least the same TNFR2 binding activity. For instance, the variant may have at least the same agonist activity. In one embodiment, a variant may have at least 50% of the activity of the original molecule. In one embodiment, it may have at least 75% of the activity of the original molecule, preferably at least 80%, more preferably at least 90% of the same activity. One preferred binding molecule comprises the sequence of SEQ ID NO: 197, but variants of that sequence may be employed, such as any of the types of variant mentioned herein. The VHH domains of SEQ ID NOs 198 to 212 are also preferred and variants of those may be employed, including any of the types of variant discussed herein.

In one embodiment, a binding molecule may comprise both sdBr binding domains and other non-sdBr binding domains. In one embodiment, a binding molecule may comprise both VHH binding domains, such as the C188 and C188 variant antigen binding domains in combination with non-VHH antigen binding domains. In one embodiment a binding molecule may comprise both a C188, C188 variant binding domain and a binding domain which is a C4 or C4 variant antigen-binding domain.

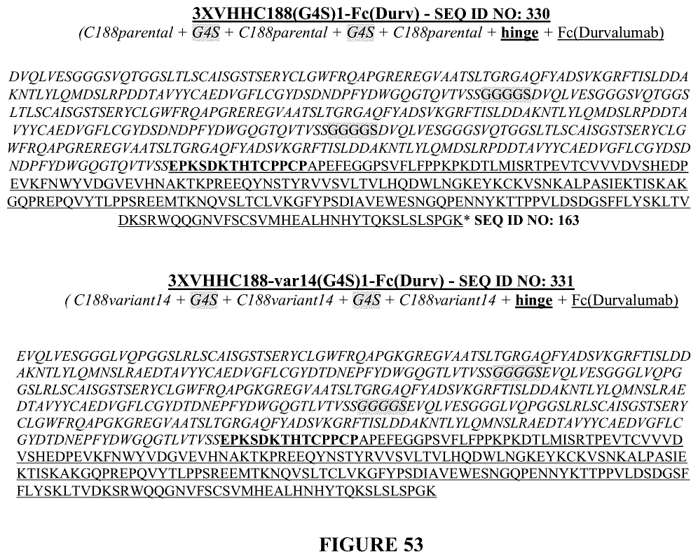

C188 and the specific C188 variants set out herein may be employed in any of the binding formats set out herein, as may variants of their CDRs, their CDR sets, or variable regions. FIG. 53 shows particularly preferred polypeptides which may be employed, as may be variants of those specific sequences.

### Further Preferred Binding Molecules

A particularly preferred binding molecule format is tetravalent. One particular preferred VH domain for employing in binding molecules, particularly tetravalent ones is the C4 VH domain. A particularly preferred format is IgG1-HC:scFv or IgG1-HC:scFv. In a preferred format the VH and VL domains in such formats are those of C4. One particularly preferred format is C4-IgG1(Durv)-HC:scFvC4(G4S)4. In a particularly preferred embodiment, such a binding molecule comprises two polypeptides, a light chain sequence of SEQ ID NO: 261 and a heavy chain sequence of SEQ ID NO: 259. In one embodiment a binding molecule is provided that comprises such polypeptides, except the VH and/or VL regions is one of the specific such regions set out herein. As described in the Examples, variants of the C4 VL sequences were generated having the sequences set out in SEQ ID NO: 262 to 265 any of those VL sequences may replace those in the C4-IgG1(Durv)-HC:scFvC4(G4S)4 molecule. As described in the Examples, variants of the C4 VH sequences were generated having the sequences set out in SEQ ID NO: 278 to 281, any of those VH sequences may replace those in the C4-IgG1(Durv)-HC:scFvC4(G4S)4 molecule. In one embodiment, both the VL and VH region sequences are ones selected from SEQ ID Nos: 262 to 265 for the light chain and SEQ ID NOs: 278 to 281 for the heavy chain. In one preferred embodiment the VL and VH sequences used are respectively those if SEQ ID NOs: 295 and 296.

### Assays

In one embodiment, a functional assay may be employed to determine if a binding molecule of the present invention has a particular property or properties, for instance such as any of those mentioned herein. Hence, functional assays may be used in evaluating a binding molecule, an in particular an antibody, of the present invention. In one embodiment, one or more of the assays described in the Examples of the present application may be employed to assess a particular binding molecule and whether it has a desired property or properties. In a particularly preferred embodiment, one of the assays described in Example 1 may be employed.

A binding molecule of the present invention is able to bind TNFR2. The ability of a binding molecule of the present invention, or a candidate binding molecule, to bind TNFR2 may be assessed in a variety of ways. For example, in one embodiment the ability of the binding molecule to bind TNFR2 is assessed by employing TNFR2 protein, such as by using techniques like surface plasmon resonance using TNFR2, or a portion thereof, bound to a chip. In a particularly preferred embodiment the ability of a binding molecule to bind TNFR2 will be assessed using; a cell expressing TNFR2 on its surface. In one embodiment, candidate molecules are labelled and then screened for their ability to bind cells expressing TNFR2, using techniques such as ELISA or flow cytometry. In another embodiment, candidate molecules may be incubated with cells expressing TNFR2 and then bound candidate molecules detected using secondary agents such as a labelled antibody specific for the species of the candidate molecules. In one embodiment, a binding molecule of present invention is labelled, for example using luciferase-tagged (e.g. Gaussia princeps luciferase (GpL)) variants of a binding molecule, an in particular antibody or the fusion proteins, for example as described in Kums et al., MAbs. 2017 April; 9(3):506-520). Such tagged binding molecules may also be used in competitive binding assays.

In one embodiment, a binding molecule of the present invention is able to: (a) oligomerise TNFR2 on the surface of a cell expressing TNFR2, preferably to form hexamers of TNFR2 or higher-order clusters of TNFR2 trimers on the cell surface; (b) trigger TNFR2 signalling; and/or (d) stimulate proliferation of leucocytes, preferably T cells, more preferably Treg cells. In one embodiment, a binding molecule of the present invention may bind pre-existing higher order complexes of TNFR2 receptors on the cell surface and in particular trimers of TNFR2 on the cell surface. For instance, in one embodiment a binding molecule of the present invention may bring about the formation of a super-cluster of TNFR2 receptors either on the same cell, or bridging two cells, or both.

In one embodiment, the ability of a candidate molecule to bring about oligomerisation of individual TNFR2 molecules on the surface of a cell expressing TNFR2 is measured. In one embodiment, a binding molecule of the present invention will be able to bring about such oligomerisation. For example, in one embodiment, a binding molecule of the present invention will be able to bring about the formation of hexamers or other oligomers of TNFR2 trimers. In one embodiment, the formation of such oligomers may be detected using high resolution microscopy. In one embodiment the formation of oligomers of TNFR2 may be detected using a technique such as PALM, dSTQRM, chemical cross-linking, or FRET.

In one preferred embodiment, the ability of a binding molecule of the present invention to activate TNFR2 is assessed by measuring the downstream effects of TNFR2 activation, rather than directly measuring the binding itself. For example, in one embodiment, whether or not a binding molecule induces IL-8 production may be used as a way to establish if a given binding molecule can induce TNFR2 signaling. NFκB activation in response to TNFR2 signaling typically leads to the production of IL-8 and so that may be used as one way to assess whether a given binding molecule is able to activate TNFR2 signaling as in a desired embodiment, a binding molecule of the present invention is able to activate such signaling. In one embodiment a molecule known to activate TNFR2 signalling may be used as a positive control, such as a binding molecule of the present invention. An example of a preferred cell line that may be used to assess the ability of a binding molecule to bind to TNFR2 and stimulate IL-8 production is HT1080-Bcl2-TNFR2. In one embodiment, the functional assay comprises contacting a binding molecule being assessed to cells expressing TNFR2, such as for instance HT1080-Bcl2-TNFR2 cells, incubating the two overnight, then analyzing a sample of the supernatant for the presence of IL-8. In one embodiment, an ELISA is employed for such assessment, such as a BD OptEIA™ IL8 ELISA kit. In one embodiment, such a method may also comprise a positive control known to stimulate TNFR2 signalling. In one embodiment, such an assay may be used to screen one or more binding molecules for their ability to bind to and activate IL-8 production, such as to screen a library of potential binding molecules and to identify those activating IL-8 product. In one embodiment, such an assay may be used to check that a variant of a binding molecule of the present invention is still as active, or more active, than a specific binding molecule of the present application.

Another assay which may be used to assess a binding molecule of the present invention is a viability assay as TNFR2 can induce in some cell lines/cell types cell death by inhibition of survival proteins (TRAF2, cIAP1, cIAP2) and concomitant upregulation of endogenously produced TNF triggering TNFR1. In a preferred embodiment Kym-1 cells (Schneider et al., 1999) are used for such an assessment. For example, cells expressing TNFR2, in particular Kym-1 cells, seeded on a plate or in wells may be incubated with a test binding molecule, followed by crystal violet staining, then OD at 595 nm. A positive control may be employed that is known to induce cell killing, such as a “cell death”—such as a control mixture (an example of which is 200 ng/ml TNF, 200 ng/ml TRAIL, 200 ng/ml CD95L, 25 μg/ml CHX, 1% (w/v) sodium azide). The values seen may be normalized compared to untreated cells and cells treated with the “death mixture”. Again, such methods may be used to screen for binding molecules with the desired properties.

In one preferred embodiment, a binding molecule of the present invention will both induce IL-8 production, for example as assessed in the assay discussed above, and also be able to induce cell death, for example as assessed in the assay discussed above. In another preferred embodiment, a binding molecule of the present invention will induce IL-8 production, but not significantly induce cell death, for instance as measured using the assays in the Examples of the present application.

In one preferred embodiment, a binding molecule of the present invention will be able to bind to TNFR2 and initiate signaling through activation of the alternative NFκB pathway. In a particularly preferred embodiment, a binding molecule of the present invention will be able to activate the alternative NFκB pathway as shown by processing of p100 to p52. In one embodiment, cells expressing TNFR2 are contacted with the binding molecule being assessed, the cells then harvested, and a lysate of the cells assessed to measure p100 and p52 levels. In one embodiment, Western blotting is performed on the cell lysates using antibodies against p100 and p52 to study whether the former is processed to the latter. Any suitable cells may be used, but in a preferred embodiment Kym-1 cells are used for the assessment. A positive control may be used, such as a binding molecule known to be active. A negative control may be also performed, such as cells that are incubated without a binding molecule and then assessed in the same way. In one preferred embodiment, a binding molecule of the present invention will be able to activate the classical NFκB pathway, for instance as measured by IL-8 production, and also be able to activate the alternative NFκB pathway, such as by assessing whether p100 is processed to p52. In another preferred embodiment, the binding molecule will also be able to activate cell killing, for example as assess using the cell viability assay as described above.

Typically, binding molecules of the present invention do not bind FcγR. In one preferred embodiment, a candidate binding molecule is assessed both for its ability to bind and activate TNFR2, but also for its ability not to bind to and activate FcγR. In one embodiment, the ability of a binding molecule of the present invention to bind Fc receptors and in particular FcγR is assessed. The lack of binding to Fc receptors may be assessed, for instance to determine whether or not CDC or ADCC activity is displayed and preferably neither will be by a binding molecule of the present invention.

In another embodiment, the ability of a binding molecule of the present invention to stimulate activation and/or expansion of cells will be assessed, for example to stimulate particular immune cells in that way, as a binding molecule of the present invention will be typically able to bring about activation and/or expansion of cells such as T cells. In one embodiment, the ability of a binding molecule of the present invention to stimulate Treg cells from PBMC is assessed. In one embodiment, the ability of a binding molecule to expand Tregs is assessed by a method comprising:
isolating PBMC and then culturing the PBMC (for example at high density, such as at 10 million cells per ml);
harvesting the cells and then seeding the PBMC (for example at a lower density of 1 million cells per ml);
incubating the cells with a candidate binding molecule for four days; and
performing analysis to determine the number of cells.

In a preferred embodiment, a negative control is performed where the cells are cultured without contacting with a candidate binding molecule. The cells may be assessed using flow cytometry in particular staining for CD4+CD25+ FoxP3+ cells. In a preferred embodiment the number of FoxP3+CD25+ cells within the CD3+CD4+ cell population is measured. The cells may also be stained with antibodies specific for CD3 and/or CD8. In one preferred embodiment, a binding molecule of the present invention will give higher numbers of CD4+CD25+ FoxP3+ cells compared to incubation without a binding molecule. In another embodiment, a candidate binding molecule may also be compared to a specific binding molecule of the present invention, for example to assess whether a variant binding molecule is also able to expand Tregs to the same or greater degree than the specific binding molecule of the present invention.

In another preferred embodiment, FoxP3-Luci mice are employed to study Treg cell expansion as the mice express luciferase under the control of the mouse FoxP3 promoter, which acts as a marker for Treg cells. For example, such mice may be injected with a candidate TNFR2 agonist then bioluminescence imaging is used to image Treg cells. A positive control with a known TNFR2 agonist may be performed, as may be a negative control. In one embodiment a variant or candidate binding molecule will be compared to a known TNFR2 agonist set out herein and if it results in an equivalent or greater level of Tregs as assessed by the bioluminescence imaging in a preferred embodiment it itself is also classified as a binding molecule of the present invention. Such assessment may also be combined with ex vivo assessment, for example by subsequently sacrificing the animal, isolating cells, and then looking at Treg numbers.

In another preferred embodiment, for testing of human-specific molecules, transgenic mice expressing human TNFR2, or in particular the extracellular domain of human TNFR2, for example obtained after random insertion of a human TNFR2 cDNA into the mouse genome or more specifically in the mouse TNFR2 gene locus, are employed to study Treg levels and in particular expansion. Such transgenic mice can be crossbred with FoxP3-Luci transgenic mice for in vivo imaging of Treg expansion. Upon sacrifice, separate tissues can also be processed via imaging for changed levels of Tregs, versus negative control animals. Treg expansion and Treg/Teff ratios can also be quantitated using flow cytometry, sourcing splenocytes, leukocytes in blood or other tissues. Alternatively, immunodeficient mice such as NSG mice can be injected with human PBMCs or human Tregs and the expansion of Tregs determined via flow cytometry.

The efficacy of a particular binding molecule may be assessed in an in vivo system such as in animal models. For example, various models of graft versus host disease (GvHD) may be employed, with a binding molecule given to such an animal model and then compared to a control animal which is the same animal model for GvHD but which has not been given the binding molecule. In one embodiment, a binding molecule of the present invention will present or reduce the GvHD in the animal model. Other animal models may be used in the same way, for example models of conditions such as psoriasis, inflammatory bowel disease (IBD), lupus, multiple sclerosis, type 1 diabetes, neurological diseases and atherosclerosis. In vivo studies may also be used to see whether a given binding molecule is able to expand Tregs in vivo.

### Conjugates, Fusion Proteins, and Effector Molecules

A binding molecule, an in particular an antibody, of the invention may be conjugated to an effector molecule. Hence, if desired a binding molecule, and in particular an antibody, for use in the present invention may be conjugated to one or more effector molecule(s). It will be appreciated that the effector molecule may comprise a single effector molecule or two or more such molecules so linked as to form a single moiety that can be attached to the binding molecules, an in particular antibodies, of the present invention. Where it is desired to obtain a binding molecule, and in particular an antibody, according to the present invention linked to an effector molecule, this may be prepared by standard chemical or recombinant DNA procedures in which the binding molecule, and in particular antibody, is linked either directly or via a coupling agent to the effector molecule. Techniques for conjugating such effector molecules to antibodies are well known in the art (see, Hellstrom et al., Controlled Drug Delivery, 2nd Ed., Robinson et al., eds., 1987, pp. 623-53; Thorpe et al., 1982, Immunol. Rev., 62:119-58 and Dubowchik et al., 1999, Pharmacology and Therapeutics, 83, 67-123). Particular chemical procedures include, for example, those described in WO 93/06231, WO 92/22583, WO 89/00195, WO 89/01476 and WO 03/031581. Alternatively, where the effector molecule is a protein or polypeptide the linkage may be achieved using recombinant DNA procedures, for example as described in WO 86/01533 and EP0392745. In one embodiment the binding molecules, in particular antibodies, of the present invention may comprise an effector molecule. The term effector molecule as used herein includes, for example, drugs, toxins, biologically active proteins, for example enzymes, antibody or antibody fragments, synthetic or naturally occurring polymers, nucleic acids and fragments thereof e.g. DNA, RNA and fragments thereof, radionuclides, particularly radioiodide, radioisotopes, chelated metals, nanoparticles and reporter groups such as fluorescent compounds or compounds which may be detected by NMR or ESR spectroscopy.

Examples of effector molecules may include cytotoxins or cytotoxic agents including any agent that is detrimental to (e.g. kills) cells. Examples include combrestatins, dolastatins, epothilones, staurosporin, maytansinoids, spongistatins, rhizoxin, halichondrins, roridins, hemiasterlins, taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Effector molecules also include, but are not limited to, antimetabolites (e.g. methotrexate, 6-mercaptopurine, 6-thioguanine, cytarabine, 5-fluorouracil decarbazine), alkylating agents (e.g. mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g. daunorubicin (formerly daunomycin) and doxorubicin), antibiotics (e.g. dactinomycin (formerly actinomycin), bleomycin, mithramycin, anthramycin (AMC), calicheamicins or duocarmycins), and anti-mitotic agents (e.g. vincristine and vinblastine).

Other effector molecules may include chelated radionuclides such as ¹¹¹In and ⁹⁰Y, Lu¹⁷⁷, Bismuth²¹³, Californium²⁵², Iridium¹⁹² and Tungsten¹⁸⁸/Rhenium¹⁸⁸; or drugs such as but not limited to, alkylphosphocholines, topoisomerase I inhibitors, taxoids and suramin. Other effector molecules include proteins, peptides and enzymes. Enzymes of interest include, but are not limited to, proteolytic enzymes, hydrolases, lyases, isomerases, transferases. Proteins, polypeptides and peptides of interest include, but are not limited to, immunoglobulins, toxins such as abrin, ricin A, pseudomonas exotoxin, or diphtheria toxin, a protein such as insulin, tumour necrosis factor, α-interferon, β-interferon, nerve growth factor, platelet derived growth factor or tissue plasminogen activator, a thrombotic agent or an anti-angiogenic agent, e.g. angiostatin or endostatin, or, a biological response modifier such as a lymphokine, interleukin-1 (IL-1), interleukin-2 (IL-2), granulocyte macrophage colony stimulating factor (GM-CSF), granulocyte colony stimulating factor (G-CSF), nerve growth factor (NGF) or other growth factor and immunoglobulins.

Other effector molecules may include detectable substances useful for example in diagnosis. Examples of detectable substances include various enzymes, prosthetic groups, fluorescent materials, luminescent materials, bioluminescent materials, radioactive nuclides, positron emitting metals (for use in positron emission tomography), and nonradioactive paramagnetic metal ions. See generally U.S. Pat. No. 4,741,900 for metal ions which can be conjugated to antibodies for use as diagnostics. Suitable enzymes include horseradish peroxidase, alkaline phosphatase, beta-galactosidase, or acetylcholinesterase; suitable prosthetic groups include streptavidin, avidin and biotin; suitable fluorescent materials include umbelliferone, fluorescein, fluorescein isothiocyanate, rhodamine, dichlorotriazinylamine fluorescein, dansyl chloride and phycoerythrin; suitable luminescent materials include luminol; suitable bioluminescent materials include luciferase, luciferin, and aequorin; and suitable radioactive nuclides include ¹²⁵I, ¹³¹I, ¹¹¹In and ⁹⁹Tc.

In another embodiment, the effector molecule may increase or decrease the half-life of the binding molecule, in particular antibody, in vivo, and/or reduce immunogenicity and/or enhance delivery across an epithelial barrier to the immune system. Examples of suitable effector molecules of this type include polymers, albumin, albumin binding proteins or albumin binding compounds such as those described in WO 05/117984. Where the effector molecule is a polymer it may, in general, be a synthetic or a naturally occurring polymer, for example an optionally substituted straight or branched chain polyalkylene, polyalkenylene or polyoxyalkylene polymer or a branched or unbranched polysaccharide, e.g. a homo- or hetero-polysaccharide. Specific optional substituents which may be present on the above-mentioned synthetic polymers include one or more hydroxy, methyl or methoxy groups. Specific examples of synthetic polymers include optionally substituted straight or branched chain poly(ethyleneglycol), poly(propyleneglycol) poly(vinylalcohol) or derivatives thereof, especially optionally substituted poly(ethyleneglycol) such as methoxypoly(ethyleneglycol) or derivatives thereof.

A binding molecule, in particular an antibody, of the present invention may be conjugated to a molecule that modulates or alters serum half-life. A binding molecule, in particular an antibody, of the invention may bind to albumin, for example in order to modulate the serum half-life. In another embodiment, a binding molecule, in particular an antibody, of the invention may include a peptide linker which is an albumin binding peptide. Examples of albumin binding peptides are included in WO 2015/197772 and WO 2007/106120 the entirety of which are incorporated by reference.

In another embodiment, a binding molecule, in particular an antibody, of the invention is not conjugated to an effector molecule. In one embodiment, a binding molecule, in particular an antibody, of the invention is not conjugated to a toxin. In another embodiment, a binding molecule, in particular an antibody, of the invention is not conjugated to a radioisotope. In another embodiment, a binding molecule, in particular an antibody, of the invention is not conjugated to an agent for imaging.

As well as using conjugation to join an antibody molecule of the present invention to an effector molecule, it may be used to join different parts of the overall antibody or, for instance, to join ligand based TNFR2 binding sites to the antibody portion of the present molecule.

In one embodiment, a binding molecule of the present invention may be a fusion protein. A “fusion protein” as referred to herein is not limited to particular types of fusion proteins as long as the parts of the fusion protein are fused by covalent bonds. For example, the parts of the fusion protein can be fused by expression in one or more single polypeptide chain(s), by one or more disulfide linkages, by chemical conjugation (preferably by chemical conjugation using click chemistry) and/or by any other covalent linkage which is known in the art as a suitable link for proteins. Preferably, the parts of the fusion protein are fused by expression in one or more single polypeptide chain(s) and/or by one or more disulfide linkages. For example, a binding molecule, and in particular an antibody, may comprise a polypeptide which comprises a heavy or light chain constant region that is fusion to the polypeptide sequence for an scFv. In another embodiment, the polypeptide may comprise a heavy chain constant region sequence which is fused to a tenascin peptide sequence, in particular with the tenascin peptide sequence at the C-terminus of the polypeptide. In other embodiments, fusion may be via by one or more disulfide linkages, by chemical conjugation (preferably by chemical conjugation using click chemistry) and/or by any other covalent linkage which is known in the art as a suitable link for proteins.

In one embodiment, a binding molecule of the present invention comprises a moiety that influences the serum half-life of the molecule. For example, a binding molecule of the present invention may comprise a Fc tail, serum albumin, and/or a moiety which is a binder of serum albumin, and PEG. In one preferred embodiment a binding molecule of the invention will either comprise, or be conjugated to, or have a binding site for serum albumin. In one embodiment, the binding molecule is conjugated to serum album or a variant thereof to alter the serum half-life of the binding molecule. In another embodiment, the binding molecule is an antibody that comprises an antigen-binding site for serum albumin. In another embodiment, the binding molecule, and in particular an antibody body, of the present invention may comprise a peptide sequence that binds to serum albumen.

In one embodiment, a binding molecule of the present invention may comprise a linker or linkers. Linkers may be employed to join parts of the binding molecule together. In one embodiment, binding sites are separated by linkers. In one preferred embodiment where a binding molecule comprises at least one linker, it will comprise a glycine/serine linker such as GS or G4S (GGGGS), or multiple copies of one or both such linkers. In one embodiment, single G4S linkers may be used. In one embodiment, a linker may comprise a multiple of the G4S unit, for instance with each linker having 1 to 7 repeats of that sequence. In one embodiment, 1 to 5 G4S linker units are used in series. Any suitable linker may be employed in the present invention, for instance in those embodiments employing the specific G4S linker an alternative embodiment is provided where the linker is not limited to the G4S linker sequence and may be any suitable linker. In one embodiment where three VHH domains are present in series on the same polypeptide linkers may be used to separate the VHH domains, for instance on either side of the middle VHH to separate it from the other VHH domains.

In one embodiment, a binding molecule of the present invention may comprise a hinge region. In case of (tetravalent) IgG formats comprising VH and VL, such hinge region typically connects the CH1 domain to the CH2 domain. In case of VHH, such hinge region typically connects the most C-terminal sdAB/VHH to the Fc portion. In one embodiment, the hinge may be that of IgG1 or 2, or 3 or 4 hinge region. It may be such a human hinge region. In one embodiment, the hinge region is that of the human IgG1 consensus sequence. In one embodiment, the hinge region is that of durvalumab. In one embodiment, the hinge region may be a variant of the durvalumab hinge region.

A number of the polypeptides presented herein include a signal or leader sequence. Where that is the case the especially preferred polypeptide is that where the signal or leader sequence has been removed.

### Pharmaceutical Compositions

In one embodiment, the present invention provides a pharmaceutical composition comprising: (a) a binding molecule, and in particular antibody, of the present invention; and (b) a pharmaceutically acceptable carrier, diluent, and/or excipient. In one embodiment, the pharmaceutical composition comprises a binding molecule of the present invention which is an antibody. In one embodiment, a pharmaceutical composition of the present invention comprises a binding molecule of the present invention as well as a carrier, a stabilizer, an excipient, a diluent, a solubilizer, a surfactant, an emulsifier, a preservative and/or adjuvant. In one embodiment, a pharmaceutical composition of the present invention is in solid or liquid form. In one embodiment, the pharmaceutical composition may be in the form of a powder, a tablet, a solution or an aerosol. In one embodiment, a pharmaceutical composition of the present invention is provided in a frozen form.

A composition of the present invention will usually be supplied as a sterile, pharmaceutical composition. A pharmaceutical composition of the present invention may additionally comprise a pharmaceutically-acceptable adjuvant. In another embodiment, no such adjuvant is present in a pharmaceutical composition of the present invention. The present invention also provides a process for preparation of a pharmaceutical or medicament composition comprising adding and mixing a binding molecule, in particular antibody, of the present invention together with one or more of a pharmaceutically acceptable excipient, diluent or carrier.

Pharmaceutically acceptable carriers in therapeutic compositions may additionally contain liquids such as water, saline, glycerol and ethanol. Such carriers may be used, for example, so that the pharmaceutical compositions to be formulated as tablets, pills, dragees, capsules, liquids, gels, syrups, slurries and suspensions, for ingestion by the patient. The term “pharmaceutically acceptable excipient” as used herein typically refers to a pharmaceutically acceptable formulation carrier, solution or additive to enhance the desired characteristics of the compositions of the present invention. Excipients are well known in the art and include buffers (e.g., citrate buffer, phosphate buffer, acetate buffer and bicarbonate buffer), amino acids, urea, alcohols, ascorbic acid, phospholipids, proteins (e.g., serum albumin), EDTA, sodium chloride, liposomes, mannitol, sorbitol, and glycerol. Solutions or suspensions can be encapsulated in liposomes or biodegradable microspheres. Suitable carriers may be large, slowly metabolised macromolecules such as proteins, polypeptides, liposomes, polysaccharides, polylactic acids, polyglycolic acids, polymeric amino acids, amino acid copolymers and inactive virus particles. Pharmaceutically acceptable salts can be used, for example mineral acid salts, such as hydrochlorides, hydrobromides, phosphates and sulphates, or salts of organic acids, such as acetates, propionates, malonates and benzoates.

In certain embodiments, the pharmaceutical composition may contain formulation materials for the purpose of modifying, maintaining or preserving certain characteristics of the composition such as the pH, osmolarity, viscosity, clarity, color, isotonicity, odour, sterility, stability, rate of dissolution or release, adsorption or penetration. A thorough discussion of pharmaceutically acceptable carriers is available in Remington's Pharmaceutical Sciences (Mack Publishing Company, N.J. 1991). Additional pharmaceutical compositions include formulations involving the binding molecule of the present invention in sustained or controlled delivery formulations. Techniques for formulating a variety of sustained- or controlled-delivery means are known to those skilled in the art. A binding molecule of the present invention may also be entrapped in microcapsules prepared, for example, by coacervation techniques or by interfacial polymerization, in colloidal drug delivery systems, or in macroemulsions. Such techniques are also disclosed in Remington's Pharmaceutical Sciences.

A subject will be typically administered a therapeutically effective amount of a pharmaceutical composition and hence binding molecule of the present invention. The term “therapeutically effective amount” typically refers to an amount of a therapeutic agent needed to treat, ameliorate or prevent a targeted disease or condition, or to exhibit a detectable therapeutic or preventative effect. The precise therapeutically effective amount for a human subject will depend upon the severity of the disease state, the general health of the subject, the age, weight and gender of the subject, diet, time and frequency of administration, drug combination(s), reaction sensitivities and tolerance/response to therapy. This amount can be determined by routine experimentation and is within the judgement of the clinician. Generally, a therapeutically effective amount will be from 0.01 mg/kg to 50 mg/kg, for example 0.1 mg/kg to 20 mg/kg per day. Alternatively, the dose may be 1 to 500 mg per day, such as 10 to 100, 200, 300 or 400 mg per day. In one embodiment, the amount in a given dose is at least enough to bring about a particular function.

In one embodiment, a binding molecule of the present invention may be given in combination with another treatment for the condition being treated and in particular with a TNF-alpha blocker. In one embodiment, the second drug that a binding molecule of the present invention is used in conjunction with is an anti-inflammatory. For example, a binding molecule of the present invention may be provided simultaneously, sequentially, or separately with such a further agent. In another embodiment, a binding molecule of the present invention may be provided in the same pharmaceutical composition as a second therapeutic agent. Examples of drugs which may be provided in the same composition or used in conjunction with a binding molecule of the present invention include TNF-alpha blockers, rapamycin, ustekinumab, or agents which are used as standard-of-care in autoimmune or inflammatory diseases. Examples of TNF-alpha blockers which may be used in conjunction with a binding molecule of the present invention include, but are not limited to, infliximab, adalimumab, etanercept, golimumab, and certolizumab. In one embodiment the second drug is an antibody specific for TNF-alpha. In one embodiment, the second drug administered may be a steroid.

In one preferred embodiment, the therapeutic agent of the invention, when in a pharmaceutical preparation, may be present in unit dose forms. Suitable doses may be calculated for patients according to their weight, for example suitable doses may be in the range of 0.01 to 20 mg/kg, for example 0.1 to 20 mg/kg, for example 1 to 20 mg/kg, for example 10 to 20 mg/kg or for example 1 to 15 mg/kg, for example 10 to 15 mg/kg. To effectively treat conditions of use in the present invention in a human, suitable doses may be within the range of 0.01 to 1000 mg, for example 0.1 to 1000 mg, for example 0.1 to 500 mg, for example 500 mg, for example 0.1 to 100 mg, or 0.1 to 80 mg, or 0.1 to 60 mg, or 0.1 to 40 mg, or for example 1 to 100 mg, or 1 to 50 mg, of a dual targeting protein of this invention, which may be administered parenterally, for example subcutaneously, intravenously or intramuscularly. Such a dose may be, if necessary, repeated at appropriate time intervals selected as appropriate by a physician. A binding molecule of the present invention may be, for instance, lyophilized for storage and reconstituted in a suitable carrier prior to use. Lyophilization and reconstitution techniques can be employed.

The binding molecules and pharmaceutical compositions of this invention may be administered by any number of routes including, but not limited to, oral, intravenous, intramuscular, intra-arterial, intramedullary, intrathecal, intraventricular, transdermal, transcutaneous (for example, see WO 98/20734), subcutaneous, intraperitoneal, intranasal, enteral, topical, sublingual, intravaginal or rectal routes. Hyposprays may also be used to administer the pharmaceutical compositions of the invention. Direct delivery of the compositions will generally be accomplished by injection, subcutaneously, intraperitoneally, intravenously or intramuscularly, or delivered to the interstitial space of a tissue. In one preferred embodiment, administration is via intravenous administration, for example, in one preferred embodiment administration is via intravenous injection. In another preferred embodiment, administration is via subcutaneous administration, for example via subcutaneous injection. The compositions can also be administered into a specific tissue of interest. In some embodiments, a binding molecule of the present invention is administered via site-specific or targeted local delivery techniques. Examples of site-specific or targeted local delivery techniques include various implantable depot sources of the binding molecule or local delivery catheters, such as infusion catheters, indwelling catheters, or needle catheters, synthetic grafts, adventitial wraps, shunts and stents or other implantable devices, site specific carriers, direct injection, or direct application.

Dosage treatment may be a single dose schedule or a multiple dose schedule. Where the product is for injection or infusion, it may take the form of a suspension, solution or emulsion in an oily or aqueous vehicle and it may contain formulatory agents, such as suspending, preservative, stabilising and/or dispersing agents. Alternatively, the binding molecule, in particular the antibody, may be in dry form, for reconstitution before use with an appropriate sterile liquid. In one embodiment, a pharmaceutical composition comprising a binding molecule of the present invention is provided in lyophilised form. If a composition is to be administered by a route using the gastrointestinal tract, the composition will need to contain agents which protect the binding protein from degradation but which release the binding molecule once it has been absorbed from the gastrointestinal tract. In another embodiment, a nebulisable formulation according to the present invention may be provided, for example, as single dose units (e.g., sealed plastic containers or vials) packed in foil envelopes. Each vial contains a unit dose in a volume, e.g., 2 ml, of solvent/solution buffer.

A pharmaceutical composition of the present invention may be provided in a receptacle that provides means for administration to a subject. In one embodiment, a pharmaceutical composition of the present invention may be provided in a prefilled syringe. The present invention therefore provides such a loaded syringe. It also provides an auto-injector loaded with a pharmaceutical composition of the present invention.

In one embodiment the formulation is provided as a formulation for topical administrations including inhalation. Suitable inhalable preparations include inhalable powders, metering aerosols containing propellant gases or inhalable solutions free from propellant gases. Inhalable powders according to the invention containing the active substance may consist solely of the abovementioned active substances or of a mixture of the abovementioned active substances with physiologically acceptable excipient. These inhalable powders may include monosaccharides (e.g. glucose or arabinose), disaccharides (e.g. lactose, saccharose, maltose), oligo- and polysaccharides (e.g. dextranes), polyalcohols (e.g. sorbitol, mannitol, xylitol), salts (e.g. sodium chloride, calcium carbonate) or mixtures of these with one another. Mono- or disaccharides are suitably used, the use of lactose or glucose, particularly but not exclusively in the form of their hydrates.

Particles for deposition in the lung require a particle size less than 10 microns, such as 1-9 microns for example from 1 to 5 μm. The particle size of the active ingredient (such as the antibody or fragment) is of primary importance. The propellant gases which can be used to prepare the inhalable aerosols are known in the art. Suitable propellant gases are selected from among hydrocarbons such as n-propane, n-butane or isobutane and halohydrocarbons such as chlorinated and/or fluorinated derivatives of methane, ethane, propane, butane, cyclopropane or cyclobutane. The above mentioned propellent gases may be used on their own or in mixtures thereof. Particularly suitable propellent gases are halogenated alkane derivatives selected from among TG 11, TG 12, TG 134a and TG227. Of the abovementioned halogenated hydrocarbons, TG134a (1,1,1,2-tetrafluoroethane) and TG227 (1,1,1,2,3,3,3-heptafluoropropane) and mixtures thereof are particularly suitable. The propellent-gas-containing inhalable aerosols may also contain other ingredients such as cosolvents, stabilisers, surface-active agents (surfactants), antioxidants, lubricants and means for adjusting the pH. All these ingredients are known in the art. The propellant-gas-containing inhalable aerosols according to the invention may contain up to 5% by weight of active substance. Aerosols according to the invention contain, for example, 0.002 to 5% by weight, 0.01 to 3% by weight, 0.015 to 2% by weight, 0.1 to 2% by weight, 0.5 to 2% by weight or 0.5 to 1% by weight of active ingredient.

Alternatively topical administrations to the lung may also be by administration of a liquid solution or suspension formulation, for example employing a device such as a nebulizer, for example, a nebulizer connected to a compressor (e.g., the Pari LC-Jet Plus® nebulizer connected to a Pari Master® compressor manufactured by Pari Respiratory Equipment, Inc., Richmond, Va.).

Nebulizable formulation according to the present invention may be provided, for example, as single dose units (e.g., sealed plastic containers or vials) packed in foil envelopes. Each vial contains a unit dose in a volume, e.g., 2 mL, of solvent/solution buffer. The present invention also provides a syringe loaded with a composition comprising a binding molecule, in particular an antibody, of the invention. In one embodiment, a pre-filled syringe loaded with a unit dose of a binding molecule, in particular of an antibody of the invention, is provided. In another embodiment, an auto injector loaded with binding molecule, in particular an antibody, of the invention is provided. In a further embodiment, an IV bag loaded with binding molecule, in particular an antibody, of the invention is provided. Also provided, is the binding molecule, in particular antibody, of the invention in lyophilised form in a vial or similar receptacle in lyophilized form.

It is also envisaged that a binding molecule of the present invention may be administered by use of gene therapy. In order to achieve this, DNA sequences encoding the necessary polypeptides, for instance the heavy and light chains of the antibody molecule, under the control of appropriate DNA components are introduced into a patient such that the antibody chains are expressed from the DNA sequences and assembled in situ.

Once formulated, the compositions of the invention can be administered directly to the subject. The subjects to be treated can be animals. However, in one or more embodiments the compositions are adapted for administration to humans. In a particularly preferred embodiment the subject is human.

In one embodiment, where a binding molecule of the present invention is administered together with another agent, the two may be given simultaneously, sequentially or separately. In one embodiment, the two may be given in the same pharmaceutical composition.

In one embodiment, a pharmaceutical composition of the present invention is administered before, at the same time, and/or a transplant. For example, it may be so administered at the same time as the transplant of cells, tissues, or organs, such as any of those referred to herein. In another embodiment, it may be that the material transplanted into a subject is treated ex vivo prior to transplantation. In one embodiment, a binding molecule of the present invention is used to prevent, or reduce, rejection of transplanted material. In one embodiment, it is used to prevent, treat, or ameliorate an immune response against transplanted material. In one particularly preferred embodiment, it is used for that purpose in relation to graft versus host disease.

The present invention also extends to a kit, comprising a binding molecule of the invention. In one embodiment a kit comprising any of the binding molecules of the invention is provided, optionally with instructions for administration. In yet another embodiment, the kit further comprises one or more reagents for performing one or more functional assays. In another embodiment, a kit containing single-chambered or multi-chambered pre-filled syringes is provided. The invention also provides a kit for a single-dose administration unit.

### Pathological Conditions, Medical, and Diagnostic Uses

Also provided is the use of a binding molecule of the present invention for use as a medicament. In another embodiment a binding molecule of the present invention is provided for use in a method of therapy of the human or animal body. Please note that, in the various therapeutic uses set out herein where reference is made to a binding molecule, a pharmaceutical composition comprising a binding molecule may also be employed and vice versa unless stated otherwise. A binding molecule of the present invention may also be used in diagnosis, including in both in vivo diagnosis and also in vitro diagnosis, for example such diagnosis performed on a sample from a subject.

As discussed further below, a binding molecule of the present invention may be employed to treat a condition. As used herein, the terms “treat” or “treatment” refer to both therapeutic treatment and prophylactic or preventative measures, wherein the object is to prevent or slow down (lessen) an undesired physiological change or disorder. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

In one particularly preferred embodiment, a binding molecule of the present invention may be used to modulate the immune system. For example, a binding molecule of the present invention may be used to stimulate cells of the immune system, for instance activating particular cells of the immune system. In one embodiment the cells may be stimulated to proliferate. In one preferred embodiment, a binding molecule of the present invention is used to so activate cells expressing TNFR2 on their surface. For example, the cells in question may be white blood cells and in particular T cells. In a particularly preferred embodiment, a binding molecule of the present invention is used to activate T reg cells. For example, a binding molecule of the present invention may be used to stimulate T reg cells which in turn suppress, reduce, or prevent an immune response.

The ability of the present invention to modulate the immune system means that it represents a particular good way to target, for example, an autoimmune disorder, or an inflammatory disorder. Hence, the present invention provides for a binding molecule or pharmaceutical composition of the present invention for use in a method of treating or preventing an autoimmune disorder, or an inflammatory disorder. The present invention provides a binding molecule or pharmaceutical composition for use in such a method wherein:
(a) the disorder is graft versus host disease (GvHD), preferably where the binding molecule is for use in a method where it is administered prior to, at the same time, or after a transplant of a cell, tissue, or organ; or
(b) the disorder is one involving dysfunction or unwanted proliferation of leukocytes, preferably of T cells, more preferably of Teff cells; such disorders may present with an imbalance of Tregs compared to Teff cells, for example due to increased numbers or activity of Teff which is not balance the numbers and/or immunosuppressive properties of Tregs and in one embodiment the present invention promotes Tregs and in particular shifts the balance with effector cell to, or towards, normal.
(c) the disorder is selected from Inflammatory Bowel Disease (such as Ulcerative Colitis, Crohn's Disease, and Celiac Disease), multiple sclerosis, myasthenia gravis, skin autoimmune diseases such as pemphigus vulgaris or bullous pemphigoid, and Type 1 Diabetes, and atherosclerosis.

The present invention may be used in treating graft versus host disease (GVHD). In one embodiment, the present invention is employed to promote Treg activity prior to a cell, tissue or organ transplant. For example, in one embodiment the present invention is use to promote Treg activity before transplantation of cells, in particular prior to transplantation of stem cells, and preferably before the transplantation of hematopoietic stem cells. In another embodiment, rather than stimulate Tregs in the recipient prior to transplantation, the invention is used to expand Tregs in the cells, tissue, or organ that is to be transplanted to the host. In a further embodiment, they are used as part of the treatment for non-malignant hematopietic diseases.

The present invention may be used to reduce, prevent or treat an immune response against a transplant, for example against transplanted cells, tissue or an organ. Hence, the invention may be used to reduce, prevent or treat graft versus host disease (GvHD). In one embodiment, the present invention may be used in that way where what is transplanted are cells such as a cell population. In one embodiment, the transplanted material is, or comprises, haematopoietic stem cells (HSCs). In another embodiment, the transplanted material may be an organ or tissue, such as the transplant of a heart, lung, kidney, cornea, or other organ. In another embodiment, the transplanted material may be a graft, such as a skin graft. In one embodiment, the present invention provides a method comprise administering a binding molecule of the present invention to treat, prevent, or ameliorate an unwanted immune response against transplanted cells, tissues or organs. In one embodiment, the method may actually further comprise performing the transplant. In another embodiment, the binding molecule of the present invention is given to the subject before, during, and/or after the transplant. In a further embodiment, rather than administration of the binding molecule to the subject the method comprises treating the material to be transplanted ex vivo with the binding molecule before it is transplanted.

In one embodiment, rather than treat, prevent, or ameliorate the disease itself, the invention is employed to help ensure that the treatment for the disease, namely the transplanted cells, tissue, or organ, is effective by preventing or reducing the severity of GvHD. Hence, the present invention may be employed in a variety of embodiments where a disease is treated by transplanting cells, tissue or organ. In one preferred embodiment, the condition may be one treated via a stem cell transplant, for example a hematopoietic stem cell (HSC) transplant. In some embodiments, the subject has or is otherwise affected by a metabolic storage disorder which is to be treated by a transplant. The subject may suffer or otherwise be affected by a metabolic disorder selected from the group consisting of glycogen storage diseases, mucopolysaccharidoses, Gaucher's Disease, Hurlers Disease, sphingolipidoses, metachromatic leukodystrophy, or any other diseases or disorders which may benefit from the treatments and therapies disclosed herein and including, without limitation, severe combined immunodeficiency, Wiscott-Aldrich syndrome, hyper immunoglobulin M (IgM) syndrome, Chediak-Higashi disease, hereditary lymphohistiocytosis, osteopetrosis, osteogenesis imperfecta, storage diseases, thalassemia major, sickle cell disease, systemic sclerosis, systemic lupus erythematosus, multiple sclerosis, juvenile rheumatoid arthritis and those diseases, or disorders described in “Bone Marrow Transplantation for Non-Malignant Disease,” ASH Education Book, 1:319-338 (2000), the disclosure of which is incorporated herein by reference in its entirety as it pertains to pathologies that may be treated by administration of hematopoietic stem cell transplant therapy. In one embodiment, where the invention concerns transplantation, it may be that the transfer is of allogenic cells, tissues, or organs. In one embodiment, the transferred cells may be cells expressing a chimeric antigen receptor (CAR). In some embodiments, the subject is in need of chimeric antigen receptor T-cell (CART) therapy. For instance, such therapy may form part of a method of the present invention. In another preferred embodiment, the invention provides a method of promoting the engraftment of a cell population, tissue, or organ in a subject by treating, reducing, or preventing an immune response against said population, tissue, or organ.

The ability of a binding molecule of the present invention to modulate the immune system is also makes it a particularly valuable approach for targeting autoimmune disease. Hence, in another embodiment, the subject to be treated has an autoimmune disorder. In one particularly preferred embodiment, the autoimmune disorder is multiple sclerosis. In a further particular preferred embodiment, the subject has ulcerative colitis. In another particularly preferred embodiment, the condition is scleroderma. In one embodiment, the condition to be treated is lupus. Further examples of autoimmune diseases include scleroderma, Crohn's disease, Type 1 diabetes, or another autoimmune pathology described herein. In one embodiment, the autoimmune disease to be treated is selected from Ulcerative Colitis, Crohn's Disease, Celiac Disease, Inflammatory Bowel Disease, multiple sclerosis, lupus, Graves' disease and Type 1 Diabetes. In one embodiment, the subject has Type 1 Diabetes and that is treated. In one embodiment, the condition is atherosclerosis.

In one preferred embodiment, the condition treated is a condition involving unwanted inflammation. In one preferred embodiment, the condition is arthritis. For example, the present invention may be used to treat rheumatoid or osteoarthritis. Non-limiting types of Examples of diseases which may be treated include rheumatoid arthritis, polyarticular juvenile idiopathic arthritis, psoriatic arthritis, and pediatric arthritis. In another preferred embodiment, the condition to be treated is selected from multiple sclerosis, ankylosing spondylitis, Crohn's disease, and ulcerative colitis.

In one embodiment, the ability of the invention to stimulate Treg cells is employed as a way to treat allergy. In another embodiment, the ability to stimulate Treg cells may be employed as a way to treat asthma.

### Detection and Diagnosis

A binding molecule of the present invention may be used to detect TNFR2. For example the present invention provides a method comprising contacting a binding molecule of the present invention with a test sample and detecting any binding of the binding molecule. A binding molecule of the present invention may be labelled or linked to an enzyme which allows the detection of the binding molecule and hence that the binding molecule has bound. In one embodiment, such detections methods may be, for instance, ELISA assays or flow cytometry as a way to detect whether or not cells in a test sample express TNFR2 on their surface. A binding molecule of the present invention may be used in in vitro detection, it may also be used in detection of TNFR2 that is in vivo.

In one embodiment, the present invention provides an in vivo method for detecting TNFR2 that comprises administering a labelled binding molecule of the present invention and then detecting the location of the antibody in the body of a subject. In another embodiment, a binding molecule of the present invention may be used in the diagnosis of a condition, for example in identifying a reduction of cells expressing TNFR2, in particular a reduction of a particular subset of cells expressing TNFR2. In one preferred embodiment, the present invention provides a method of patient stratification comprising subdividing patients on the basis of the level of TNFR2, for example based on the level of a particular cell type expressing TNFR2.

The present invention also provides a kit for detecting TNFR2 comprising a binding molecule of the present invention and optionally instructions for employing the binding molecule in a method of detecting TNFR2.

In one embodiment, the present invention provides a binding molecule of the present invention as a companion diagnostic, for instance to determine whether or not to administer a drug to a subject based on detection of TNFR2, such as levels of TNFR2, for instance the number of particular cell types expressing TNFR2 or their location.

A binding molecule of the invention may be used in imaging. In one embodiment, a binding molecule may be used in imaging in vivo. Hence, also provided is a method of imagining comprising administering a labelled binding molecule of the present invention to a subject and then detecting the label. In one embodiment, the imaging may allow detection of cells expressing TNFR2 in vivo. In one embodiment, such methods may be used to identify localisation or build-ups of such cells. In another embodiment, the binding molecules may be used in a method of in vitro imaging.

As discussed herein, the present invention may be used to treat a subject. By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, and zoo, sports, or pet animals such as dogs, cats, guinea pigs, rabbits, rats, mice, horses, cattle, cows, and so on. In an especially preferred embodiment, the subject is human.

The binding molecules of the present invention may also be used to stimulate T cell proliferation and in particular T reg cell proliferation. In one embodiment, such a method is performed in vitro or ex vivo, for example by contacting T reg cells with a binding molecule of the present invention. In another embodiment, the present invention may further comprise then transferring the cells to a subject, for example after the T reg population has been expanded or after contact with the binding molecule so that the proliferation occurs in vivo after the cells have been transferred to the subject.

### Further Embodiments

The present invention also provides a method of treating or preventing an autoimmune disorder, or an inflammatory disorder of the present invention to a subject suffering from, or at risk of, the disorder.

The present invention also provides such a method wherein the method is for treating or preventing graft versus host disease (GvHD), with the method comprising administering the binding molecule to the subject and transplanting a cell, tissue, or organ to the subject, wherein the binding molecule is administered to the subject before, during, or after the transplant.

The present invention further provides such a method wherein:
(a) the disorder is one involving reduced numbers, dysfunction or unwanted proliferation of leukocytes, preferably of T cells, more preferably of T reg cells; or
(b) the disorder is one that can benefit from increasing numbers and/or function of Tregs
(b) the disorder is selected from Inflammatory Bowel Disease (such as Ulcerative Colitis, Crohn's Disease, and Celiac Disease), atherosclerosis, lupus, multiple sclerosis, Type 1 Diabetes, myasthenia gravis, pemphigus vulgaris, and bullous pemphigoid.

The present invention also provides the use of a binding molecule of the present invention for manufacturing a medicament for use in a method of treating or preventing an autoimmune disorder, or an inflammatory disorder.

The present invention further provides such a use wherein the medicament is for use in a method of treating:
(a) the disorder is one involving dysfunction or unwanted proliferation of leukocytes, preferably of T cells, more preferably of Treg cells; or
(b) the disorder is selected from Inflammatory Bowel Disease (such as Ulcerative Colitis, Crohn's Disease, or Celiac Disease), lupus, multiple sclerosis, Type 1 Diabetes, atherosclerosis, myasthenia gravis, pemphigus vulgaris, and bullous pemphigoid.

The present invention also provides a diagnostic method comprising administering a binding molecule of the present invention to a subject and detecting binding of the binding molecule to TNFR2, preferably wherein the binding molecule is labelled and the binding of the binding molecule to TNFR2 is detected via the label.

### Numbered Embodiments

The following represent further preferred embodiments of the present invention:

[1]. A binding molecule that specifically binds TNFR2, but not TNFR1, where the binding molecule is an FcγR independent agonist of TNFR2 and has a valency of at least two for binding TNFR2.

[2]. The binding molecule of [1], wherein the binding molecule is able to:
(a) oligomerise TNFR2 on the surface of a cell expressing TNFR2, preferably to cluster six or more TNFR2 molecules on the cell surface;
(b) trigger TNFR2 signalling; and/or
(c) stimulate proliferation of leucocytes, preferably T cells, more preferably Treg cells.

[3]. The binding molecule of [1] or [2], wherein:
(a) the binding molecule comprises a moiety that alters serum half-life, preferably wherein the moiety is selected from the group consisting of a Fc tail, serum albumin, a moiety which is a binder of serum albumin, and PEG;
(a) the binding molecule is bivalent for TNFR2 binding;
(b) the binding molecule is trivalent for TNFR2 binding;
(c) the binding molecule is tetravalent for TNFR2 binding; or
(d) the binding molecule which has a valency of at least four for TNFR2 binding.

[4]. The binding molecule of any one of [1] to [3] wherein:
(a) the binding molecule is an antibody;
(b) the binding molecule is an antibody which comprises a ligand for TNFR2;
(c) the binding molecule comprises at least part of an antibody constant region, preferably an Fc region; or
(d) the binding molecule comprises a ligand for TNFR2, preferably wherein the ligand is derived from TNFα or lymphotoxin-alpha, preferably where the binding molecule is:
(a) an antibody that comprises a TNFR2 binding site at both the N-terminus and C-terminus of one or more heavy chain of the antibody; or
(b) an antibody that comprises a TNFR2 binding site at both the N-terminus and C-terminus of one or more light chain of the antibody.

[5]. The binding molecule of [4], wherein:
(a) the antibody is a bivalent antibody;
(b) the antibody is a trivalent antibody;
(c) the antibody is tetravalent antibody;
(d) the antibody is a hexavalent antibody;
(e) the antibody is a heavy chain only antibody;
(f) the antibody is a single domain antibody, such as a single domain Ab (sdAb), VHH, or a Nanobody;
(g) the antibody is a multimer of at least two antibodies;
(h) the antibody comprises, or is linked to, a multimerizing moiety preferably tenascin-c peptide (TNC), preferably where the antibody is a multimer of at least two antibodies associated through the tenascin-c peptide sequence;
(i) the antibody is linked to one or more molecules derived from a ligand of TNFR2, preferably derived from TNF-α or lymphotoxin alpha.

[6]. The binding molecule of claim [4] or [5] wherein:
(a) the antibody lacks an Fc region; or
(b) the antibody comprises a modified Fc region that is unable to bind Fc receptors.

[7]. The binding molecule of any one of the preceding claims, wherein the binding molecule is an antibody in an antibody format selected from:
IgG with an Fc modification eliminating Fc receptor binding;
scFv:IgG where the IgG has an Fc modification eliminating Fc receptor binding;
IgG-HC:TNC, where the IgG has a modification eliminating Fc receptor binding;
scFv:IgG-HC:TNC where the IgG has a modification eliminating Fc receptor binding; and
hexameric IgG;
wherein:
HC represents a heavy chain of an IgG;
LC represents a light chain of an IgG;
scFv:IgG represents a scFv either linked to, or forming part of an IgG molecule;
and
HC:TNC indicates a heavy chain (HC) comprising, or linked to, tenascin-c trimerization domain peptide (TNC).

[8]. The binding molecule of any one of [1] to [7], wherein the binding molecule is an antibody in an antibody format selected from the group consisting of:
Fab-LC:scFv;
IgG-HC:scFv where the IgG has a modification eliminating Fc receptor binding, preferably N297A;
IgG-LC:scFv where the IgG has a modification eliminating Fc receptor binding, preferably N297A;
IgG-LC:scFv-HC:scFv;
LC:scFv-HC:scFv-IgG-HC:scFv;
LC:scFv-HC:scFv-IgG-LC:scFv;
LC:scFv-HC:scFv-IgG-LC:scFv-HC:scFv; and
IgG-HC-TNC-scFv;
sdAb/VHH
   wherein:
HC represents a heavy chain of an IgG;
LC represents a light chain of an IgG;
LC:scFv indicates a scFv linked to, or forming part of, a light chain;
HC:scFv indicates a scFv linked to, or forming part of, a heavy chain;
HC:TNC indicates a heavy chain (HC) comprising, or linked to, tenascin (TNC);
TNC-scFv indicating tenascin linked or bound to a scFv;
sdAb indicates a single domain antibody; and
VHH indicates the variable heavy chain region of a camelid antibody,
preferably wherein the binding molecule is an antibody and is in an antibody format selected from:
a Fab-scFv;
IgG-HC:scFv;
LC:scFv-HC:scFv-IgG1; and
LC:scFv-HC:scFv-IgG1-TNC.

[9]. A binding molecule that specifically binds TNFR2, but not TNFR1, where the binding molecule is an FcγR independent agonist of TNFR2, has a valency of at least two for binding TNFR2, and comprises polypeptides comprising mutant TNF-alpha that bind TNFR2, but not TNFR1, and an Fc region, or a tenascin peptide sequence, or both, that oligomerises the polypeptides,
preferably wherein the binding molecule is one:
(a) which does not comprise antibody sequences;
(b) which comprises polypeptides comprising mutant TNF-alpha that binds TNFR2, but not TNFR1, and a tenascin peptide;
(c) wherein the TNF-alpha mutants are TNF80; and/or
(d) has a valency for TNFR2 of at least three, preferably having a valency for TNFR2 of six

[10]. The binding molecule of any one of [1] to [10] for use as a medicament.

[11]. The binding molecule of any one of [1] to [9] for use in a method of treating or preventing an autoimmune disorder, or an inflammatory disorder, preferably wherein:
(a) the disorder is graft versus host disease (GvHD), preferably where the binding molecule is for use in a method where it is administered prior to, at the same time, or after a transplant of a cell, tissue, or organ; or
(b) the disorder is one involving dysfunction or unwanted proliferation of leukocytes, preferably of T cells, more preferably of Treg cells; or
(c) the disorder is selected from Inflammatory Bowel Disease (such as Ulcerative Colitis, Crohn's Disease, or Celiac Disease), atherosclerosis, lupus, multiple sclerosis, Type 1 Diabetes, myasthenia gravis, pemphigus vulgaris, and bullous pemphigoid.

[12]. A method of stimulating cell proliferation comprising contacting a target cell expressing TNFR2 with a binding molecule according to any one of [1] to [9].

[13]. A pharmaceutical composition comprising a binding molecule according to any one of [1] to [9] and a pharmaceutically acceptable carrier.

[14]. A method of detecting TNFR2 comprising contacting a test sample with a binding molecule according to any one of [1] to [9] and detecting binding of the binding molecule to TNFR2,
preferably wherein the binding molecule is labelled and the binding of the binding molecule to TNFR2 is detected via the label.

[15]. A binding molecule according to any one of [1] to [9] for use in a method of diagnosis, the method comprising administering a binding molecule according to any one of claims 1 to 15 to a subject and detecting binding of the binding molecule to TNFR2,
preferably wherein the binding molecule is labelled and the binding of the binding molecule to TNFR2 is detected via the label.

All documents referred to herein are incorporated by reference. Reference herein to the singular, using terms such as “a” and “an” also encompasses the plural unless specifically stated otherwise. Where something is referred to herein as “comprising” in another embodiment what the invention may “consist essentially of” what is set out. In another embodiment, it may “consist of” what is set out. Unless defined otherwise, all technical and scientific terms used herein have the meaning commonly understood by a person skilled in the art to which this invention belongs.

### EXAMPLES

### Example 1—Materials and Methods

### Introduction

The following provides a summary of the materials and methods that were employed when performing the experiments described in the subsequent experiments.

### Cell Lines and Cell Culture Conditions

HEK293T cells, HeLa cells, HeLa-TNFR2 (Weiss et al., 1997) cells, HT1080-TNFR2 (Gerspach et al., 2006, Cell Death Differ., 13(2): 273-284), and Kym-1 cells were cultured in RPMI 1640 media (Sigma-Aldrich, Irvine, United Kingdom) supplemented with 10% FCS (GIBCO, EU Approved, South America). Cells were cultured at standard conditions (5% CO₂, 37° C.).

### Antibody Cloning, Production, and Purification

Heavy and light chain encoding cDNA fragments of in-house generated TNFR2-specific antibodies (Medler et al., 2019) were generated, sequenced and subsequently cloned into the pCR3 expression vector (Invitrogen) using standard PCR and cloning techniques. Variants (mutants, fusion proteins) of antibody chains were generated using synthetic DNA fragments encoding the aa sequences of interest and standard cloning techniques. Expression plasmids for TNFR2-specific antibodies typically contain a FLAG-tag.

Antibodies and antibody variants were produced in HEK293T cells by use of the transfection reagent PEI (polyethylenimine; Polyscience Inc., Warrington, USA) as described for example in Medler and Wajant (2021). To quantify the amount of protein secreted into the supernatant, SDS-PAGE and anti-FLAG western blotting with an in house produced FLAG-tagged standard protein was performed (ANTI-FLAG M2 antibody, Sigma-Aldrich, St. Louis, USA).

The supernatant containing the produced TNFR2-specific antibodies were purified by anti-FLAG agarose affinity chromatography. After binding of the FLAG-tagged antibodies to anti-FLAG M2 affinity gel (Sigma-Aldrich, St. Louis, USA), the antibodies were eluated using an excess of FLAG-peptide (100 μg/ml) Sigma-Aldrich, St. Louis, USA) and fractionally collected. Purification was performed in a TBS-buffered system. To exchange TBS against PBS, proteins were afterwards dialyzed overnight at +4° C.

Non-FLAG-tagged antibodies were purified via ProteinA chromatography followed by size exclusion chromatography. Purity and concentration of purified proteins/antibodies were determined by SDS-PAGE and silverstaining in comparison to Amersham's “Low Molecular Weight Calibration Kit for SDS Electrophoresis” (GE Healthcare UK Limited, Little Chalfont, UK) containing defined amounts of proteins of known molecular weight. The silver staining kit was purchased from Thermo Scientific (Rockford, USA).

### Antibody Formats

FIG. 1 shows the antibody formats where the TNFR2 binding sites are unidirectionally orientated N-terminal, giving the nomenclature and domain architecture for each. The abbreviations used in the names given for the various antibody formats in the Figure are CI=clone identifier, e.g. C4, C19 etc.; N297A refers to the corresponding mutation in the heavy chain of human IgG1 reducing FcγR interaction; N-RGY refers to the N297A mutation plus mutations promoting IgG1 hexamerization; and TNC refers to a Tenascin-C trimeization domain peptide present at the C-terminus of the heavy chains act to allow three individual antibodies to be brought together in a trimeric form. The antibody formats and the number of N-terminal antigen bindings each is summarized in the Table below.

### Tables Summarizing Information

TABLE 1
Antibodies from FIG. 1 and their valency

| Antibody format | Valency for TNFR2 |
|---|---|
| CI-Fab | 1 |
| CI-Fab2 | 2 |
| CI-IgG1(N297A) | 2 |
| ScFv:CI-IgG1(297A) | 4 |
| CI-IgG1(N297A)-HC:TNC | 6 |
| ScFv:CI-IgG1(N297A)-HC:TNC | 12 |
| CI-IgG1(N-RGY) | 12 |

FIG. 7 shows the antibody formats where the TNFR2 binding sites are organized both N-terminal and C-terminal, with the nomenclature and domain architecture shown for each. The same abbreviations for clone identifier, the presence of a N297A modification, and the presence of tenascin trimerization peptide are used as in FIG. 1.

TABLE 2
Antibodies from FIG. 1 and their valency

| | Number of N and C terminal antigen |
|---|---|
| | binding sites for TNFR2, as well as |
| Antibody format | overall valency |
| CI-Fab-LC:scFvCI | 2 (1 N terminal and 1 C Terminal) |
| CI-IgG1(N297A)-HC:scFvCI | 4 (2 N terminal and 2 C Terminal) |
| CI-IgG1(N297A)-LC:scFvCI | 4 (2 N terminal and 2 C Terminal) |
| CI-IgG1(N297A)-LC:scFvCI- | 6 (2 N terminal and 4 C Terminal) |
| HC:scFvCI | |
| LC:scFvCI-HC:scFvCI- | 6 (4 N terminal and 2 C Terminal) |
| IgG1(N297A)-HC:scFvCI | |
| LC:scFvCI-HC:scFvCI- | 6 (4 N terminal and 2 C Terminal) |
| IgG1(N297A)-LC:scFvCI | |
| LC:scFvCI-HC:scFvCI- | 8 (4 N terminal and 4 C Terminal) |
| IgG1(N297A)-LC:scFvCI- | |
| HC:scFvCI | |
| CI-IgG1(297A)-HC:TNC- | 12 (6 N terminal and 6 C Terminal) |
| scFvCI | |

Table 3 below summarizes the binding molecules which are referred to in FIGS. 1 to 26 and discussed in the subsequent Examples, including the two expressions vectors used to express the polypeptides for each antibody.

TABLE 3
Proteins used in FIG. 1 to 27: Amino acid sequences of the light and
heavy chains encoded by the listed pDualyx expression plasmids

| | | | Polypeptides |
|---|---|---|---|
| Protein name | Expression plasmid(s) | | expressed |
| C4-IgG1(N297A) | pDualyx- | pDualyx- | SEQ ID NO: 3 & |
| | 010pre | 011pre | SEQ ID NO: 28 |
| C4-IgG1-LC:GpL | pDualyx- | pDualyx- | SEQ ID NO: 4 & |
| | 030pre | 029pre | SEQ ID NO: 29 |
| C4-IgG1(N297A)- | pDualyx- | pDualyx- | SEQ ID NO: 3 & |
| HC:scFvC4 | 010pre | 012pre | SEQ ID NO: 30 |
| C4-IgG1(N297A)- | pDualyx- | pDualyx- | SEQ ID NO: 5 & |
| LC:scFvC4 | 013pre | 011pre | SEQ ID NO: 28 |
| C4-IgG1(N297A)- | pDualyx- | pDualyx- | SEQ ID NO: 5 & |
| LC:scFvC4-HC:scFvC4 | 013pre | 012pre | SEQ ID NO: 30 |
| C4-Fab1 | pDualyx- | pDualyx- | SEQ ID NO: 3 & |
| | 010pre | 019pre | SEQ ID NO: 31 |
| C4-Fab1-LC:scFvC4 | pDualyx- | pDualyx- | SEQ ID NO: 5 & |
| | 013pre | 019pre | SEQ ID NO: 31 |
| C4-Fab2 | pDualyx- | pDualyx- | SEQ ID NO: 3 & |
| | 010pre | 020pre | SEQ ID NO: 32 |
| C4-Fab2-LC:scFvC4 | pDualyx- | pDualyx- | SEQ ID NO: 5 & |
| | 013pre | 020pre | SEQ ID NO: 32 |
| C4-IgG1(N297A)- | pDualyx- | pDualyx- | SEQ ID NO: 3 & |
| HC:TNC | 010pre | 044 | SEQ ID NO: 33 |
| C4-IgG1(N297A)- | pDualyx- | pDualyx- | SEQ ID NO: 3 & |
| HC:RGY | 010pre | 118 | SEQ ID NO: 34 |
| C4-IgG1(N297A)- | pDualyx- | pDualyx- | SEQ ID NO: 3 & |
| HC:TNC-scFvC4 | 010pre | 119 | SEQ ID NO: 35 |
| C4(scFv)- | pDualyx- | pDualyx- | SEQ ID NO: 6 & |
| IgG1(N297A) | 022 | 021 | SEQ ID NO: 36 |
| C4(scFv)-IgG1(N297A)- | pDualyx- | pDualyx- | SEQ ID NO: 7 & |
| LC:scFvC4 | 023 | 021 | SEQ ID NO: 36 |
| C4(scFv)-IgG1(N297A)- | pDualyx- | pDualyx- | SEQ ID NO: 6 & |
| HC:scFvC4 | 022 | 024 | SEQ ID NO: 37 |
| C4(scFv)-IgG1(N297A)- | pDualyx- | pDualyx- | SEQ ID NO: 7 & |
| LC:scFvC4-HC:scFvC4 | 023 | 024 | SEQ ID NO: 37 |
| C4(scFv)-IgG1(N297A)- | pDualyx- | pDualyx- | SEQ ID NO: 6 & |
| HC:TNC | 022 | 051 | SEQ ID NO: 38 |
| C4-IgG1(N297A)- | pDualyx- | pDualyx- | SEQ ID NO: 3 & |
| HC:scFvC19 | 010pre | 080 | SEQ ID NO: 39 |
| C4-IgG1(N297A)- | pDualyx- | pDualyx- | SEQ ID NO: 3 & |
| HC:scFvC27 | 010pre | 082 | SEQ ID NO: 40 |
| C4-IgG1(N297A)- | pDualyx- | pDualyx- | SEQ ID NO: 3 & |
| HC:scFvC40 | 010pre | 063 | SEQ ID NO: 41 |
| C19-muIgG1(D265A) | pDualyx- | pDualyx- | SEQ ID NO: 8 & |
| | 005 | 006 | SEQ ID NO: 42 |
| C19-IgG1-LC:GpL | pDualyx- | pDualyx- | SEQ ID NO: 9 & |
| | 032 | 031 | SEQ ID NO: 43 |
| C27-IgG1(N297A) | pDualyx- | pDualyx- | SEQ ID NO: 10 & |
| | 092 | 091 | SEQ ID NO: 44 |
| C27-IgG1(N297A)- | pDualyx- | pDualyx- | SEQ ID NO: 11 & |
| LC:GpL | 093 | 091 | SEQ ID NO: 44 |
| C40-IgG1(N297A) | pDualyx- | pDualyx- | SEQ ID NO: 12 & |
| | 077 | 075 | SEQ ID NO: 45 |
| C40-IgG1(N297A)- | pDualyx- | pDualyx- | SEQ ID NO: 13 & |
| LC:GpL | 076 | 075 | SEQ ID NO: 45 |
| C19-gG1(N297A)- | pDualyx- | pDualyx- | SEQ ID NO: 14 & |
| HC:scFvC19 | 016pre | 017pre | SEQ ID NO: 46 |
| C27-IgG1(N297A)- | pDualyx- | pDualyx- | SEQ ID NO: 10 & |
| HC:scFvC27 | 092 | 108 | SEQ ID NO: 47 |
| C40-IgG1(N297A)- | pDualyx- | pDualyx- | SEQ ID NO: 12 & |
| HC:scFvC40 | 077 | 111 | SEQ ID NO: 48 |
| C1-IgG1-LC:GpL | pDualyx- | pDualyx- | SEQ ID NO: 15 & |
| | 028 | 027 | SEQ ID NO: 49 |
| C9-IgG1(N297A)- | pDualyx- | pDualyx- | SEQ ID NO: 16 & |
| LC:GpL | 087 | 085 | SEQ ID NO: 50 |
| C29-IgG1(N297A)- | pDualyx- | pDualyx- | SEQ ID NO: 17 & |
| LC:GpL | 090 | 088 | SEQ ID NO: 51 |
| C31-2-IgG1(N297A)- | pDualyx- | pDualyx- | SEQ ID NO: 18 & |
| LC:GpL | 071 | 069 | SEQ ID NO: 52 |
| 68/69-IgG1(N297A)- | pDualyx- | pDualyx- | SEQ ID NO: 19 & |
| LC:GpL | 040pre | 041pre | SEQ ID NO: 53 |
| C19-IgG1(N297A)- | pDualyx- | pDualyx- | SEQ ID NO: 14 & |
| HC:TNC | 016 | 149 | SEQ ID NO: 54 |
| C19(scFv)- | pDualyx- | pDualyx- | SEQ ID NO: 25 & |
| IgG1(N297A) | 151 | 150 | SEQ ID NO: 55 |
| C19(scFv)-IgG1(N297A)- | pDualyx- | pDualyx- | SEQ ID NO: 25 & |
| HC:TNC | 151 | 152 | SEQ ID NO: 56 |
| C19(scFv)-Fab2- | pDualyx- | pDualyx- | SEQ ID NO: 25 & |
| HC:TNC | 151 | 153 | SEQ ID NO: 57 |
| C40-IgG1(N297A)- | pDualyx- | pDualyx- | SEQ ID NO: 12 & |
| HC:TNC | 077 | 159 | SEQ ID NO: 62 |
| C40(scFv)- | pDualyx- | pDualyx- | SEQ ID NO: 27 & |
| IgG1(N297A) | 161 | 160 | SEQ ID NO: 63 |
| C40(scFv)-IgG1(N297A)- | pDualyx- | pDualyx- | SEQ ID NO: 27 & |
| HC:TNC | 161 | 162 | SEQ ID NO: 64 |
| C40(scFv)-Fab2- | pDualyx- | pDualyx- | SEQ ID NO: 27 & |
| HC:TNC | 161 | 163 | SEQ ID NO: 65 |
| C27-IgG1(N297A)- | pDualyx- | pDualyx- | SEQ ID NO: 10 & |
| HC:TNC | 092 | 154 | SEQ ID NO: 58 |
| C27(scFv)- | pDualyx- | pDualyx- | SEQ ID NO: 26 & |
| IgG1(N297A) | 156 | 155 | SEQ ID NO: 59 |
| C27(scFv)-IgG1(N297A)- | pDualyx- | pDualyx- | SEQ ID NO: 26 & |
| HC:TNC | 156 | 157 | SEQ ID NO: 60 |
| C27(scFv)-Fab2- | pDualyx- | pDualyx- | SEQ ID NO: 26 & |
| HC:TNC | 156 | 158 | SEQ ID NO: 61 |
| C4-IgG1(N297A)- | pDualyx- | pDualyx- | SEQ ID NO: 4 & |
| LC:GpL-HC:scFvC4 | 030 | 012 | SEQ ID NO: 30 |
| C4-Fab1- | pDualyx- | pDualyx- | SEQ ID NO: 20 & |
| LC:scFvC9 | 112 | 019 | SEQ ID NO: 31 |
| C4-Fab1- | pDualyx- | pDualyx- | SEQ ID NO: 21 & |
| LC:scFvC27 | 113 | 019 | SEQ ID NO: 31 |
| C4-Fab1- | pDualyx- | pDualyx- | SEQ ID NO: 22 & |
| LC:scFvC29 | 114 | 019 | SEQ ID NO: 31 |
| C4-Fab1- | pDualyx- | pDualyx- | SEQ ID NO: 24 & |
| HC:scFvC40 | 116 | 019 | SEQ ID NO: 31 |
| C4-Fab1- | pDualyx- | pDualyx- | SEQ ID NO: 23 & |
| HC:scFv31.1 | 115 | 019 | SEQ ID NO: 31 |

Table 4 below gives the SEQ ID NOs of the light and heavy chain polypeptides encoded by the expression constructs referred to in Table 3 above.

TABLE 4
SEQ ID NOs for proteins encoded by the expression
plasmids referred to in Table 3

| Expression plasmid | LC/HC | SEQ ID NO: |
|---|---|---|
| pDualyx-010pre | LC | SEQ ID NO: 3 |
| pDualyx-030pre | LC | SEQ ID NO: 4 |
| pDualyx-013pre | LC | SEQ ID NO: 5 |
| pDualyx-022 | LC | SEQ ID NO: 6 |
| pDualyx-023 | LC | SEQ ID NO: 7 |
| pDualyx-005 | LC | SEQ ID NO: 8 |
| pDualyx-032 | LC | SEQ ID NO: 9 |
| pDualyx-092 | LC | SEQ ID NO: 10 |
| pDualyx-093 | LC | SEQ ID NO: 11 |
| pDualyx-077 | LC | SEQ ID NO: 12 |
| pDualyx-076 | LC | SEQ ID NO: 13 |
| pDualyx-016pre | LC | SEQ ID NO: 14 |
| pDualyx-028 | LC | SEQ ID NO: 15 |
| pDualyx-087 | LC | SEQ ID NO: 16 |
| pDualyx-090 | LC | SEQ ID NO: 17 |
| pDualyx-071 | LC | SEQ ID NO: 18 |
| pDualyx-040pre | LC | SEQ ID NO: 19 |
| pDualyx-112 | LC | SEQ ID NO: 20 |
| pDualyx-113 | LC | SEQ ID NO: 21 |
| pDualyx-114 | LC | SEQ ID NO: 22 |
| pDualyx-115 | LC | SEQ ID NO: 23 |
| pDualyx-116 | LC | SEQ ID NO: 24 |
| pDualyx-151 | LC | SEQ ID NO: 25 |
| pDualyx-156 | LC | SEQ ID NO: 26 |
| pDualyx-161 | LC | SEQ ID NO: 27 |
| pDualyx-011pre | HC | SEQ ID NO: 28 |
| pDualyx-029pre | HC | SEQ ID NO: 29 |
| pDualyx-012pre | HC | SEQ ID NO: 30 |
| pDualyx-019pre | HC | SEQ ID NO: 31 |
| pDualyx-020pre | HC | SEQ ID NO: 32 |
| pDualyx-044 | HC | SEQ ID NO: 33 |
| pDualyx-118 | HC | SEQ ID NO: 34 |
| pDualyx-119 | HC | SEQ ID NO: 35 |
| pDualyx-021 | HC | SEQ ID NO: 36 |
| pDualyx-024 | HC | SEQ ID NO: 37 |
| pDualyx-051 | HC | SEQ ID NO: 38 |
| pDualyx-080 | HC | SEQ ID NO: 39 |
| pDualyx-082 | HC | SEQ ID NO: 40 |
| pDualyx-063 | HC | SEQ ID NO: 41 |
| pDualyx-006 | HC | SEQ ID NO: 42 |
| pDualyx-031 | HC | SEQ ID NO: 43 |
| pDualyx-091 | HC | SEQ ID NO: 44 |
| pDualyx-075 | HC | SEQ ID NO: 45 |
| pDualyx-017pre | HC | SEQ ID NO: 46 |
| pDualyx-108 | HC | SEQ ID NO: 47 |
| pDualyx-111 | HC | SEQ ID NO: 48 |
| pDualyx-027 | HC | SEQ ID NO: 49 |
| pDualyx-085 | HC | SEQ ID NO: 50 |
| pDualyx-088 | HC | SEQ ID NO: 51 |
| pDualyx-069 | HC | SEQ ID NO: 52 |
| pDualyx-041pre | HC | SEQ ID NO: 53 |
| pDualyx-149 | HC | SEQ ID NO: 54 |
| pDualyx-150 | HC | SEQ ID NO: 55 |
| pDualyx-152 | HC | SEQ ID NO: 56 |
| pDualyx1-153 | HC | SEQ ID NO: 57 |
| pDualyx-154 | HC | SEQ ID NO: 58 |
| pDualyx-155 | HC | SEQ ID NO: 59 |
| pDualyx-157 | HO | SEQ ID NO: 60 |
| pDualyx-158 | HC | SEQ ID NO: 61 |
| pDualyx-159 | HC | SEQ ID NO: 62 |
| pDualyx-160 | HC | SEQ ID NO: 63 |
| pDualyx-162 | HC | SEQ ID NO: 64 |
| pDualyx-163 | HC | SEQ ID NO: 65 |

Table 5 below provides the CDR sequences of the specific antibody clones whose light and heavy chain variable regions and CDR sequences were used as a starting point for the antigen-binding sites of the various antibody formats employed in the present Examples other than the VHH based antibodies discussed in later Examples. The shading is included simply to help indicate light and heavy chain CDRs from the same clone.

TABLE 5
CDR amino acid sequences of specific anti-TNFR2 antibody clones#

| Antibody | Domain | CDR1 | CDR2 | CDR3 |
|---|---|---|---|---|
| C1 | VL | RSSTGAVTTSNYA | GTNNRTP | ALWYSNHWV (SEQ |
| | | NGTNNRTP (SEQ | (SEQ ID NO: 67) | ID NO: 68) |
| | | ID NO: 66) | | |
| C1 | VH | DDYIH | WIDPENSDTEYAS | TTGLWLRTAIDY |
| | | (SEQ ID NO: 99) | KFQGKAT | (SEQ ID NO: 101) |
| | | | (SEQ ID NO: 100) | |
| C4 | VL | KASQDVDTAVA | WASTRHT | QQYYSVPPT |
| | | (SEQ ID NO: 69) | (SEQ ID NO: 70) | (SEQ ID NO: 71) |
| C4 | VH | SYDIN | WIYPRDGDTKYN | LTGPYWYFDV |
| | | (SEQ ID NO: 102) | EKFKGKAI | (SEQ ID NO: 104) |
| | | | (SEQ ID NO: 103) | |
| C8 | VL | KASQDVDTAV | WASTRHT | QQYYSVPPT |
| | | (SEQ ID NO: 72) | (SEQ ID NO: 73) | (SEQ ID NO: 74) |
| C8 | VH | DDYIH | WIDPENSDTEYAS | GLWLRTAIDY |
| | | (SEQ ID NO: 105) | KFQGKAT | (SEQ ID NO: 107) |
| | | | (SEQ ID NO: 106) | |
| C9 | VL | SASSSVSYMH | STSNLAS | QQRSSYPPT |
| | | (SEQ ID NO: 75) | (SEQ ID NO: 76) | (SEQ ID NO: 77) |
| C9 | VH | TAGMQ | WINTHISGEPKYAE | WDGTGY |
| | | (SEQ ID NO: 108) | DFKGRFA | (SEQ ID NO: 110) |
| | | | (SEQ ID NO: 109) | |
| C15 | VL | RASENIDNYGISF | AASNQGS | QQSKEVPYT |
| | | MY | (SEQ ID NO: 79) | (SEQ ID NO: 80) |
| | | (SEQ ID NO: 78) | | |
| C15 | VH | DYEMH | VVDPGTGDTASN | RYYGSRESMDY |
| | | (SEQ ID NO: 111) | QKFKG | (SEQ ID NO: 113) |
| | | | (SEQ ID NO: 112) | |
| C16 | VL | KASQNVGTNVA | SASFRYS | QHFNSFPLT |
| | | (SEQ ID NO: 81) | (SEQ ID NO: 82) | (SEQ ID NO: 83) |
| C16 | VH | RYWMS | ESDPDSSTINYAPS. | EGYYDNEGYFDV |
| | | (SEQ ID NO: 114) | LKDKFI | (SEQ ID NO: 116) |
| | | | (SEQ ID NO: 115) | |
| C19 | VL | KASQNVGTNVA | SASYRYS | QQFDSHPLT |
| | | (SEQ ID NO: 84) | (SEQ ID NO: 85) | (SEQ ID NO: 86) |
| C19 | VH | IHGMS | WINTYSGVPTYAN | ARDEVRRGFGFAY |
| | | (SEQ ID NO: 117) | DFKGRFA | (SEQ ID NO: 119) |
| | | | (SEQ ID NO: 118) | |
| C25 | VL | KASQNVGSNVD | SASYRYS | QQYNSHPLT |
| | | (SEQ ID NO: 87) | (SEQ ID NO: 88) | (SEQ ID NO: 89) |
| C25 | VH | DLYIH | WIDPETDNTIYDP | STGLLQWYFDV |
| | | (SEQ ID NO: 120) | KFQGKAS | (SEQ ID NO: 122) |
| | | | (SEQ ID NO: 121) | |
| C31-1 | VL | RASQSLVHSNGNT | KVSNRFS | SQSTHVPFT |
| | | YLQ | (SEQ ID NO: 91) | (SEQ ID NO: 92) |
| | | (SEQ ID NO: 90) | | |
| C31 | VH | GYYWN | YITNDGSNNYNPS | DGGWFHYFDY |
| | | (SEQ ID NO: 123) | LKNRIS | (SEQ ID NO: 125) |
| | | | (SEQ ID NO: 124 | |
| C31-2 | VL | RSSTGAVTTSNYA | TNNRAP | ALWYSNHWV |
| | | N | (SEQ ID NO: 94) | (SEQ ID NO: 95) |
| | | (SEQ ID NO: 93) | | |
| C31 | VH | GYYWN | YITNDGSNNYNPS | DGGWFHYFDY |
| | | (SEQ ID NO: 126) | LKNRIS | (SEQ ID NO: 128) |
| | | | (SEQ ID NO: 127 | |
| C40 | VL | KASQDINKFIA | YTSTLQP | LQYDNLYT |
| | | (SEQ ID NO: 96) | (SEQ ID NO: 97) | (SEQ ID NO: 98) |
| C40 | VH | GMGVG | HIWWDDDKYYNP | IAGTRYFDV |
| | | (SEQ ID NO: 129) | ALKSRLT | (SEQ ID NO: 131) |
| | | | (SEQ ID NO: 130) | |

TABLE 6
Specific VH and VL variable region sequences from clones

| Antibody | Domain | Seq id no |
|---|---|---|
| C1 | VL | 294 |
| C1 | VH | 305 |
| C4 | VL | 295 |
| C4 | VH | 306 |
| C8 | VL | 296 |
| C8 | VH | 307 |
| C9 | VL | 297 |
| C9 | VH | 308 |
| C15 | VL | 298 |
| C15 | VH | 309 |
| C16 | VL | 299 |
| C16 | VH | 310 |
| C19 | VL | 300 |
| C19 | VH | 311 |
| C25 | VL | 301 |
| C25 | VH | 312 |
| C31-1 | VL | 302 |
| C31 | VH | 313 |
| C31-2 | VL | 303 |
| C31 | VH | 314 |
| C40 | VL | 304 |
| C40 | VH | 315 |

Table 7 provides SEQ ID NOs for further relevant proteins.

TABLE 7
SEQ ID NOs for TNFR2, TNFα variants, LTα, and other protein

| SEQ ID NO: | Protein |
|---|---|
| SEQ ID NO: 1 | Human TNFR2 - Accession No: NP_001057 |
| SEQ ID NO: 2 | Tenascin peptide - Amino acids 110-139 of chicken |
| | Tenascin |
| SEQ ID NO: 132 | Human TNF-α - Accession No. NP_000585 |
| SEQ ID No: 133 | Human Lymphotoxin-Alpha - Accession No: |
| | NP_000586 |
| SEQ ID No: 134 | TNC-sc(mu)TNF(143N/145R) |
| SEQ ID No: 135 & | Durvalumab heavy chain constant region sequence: |
| SEQ ID 136 | SEQ ID NO: 135 |
| | Durvalumab light chain constant region sequence: |
| | SEQ ID NO: 136 |
| SEQ ID: 316 | Durvalumab heavy chain constant region sequence |
| | lacking the CH1 region: SEQ ID NO: 316 |
| SEQ ID No: 137 | TNFR2(mu)-pCMV-SPORT6 (Tnfrsf1b) (Origene) |
| | cDNA clone IMAGE: 3710793; GenBank: |
| | BC039127.1 |
| SEQ ID No: 138 | TNFR2-cyno; Macaca fascicularis; GenBank: |
| | EHH62644.1 |
| SEQ ID No: 139 | Human Tenascin peptide - Amino acids 110-139 |
| | of human Tenascin (NP 002151.2) |

TABLE 8
SEQ ID Nos for VHH based sequences

| SEQ ID NO: | Protein |
|---|---|
| SEQ ID NOs: | Fourteen VHH sequences generated specific for |
| 140 to 153 | TNFR2 |
| SEQ ID NO: | C188 variants VHH sequences: |
| 198 to 212 | C188-VHH_1: SEQ ID NO 148/C188-VHH_2: |
| | SEQ ID NO 149/C188-VHH_3: SEQ ID NO |
| | 150/C188-VHH_4: SEQ ID NO 151/C188-VHH_5: |
| | SEQ ID NO 152/C188-VHH_6: SEQ ID NO 153/ |
| | C188-VHH_7: SEQ ID NO 154/C188-VHH_8: |
| | SEQ ID NO 155/C188-VHH_9: SEQ ID NO 156/ |
| | C188-VHH_10: SEQ ID NO 157/C188-VHH_11: |
| | SEQ ID NO 158/C188-VHH_12: SEQ ID NO 159/ |
| | C188-VHH_13: SEQ ID NO 160/C188-VHH_14: |
| | SEQ ID NO 161/C188-VHH_15: SEQ ID NO 162 |
| SEQ ID NO: 197 | 3XVHHC188(G4S)1-Fc(Durv) |

TABLE 9
C4 based variant sequences

| SEQ ID NO: | Protein |
|---|---|
| SEQ ID NOs: | C4-IgG1(Durv)-HC:scFvC4(G4S)4 sequences: |
| 261 and 258 | Light chain (signal peptide + C4-VL + |
| | durvaCL) − SEQ ID NO: 261 |
| | Heavy chain (signal peptide + C4-VH + |
| | DURVA[CH1 + Hinge + CH2 + CH3] + |
| | scFvC4(C4-Vh-linker-C4-VL): SEQ ID NO: 258 |
| SEQ ID NO: | Light chain variant sequences for C4: |
| 262 to 265 | C4_VL_1 - SEQ ID NO: 262/C4_VL_2 - |
| | SEQ ID NO: 263/C4_VL_3 - SEQ ID NO: |
| | 264/C4_VL_1 - SEQ ID NO: 265 |
| SEQ ID NO: | Light chain variant sequences for C4: |
| 278 to 281 | C4_VH_1 - SEQ ID NO: 278/C4_VH_2 - |
| | SEQ ID NO: 279/C4_VH_3 - SEQ ID NO: |
| | 280/C4_VH_4 - SEQ ID NO: 281 |

TABLE 10
C4 sequence optimized variants: permutations
of C4_VH and C4_VL variants:

| Variant Name | Heavy chain | Light chain |
|---|---|---|
| C4 | C4_VH | C4_VL |
| C4_var1 | C4_VH_1 | C4_VL_1 |
| C4_var2 | C4_VH_1 | C4_VL_2 |
| C4_var3 | C4_VH_2 | C4_VL_1 |
| C4_var4 | C4_VH_2 | C4_VL_2 |
| C4_var5 | C4_VH_1 | C4_VL_3 |
| C4_var6 | C4_VH_1 | C4_VL_4 |
| C4_var7 | C4_VH_2 | C4_VL_3 |
| C4_var8 | C4_VH_2 | C4_VL_4 |
| C4_var9 | C4_VH_3 | C4_VL_1 |
| C4_var10 | C4_VH_3 | C4_VL_2 |
| C4_var11 | C4_VH_4 | C4_VL_1 |
| C4_var12 | C4_VH_4 | C4_VL_2 |
| C4_var13 | C4_VH_3 | C4_VL_3 |
| C4_var14 | C4_VH_3 | C4_VL_4 |
| C4_var15 | C4_VH_4 | C4_VL_3 |
| C4_var16 | C4_VH_4 | C4_VL_4 |

### Gel Filtration

Purified antibody fusion proteins (50-200 μg) were analyzed for their native weight and potential protein aggregation by gel filtration on a MabPac SEC-1 column (Thermo Fisher) using the UltiMate 3000 HPLC system (Thermo Fisher) and the aqueous SEC-1 column performance check standard (#AL0-3042, Phenomenex, Torrance, CA, USA). Alternatively, Gravitrap or MAbSelect columns were used.

### Cellular Binding Studies

Affinity (K_{D}-values) of TNFR2-targeting antibodies to human, cynomolgus monkey, and murine TNFR2 was evaluated by cellular equilibrium binding studies. Therefore, HEK293T cells were transiently transfected with human, cynomolgus or mouse TNFR2 encoding expression plasmids or an empty vector control (EV) using the PEI method. Aliquots of HEK293T transfectants were treated with increasing concentration of the indicated GpL (Gaussia princeps luciferase)-tagged anti-TNFR2 antibodies, incubated for one hour at standard cell culture conditions and subsequently washed two times with ice-cold PBS. Cells were finally resuspended in 50 μl RPMI 1640 media supplemented with 0.5% FCS and transferred into a 96-black well plate. Cell associated luciferase activity (RLU; relative light units) was detected with LUmo luminometer (anthos Mikrosysteme GmbH, Friesoythe, Germany) directly after adding 1.5 μM of the substrate coelenterazin (Carl Roth, Karlsruhe, Germany).

Affinity of TNFR2-specific antibodies and antibody variants were also evaluated with HeLa cells and HeLa cells stably expressing human TNFR2. HeLa and HeLa-TNFR2 cells were seeded out in 24-well plates. Next day, the cells were challenged with the indicated concentrations of GpL-labelled TNFR2 antibodies for 1 hour at 37° C. To eliminate the unbound antibodies, the cells were washed 10 times with ice-cold PBS. The cells were then scratched and collected in 50 μl of medium (RPMI, 0.5% FCS) and transferred to black 96-well plates for measurement as mentioned above. The specific binding of different TNFR2-specific antibodies/antibody variants was calculated by subtracting the unspecific binding (values obtained from HeLa cells) from total binding (obtained from HeLa-TNFR2). The resulting values were fitted using the non-linear regression analysis option of GraphPad Prism 5.

### Analysis of IL8 Induction

Cytokine induction is a well-established readout for NFκB activation in response to TNFR2 signaling. We analyzed the strength of NFκB activation by measuring the IL8 amount after TNFR2 stimulation with the help of a commercially available IL8 ELISA kit (IL8 ELISA Set, BD OptEIA™, San Diego, USA).

HT1080-Bcl2-TNFR2 cells responding to TNFR2 stimulation with strong IL8 induction were seeded in 96-well plates (20.000 cells/well). The next day, cells were stimulated with the TNFR2-targeting reagents of interest and oligomerized TNF80 (=TNC-sc(mu)TNF(143N/145R) a highly potent TNFR2-selective TNF variant (Chopra et al., 2015) as a positive control. After overnight incubation 50 μl of the supernatant was analyzed with the help of BD OptEIA™ IL8 ELISA kit as described in the manufacturer's manual.

### Viability Assay

TNFR2 can induce in some cell lines/cell types cell death by inhibition of survival proteins (TRAF2, cIAP1, cIAP2) and concomitant upregulation of TNF triggering TNFR1. This type of response has been demonstrated among others in Kym-1 cells (Schneider et al., 1999). To test whether the TNFR2-targeting constructs are able to trigger this TNFR2 response, Kym-1 cells were seeded in 96-well plates (20.000 cells/well) and were stimulated the next day with the TNFR2-targeting reagents of interest. To define complete cell killing an aliquot of cells were also treated with a “cell death”-control mixture ((200 ng/ml TNF, 200 ng/ml TRAIL, 200 ng/ml CD95L, 25 μg/ml CHX, 1% (w/v) sodium azide)). Remaining plastic-attached cells were finally stained with 70 μl crystal-violet solution. After 20 minutes plates were washed once with dH₂O and dried on air. After solving in methanol the OD at 595 nm was measured with a PHOmo photometer (anthos Mikrosysteme GmbH, Friesoythe, Germany) to quantify viability. Values were normalized according to untreated cells (100% viability) and cells treated with the cytotoxic mixture (0% viability).

### Activation of the Alternative NFκB Pathway

p100 processing to p52 is the main characteristic of the activity of the alternative NFκB pathway. To evaluate the ability of anti-TNFR2 antibodies to induce p100 processing, 1×10⁶ Kym-1 cells were seeded per well of a 6-well tissue culture plate. Next day, cells were stimulated with the TNFR2-targeting reagent of interest. After overnight incubation at standard cell culture conditions total cell lysates were prepared (1×wash PBS, scraping cells in 1 ml PBS, centrifugation (2 min, 12000 rpm, cell pellet lysed in 4× Laemmli sample buffer, sonicated for 25 see with maximal amplitude (UP100H Ultrasonic Processor, Hielscher, Germany), heating for 5 min at 95° C. and removal of debris by centrifugation (2 min, 12000 rpm). For evaluation of p100 processing western blotting with standard techniques and standard equipment were performed using the anti-p100/p52 antibody of from Sigma Aldrich (Darmstadt, Germany).

### REFERENCES FOR MATERIALS & METHODS

Chopra M, Biehl M, Steinfatt T, Brandl A, Kums J, Amich J, Vaeth M, Kuen J, Holtappels R, Podlech J, Mottok A, Kraus S, Jordin-Garrote A L, Bauerlein C A, Brede C, Ribechini E, Fick A, Seher A, Polz J, Ottmuller K J, Baker J, Nishikii H, Ritz M, Mattenheimer K, Schwinn S, Winter T, Schafer V, Krappmann S, Einsele H, Müller TD, Reddehase M J, Lutz M B, Mannel D N, Berberich-Siebelt F, Wajant H, Beilhack A. Exogenous TNFR2 activation protects from acute GvHD via host T reg cell expansion. J Exp Med. 2016 Aug. 22; 213(9):1881-900.
Medler, J., Nelke, J., Weisenberger, D. et al. TNFRSF receptor-specific antibody fusion proteins with targeting controlled FcγR-independent agonistic activity. Cell Death Dis 10, 224 (2019). https://doi.org/10.1038/s41419-019-1456-x
Medler J, Wajant H. Analysis of FcγR-Dependent Agonism of Antibodies Specific for Receptors of the Tumor Necrosis Factor (TNF) Receptor Superfamily (TNFRSF). Methods Mol Biol. 2021; 2248:81-90.
Schneider P, Schwenzer R, Haas E, Muhlenbeck F, Schubert G, Scheurich P et al. (1999). TWEAK can induce cell death via endogenous TNF and TNF receptor 1. Eur J Immunol 29: 1785-1792.
Weiss, T. et al. Enhancement of TNF receptor p60-mediated cytotoxicity by TNF receptorp80: requirement of the TNF receptor-associated factor-2 binding site. J. Immunol. 158, 2398-2404 (1997).

### Example 2: Western Blot Analysis of Variants of the Anti-TNFR2 Antibody C4 with Parallel Organized (Unidirectionally Orientated) N-Terminal TNFR2 Binding Sites

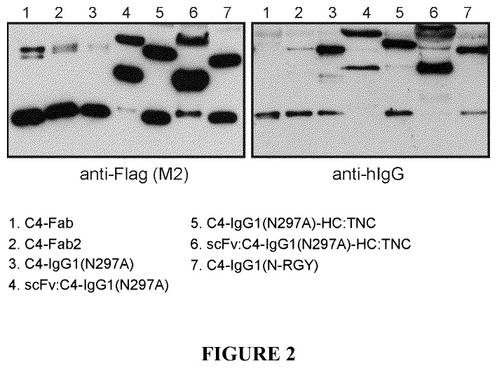

Flag-tagged variants of anti-TNFR2 antibody C4 with unidirectionally orientated (parallel organized) N-terminal TNFR2 binding sites were expressed in HEK293T cells as described in Example 1. The antibody formats assessed are those shown in FIG. 1, with the specific antibodies employed having the heavy and light chain variable regions from the clone 4 TNFR2 antibody, so that the “Clone Identifier” indicated in the Figure is “C4”. Plasmids encoding the chains (HC, LC) of each of the various C4 construct were transfected into the cells. Antibody variant containing supernatants along with various amounts of an antibody standard of known concentration were analyzed by WB and the concentration of the antibody variants in the supernatants were concluded from the standard antibody signals. A volume of supernant containing 200 ng for each antibody variant was then again subjected to Western blotting with anti-human IgG and anti-Flag antibodies. The results obtained are shown in FIG. 2 with all of the antibody formats successfully expressed and found in the supernatant recovered from the cells.

### Example 3: TNFR2-Mediated IL8 Production after Treatment with C4 Variants with Unidirectionally Orientated (Parallel Organized) N-Terminal TNFR2 Binding Sites

The ability of antibody variants with unidirectionally orientated (parallel organized) N-terminal TNFR2 binding sites was assessed using the antibody variants comprising the heavy and light chain variable regions from the original Clone 4 antibody specific for TNFR2. Again, the structure of the variants is that shown in FIG. 1, but with the “Clone Identifier” being C4 denoting the origin of the antigen-binding sites. A positive control was performed using a highly active oligomeric form of the TNFR2-specific TNF mutant TNF(143N/145R).

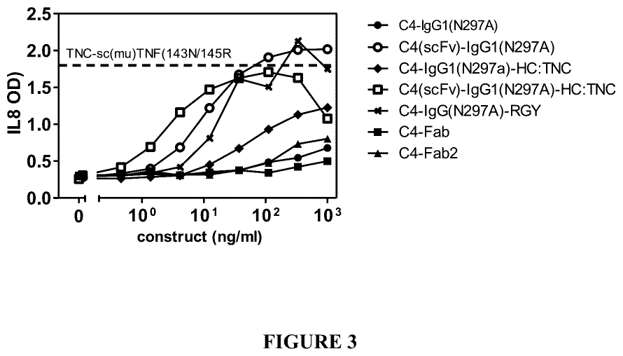

In order to perform the assessment, HT1080-Bcl2-TNFR2 cells were stimulated with supernatants containing the concentrations of the various C4 constructs indicated in FIG. 3. Next day, supernatants were harvested and analyzed by ELISA for their IL8 content. The results obtained are shown in FIG. 3. The maximum response induced was with the highly active oligomeric form of the TNFR2-specific TNF mutant TNF(143N/145R) included as a positive control, with the results for that indicated by a dotted line in the graphs.

As can be seen from FIG. 3, the antibody variants that were most active in the assay were those with multiple unidirectionally orientated (parallel) organized N-terminal TNFR2 binding sites. The lowest level of IL-8 production was seen with the C4-Fab, C4-Fab2, and C4-IgG1(N297A) variants which have two or less TNFR2 binding sites.

### Example 4: TNFR2-Mediated Cell Death in Kym-1 Cells after Treatment with C4 Variants with Unidirectionally Orientated (Parallel Organized) N-Terminal TNFR2 Binding Sites

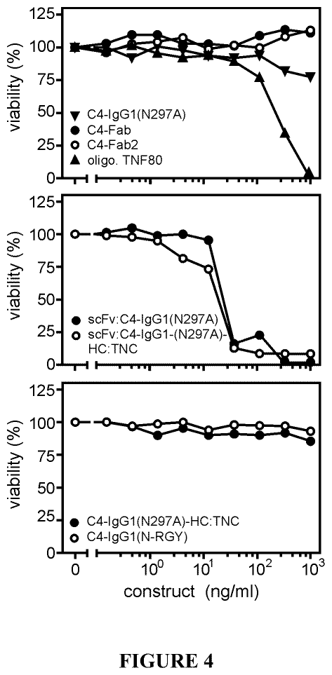

The ability of the antibody formats with N-terminal TNFR2 binding sites to trigger cell death in Kym-1 cells was then assessed, again using the Clone 4 light and heavy chain variable regions to confer specificity against TNFR2. The results obtained are shown in FIG. 4 with the abbreviations used again those from FIG. 1 apart from using “C4” to denote the origin of the antigen-binding sites.

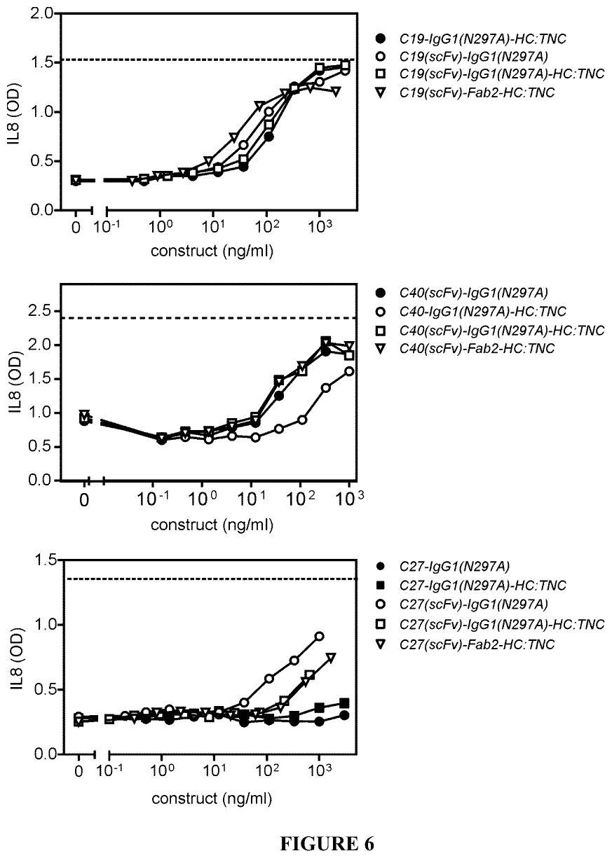

Kym-1 cells were stimulated with supernatants containing the concentrations of the various C4 constructs indicated in FIG. 4. Next day, cell viability was determined using crystal violet staining. The results obtained are shown in FIG. 4. A highly active oligomeric form of the TNFR2-specific TNF mutant TNF(143N/145R) again served as a positive control for TNFR2 engagement. The most effective antibody variants in inducing apoptosis were the scFv:C4-IgG1(N297A) and scFv:C4-IgG1-(N297A)-HC:TNC variants the results for which are showing in the middle panel of FIG. 4. Results for binding molecules based on the binding sites from the clones C19, C27, and C40 are given in FIG. 6.

### Example 5: TNFR2-Mediated p100 Processing in Kym-1 Cells after Treatment with C4 Variants with Unidirectionally Orientated (Parallel) Organized N-Terminal TNFR2 Binding Sites

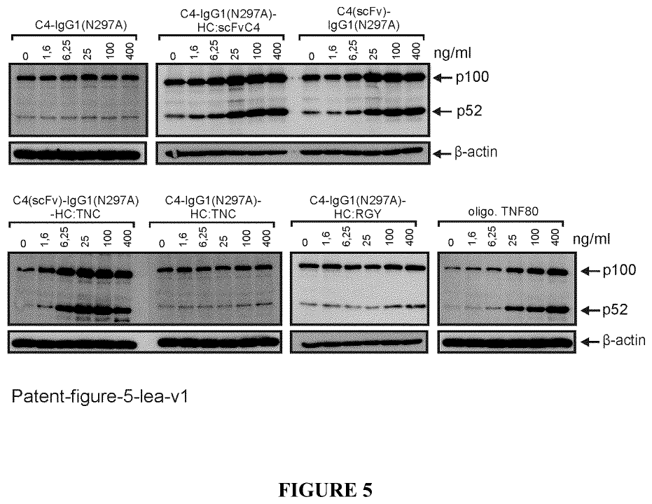

The ability of the C4 variants with unidirectionally orientated (parallel) organized N-terminal TNFR2 binding sites to trigger processing of p100 to p52 and hence indicate triggering of signal transduction through TNFR2 was assessed. Kym-1 cells were stimulated overnight with supernatants containing the concentrations of the various C4 variants indicated in FIG. 5. The structures of the variants employed are shown in FIG. 1 with “C4” denoting the “clone Identifier”. Treatment was performed in the presence of 20 μM ZVAD to prevent TNFR2-induced cell death. A nonameric TNFR2-specific mutant of mouse TNF (oligo TNF80) was used as a positive control. Western blotting was performed using antibodies specific for the indicated proteins, p100, p52, and as a control β-actin. The results obtained are shown in FIG. 5.

### Example 6: Western Blot Analysis of C4 Variants with N-Terminal and C-Terminal Organized TNFR2 Binding Sites

The experiments described in Example 2 for antibody variants with unidirectionally orientated (parallel organized) N-terminal TNFR2 binding sites were also performed for the C4 antibody variants depicted in FIG. 7 that have both N-terminal and C-terminal TNFR2 binding sites. The variants included FLAG tags in the heavy and light chains, except that the heavy chain of the C4-IgG1(N297A)-HC:scFvC4 variant which did not contain a Flag tag.

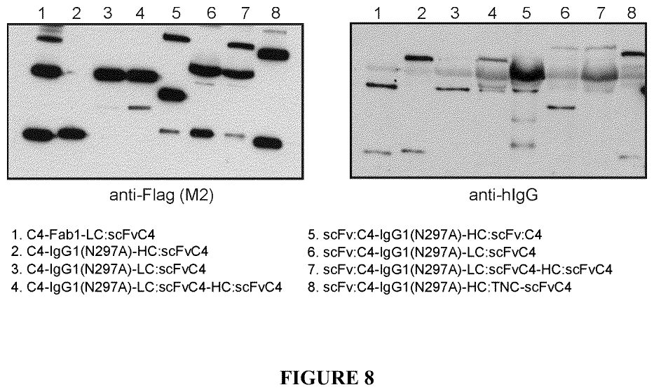

Each C4 construct was transfected into the cells and the antibody variants purified from the supernatants of the cells. 200 ng of each antibody variant was used in Western blotting. The supernatants containing the Flag-tagged variants were analyzed by Western blotting with anti-human IgG and anti-Flag antibodies. The results obtained are shown in FIG. 8 with all of the antibody formats successfully expressed and found in the supernatant recovered from the cells.

### Example 7: TNFR2-Mediated IL8 Production in HT1080-Bcl2-TNFR2 Cells after Treatment with C4 Constructs with N-Terminal and C-Terminal Organized TNFR2 Binding Sites

Equivalent experiments to those set out in Example 3 were also performed with the C4 variants with both N-terminal and C-terminal organized TNFR2 binding sites shown in FIG. 7. In order to provide a comparison, a number of the antibody variants with only N-terminal TNFR2 binding sites were also tested side-by-side with those with both N-terminal and C-terminal TNFR2 binding sites.

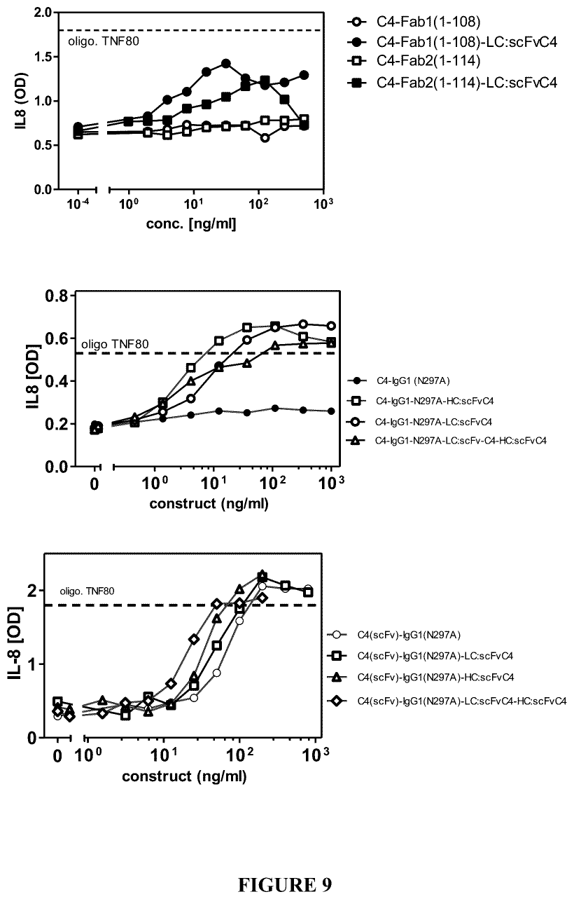

HT1080-Bcl2-TNFR2 cells were stimulated with supernatants containing the concentrations of the various C4 constructs indicated in FIG. 9. Next day, supernatants were harvested and analyzed by ELISA for their IL8 content. The results obtained are shown in FIG. 9. The data shown are derived from parallel processed experiments which, for better resolution, are shown in three diagrams. The maximum response induced with a positive control (highly active oligomeric form of the TNFR2-specific TNF mutant TNF(143N/145R)) was indicated by a dotted line.

As shown in FIG. 9, the antibody variants with both N- and C-terminal TNFR2 binding sites tended to give the most positive results, with valency though also having an impact. For instance, the results in the middle panel show such antibody formats triggering a higher level of IL-8 production than the C4-IgG1(N297A) variant with just N-terminal TNFR2 binding sites.

### Example 8: TNFR2-Mediated Cell Death in Kym-1 Cells after Treatment with C4 Constructs with N-Terminal and C-Terminal Organized TNFR2 Binding Sites

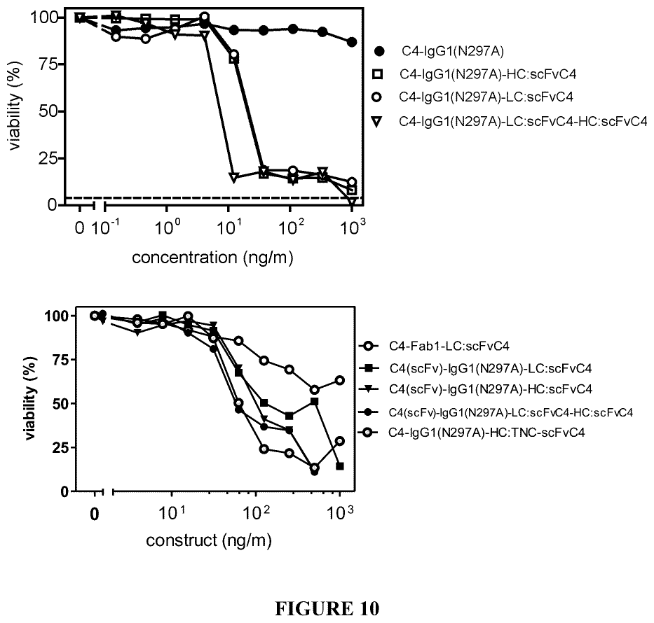

Equivalent experiments to those performed in Example 4 were also carried out for C4 antibody variants where the antibody has both N and C-terminal TNFR2 binding sites. Kym-1 cells were stimulated with supernatants containing the concentrations of the various C4 constructs indicated in FIG. 10. Next day, cell viability was determined using the MTT assay. A highly active oligomeric form of the TNFR2-specific TNF mutant TNF(143N/145R) was used as a positive control. The results obtained are shown in FIG. 10.

### Example 9: Binding Properties of TNFR2-Specific Antibodies to HeLa Cells Expressing Human TNFR2

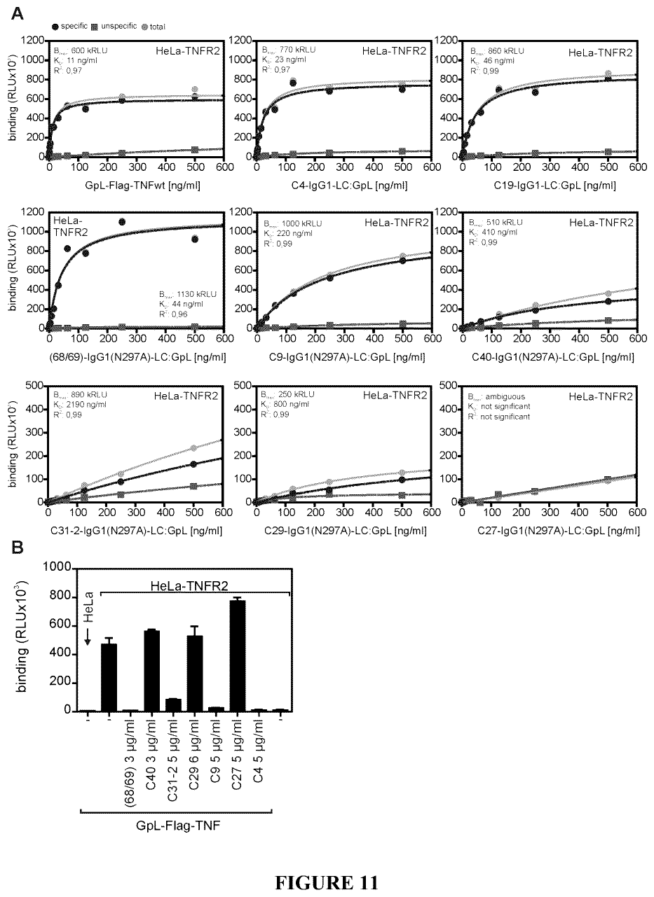

The binding properties of a number of the TNFR2-specific antibodies derived from various clones was assessed using Hela cells expressing TNFR2. Equilibrium binding of the anti-TNFR2 antibody GpL fusion proteins to cells expressing TNFR2 at 37° C. was assessed with the results obtained shown in FIG. 11A. The level of specific, unspecific, and total binding is shown for each antibody. The first graph shows the results for a GpL-Flag-TNFwt positive control for binding to TNFR2.

A further experiment was performed where rather than GpL labelled antibody GpL labelled TNF was used. HeLa-TNFR2 cells were pretreated with the indicated amounts of anti-TNFR2 antibodies or remained untreated. 5 ng/ml GpL-TNF was added and binding was measured after 1 hour incubation at 37° C. Binding to untransfected HeLa cells was then analyzed to obtain unspecific binding values. The results obtained are shown in FIG. 11B.

### Example 10: Competition of αTNFR2 Antibodies

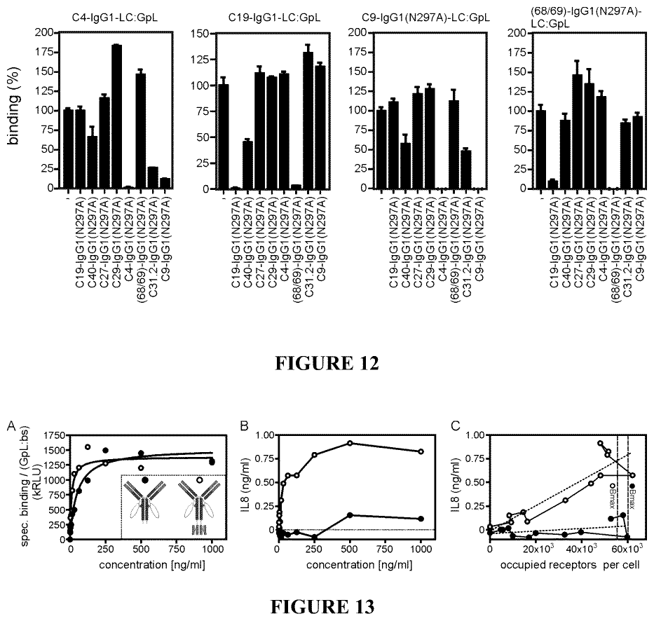

HeLa-TNFR2 cells were pretreated with the 3-10 μg of the anti-TNFR2 antibodies shown in FIG. 12 or remained untreated. 50 ng/ml of the various αTNFR2-IgG1(N297A)-LC:GpL variants were added and binding was measured after 1 hour as discussed in the Materials & Methods Example 1. The results obtained are shown in FIG. 12.

### Example 11: Tetravalent mAb Occupied TNFR2 Molecules Trigger Much Stronger IL8 Induction than TNFR2 Molecules Occupied by the Conventional Bivalent mAb Variant

TNFR2-negative Hela cells and HeLa-TNFR2 cells were pairwise stimulated for 8 hours with the concentrations indicated in (A) and (B) of FIG. 13 of the GpL fusion proteins of the bi- and tetravalent version of the TNFR2-specific mAb C4 (scheme see inset). Supernatants of HeLa-TNFR2 were then analyzed for TNFR2-induced IL8 production with the results shown in (B). Repeatedly washed cells (HeLa, and HeLa-TNFR2) were analyzed for cell-associated GpL activity. Specific binding to TNFR2, was calculated by subtraction of the unspecific binding values obtained from the HeLa cells from the total binding values derived of the HeLa-TNFR2 cells with the results obtained shown in (A). Please note, luciferase activity was normalized according to the ratio of the number of GpL reporter domains to the number of TNFR2-binding domains within the two mAb variants (1 versus 0.5).

IL8 production by the mAb variants were directly plotted as a function of their specific binding. The latter was transformed into “occupied receptors per cells” by help of the number of cells in the assay and the measured specific activity (RLU/molecule) of the GpL constructs. The results are shown in (C). Maximum binding of the two constructs obtained from A is indicated by dashed vertical lines. The dotted lines indicate linear regression of the IL8 production as function of receptor occupancy.

### Example 12: Binding Properties of TNFR2-Specific Antibodies to HEK293T Cells Expressing Human TNFR2

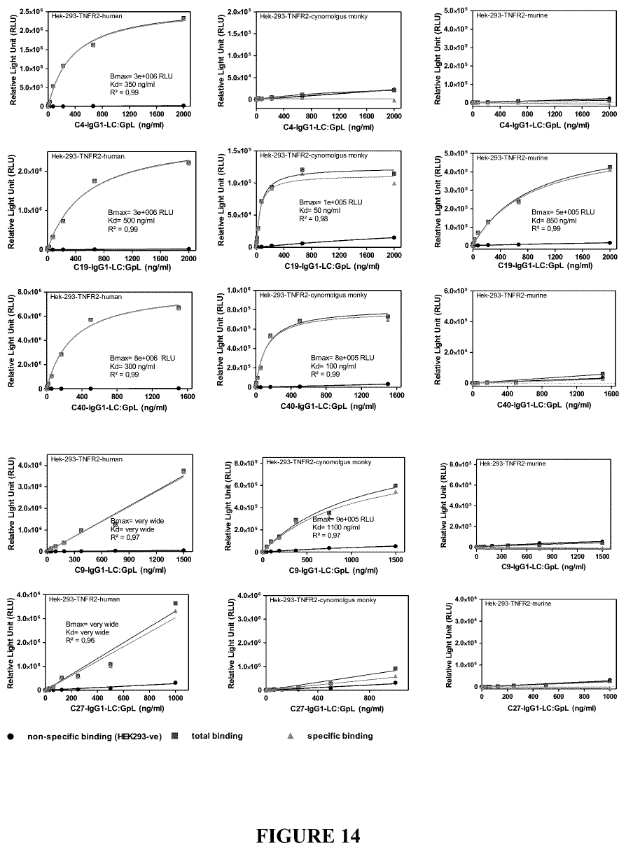

The binding properties of anti-TNFR2 antibodies from different clones were assessed. HEK293 cells were transiently transfected with human, murine and cynomolgus TNFR2 or empty vector. Equilibrium binding of the indicated anti-TNFR2 antibody GpL fusion proteins was then determined at 37° C. Binding to empty vector transfected cells was determined to obtain unspecific binding values. The results obtained are shown in FIG. 14. The antibody binding specifically to all three of human, murine and cynomolgus TNFR2 was the C19-IgG1-LC-GpL antibody. The C4-IgG1-LC-GpL antibody bound to human TNFR2, but not monkey or murine TNFR2. The C40-IgG1-LC-GpL antibody bound to human and monkey TNFR2 but not to murine TNFR2.

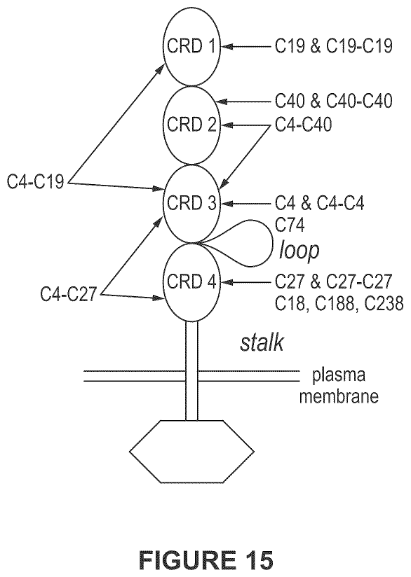

FIG. 15 shows the domain architecture of TNFR2, indicating the extracellular TNFR2 parts recognized by the anti-TNFR2 antibodies C4, C19, C27 and C40 and tetravalent CI-IgG1(N297A)-HC:scFvCI variants derived thereof.

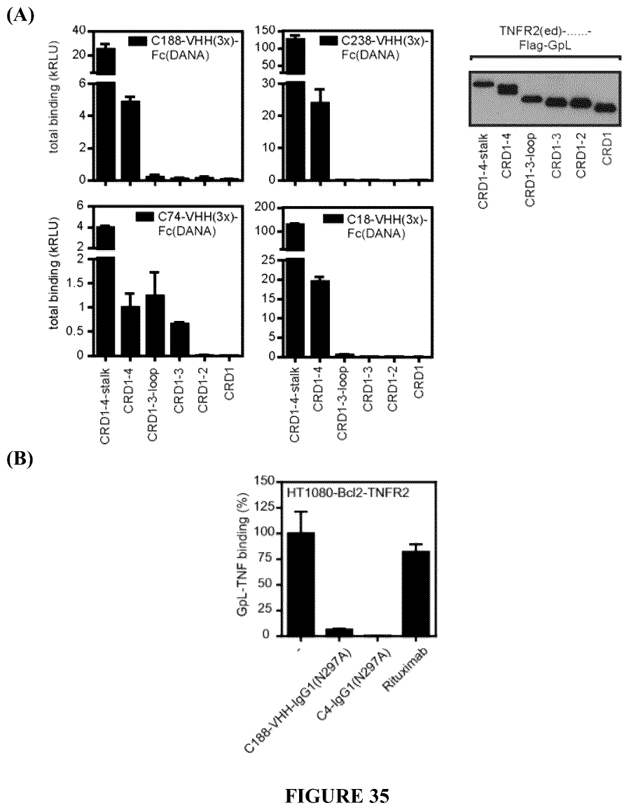

In addition, the recognition of the extracellular part of TNFR2 by the anti-TNFR2 VHHs VHH:C18, VHH:C74, VHH:C188 and VHH:C238 clones, and constructs derived thereof, is schematically represented, as based on FIG. 35.

### Example 13: Western Blot Analysis of Mono- and Bispecific TNFR2-Specific CI-IgG-HC:scFvCI Variants

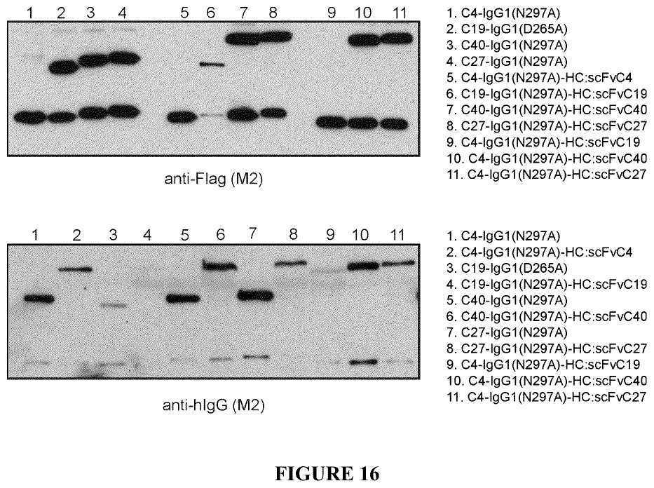

A number of different antibody variants were expressed and analyzed by Western blotting in an equivalent manner to the approach used in Examples 2 and 6. The eleven variants analysed had TNFR2 binding sites from different clones, different heavy chain modifications, and a number of them also had C-terminal TNFR2 binding sites as well, with the variants studied indicated in FIG. 16. The variants were expressed and analyzed in the equivalent way to that set out in Examples 2 and 6.

The top panel of FIG. 16 showing the results of blotting with the anti-FLAG antibody against the FLAG tag that the antibodies were labelled with. Please note that: i) the heavy chains of C4-IgG1(N297A), C4-IgG1(N297A)-HC:scFvC4 and C4-IgG1(N297A)-HC:scFvC19 do not contain a Flag tag; and ii) the conventional C19 variant used was in the murine IgG1(D265A) isoform. The bottom panel of FIG. 16 shows the results of the Western blot with anti-hIgG.

### Example 14: TNFR2-Mediated IL8 Production in HT1080-Bcl2-TNFR2 Cells

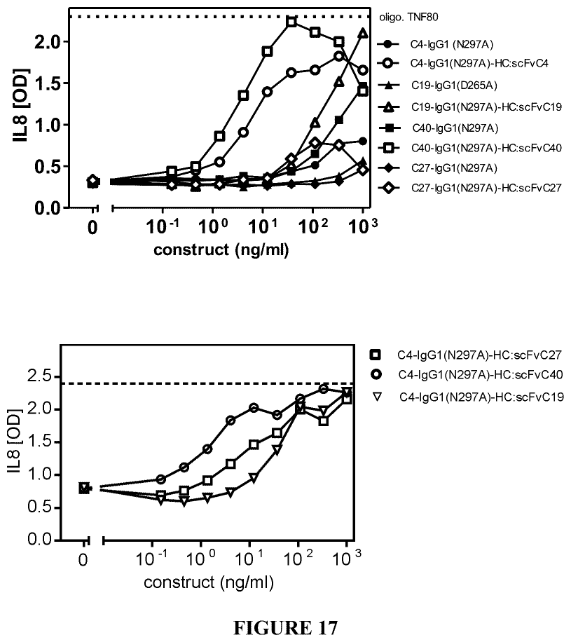

Analysis of the ability of anti-TNFR2 antibodies to stimulate IL-8 production in HT1080-Bcl2-TNFR2 cells was performed in an equivalent manner to Examples 3 and 7. A number of monospecific antibody variants was assessed, as well as a number of bispecific antibodies with two different specificities for TNFR2. In order to perform the assessment, HT1080-Bcl2-TNFR2 cells were stimulated with supernatants containing the concentrations of the various constructs indicated in FIG. 17. Next day, supernatants were harvested and analyzed by ELISA for their IL8 content. The results obtained are shown in FIG. 17, with the top panel showing the results for the monospecific antibody and the bottom panel the results for the bispecific antibody.

The top panel shows that in each instance the antibody variant with both an N-terminal and C-terminal TNFR2 binding site stimulated a higher level of TNF production than the corresponding antibody variant with just N-terminal TNFR2 binding site. The results for the C40-IgG1(N297A)-HC:scFvC40 binding molecule were particularly positive in the results shown in the top panel.

### Example 15: TNFR2-Mediated Cell Death in Kym-1 Cells after Treatment with Mono-Specific and Bispecific TNFR2-Specific CI-IgG-HC:scFvCI Variants

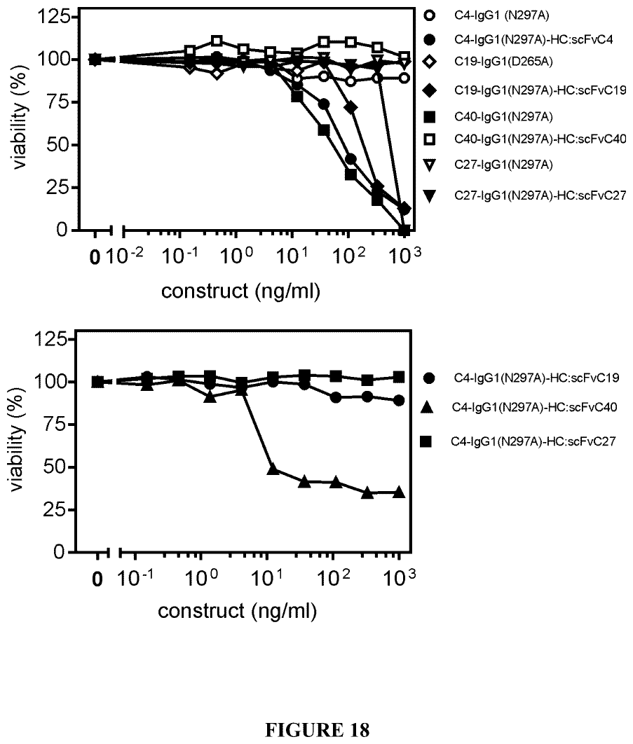

The ability of monospecific and bispecific antibodies to induce cell death was assessed in an equivalent manner to the approach used in Examples 4 and 8. Kym-1 cells were stimulated with supernatants containing the concentrations of the various C4 constructs indicated in FIG. 18. Next day, cell viability was determined using crystal violet staining. The results obtained are shown in FIG. 18. The results for the monospecific antibodies are shown in the top panel, with those for the biparatopic antibodies shown in the bottom panel.

### Example 16: TNFR2-Mediated p100 Processing in Kym-1 Cells after Treatment with Monospecific and Bispecific TNFR2-Specific CI-IgG-HC:scFvCI Variants

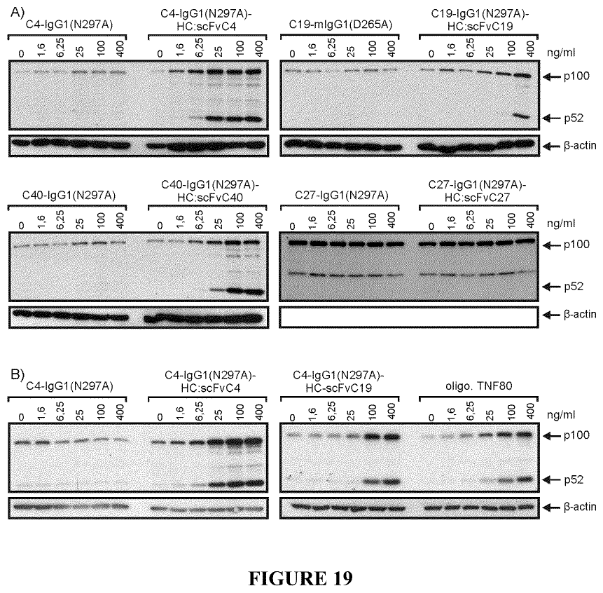

The ability of monospecific and bispecific antibodies to bring about signal transduction through TNFR2 as assessed by looking at p100 processing in an equivalent manner to Examples 5 and 9. Kym-1 cells were stimulated overnight with supernatants containing the concentrations of the various C4 variants indicated in FIG. 19. Treatment was performed in the presence of 20 μM ZVAD to prevent TNFR2-induced cell death. A nonameric TNFR2-specific mutant of mouse TNF (oligo. TNF80) was used as a positive control. Western blotting was performed using antibodies specific for the indicated proteins, p100, p52, and as a control β-actin. The results obtained are shown in FIG. 19.

### Example 17: Purification and Functional Analysis of C4-IgG1(N297A)-HC:scFvC4, C4-IgG1(N297A)-LC:scFvC4 and C4-IgG1(N297A)-LC:scFvC4-HC:scFvC4

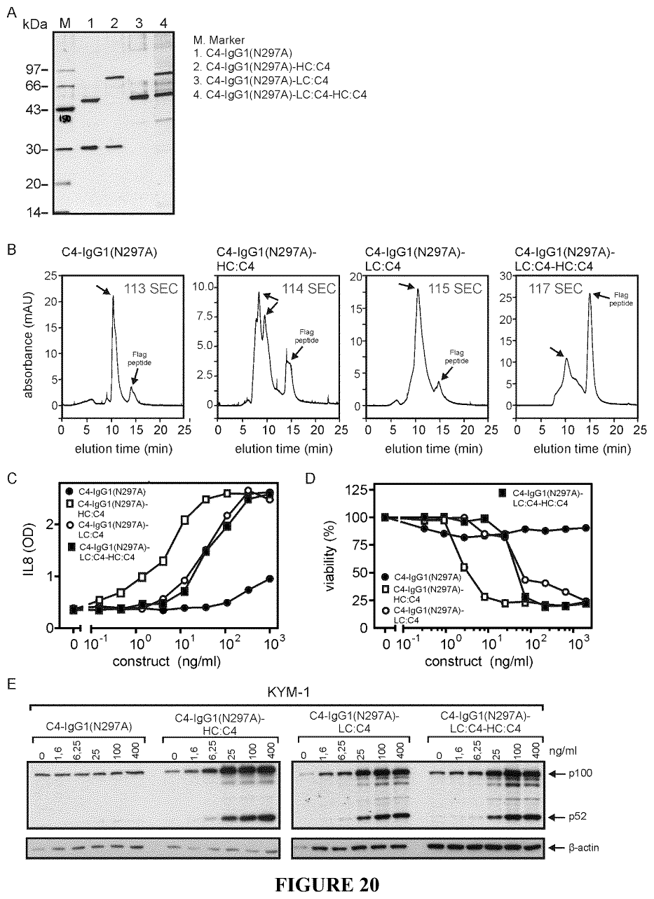

The constructs indicated in FIG. 20 were assessed using the methods discussed in Example 1 and employed in the earlier Examples. The results obtained are shown in FIG. 20. (A) Silver stained SDS-PAGE gel. (B) Gelfiltration analysis. (C) IL8 induction in HT1080-Bcl2-TNFR2 cells. (D) Cell death induction in Kym-1 cells. (E) Induction of p100 processing in Kym-1 cells. ZVAD was added to prevent cell death.

### Example 18: Activity

Table 7 below summarises the activity of various antibody formats employed in earlier Examples produced as set out in the Materials and Methods Section in Example 1. The antibody formats assessed were those having antigen-binding sites derived from the C4 clone “Bs” in the table indicates the number of “North” and “South” antigen binding sites of the antibody. The Table summarises the activity seen in the IL-8 assay (Activity I—classical NFκB pathway>IL8) or Activity II (TRAF2 depletion>alternative NFκB pathway (=p100 processing) and Kym-1 killing)).

TABLE 7
Summary of activity for C4 based antibody formats

| | | | Activity I | Activity II |
|---|---|---|---|---|
| No. | Ab format | Bs | (IL8) ng/ml | (tox) ng/ml |
| 1 | Fab1 | 1 + 0 | 0 | 0 |
| 2 | Fab1-scFv | 1 + 1 | 10 | 20 |
| 3 | Fab2 | 2 + 0 | 0 | 0 |
| 4 | Fab2-scFv | 2 + 2 | 50 | |
| 5 | IgG1 | 2 | 0 | 0 |
| 6 | irrIgG1-HC:scFv | 0 + 2 | 0 | |
| 7 | IgG1-HC:scFv | 2 + 2 | 5-20 | 10-20 |
| 8 | IgG1-LC:scFv | 2 + 2 | 5-20 | 10-20 |
| 9 | IgG1-LC:scFv- | 2 + 4 | 5-20 | 10-20 |
| | HC:scFv | | | |
| 10 | IgG1-TNC | 6 + 0 | 100 | Very low |
| 11 | IgG1-RGY* | 12 + 0 | 20-100 | Low |
| 12 | LC:scFv-HC:scFv- | 4** + 0 | 10-20 | 20 |
| | IgG1 | | | |
| 13 | L/H:scFv-IgG1- | 4** + 2 | 5-10 | 5-10 |
| | LC:scFv | | | |
| 14 | L/H:scFv-IgG1- | 4** + 2 | 5-10 | 5-10 |
| | LC:scFv | | | |
| 15 | L/H:scFv-IgG1- | 4** + 4 | 5-10 | 5-10 |
| | L/HC:scFv | | | |
| 16 | LC:scFv-HC:scFv- | 12** + 0 | 5-10 | 10 |
| | IgG1-TNC | | | |
| 17 | scFv-Fc-TNC | 12 + 0 | 10 | |
| 18 | scFv-TNC-Fc | 12 + 0 | 5-20 | 20 |
| 19 | IgG1-TNC-HC:scFv | 6 + 6 | 5-10 | 10-20 |
| | Star2 | 9 + 0 | 5-20 | 100-200 |

### Example 19: Ex Vivo Assessment of Treg Expansion

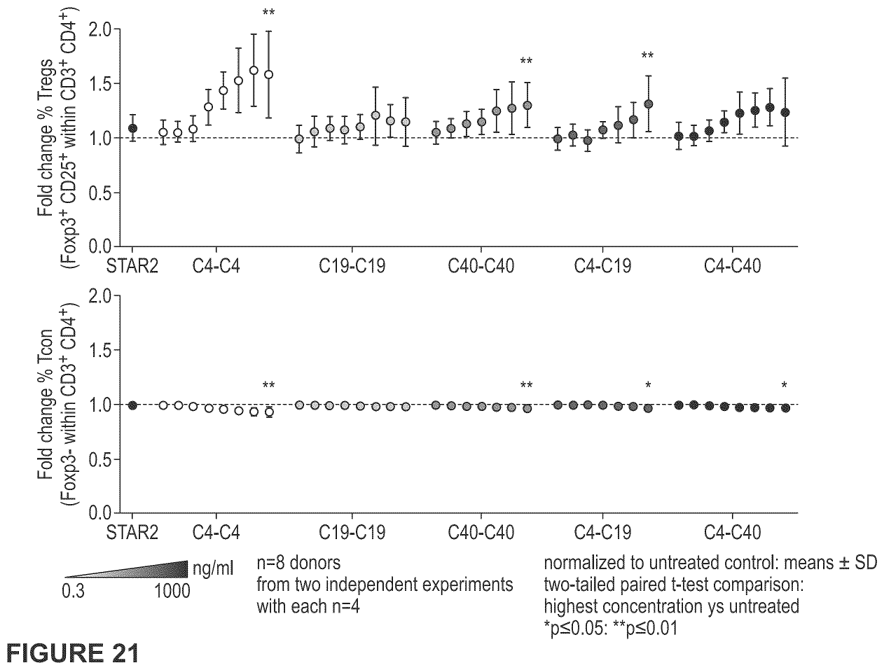

The ability of particular TNFR2 binding molecules to expand regulatory T cells was assessed. Tetravalent antibodies in the Fab-HC-scFv format, where the heavy chains of the Fc regions had the N297A amino acid mutation to eliminate Fc function, and the basic Fab with an IgG1 Fab. PBMCs were isolated from four donors, with the cells from each donor assessed separately. The PBMC were cultured for two days at a density of 10⁷ cells/ml. The PBMCs were then harvested and seeded into multi-well plates at a density of 10′ cells/ml, with 500,000 cells per well. The cells were then subdivided into: (a) untreated control cells; (b) cells which were treated with a STAR2 agonist molecule at 100 ng/ml; (c) cells which were treated with tetravalent antibodies with just one specificity (1000-0.3 ng/ml); and (d) cells which were treated with tetravalent antibodies with two specificities for TNFR2 (1000-0.3 ng/ml). Following culturing for four days the viability of the cells was assessed. The cells were stained with antibodies specific for CD3, CD8, CD4, CD25 and FoxP3. The cells were then assessed via flow cytometry. FIG. 21 shows the results obtained in the top panel for Foxp3+CD25+ cells within CD3+CD4+ cells to measure Treg cells and their expansion. The bottom panel of FIG. 21 shows Foxp3− cells within CD3+ CD4+ cells to measure Tcon cells. Both show percentage change compared to the number of cells prior to the four day incubation.

### Example 20: In Vivo Treg Expansion

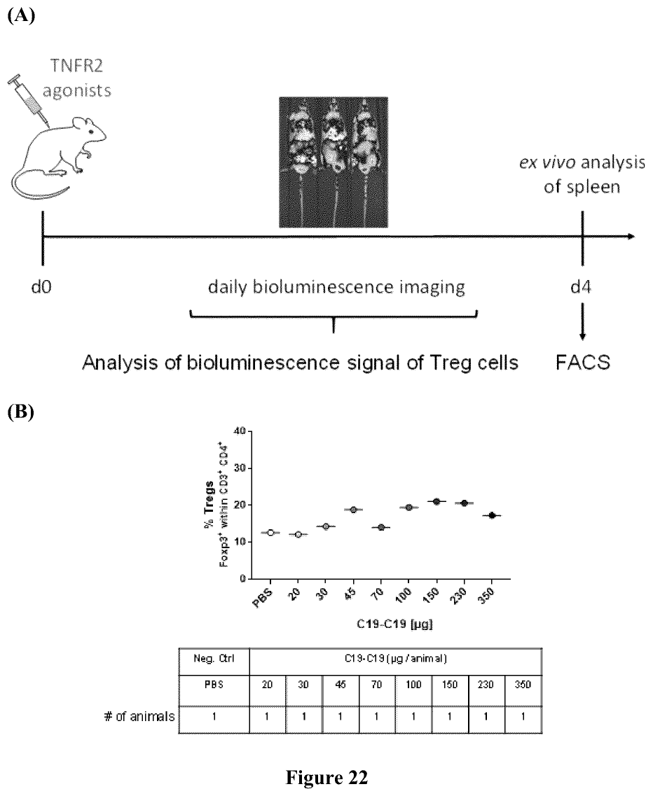

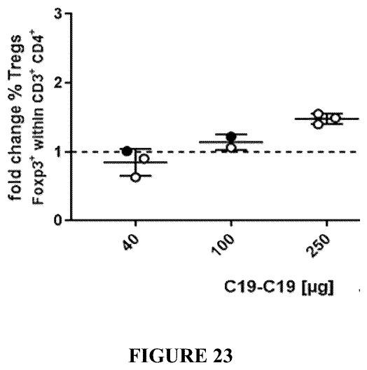

The ability of the TNFR2 agonists provided to expand Treg cells was also assessed using syngeneic FoxP3-Luci mice. FoxP3-Luci mice are transgenic mice that express a fusion protein of FoxP3 and luciferase. This effectively means that it is possible to image the sites of Treg cells in vivo. The mice were given a TNFR2 agonist with daily bioluminescence imaging. After four days the mice were sacrificed and ex vivo analysis performed on cells isolated from the spleens of the mice, with flow cytometry used to assess Foxp3, CD3, and CD4. The time line for the experiment is summarized in FIG. 22 in the top panel (A). The results of the ex vivo analysis are shown in FIG. 22 in the bottom panel (B). The experiment was repeated with multiple mice per treatment group, as shown in FIG. 23.

### Example 21: Production and Analysis of TNFR2-Specific TNF Constructs

The TNF-alpha based molecules shown in FIG. 24, part (A) were generated. The molecules all comprised the TNF80 molecule which represents a mutated form of TNF-alpha that lost TNFR1 binding and is thus specific for TNFR2. The structures shown from left to right in part (A) are:
Fc-TNC-TNF80—comprising three Fc regions where the C-terminal region of each polypeptide comprises a tenascin peptide (TNC) followed by a single TNF80 sequence (TNF80 corresponds to the TNFR2-specific mutant TNF(143N/145R). The tenascin peptides form trimers which in turns means that there are two trimers of the mutant TNF-alpha present.
Fc-TNF80—comprising three Fc regions where the C-terminal region of each polypeptide comprises a single TNF80 sequence, which results in two trimers of the mutant TNF-alpha being present in the molecule.
Fc(DANA)-TNC-TNF80—corresponding to Fc-TNC-TNF80 with the heavy chain modifications D265A and N297A (DANA).
TNF80—corresponding to the basic TNF80 mutant in trimeric form.
TNC-scTNF80—corresponding to single chain molecules comprising three TNF80 mutant molecules, where the chains further comprise a TNC peptide resulting in trimerisation of the three single chain polypeptides.

Murine soluble TNF80 based constructs for each of the above were used to express the molecules. The constructs were then purified by anti-Flag affinity purification utilizing a Flag tag contained in all constructs. They were then analyzed by SDS-PAGE and silver staining with the results obtained shown in FIG. 24, part (B). Gel filtration analysis of the purified proteins shown in FIG. 24, panel (B) was then performed and the results obtained are shown in FIG. 24 panel (C). The gel filtration results show that the expressed binding molecules are not aggregated and represent a homogeneous protein species. The methodology used in this Example was as set out in the Materials & Methods in Example 1 above.

### Example 21: Agonistic Activity of Different Formats of Soluble Murine TNF80

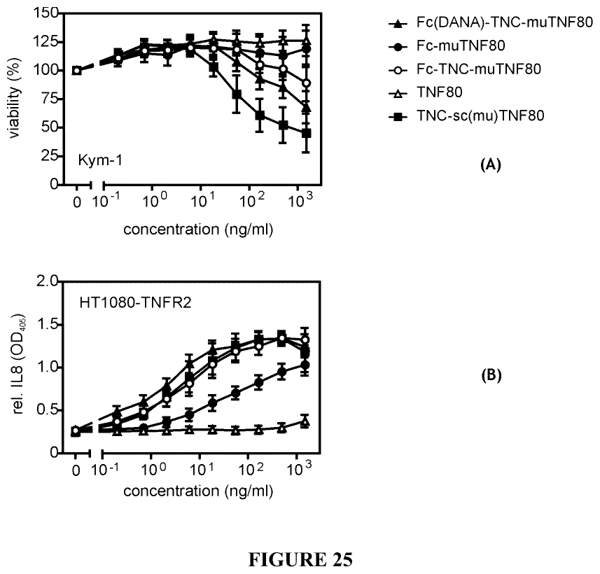

The ability of the constructs discussed in Example 20 to act as agonists of TNFR2 was next assessed using Kym-1 cells to determine the ability of the binding molecules to induce cell death and HT1080-TNFR2 cells to determine the ability of the binding molecules to induce IL-8 release. The methodology set out above in the Materials & Methods of Example 1 was employed. The results obtained are shown in FIG. 25.

The upper panel of FIG. 25 shows the results for Kym-1 cells expressing TNFR2 stimulated with the indicated concentrations of the various muTNF80 variants and then assessed the next day using crystal violet staining to determine the ability of the constructs to induce cell death. As can be seen the TNC-sc(mu)TNF80 binding molecule gave the biggest drop in cell viability followed by the Fc(DANA)-TNC-TNF80 binding molecule.

The lower panel of FIG. 25 shows the results for HT1080-Bcl2-TNFR2 cells. The HT1080-Bcl2-TNFR2 cells were stimulated with the indicated concentrations of the various TNF80 variants shown in FIG. 1. Next day, supernatants were harvested and analyzed by ELISA for their IL8 content. The data shown are derived from four independent experiments. As can be seen, the basic murine TNF80 gave the lowest level of induction of IL8 with the other binding molecules stimulating a higher level of IL-8 release.

### Example 22: In Vivo Treg Expansion by Fc(DANA)-TNC-muTNF80

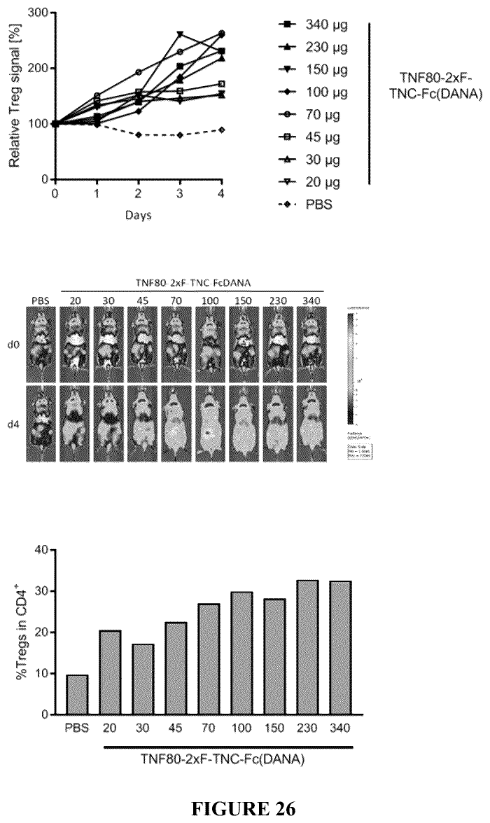

In order to show the ability of the binding molecules comprising TNF80 to act as agonists of TNFR2 in vivo experiments were next performed in mice using the Fc(DANA)-TNC-muTNF80 binding molecule. The results obtained are shown in FIG. 26. Mice with luciferase under the control of the Foxp3 promoter were injected i.v with a single injection of the indicated amount of Fc(DANA)-TNC-muTNF80 and were analyzed daily by bioluminescence imaging (BLI) and were also assessed on day 4 for Treg frequency using FACs analysis. The results of the BLI analysis are shown in the upper panel of FIG. 26. Day 0 and day 4 BLI pictures are shown in the middle panel of FIG. 26. Results for the FACs analysis performed on day 4 are shown in the lower panel of FIG. 26.

### Example 22: Demonstration of Specific Binding for TNFR2

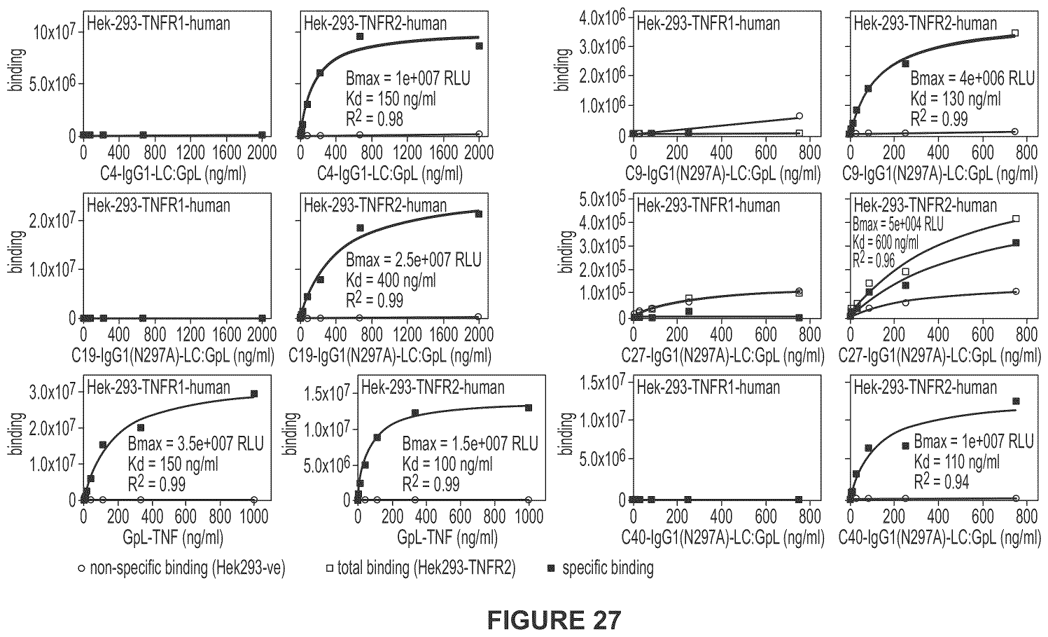

An experiment was performed to illustrate the ability of the binding molecules provided to bind TNFR2, but not TNFR1. HEK293 cells were transiently transfected with: (i) a TNFR2 expression plasmid; (ii) an expression plasmid encoding the extracellular domain of TNFR1 with a GPI anchor domain; or (iii) empty vector (ev). Next day various binding molecules with a GpL tag were assessed for their ability to bind TNFR2, but not significantly bind TNFR1. The results obtained are shown in FIG. 27. The proteins indicated in FIG. 27 were added at the indicated concentration to the HEK293 cells. After incubation for 1 hour at 37° C., followed by two washes with PBS, the remaining cell bound GpL activity was determined. GpL-TNF served as positive control binding to both TNF receptor types.

### Example 23: Reformatting of the Known 005-B08 Antibody

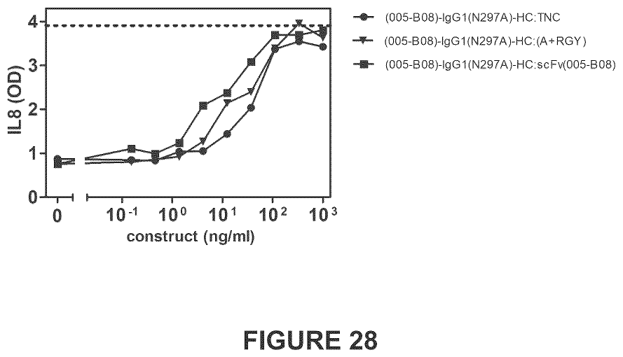

In order to illustrate that “reformatting” known anti TNFR2 antibodies to place them in the formats discussed herein results in useful agonists against TNFR2, even where the known antibodies are anti-TNFR2 antagonist antibodies, a known TNFR2 antagonist was reformatted into some of the tetravalent formats provided. The 005-B08 antibody disclosed in WO2020/089474 was reformatted into several of the formats described herein. Hence, antibodies in the formats IgG1(N297A)-HC:scFv, IgG1(N297A)-HC:TNC, and IgG1(N-RGY) were generated where all the antigen-binding sites originated from the 005-B08 antibody. HT1080-Bcl2-TNFR2 cells were stimulated with supernatants containing the antibodies. Next day, supernatants were analyzed by ELISA for their IL8 content as a measure of agonist activity. The results obtained are presented graphically in FIG. 28. The maximum response induced with C4-IgG1(N297A)-HC:C4 as positive control is indicated by a dotted line in the graph. Even though the original 005-B08 antibody is an antagonist of TNFR2, surprisingly the reformatted tetravalent constructs all displayed good agonist activity showing the impact of the antibody format.

### Example 24: Varying Linker Length and scFv Position in Tetravalent Formats

The possible impact of linker length within the scFv and whether “Southern” scFv groups are linked to the C-terminus of the light chain or heavy chain was studied for the “North” and “South” antibody structures shown in FIG. 29A. The different antibody variants differed in the length of the linker joining the variable heavy and light chains of the scFv, with either three ((G4S)3) or four ((G4S)4) repeats of the basic GGGS (G4S) linker present, as well as whether the scFv groups were joined to the antibody backbone light or heavy chain.

Gel filtration of purified C4-IgG1(Durv)-HC:scFvC4(G4S)4 and C4-IgG1(Durv)-LC:scFvC4(G4S) was performed with the results obtained shown in FIG. 29B. The ability of the constructs to agonise TNFR2 was also studied using IL-8 release as a measure of agonist activity, with the results obtained shown in FIG. 29C. In order to obtain those results HT1080-Bcl2-TNFR2 cells were stimulated with supernatants from HEK293 cells transfected with the indicated constructs. Next day, supernatants were analyzed by ELISA for their IL8 content. The maximum response induced with a highly active oligomeric form of the TNFR2-specific TNF mutant TNF(143N/145R) positive control was indicated by a dotted line. The tetravalent constructs worked well irrespective of the length of linker and whether the scFv groups were linked to the heavy or light chains, illustrating the robust nature of the tetravalent antibody formats provided.

### Example 25: Comparison of C4 Variant Sequences in C4-IgG1(Durv)-HC/LC:scFvC4 Formats

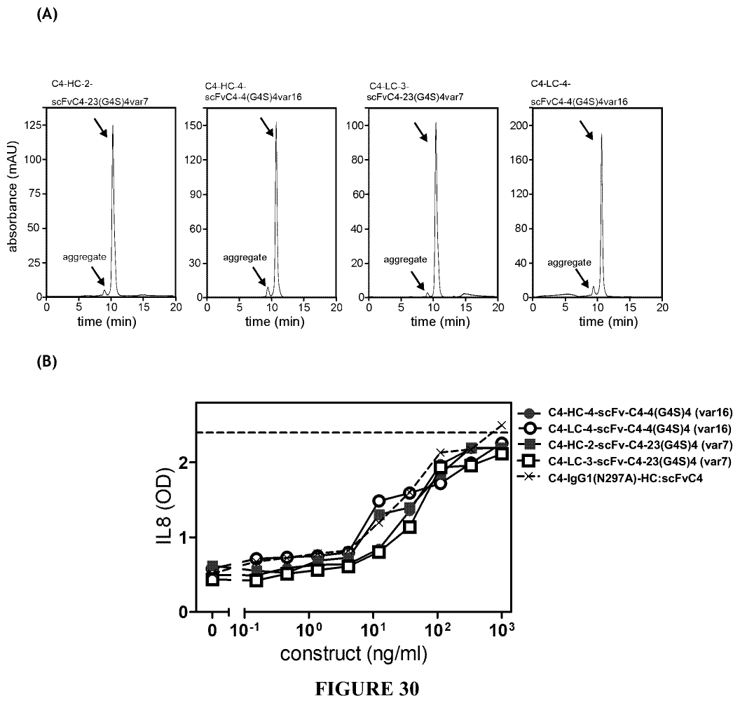

Variants of the C4 variable region were generated in silico, optimizing the sequences in terms of humanization, deimmunization and removal of PTM liabilities with the aim of helping to optimise the sequences further. The C4 variant sequences were used in the C4-IgG1(Durv)-HC/LC:scFvC4 formats. Proteins were produced using HEK293 cells and purified via ProteinA and size exclusion chromatography. The purified variants were assessed using gel filtration chromatography (results shown in FIG. 30, Panel A) and IL-8 release as a measure of TNFR2 agonist activity (results shown in FIG. 30, Panel B). In order to measure IL-8 release, HT1080-Bcl2-TNFR2 cells were stimulated with the indicated constructs. The parental construct C4-IgG1(N297A)-HC:scFvC4 was included for comparison. Next day, supernatants were analyzed by ELISA for their IL8 content. The maximum response induced with a highly active oligomeric form of the TNFR2-specific TNF mutant TNF(143N/145R) positive control is indicated by a dotted line. As can be seen from the results shown in FIG. 30, the C4 variants were highly effective in the IL-8 assay.

### Example 26: Generation and Assessment of VHH Antibodies

In order to provide further options for generating TNFR2 antibodies, a VHH discovery campaign was initiated. Two TNFR2 target proteins (Human-ECD-Fc and Murine-CD-Flag-Fc) and a control protein was used for phage display screening of a single domain antibody (VHH) library (Creative Biolabs library CaVHHL-4). Alternative biopannings were performed for both targets in four rounds and specific VHHs were identified for both targets, while removing negative binders for the control Fc tag. DNA sequencing were performed for 153 clones and 14 unique positive sequences were identified.

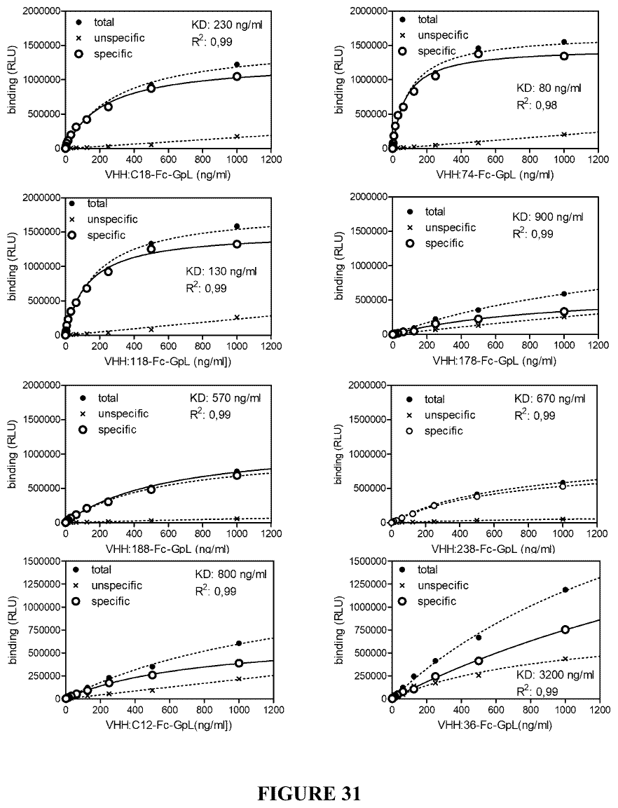

Eight of the 14 clones demonstrated specific binding to human TNFR2 expressed on the cell surface. The ability of the VHH antibodies to bind TNFR2 was studied using VHH-Fc-GpL format proteins each differing in the specific VHH present with the results obtained shown in FIG. 31. The GpL reporter domain was cloned to the C-terminus of the Fc domain of the antibodies. An equilibrium binding assay for the TNFR2-specific VHH-Fc-GpL sdAb constructs was performed using cells expressing human TNFR2 at 37° C., with HEK293 cells used as untransfected control cells. The results obtained are shown in FIG. 31. Specific binding (open circles) was obtained by subtraction of the non-specific binding values (Hek293 cells—crosses) from the binding values for transfectants expressing human TNFR2 (total binding—closed circles). Specific binding was analyzed with the “nonlinear regression to a one-site specific binding curve” function of the GraphPad Prism5 software. As can be seen from FIG. 31, the variants displayed good specific binding to TNFR2.

The equivalent experiment was also performed using TNFR2 from different species, namely from Mus musculus (mouse), Macaca fascicularis (cynomolgus monkey), and Chlorocebus sabaeus (African green monkey) to study the ability of the VHH regions to bind to TNFR2 from different species. The ability to cross react to TNFR2 from different species can be helpful in allowing the antibodies to be studied in various models before assessment in human patients. The results obtained from the experiments are presented in Table 8 below, “n.s.b.” in the Table indicates no specific binding.

TABLE 8
Binding of VHH antibodies to TNFR2
proteins from different species

| | Affinity (ng/ml) | | |
|---|---|---|---|
| VHH | mouse | cyno | Green monkey |
| C12 | n.s.b. | n.s.b. | 80 |
| C18 | >3000 | 50 | 60 |
| C36 | n.s.b. | 90 | 80 |
| C74 | n.s.b. | n.s.b. | 60 |
| C118 | n.s.b. | 50 | 100 |
| C178 | n.s.b. | n.s.b. | 60 |
| C188 | 950 | 450 | 150 |
| C238 | 1050 | 200 | 170 |

### Example 27: Hexavalent Antibodies Using VHH Antibody Domains

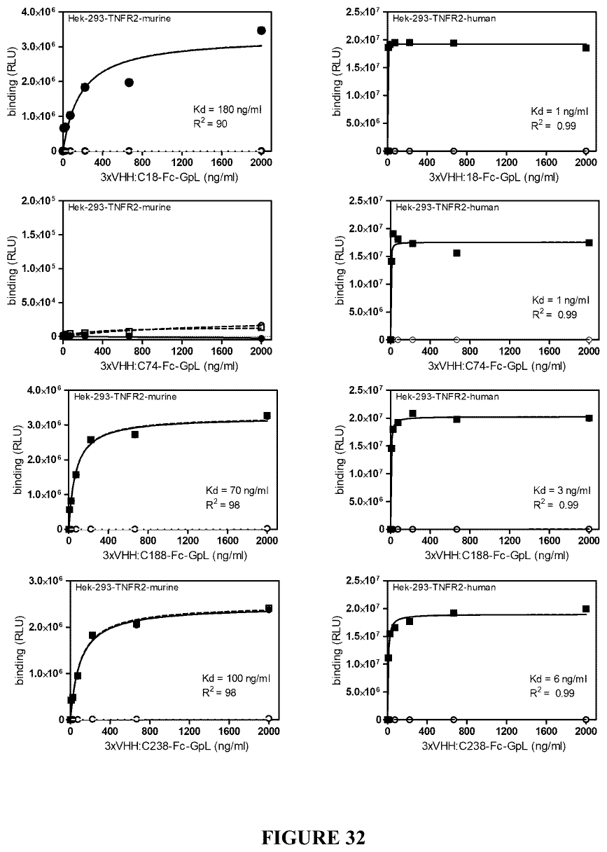

In order to study the impact of antibody format on binding affinity, the VHH domain antibodies were next used to make hexavalent antibodies with 3×VHH-Fc-GpL format antibodies generated for a number of the VHH antibody domains. All of the VHH domains within a given heaxavalent antibody were the same. The equilibrium binding of those antibodies was then studied using the same approach as in Example 26. FIG. 32 shows the results of equilibrium binding of the indicated TNFR2-specific 3×VHH-Fc-GpL sdAb constructs to cells expressing human TNFR2 at 37° C. The GpL reporter domain has been cloned to the C-terminus of the Fc domain. Strikingly, the results obtained showed that the hexavalent format antibodies displayed high affinity binding with the Kd variation between different VHH being generally much lower as compared to the results for the bivalent antibodies shown in FIG. 31. It is thought that the result of having multiple binding sites in close spatial proximity in the hexavalent and higher valency formats may be more important than the specific (bivalent) VHH when it comes to binding affinity, illustrating the robust binding nature of the hexavalent format.

### Example 28: Assessment of Blocking the Binding of TNFα to TNFR2

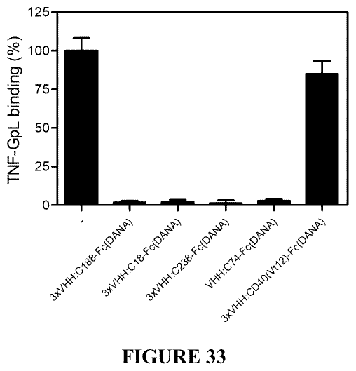

VHH-Fc(DANA) hexavalent format antibodies with VHH:C18, VHH:C74, VHH:C188 or VHH:C238 VHH domains as the binding sites were compared for their ability to block the binding of TNFα to TNFR2. The results obtained are shown in FIG. 33. HT1080-Bcl2-TNFR2 cells were pretreated with the indicated amounts of TNFR2-specific sdAb constructs or with the CD40-specific construct 3×VHH:CD40(V12t)-Fc(DANA) as a negative control as the construct is specific for CD40 not TNFR2. 5 ng/ml GpL-TNF were added to all of the samples and binding was then measured after 1 hour incubation at 37° C. The left most column in FIG. 33 shows the results for the positive control, i.e. in the absence of another construct and hence 100% binding of TNFα to TNFR2. The subsequent columns show the results for the indicated constructs with the activity expressed as a percentage of the value for the negative control. All of the 3×VHH-Fc(DANA) variants of VHH:C18, VHH:C74, VHH:C188 and VHH:C238 in the hexavalent antibody format displayed the ability to potently block TNF binding to the TNFR2 receptor. The V12t CD40-VHH is from “A Bispecific Antibody Antagonizes Prosurvival CD40 Signaling and Promotes Vg9Vd2 T cell-Mediated Antitumor Responses in Human B-cell Malignancies A. C Iris de Weerdt, Roeland Lameris, George L. Scheffer, Jana Vree, Renate de Boer, Anita G. Stam, Rieneke van de Ven, Mark-David Levin, Steven T. Pals, Rob C. Roovers, Paul W. H. I. Parren, Tanja D. de Gruijl, Arnon P. Kater, and Hans J. van der Vliet”.

### Example 29: Ability of Hexavalent Constructs to Specifically Bind TNFR2, Whilst not Binding TNFR1

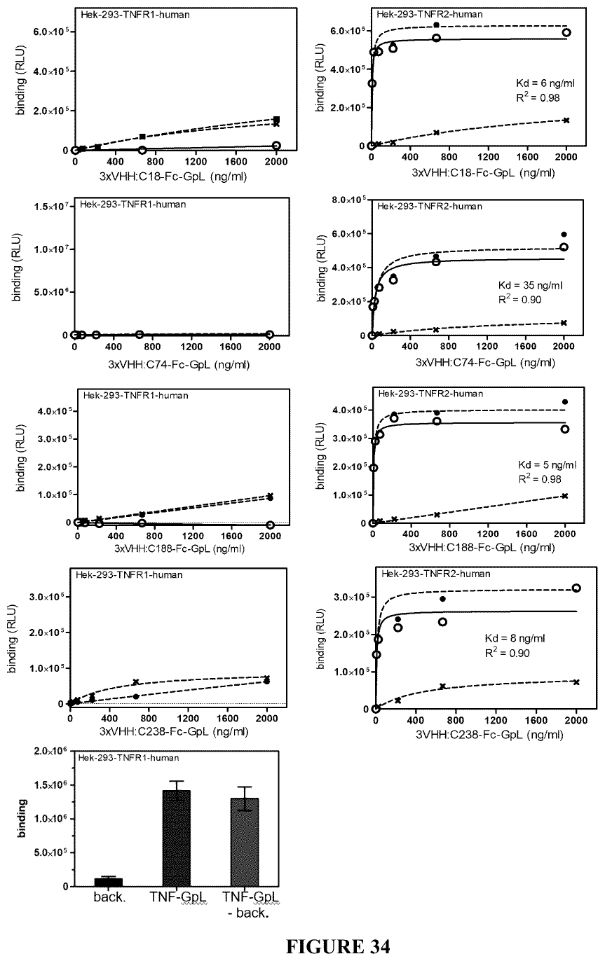

HEK293 cells were transiently transfected with expression vectors encoding TNFR1 and TNFR2 and were then analyzed for binding of various hexavalent constructs with the results obtained shown in FIG. 34. The hexavalent constructs assessed are indicated in FIG. 34. Binding to empty vector transfected cells served as a negative control for non-specific binding. TNF-GpL served as a positive control for successful TNFR1 binding. By comparing the results in the left hand graphs for TNFR1 binding with the corresponding graph on the right hand side for TNFR2 binding, it can be seen that the hexavalent constructs all showed specific binding for TNFR2 compared to TNFR1 allowing selective targeting of the former.

### Example 30: VHH:C18, VHH:C188 and VHH:C238 Bind CRD4 of TNFR2 and VHH:C74 Bind CRD3

Black 96-well plates were coated with protein G and then loaded with the 3×VHH-Fc(DANA) versions of the indicated TNFR2-specific VHH. After removal of the unbound proteins, immobilized antibody constructs were analyzed for binding of the GpL-tagged deletion mutants of TNFR2(ed) shown in the western blot in the right panel. Finally, binding of these GpL fusion proteins were quantified, as shown in FIG. 35. Binding of the VHH clones to the different CRD domains is schematically represented in FIG. 15.

### Example 31: Tetravalent N-Terminal-Only VHH Based Constructs are Strong TNFR2 Agonists

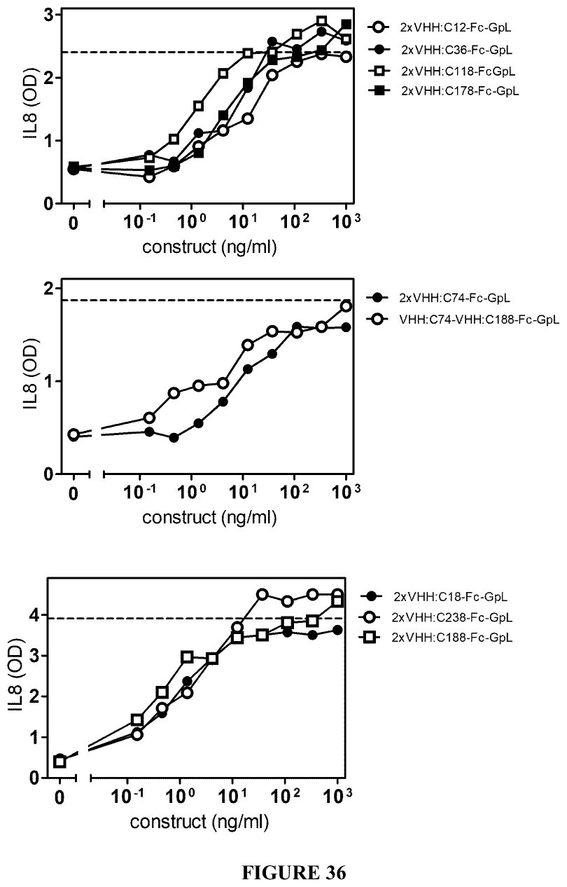

The ability of tetravalent “north-only” TNFR2-specific sdAb variants to act as TNFR2 agonists was next studied with the results obtained shown in FIG. 36. HT1080-Bcl2-TNFR2 cells were stimulated with supernatants containing the indicated mono- or bi-paratopic constructs. Next day, supernatants were analyzed by ELISA for their IL8 content. The maximum response induced with a highly active oligomeric form of the TNFR2-specific TNF mutant TNF(143N/145R) (upper panel) or with C4-IgG1(N297A)-HC:C4 (middle and lower panel) served as positive control and is indicated by a dotted line. The tetravalent “north” only constructs were potent agonists.

### Example 32: Hexavalent N-Terminal-Only TNFR2-Specific sdAb Variants are Strong TNFR2 Agonists

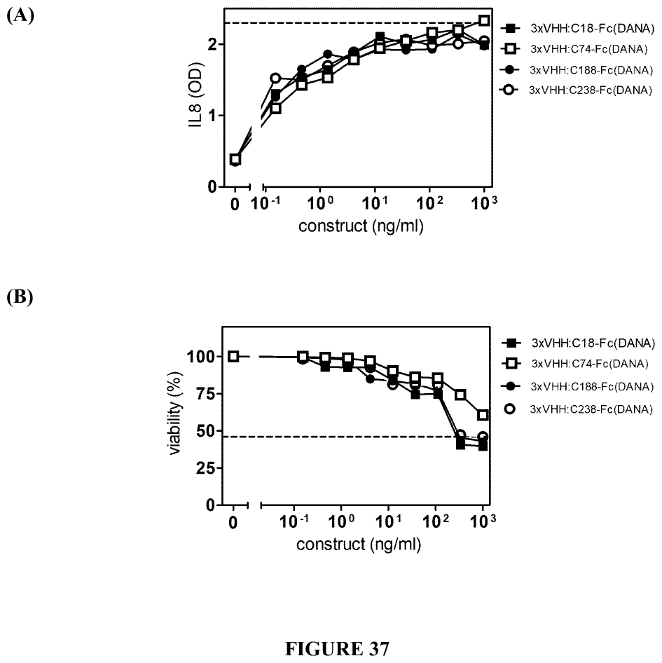

The ability of hexavalent “north-only” VHH antibodies to act as TNFR2 agonists was next studied using both IL-8 release and cell viability as markers. The results obtained are shown in FIG. 37. For the results shown in Panel (A), HT1080-Bcl2-TNFR2 cells were stimulated with the indicated constructs. Next day, supernatants were analyzed by ELISA for their IL8 content. The maximum response induced with a highly active oligomeric form of the TNFR2-specific TNF mutant TNF(143N/145R) positive control is indicated by a dotted line as a positive control. For the results shown in Panel (B) Kym-1 cells were stimulated with the various hexavalent variants and the next day cell viability was determined using the MTT assay. Maximum killing induced by 200 ng/ml of a highly active oligomeric form of the TNFR2-specific TNF mutant TNF(143N/145R) is again indicated by dotted line. This assay measures TNFR2 activation and signaling via a separate pathway to that assessed by measuring IL-8 release. Overall, the results show that the hexavalent “north-only” format displays potent TNFR2 activity with the VHH domains.

### Example 33: Side-by-Side Comparison of the Agonistic Activity of VHH:C188-Fc, 2×VHH:C188-Fc-GpL and 3×VHH:C188-Fc

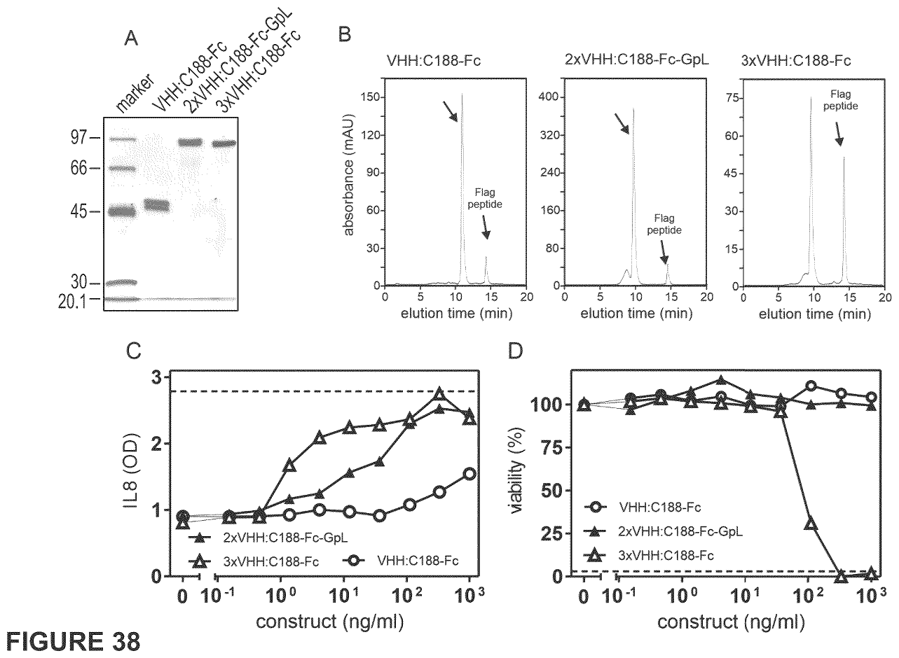

The ability of different antibody formats employing the same VHH domain was compared. The VHH:C188-Fc, 2×VHH:C188-Fc-GpL and 3×VHH:C188-Fc formats were compared, where each employs the C188 VHH domains, with the results obtained shown in FIG. 38. FIG. 38, Panels (A) and (B) show the results for the purified proteins analyzed by SDS-PAGE (A) and gel filtration (B). Panel (C) shows the results for IL-8 release. HT1080-Bcl2-TNFR2 cells were stimulated with the indicated constructs. Next day, supernatants were analyzed by ELISA for their IL8 content. The maximum response induced with a highly active oligomeric form of the TNFR2-specific TNF mutant TNF(143N/145R) positive control is indicated by a dotted line. Panel (D) shows the results for cell viability in Kym-1 cells stimulated with the various VHH:C188 variants indicated. After stimulation the next day cell viability was determined using the MTT assay. Maximum killing induced by C4-IgG1(N297A)-HC:scFvC4 is indicated by a dotted line. The most potent of the three formats was the hexavalent 3×VHH:C188-Fc antibody format, showing the biggest stimulation of IL-8 release and reduction in cell viability.

### Example 34: Side by Side Comparison of IL8- and Cell Death-Inducing Activity of the VHH-Fc-GpL, 2×VHH-Fc-GpL and 3×VHH-Fc-GpL Variants of C18 and C74

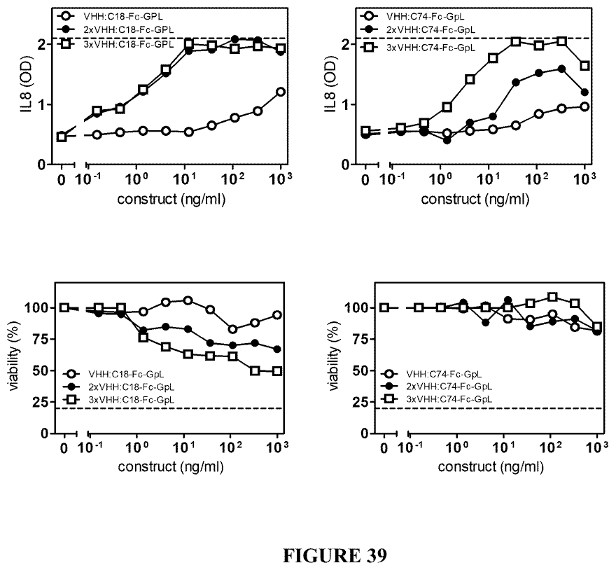

A further comparison of different antibody formats with the same VHH binding domain was performed for VHH-Fc-GpL, 2×VHH-Fc-GpL and 3×VHH-Fc-GpL variants of C18 and C74 using the same IL-8 and cell viability assays in the earlier Examples. The results obtained are shown in FIG. 39. HT1080-Bcl2-TNFR2 cells were stimulated with the indicated constructs. Panel (A) shows the results for supernatants analyzed by ELISA for their IL8 content. Panel (B) shows the results of the cell viability assay, where Kym-1 cells were stimulated with the various variants and the next day, cell viability was determined using the MTT assay. The maximum responses induced with a highly active oligomeric form of the TNFR2-specific TNF mutant TNF(143N/145R) is indicated. Interestingly, the hexavalent format was most active in both assays for the C18 variant. The hexavalent format for C74 was the best format for stimulating IL-8 release, but none of the formats triggered a marked drop in cell viability. It is thought this indicates that the C74 VHH antibody does not trigger both TNFR2 mediated signaling pathways, only stimulating signaling in the pathway that brings about IL-8 release.

### Example 35: Linker Length Between VHH Domains of 3×VHH:C188-IgG1(Durv) Variants does not Affect TNFR2 Agonism

The impact of separating VHH domains with progressively longer linkers was next studied with the 3×VHH:C188-IgG1(Durv) antibody format employed with progressively longer linkers. The different antibodies compared either had a single G4S linker (GSSSS) between the VHH domains, three linkers between the domains (G4S)3, or five linkers between the domains (G4S)5. The results obtained are shown in FIG. 40. Panel (A) shows the domain architecture of constructs. Panel (B) shows SDS-PAGE analysis of purified constructs and panel (C) the results of the gel filtration analysis. Panel (D) shows the results of the IL-8 release assay, with HT1080-Bcl2-TNFR2 cells were stimulated with supernatants containing the indicated constructs. Next day, supernatants were analyzed by ELISA for their IL8 content. The maximum response induced with a highly active oligomeric form of the TNFR2-specific TNF mutant TNF(143N/145R) is indicated by a dotted line. The parental molecule 3×VHH:C188-Fc(DANA) was included for comparison. As can be seen from FIG. 40, Panel (D) increase in overall linker length had no impact on TNFR2 agonism as measured by IL-8 release, further illustrating the robust nature of the hexavalent format.

### Example 36: Constructs of Different Domain Architecture with 2, 3, 4, 6 or 9 VHH:C188 Domains Elicit Strong TNFR2 Agonism

Different antibody formats with were further compared using the C188 VHH domain. The results obtained are shown in FIG. 41. Panel (A) shows the domain architecture of antibodies. TNC refers to the trimerization domain of tenascin-C. Panels (B) shows the results for TNFR2 agonism activity as measured by IL-8 release. HT1080-Bcl2-TNFR2 cells were stimulated with the indicated constructs. Next day, supernatants were analyzed by ELISA for their IL8 content. The maximum response induced with a highly active oligomeric form of the TNFR2-specific TNF mutant TNF(143N/145R) is indicated by a dotted line. The constructs with at least four VHH domains in general gave the best results, particularly for those antibodies where at least two VHH domains were present on the same polypeptide.

### Example 37: Protein G Enhances the Agonistic Activity of Sequence Optimized Variants of the Anti-TNFR2 mAb C4

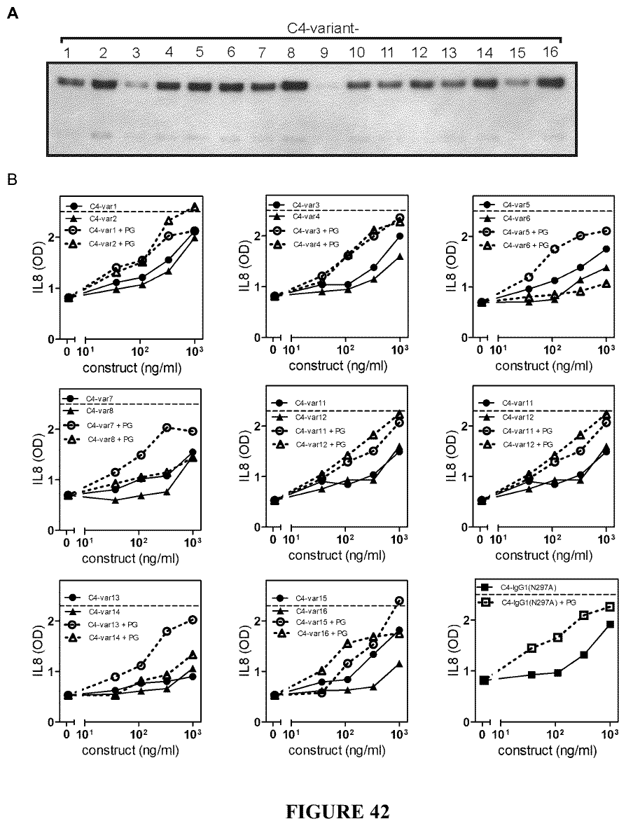

As shown in FIG. 42, enhanced activity in the presence of protein G was obtained for C4 variant Abs, which indicates the presence of latent agonism that can be realized by multimerization of monoclonal antibodies. (A) Western blot analysis with anti-human IgG1 antibody of cell culture supernatants of HEK293 cells transiently transfected with expression plasmids encoding the light and heavy chain of the indicated antibody variants. (B) HT1080-TNFR2 cells were challenged with the indicated concentrations of the C4 variants in the presence and absence of 500 ng/ml protein G. Next day, cell supernatants were analyzed for IL8 production by ELISA. The maximum response induced with a highly active oligomeric form of the TNFR2-specific TNF mutant TNF(143N/145R) is indicated by a dotted line.

### Example 38: TNFR2 Agonism of Sequence Optimized Variants of 3×VHH:C188-Fc(Durv)

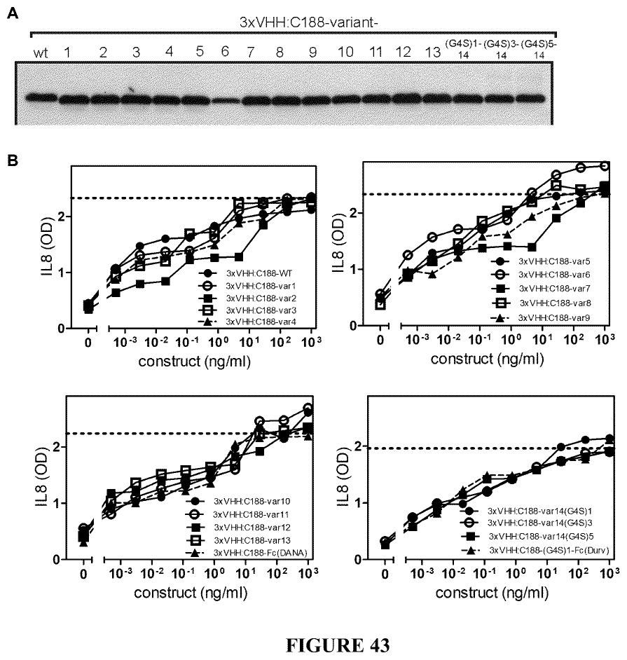

Fifteen variants were generated in silico with optimized sequences in terms of humanization, deimmunization, removal of PTM liabilities. For one variant (variant 14) constructs were also made with varying linker length between the different VHHs. As shown in FIG. 43, panel (A), all variant constructs were successfully produced: Western blot analysis with anti-human IgG1 antibody of cell culture supernatants of HEK293 cells transiently transfected with expression plasmids encoding the indicated 3×VHH:C188-Fc(Durv) variants. In the 3×VHH:C188-Fc(Durv) format, all variants were bioactive as can be seen in panel (B) showing the results of the IL-8 release assay, in which HT1080-Bcl2-TNFR2 cells were stimulated with supernatants containing the indicated constructs: HT1080-TNFR2 cells were challenged with the indicated concentrations of the 3×VHH:C188-Fc(Durv) variants and the parental 3×VHH:C188-Fc(DANA) molecule. Next day, cell supernatants were analyzed for IL8 production by ELISA. The maximum response induced with a highly active oligomeric form of the TNFR2-specific TNF mutant TNF(143N/145R) is indicated by a dotted line.

### Example 39: TNFR2-Agonists Expand Tregs within Human Peripheral Blood Mononuclear Cells (PBMCs)

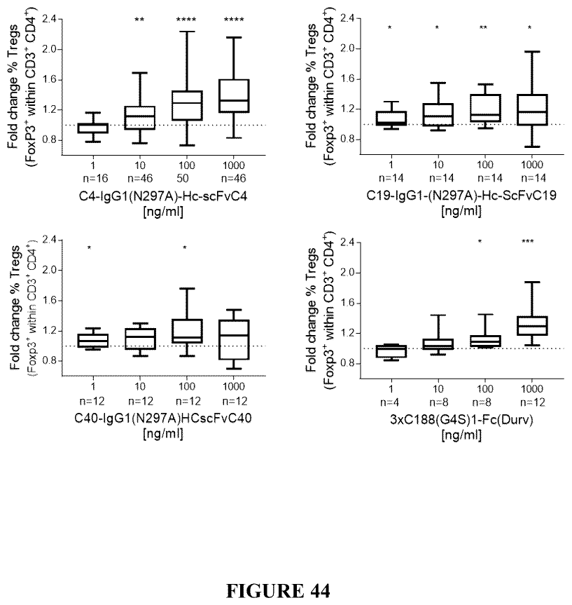

The ability of particular TNFR2 binding molecules to expand regulatory T cells with different TNFR2 binding sites to expand peripheral blood mononuclear cells was assessed. Results obtained with tetravalent antibodies in the Fab-HC-scFv format (C4-IgG1(N297A)-HC:scFvC4, C19-IgG1(N297A)-HC:scFvC19, and C40-IgG1(N297A)-HC:scFvC40), and hexavalent VHH constructs in 3×VHH-Fc format (3×VHH:C188-Fc(Durv) are shown in FIG. 44, and are an expansion on the data shown in FIG. 21. PBMCs were isolated from the indicated number of donors per condition, with the cells from each donor assessed separately. The PBMC were cultured for two days at a density of 10⁷ cells/ml. The PBMCs were then harvested and seeded into multi-well plates at a density of 10⁶ cells/ml, with 500,000 cells per well. Following culturing for four days, cells were stained with antibodies specific for CD3, CD8, CD4, CD25 and FoxP3 for flow cytometric analysis. FIG. 44 shows fold change of Treg frequency of human peripheral blood mononuclear cells (PBMCs) after 4 days of stimulation with indicated TNFR2-agonist relative to untreated control of the same donor. Samples were measured with flow cytometry and Tregs were defined as FoxP3⁺CD4⁺CD3⁺ T cells. Number of individual donors are indicated with n. Significance was tested with one-sample t test with a hypothetical value 1 and is indicated with *P=0.05, **P=0.01, ***P=0.001, ****P<0.0001.

### Example 40: C4-IgG1(N297A)-HC:scFvC4 Activate Tregs

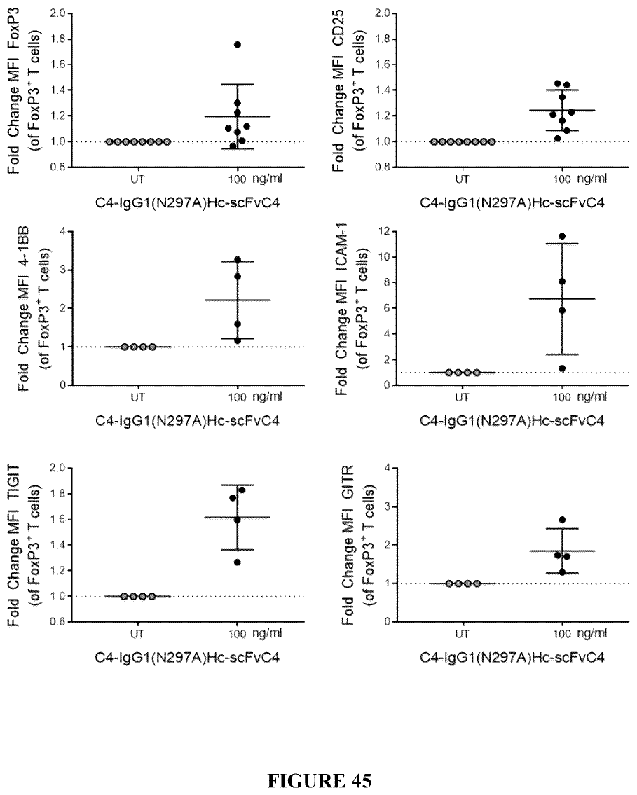

The ability of IgG1(N297A)-HC:scFv format antibodies with C4 TNFR2 binding sites to activate Tregs was further studied. The results obtained are shown in FIG. 45. Human PBMCs were stimulated for 4 days with 100 ng/ml C4-IgG1(N297A)-HC:scFvC4 and expression of markers related to Treg activity was measured with flow cytometry. Fold change expression relative to untreated control of the same donor is shown. Tregs were defined as FoxP3⁺ CD4⁺ CD3⁺ T cells. Mean±SD and each dot represents individual donor.

### Example 41: 3×VHHC188(G4S)1-Fc(Durv) Activates Tregs

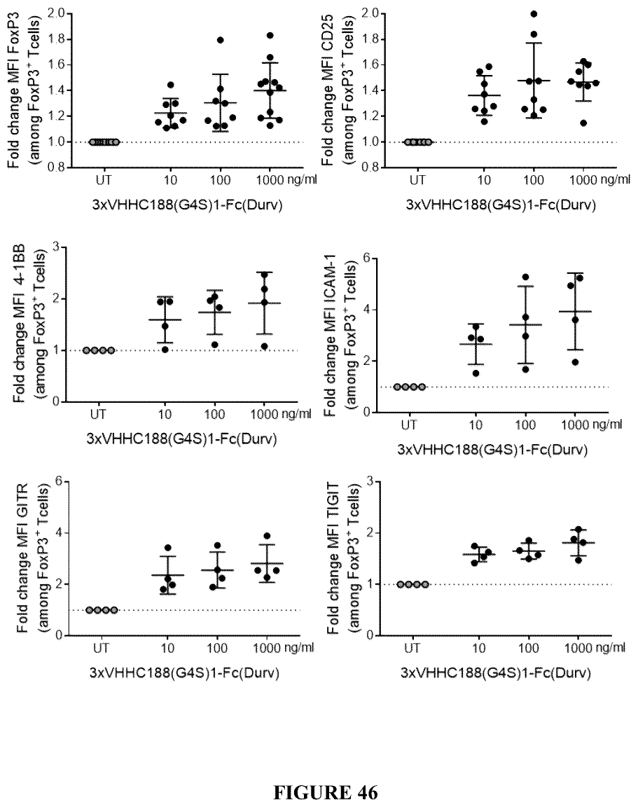

The ability of a hexavalent antibody format comprising VHH domains to activate Tregs was studied using the 3×VHHC188(G4S)1-Fc(Durv) format antibody. The results obtained are shown in FIG. 46. Human PBMCs were stimulated for 4 days with indicated concentration of 3×VHHC188(G4S)1-Fc(Durv) and expression of markers related to Treg activity was measured with flow cytometry. Fold change expression relative to untreated control of the same donor is shown. Tregs were defined as FoxP3⁺ CD4⁺ CD3⁺ T cells. Mean±SD and each dot represents individual donor.

### Example 42: TNFR2 Agonists Expand Human-TNFR2-Expressing CD4 T Cells In Vitro

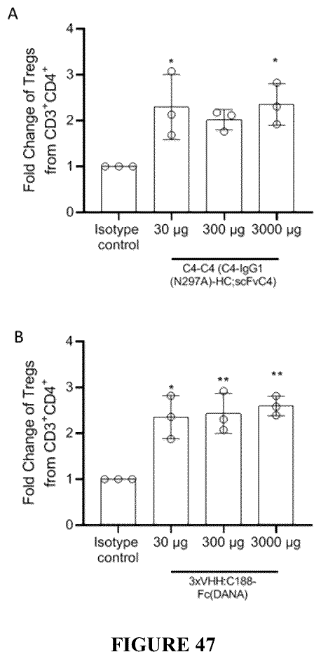

Enriched CD4 T cells obtained from human-TNFR2 knockin mice were stimulated with different concentrations of (A) C4-C4 (C4-IgG1(N297A)-HC:scFvC4) or (B) 3×VHH:C188-Fc(DANA) increased Treg frequencies after 4 days of culture with the respective agonists. Data in FIG. 47 are represented as fold change increase of CD25^{high}FoxP3^{high} CD4 T cells over untreated (isotype control) samples.

### Example 43: Serum Retention of Fc(DANA)-TNC-muTNF80

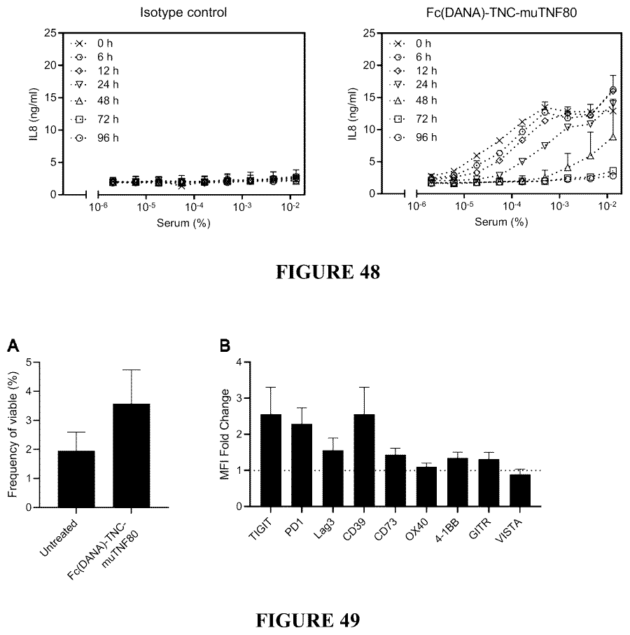

Serum retention of Fc(DANA)-TNC-muTNF80 was next studied with the results obtained shown in FIG. 48. IL-8 concentrations measured by ELISA of supernatants of HT1080-Bcl2-TNFR2 cell cultures stimulated with serum isolated from peripheral blood at the given time points from wild type mice injected at 0 hours with 75 μg of Fc(DANA)-TNC-muTNF80 or isotype control antibody. Data are shown in FIG. 48.

### Example 44: Ligand-Based TNFR2 Agonists Expand Tregs In Vivo and Upregulate Activation Markers

The ability of ligand-based TNFR2 agonists to expand Tregs in vivo and upregulate activation markers was next studied with the results obtained shown in FIG. 49. (A) Treg frequency of viable cells in spleens isolated four days after stimulation with 75 μg Fc(DANA)-TNC-muTNF80 in wild type mice. (B) Fold change of selected extracellular markers defined as the mean fluorescence intensity (MFI) measured for Tregs and formalized against isotype control antibody samples. Data are shown in FIG. 49.

### Example 45: TNFR2 Agonist Increases Splenic Tregs In Vivo

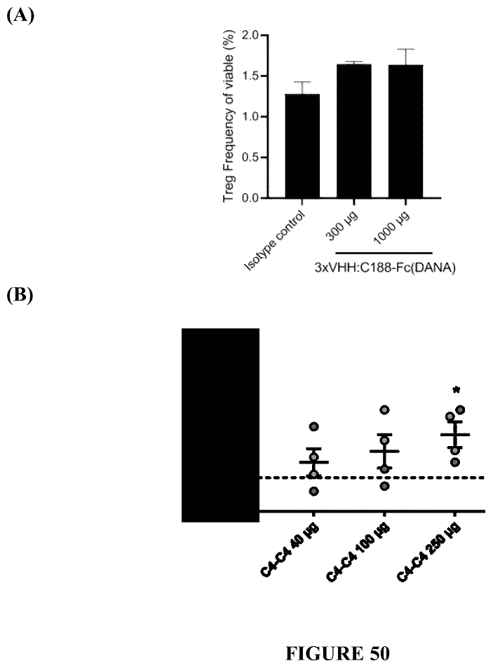

Treg frequency was determined in spleens from huTNFR2-expressing mice isolated four days after treatment of the mice with 3×VHH:C188-Fc(DANA), or isotype control antibody, or C4-IgG1(N297A)-HC:scFv:C4. Fold change was calculated relative to isotype control or untreated control in the same experiment. As shown in FIG. 50, TNFR2 agonist can in vivo expand Treg in the spleen of human TNFR2 KI mice. Comparison of means between treatment groups and control done through unpaired t-Test. * p≤0.05, ns=not.

### Example 46: TNFR2 Agonists Increase Survival after Allogeneic Hematopoietic Cell Transplantation (Allo-HCT) Induced Acute Graft-Versus-Host Disease

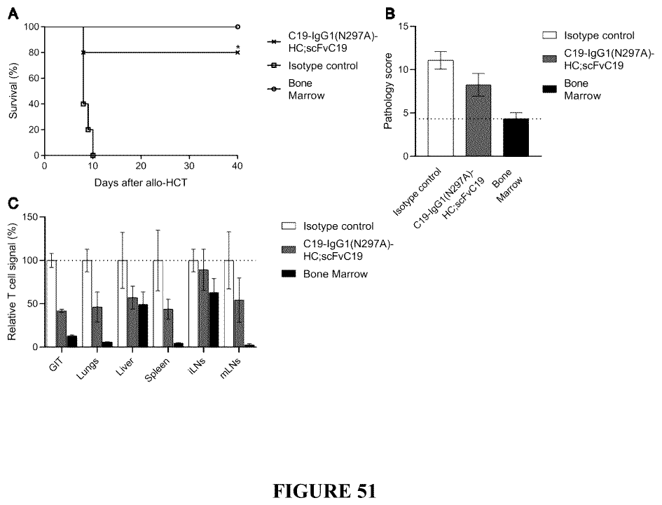

The impact of TNFR2 agonists in a model of graft-versus host disease (GvHD) was studied, with the results obtained shown in FIG. 51. Panel (A) shows survival frequencies after allo-HCT induced experimental acute GvHD in mice (FVB/N H-2-C57BL/6 H-2^{b}, MHC major mismatch model). Statistical significance assessed with Log-Rank test between each TNFR2 agonist treatment group and isotype control antibody, * p≤0.05. Panel (B) shows pathology score of intestinal samples isolated on day 6 after allo-HCT based on immune cell infiltrates and intestinal crypt damage.

### Example 47: TNFR2 Agonist Expands Treg from African Green Monkey (Vervet) Peripheral Blood Mononuclear Cells (PBMCs)

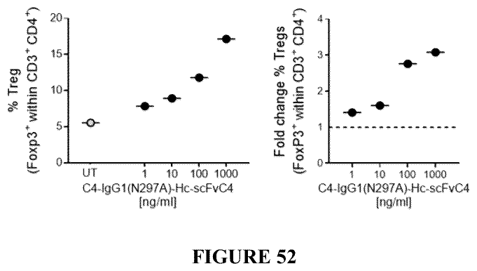

Frozen peripheral blood mononuclear cells of African Green monkey were thawed, cultured at 2×10⁵ cells per well and either left untreated or treated with 1, 10, 100, 1000 ng/mL C4-IgG1(N297A)-HC:scFvC4 for 5 days at 37° C. Samples were measured with flow cytometry and Tregs were characterized as CD3+ CD4+ FoxP3+ T cells. As shown in FIG. 52, TNFR2 agonist treatment results in dose dependent expansion of Treg from African green monkey PBMC.

### Example 48: Clones C188 and C4 Compete with TNF for Binding to Human TNFR2

HT1080-Bcl2-TNFR2 cells were pretreated with the indicated amounts of TNFR2-specific VHH constructs or with the CD20-specific mAb Rituximab as a negative control or remained untreated. 5 ng/ml GpL-TNF were added and binding was measured after 1 h incubation at 37° C. The amount of binding to TNF-GpL to the HT1080-Bcl2-TNFR2 cells is abrogated by the presence of clone C188 as C188-VHH-IgG1(N297A) VHH-Fc format and clone C4 as C4-IgG1(N297A) antibody format, as shown in FIG. 35 panel B.

1. A binding molecule that specifically binds TNFR2, but not TNFR1, where the binding molecule is an FcγR independent agonist of TNFR2 and has a valency of at least two for binding TNFR2.

2. The binding molecule of claim 1, wherein the binding molecule has a valency of at least four.

3. The binding molecule of claim 1 or 2, wherein:
(a) the binding molecule has a valency of six for TNFR2; or
(b) the binding molecule has a valency of four for TNFR2.

4. The binding molecule of any one of the preceding claims, wherein at least one antigen-binding domain specific for TNFR2 of the binding molecule is a single domain binder (sdB) specific for TNFR2, preferably a single domain antibody (sdAb) specific for TNFR2.

5. The binding molecule of claim 4, wherein the at least one sdBr (single domain binder) specific for TNFR2 is a VHH.

6. The binding molecule of any one of the preceding claims, wherein the binding molecule comprises six TNFR2 specific antigen-binding sites that are each VHH antigen-binding domains specific for TNFR2.

7. The binding molecule of claim 6, wherein:
(a) the binding molecule comprises six VHH antigen-binding domains and an Fc region, preferably wherein the binding molecule is a hexavalent antibody;
(b) the binding molecule comprises two polypeptides, wherein each polypeptide comprises three VHH binding domains specific for TNFR2, preferably wherein the binding molecule is a hexavalent antibody;
(c) the binding molecule comprises two polypeptides, wherein each polypeptide comprises three VHH binding domains specific for TNFR2 with each polypeptide further comprising in its C-terminal portion a heavy chain constant region sequence comprising CH2-CH3, but not CH1, preferably wherein the binding molecule is a hexavalent antibody;
or
(d) the binding molecule comprises two polypeptides, where each polypeptide comprises three VHH binding domains specific for TNFR2 with each polypeptide further comprising in its C terminal portion a heavy chain constant region sequence comprising CH2-CH3, but not CH1, and modifications meaning that the Fc domain has reduced effector function, preferably wherein the binding molecule is a hexavalent antibody.

8. The binding molecule of any one of the preceding claims, wherein:
(a) the binding molecule comprises an antigen-binding site comprising a VHH domain comprising a set of three CDRs selected from the sets of SEQ ID NOs: 213/214/215; 216/217/218; 219/220/221; 222/223/224; 225/226/227; 228/229/230; 231/232/233; 234/235/236; 237/238/239; 240/241/242; 243/244/245; 246/247/248; 249/250/251; 252/253/254; or a variant set of CDRs of any of those sets with up to a total of five amino acid sequence changes, with the variant antigen-binding site retaining the ability to bind TNFR2;
(b) the binding molecule comprises an antigen-binding site comprising a VHH domain comprising the three CDRs of SEQ ID NOs: 154/155/156 or a variant set of those three CDRs with up to a total of five amino acid sequence changes with the variant antigen-binding site retaining the ability to bind TNFR2;
(c) the binding molecule comprises an antigen-binding site comprising a set of three CDRs selected from 154/155/156; 160/161/162; 163/164/165; 166/167/168; 169/170/171; 181/182/183; 187/188/189; 193/194/195 or a variant set of any thereof with up to five amino acid sequence changes with the antigen-binding site retaining the ability to bind TNFR2;
(d) the binding molecule is hexavalent comprising six VHH antigen-binding sites which are all the same and are an antigen-binding site as defined in any of (a) to (c);
(e) the binding molecule is hexavalent comprising two polypeptides, wherein each comprises three VHH antigen-binding sites, wherein all six antigen-binding sites are the same antigen-binding sites which is as defined in any of (a) to (c);
(f) the binding molecule is hexavalent comprising six VHH antigen-binding sites, wherein at least two of the VHH antigen-binding sites have a different specificity for TNFR2, with at least one of those specificities conferred by a VHH antigen-binding site as defined in any of (a) to (c);
(g) the binding molecule is hexavalent comprising six VHH antigen-binding sites, wherein at least three of the VHH antigen-binding sites have a different specificity for TNFR2, with at least one of those specificities conferred by a VHH antigen-binding site as defined in any of (a) to (c); or
(h) the binding molecule is hexavalent comprising six VHH antigen-binding sites, wherein all six VHH antigen-binding sites have a different specificity for TNFR2, with at least one of those specificities conferred by a VHH antigen-binding site as defined in any of (a) to (c).

9. The binding molecule of any one of claims 1 to 8, wherein:
(a) the binding molecule comprises at least one VHH antigen-binding domain of SEQ ID No: 198 to 211 or a variant thereof with at least 90% sequence identity which is still able to bind TNFR2;
(b) the binding molecule comprises at least one VHH antigen-binding domain selected from SEQ ID No: 140 or a variant of any thereof with at least 90% sequence identity;
(c) the binding molecule comprises at least one VHH antigen-binding domain selected from those of SEQ ID Nos: 140, 142 to 145, 149, 151, 153, or a variant of any thereof with at least 90% sequence identity which is still able to bind TNFR2;
(d) the binding molecule comprises two polypeptides which each comprise at least one VHH antigen-binding domain as defined in (a), (b), to (c);
(e) the binding molecule comprises two polypeptides where each comprise three VHH antigen-binding domains as defined in (a), (b) or (c);
(f) the binding molecule comprises two polypeptides where each comprise three VHH antigen-binding domains as defined in (a), (b) or (c), wherein all of the antigen-binding domains of the binding molecule specific for TNFR2 are the same; or
(g) the binding molecule comprises two polypeptides where each comprise three VHH antigen-binding domains as defined in (a), (b) or (c), wherein not all of the VHH antigen-binding domains of the binding molecule specific for TNFR2 are the same.

10. The binding molecule of any one of claims 1 to 7, wherein:
(a) the binding molecule comprises two polypeptides which each have:
(i) the amino acid sequence of SEQ ID NO: 197, except that the VHH domain corresponding to SEQ ID NO: 140 within that sequence is instead the sequence of one of SEQ ID NOs: 198 to 211; or
(ii) a variant amino acid sequence with at least 90% sequence identity to (i) and which is still able to bind TNFR2;
(b) the binding molecule comprises two polypeptides which each have the amino acid sequence of SEQ ID NO: 197 or a variant amino acid sequence with at least 90% sequence identity which is still able to bind TNFR2, preferably where each polypeptide comprises three such VHH binding domains;
(c) the binding molecule comprises two polypeptides which each have:
(i) the amino acid sequence of SEQ ID NO: 197, except that the VHH domain corresponding to SEQ ID NO: 140 within that sequence is instead the sequence of one of SEQ ID NOs: 140, 142 to 145, 149, 151, or 153;
(ii) an amino acid sequence with at least 90% sequence identity to (i) and which is still able to bind TNFR2;
(d) any one of (a) to (c) wherein the variant amino acid sequence has from one to five amino acid sequence changes compared to the specific sequence and the binding molecule retains the ability to bind TNFR2, preferably wherein the variant has only one, two or three, and more preferably one amino acid sequence change compared to the specific sequence; or
(e) any one of (a) to (c) wherein the variant amino acid sequence has the sequence of amino acids 302 to 405 of SEQ ID NO: 197 replaced with the amino acid sequence of SEQ ID NO: 327.

11. The binding molecule of any one of claims 1 to 7, wherein:
(a) the binding molecule comprises a polypeptide having the amino acid sequence of SEQ ID NO: 330 or a variant thereof with at least 90% sequence identity that is still able to bind TNFR2;
(b) the binding molecule comprises a polypeptide having the amino acid sequence of SEQ ID NO: 331 or a variant thereof with at least 90% sequence identity that is still able to bind TNFR2; or
(c) the binding molecule comprises three polypeptides of (a) or three polypeptides of (b).

12. The binding molecule of any one of claims 1 to 3, wherein the binding molecule is an antibody with a valency of four with both “N-terminal” and “C-terminal” orientation binding sites for TNFR2.

13. The binding molecule of any one of claims 1 to 3 or 12, wherein:
(a) the binding molecule comprises an antigen-binding site comprising a set of six CDRs wherein:
(i) the light chain CDRs are selected from a set of CDR1/CDR2/CDR3 selected from SEQ ID NOs: 266/267/268; 269/270/271; 272/273/274; 275/276/277 and the heavy chain CDRs are selected from a set of CDR1/CDR2/CDR3 selected from 282/283/284; 285/286/287; 288/289/290; or 291/292/293; or
(ii) the set of six CDRs is a variant of a set of six CDRs of (i) with no more than a total of five amino acid changes compared to the specific six CDRs and wherein the antigen-binding site retains the ability to bind TNFR2;
(b) the binding molecule comprises an antigen-binding site comprising a set of six CDRs, wherein:
(i) the light chain CDR1/CDR2/CDR3 are SEQ ID NOs 69/70/71 and the heavy chain CDR1/CDR2/CDR3 are SEQ ID NOs: 102/103/104;
(ii) the set of six CDRs is a variant of a set of six CDRs of (i) with no more than a total of five amino acid changes compared to the specific six CDRs and wherein the antigen-binding site retains the ability to bind TNFR2;
(c) the binding molecule comprises an antigen-binding site comprising a set of three light chain CDRs and/or a set of three heavy chain CDRs from those set out in Table 5;
(d) the binding molecule comprises a set of six CDRs from a set of six set out in Table 5 or a variant set of six CDRs with no more than five amino acid sequence changes and the variant retaining the ability to bind TNFR2;
(f) the binding molecule comprises four antigen-binding sites as set out in any one of (a) to (d) with all four antigen-binding sites being the same;
(g) the binding molecule comprises four antigen-binding sites as set out in any one of (a) to (d) with all four antigen-binding sites being the same, with the antibody being tetravalent for TNFR2; or
(h) the binding molecule comprises four antigen-binding sites as set out in any one of (a) to (d) with all four antigen-binding sites being the same, with the antibody being tetravalent for TNFR2, with two N-terminal orientated antigen-binding sites and two C-terminal orientated antigen-binding sites.

14. The binding molecule of any one of claims 1 to 3 or 12, wherein the binding molecule is an antibody comprising:
(a) two light chain polypeptides which are:
(i) two light chain polypeptides wherein each light chain polypeptide comprises an amino acid sequence having the sequence of SEQ ID NO: 261 except that the amino acid sequence corresponding to SEQ ID NO: 295 is replaced with a sequence selected from one of SEQ ID NOs: 262 to 265; or
(ii) two variant light chain polypeptides which each have at least 90% sequence identity to (i); and
(b) two heavy chain polypeptides which are:
(iii) two heavy chain polypeptides wherein each heavy chain polypeptide comprises an amino acid sequence having the sequence of SEQ ID NO: 259 except that the amino acid sequence corresponding to SEQ ID NO: 296 is replaced with a sequence selected from one of SEQ ID NOs: 278 to 281; or
(iv) two variant heavy chain polypeptides which each have at least 90% sequence identity to (iii).

15. The binding molecule of any one of claims 1 to 3 or 12, wherein the binding molecule is an antibody comprising:
(a) two light chain polypeptides having the sequence of SEQ ID NO: 261 or an amino acid sequence having at least 90% amino acid sequence identity to SEQ ID NO: 261; or
(b) two heavy chain polypeptides having the sequence of SEQ ID NO: 259 or an amino acid sequence having at least 90% amino acid sequence identity to SEQ ID NO: 259.

16. The binding molecule of any one of claims 1 to 3 or 12, wherein the binding molecule:
(a) comprises a light chain of SEQ ID NO: 332 and heavy chain of SEQ ID NO: 333 or variants thereof with at least 90% sequence identity where the binding molecule is still able to act as a TNFR2 agonist;
(b) comprises a light chain of SEQ ID NO: 334 and heavy chain of SEQ ID NO: 335 or variants thereof with at least 90% sequence identity where the binding molecule is still able to act as a TNFR2 agonist;
(c) comprises a light chain of SEQ ID NO: 336 and heavy chain of SEQ ID NO: 337 or variants thereof with at least 90% sequence identity where the binding molecule is still able to act as a TNFR2 agonist; or
(d) comprises two light and two heavy chains all as defined in (a), (b), or (c).

17. The binding molecule of any one of the preceding claims which is able to:
(a) oligomerise TNFR2 on the surface of a cell expressing TNFR2, preferably to cluster six or more TNFR2 molecules on the cell surface;
(b) trigger TNFR2 signalling; and/or
(c) stimulate proliferation of leucocytes, preferably T cells, more preferably Treg cells.

18. The binding molecule of claim 1, wherein:
(a) the binding molecule comprises a moiety that alters serum half-life, preferably wherein the moiety is selected from the group consisting of a Fc tail, serum albumin, a moiety which is a binder of serum albumin, and PEG;
(a) the binding molecule is bivalent for TNFR2 binding;
(b) the binding molecule is trivalent for TNFR2 binding;
(c) the binding molecule is tetravalent for TNFR2 binding; or
(d) the binding molecule which has a valency of at least four for TNFR2 binding.

19. The binding molecule of claim 1, wherein:
(a) the binding molecule is an antibody;
(b) the binding molecule is an antibody which comprises a ligand for TNFR2;
(c) the binding molecule comprises at least part of an antibody constant region, preferably an Fc region; or
(d) the binding molecule comprises a ligand for TNFR2, preferably wherein the ligand is derived from TNFα or lymphotoxin-alpha,
preferably where the binding molecule is:
(a) an antibody that comprises a TNFR2 binding site at both the N-terminus and C-terminus of one or more heavy chain of the antibody; or
(b) an antibody that comprises a TNFR2 binding site at both the N-terminus and C-terminus of one or more light chain of the antibody.

20. The binding molecule of claim 19, wherein:
(a) the antibody is a bivalent antibody;
(b) the antibody is a trivalent antibody;
(c) the antibody is tetravalent antibody;
(d) the antibody is a hexavalent antibody;
(e) the antibody is a heavy chain only antibody;
(f) the antibody is a single domain antibody, such as a single domain Ab (sdAb), VHH, or a Nanobody;
(g) the antibody is a multimer of at least two antibodies;
(h) the antibody comprises, or is linked to, a multimerizing moiety preferably tenascin-c peptide (TNC), preferably where the antibody is a multimer of at least two antibodies associated through the tenascin-c peptide sequence;
(i) the antibody is linked to one or more molecules derived from a ligand of TNFR2, preferably derived from TNF-α or lymphotoxin alpha.

21. The binding molecule of claim 19 or 20 wherein:
(a) the antibody lacks an Fc region;
(b) the antibody comprises a modified Fc region that is unable to bind Fc receptors; or
(c) the antibody comprises heavy chain constant regions lacking CH1.

22. The binding molecule of claim 1, wherein the binding molecule is an antibody in an antibody format selected from:
IgG with an Fc modification eliminating Fc receptor binding;
scFv:IgG where the IgG has an Fc modification eliminating Fc receptor binding;
IgG-HC:TNC, where the IgG has a modification eliminating Fc receptor binding;
scFv:IgG-HC:TNC where the IgG has a modification eliminating Fc receptor binding;
hexameric IgG;
3(VHH-TNC);
wherein:
HC represents a heavy chain of an IgG;
LC represents a light chain of an IgG;
scFv:IgG represents a scFv either linked to, or forming part of an IgG molecule;
HC:TNC indicates a heavy chain (HC) comprising, or linked to, tenascin-c trimerization domain peptide (TNC);
VHH-TNC indicates tenascin linked or bound to a VHH.

23. The binding molecule of claim 1, wherein the binding molecule is an antibody in an antibody format selected from the group consisting of:
Fab-LC:scFv;
IgG-HC:scFv where the IgG has a modification eliminating Fc receptor binding, preferably N297A;
IgG-LC:scFv where the IgG has a modification eliminating Fc receptor binding, preferably N297A;
IgG-LC:scFv-HC: scFv;
LC:scFv-HC: scFv-IgG-HC: scFv;
LC:scFv-HC:scFv-IgG-LC:scFv;
LC: scFv-HC:scFv-IgG-LC: scFv-HC: scFv; and
IgG-HC-TNC-scFv;
sdAb/VHH;
3(VHH-TNC),
wherein:
HC represents a heavy chain of an IgG;
LC represents a light chain of an IgG;
LC:scFv indicates a scFv linked to, or forming part of, a light chain;
HC:scFv indicates a scFv linked to, or forming part of, a heavy chain;
HC:TNC indicates a heavy chain (HC) comprising, or linked to, tenascin (TNC);
TNC-scFv indicating tenascin linked or bound to a scFv;
TNC-VHH indicating tenascin linked or bound to a VHH;
sdAb indicates a single domain antibody;
VHH indicates the variable region of a camelid antibody;
VHH-TNC indicates tenascin linked or bound to a VHH,
preferably wherein the binding molecule is an antibody and is in an antibody format selected from:
a Fab-scFv;
IgG-HC:scFv;
LC:scFv-HC:scFv-IgG1; and
LC:scFv-HC:scFv-IgG1-TNC.

24. The binding molecule of claim 1 wherein:
(a) the binding molecule comprises at least two antigen-binding sites with a different specificity for TNFR2;
(b) all of the antigen-binding sites of the binding molecule that are specific for TNFR2 each have a different specificity for TNFR2;
(c) the binding molecule comprises two polypeptides, wherein each polypeptide comprises three VHH antigen-binding domains specific for TNFR2 wherein at least two of those three VHH antigen-binding domains have a different specificity for TNFR2; or
(d) the binding molecule comprises two polypeptides, wherein each polypeptide comprises three VHH antigen-binding domains specific for TNFR2 wherein the three VHH antigen-binding domains have a different specificity for TNFR2.

25. A binding molecule that specifically binds TNFR2, but not TNFR1, where the binding molecule is an FcγR independent agonist of TNFR2, has a valency of at least two for binding TNFR2, and comprises polypeptides comprising mutant TNF-alpha that bind TNFR2, but not TNFR1, and an Fc region, or a tenascin peptide sequence, or both, that oligomerises the polypeptides,
preferably wherein the binding molecule is one:
(a) which does not comprise antibody sequences;
(b) which comprises polypeptides comprising mutant TNF-alpha that binds TNFR2, but not TNFR1, and a tenascin peptide;
(c) wherein the TNF-alpha mutants are TNF80; and/or
(d) has a valency for TNFR2 of at least three, preferably having a valency for TNFR2 of six

26. The binding molecule of any one of the preceding claims for use as a medicament.

27. The binding molecule of any one of claims 1 to 25 for use in a method of treating or preventing an autoimmune disorder, or an inflammatory disorder,
preferably wherein:
(a) the disorder is graft versus host disease (GvHD), preferably where the binding molecule is for use in a method where it is administered prior to, at the same time, or after a transplant of a cell, tissue, or organ; or
(b) the disorder is one involving dysfunction or unwanted proliferation of leukocytes, preferably of T cells, more preferably of Treg cells; or
(c) the disorder is selected from Inflammatory Bowel Disease (such as Ulcerative Colitis, Crohn's Disease, or Celiac Disease), atherosclerosis, lupus, multiple sclerosis, Type 1 Diabetes, myasthenia gravis, pemphigus vulgaris, and bullous pemphigoid.

28. A method of stimulating cell proliferation comprising contacting a target cell expressing TNFR2 with a binding molecule according to any one of claims 1 to 25.

29. A pharmaceutical composition comprising a binding molecule according to any one of claims 1 to 25 and a pharmaceutically acceptable carrier.

30. A method of detecting TNFR2 comprising contacting a test sample with a binding molecule according to any one of claims 1 to 25 and detecting binding of the binding molecule to TNFR2,
preferably wherein the binding molecule is labelled and the binding of the binding molecule to TNFR2 is detected via the label.

31. A binding molecule according to any one of claims 1 to 25 for use in a method of diagnosis, the method comprising administering a binding molecule according to any one of claims 1 to 23 to a subject and detecting binding of the binding molecule to TNFR2,
preferably wherein the binding molecule is labelled and the binding of the binding molecule to TNFR2 is detected via the label.


SEQUENCE LISTING (338 sequences)

SEQ ID NO: 1 - protein, 461 aa, Homo sapiens
MAPVAVWAALAVGLELWAAAHALPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDFALPVGLIVGVTALGLLIIGVVNCVIMTQVKKKPLCLQREAKVPHLPADKARGTQGPEQQHLLITAPSSSSSSLESSASALDRRAPTRNQPQAPGVEASGAGEARASTGSSDSSPGGHGTQVNVTCIVNVCSSSDHSSQCSSQASSTMGDTDSSPSESPKDEQVPFSKEECAFRSQLETPETLLGSTEEKPLPLGVPDAGMKPS

SEQ ID NO: 2 - protein, 30 aa, Gallus Gallus Domesticus
ACGCAAAPDIKDLLSRLEELEGLVSSLREQ

SEQ ID NO: 3 - protein, 254 aa, artificial sequence
  pDualyx-010pre LC
MNFGFSLIFLVLVLKGVQCEVKLVPRQLDYKDDDDKELDIVMTQSHKFMSTSVGDRVSITCKASQDVDTAVAWYQQKPGQSPKLLIYWASTRHTGVPDRFTGSGSGTDYTLTISSVQAEDLARYYCQQYYSVPPTFGGGTKLGSEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 414 aa, artificial sequence
  pDualyx-030pre LC
MNFGFSLIFLVLVLKGVQCEVKLVPRQLDIVMTQSHKFMSTSVGDRVSITCKASQDVDTAVAWYQQKPGQSPKLLIYWASTRHTGVPDRFTGSGSGTDYTLTISSVQAEDLARYYCQQYYSVPPTFGGGTKLGSEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECLEKPTENNEDFNIVAVASNFATTDLDADRGKLPGKKLPLEVLKEMEANARKAGCTRGCLICLSHIKCTPKMKKFIPGRCHTYEGDKESAQGGIGEAIVDIPEIPGFKDLEPMEQFIAQVDLCVDCTTGCLKGLANVQCSDLLKKWLPQRCATFASKIQGQVDKIKGAGGD

SEQ ID NO: 5 - protein, 501 aa, artificial sequence
  pDualyx-013pre LC
MNFGFSLIFLVLVLKGVQCEVKLVPRQLDYKDDDDKELDIVMTQSHKFMSTSVGDRVSITCKASQDVDTAVAWYQQKPGQSPKLLIYWASTRHTGVPDRFTGSGSGTDYTLTISSVQAEDLARYYCQQYYSVPPTFGGGTKLGSEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECLEQVQLLQSGPELVKPGASVKLSCKASGYSFTSYDINWVKQRPGQGLEWVGWIYPRDGDTKYNEKFKGKAILTVDTSSNTAYMNLHSLTSEDSAVYFCARLTGPYWYFDVWGTGTTVTVSSRSSTKGPKLEEGEFSEAQLDIVMTQSHKFMSTSVGDRVSITCKASQDVDTAVAWYQQKPGQSPKLLIYWASTRHTGVPDRFTGSGSGTDYTLTISSVQAEDLARYYCQQYYSVPPTFGGGTKLEIK

SEQ ID NO: 6 - protein, 395 aa, artificial sequence
  pDualyx-022 LC
MNFGFSLIFLVLVLKGVQCEVKLVPRQLDYKDDDDKEFQVQLLQSGPELVKPGASVKLSCKASGYSFTSYDINWVKQRPGQGLEWVGWIYPRDGDTKYNEKFKGKAILTVDTSSNTAYMNLHSLTSEDSAVYFCARLTGPYWYFDVWGTGTTVTVSSRSSTKGPKLEEGEFSEAQLDIVMTQSHKFMSTSVGDRVSITCKASQDVDTAVAWYQQKPGQSPKLLIYWASTRHTGVPDRFTGSGSGTDYTLTISSVQAEDLARYYCQQYYSVPPTFGGGTKLEIKGSEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 642 aa, artificial sequence
  pDualyx-023 LC
MNFGFSLIFLVLVLKGVQCEVKLVPRQLDYKDDDDKEFQVQLLQSGPELVKPGASVKLSCKASGYSFTSYDINWVKQRPGQGLEWVGWIYPRDGDTKYNEKFKGKAILTVDTSSNTAYMNLHSLTSEDSAVYFCARLTGPYWYFDVWGTGTTVTVSSRSSTKGPKLEEGEFSEAQLDIVMTQSHKFMSTSVGDRVSITCKASQDVDTAVAWYQQKPGQSPKLLIYWASTRHTGVPDRFTGSGSGTDYTLTISSVQAEDLARYYCQQYYSVPPTFGGGTKLEIKGSEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECLEQVQLLQSGPELVKPGASVKLSCKASGYSFTSYDINWVKQRPGQGLEWVGWIYPRDGDTKYNEKFKGKAILTVDTSSNTAYMNLHSLTSEDSAVYFCARLTGPYWYFDVWGTGTTVTVSSRSSTKGPKLEEGEFSEAQLDIVMTQSHKFMSTSVGDRVSITCKASQDVDTAVAWYQQKPGQSPKLLIYWASTRHTGVPDRFTGSGSGTDYTLTISSVQAEDLARYYCQQYYSVPPTFGGGTKLEIK

SEQ ID NO: 8 - protein, 256 aa, artificial sequence
  pDualyx-005 LC
MNFGFSLIFLVLVLKGVQCEVKLVPRQLDYKDDDDKELDIVMAQSQKFMSVSVGDRVSVTCKASQNVGTNVAWYQQRPGQSPKALIYSASYRYSGVPDRFTGSGSGTDFTLTISNVQSEDLAEYFCQQFDSHPLTFGAGTKLELKGSIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 9 - protein, 417 aa, artificial sequence
  pDualyx-032 LC
MNFGFSLIFLVLVLKGVQCEVKLVPRQLDIVMAQSQKFMSVSVGDRVSVTCKASQNVGTNVAWYQQRPGQSPKALIYSASYRYSGVPDRFTGSGSGTDFTLTISNVQSEDLAEYFCQQFDSHPLTFGAGTKLELKGSEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECLEKPTENNEDFNIVAVASNFATTDLDADRGKLPGKKLPLEVLKEMEANARKAGCTRGCLICLSHIKCTPKMKKFIPGRCHTYEGDKESAQGGIGEAIVDIPEIPGFKDLEPMEQFIAQVDLCVDCTTGCLKGLANVQCSDLLKKWLPQRCATFASKIQGQVDKIKGAGGD

SEQ ID NO: 10 - protein, 257 aa, artificial sequence
  pDualyx-092 LC
MNFGFSLIFLVLVLKGVQCEVKLVPRQLDYKDDDDKELNIVMTQSPKSLSMSVGERVTLNCKASENVKTYVSWYQQKPEQSPKLLIYGASNRYTGVPDRFTGSGSATDFTLTISSVQAEDLADYHCGQSYSYPFTFGAGTKLERKGSEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 427 aa, artificial sequence
  pDualyx-093 LC
MNFGFSLIFLVLVLKGVQCEVKLVPRQLDYKDDDDKELNIVMTQSPKSLSMSVGERVTLNCKASENVKTYVSWYQQKPEQSPKLLIYGASNRYTGVPDRFTGSGSATDFTLTISSVQAEDLADYHCGQSYSYPFTFGAGTKLERKGSEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECLEKPTENNEDFNIVAVASNFATTDLDADRGKLPGKKLPLEVLKEMEANARKAGCTRGCLICLSHIKCTPKMKKFIPGRCHTYEGDKESAQGGIGEAIVDIPEIPGFKDLEPMEQFIAQVDLCVDCTTGCLKGLANVQCSDLLKKWLPQRCATFASKIQGQVDKIKGAGGD

SEQ ID NO: 12 - protein, 256 aa, artificial sequence
  pDualyx-077 LC
MNFGFSLIFLVLVLKGVQCEVKLVPRQLDYKDDDDKELDIQMTQSPSSLSASLGGKVTITCKASQDINKFIAWYQHKPGKGPRLLIHYTSTLQPGIPSRFSGSGSGRDYSFSISNLEPEDIATYYCLQYDNLYTFGGGTKLEIKGSEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 426 aa, artificial sequence
  pDualyx-076 LC
MNFGFSLIFLVLVLKGVQCEVKLVPRQLDYKDDDDKELDIQMTQSPSSLSASLGGKVTITCKASQDINKFIAWYQHKPGKGPRLLIHYTSTLQPGIPSRFSGSGSGRDYSFSISNLEPEDIATYYCLQYDNLYTFGGGTKLEIKGSEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECLEKPTENNEDFNIVAVASNFATTDLDADRGKLPGKKLPLEVLKEMEANARKAGCTRGCLICLSHIKCTPKMKKFIPGRCHTYEGDKESAQGGIGEAIVDIPEIPGFKDLEPMEQFIAQVDLCVDCTTGCLKGLANVQCSDLLKKWLPQRCATFASKIQGQVDKIKGAGGD

SEQ ID NO: 14 - protein, 257 aa, artificial sequence
  pDualyx-016pre LC
MNFGFSLIFLVLVLKGVQCEVKLVPRQLDYKDDDDKELDIVMAQSQKFMSVSVGDRVSVTCKASQNVGTNVAWYQQRPGQSPKALIYSASYRYSGVPDRFTGSGSGTDFTLTISNVQSEDLAEYFCQQFDSHPLTFGAGTKLELKGSEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 420 aa, artificial sequence
  pDualyx-028 LC
MNFGFSLIFLVLVLKGVQCEVKLVPRQLQAVVTQESALTTSPGETVTLTCRSSTGAVTTSNYANWIQEKPDHLFTGLIGGTNNRTPGVPARFSGSLIGDKAALTVTGAQTEDEAVYFCALWYSNHWVFGGGTKLTVLGRSEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECLEKPTENNEDFNIVAVASNFATTDLDADRGKLPGKKLPLEVLKEMEANARKAGCTRGCLICLSHIKCTPKMKKFIPGRCHTYEGDKESAQGGIGEAIVDIPEIPGFKDLEPMEQFIAQVDLCVDCTTGCLKGLANVQCSDLLKKWLPQRCATFASKIQGQVDKIKGAGGD

SEQ ID NO: 16 - protein, 426 aa, artificial sequence
  pDualyx-087 LC
MNFGFSLIFLVLVLKGVQCEVKLVPRQLDYKDDDDKELQIVLTQSPAIMSASPGEKVTITCSASSSVSYMHWFQQKPGTSPKLWIYSTSNLASGVPARFSGSGSGTSYSLTISRMEAEDAATYYCQQRSSYPPTFGGGTKLEIKGSEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECLEKPTENNEDFNIVAVASNFATTDLDADRGKLPGKKLPLEVLKEMEANARKAGCTRGCLICLSHIKCTPKMKKFIPGRCHTYEGDKESAQGGIGEAIVDIPEIPGFKDLEPMEQFIAQVDLCVDCTTGCLKGLANVQCSDLLKKWLPQRCATFASKIQGQVDKIKGAGGD

SEQ ID NO: 17 - protein, 432 aa, artificial sequence
  pDualyx-090 LC
MNFGFSLIFLVLVLKGVQCEVKLVPRQLDYKDDDDKELDVVMTQTPLSLPVSLGDQASISCRASQSLVHSNGNTYLQWYLQKPGQSPKLLIYKVSNRFSGVPDRFSGSGSGTDFTLKISRVEAEDLGVFFCSQSTHVPFTFGSGTKLEIKGSEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECLEKPTENNEDFNIVAVASNFATTDLDADRGKLPGKKLPLEVLKEMEANARKAGCTRGCLICLSHIKCTPKMKKFIPGRCHTYEGDKESAQGGIGEAIVDIPEIPGFKDLEPMEQFIAQVDLCVDCTTGCLKGLANVQCSDLLKKWLPQRCATFASKIQGQVDKIKGAGGD

SEQ ID NO: 18 - protein, 430 aa, artificial sequence
  pDualyx-071 LC
MNFGFSLIFLVLVLKGVQCEVKLVPRQLDYKDDDDKELQAVVTQESALTTSPGETVTLTCRSSTGAVTTSNYANWVQEKPDHLFTGLIGGTNNRAPGVPARFSGSVIGDKAALTITGAQTEDEAIYFCALWYSNHWVFGGGTKLTVLGGSEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECLEKPTENNEDFNIVAVASNFATTDLDADRGKLPGKKLPLEVLKEMEANARKAGCTRGCLICLSHIKCTPKMKKFIPGRCHTYEGDKESAQGGIGEAIVDIPEIPGFKDLEPMEQFIAQVDLCVDCTTGCLKGLANVQCSDLLKKWLPQRCATFASKIQGQVDKIKGAGGD

SEQ ID NO: 19 - protein, 426 aa, artificial sequence
  pDualyx-040pre LC
MNFGFSLIFLVLVLKGVQCEVKLVPRQLDYKDDDDKELDVQMTQSPSSLSASVGDRVTITCKASQDINKFIAWYQQKPGKAPKLLIHYTSTLQPGIPSRFSGSGSGRDYTFTISSLQPEDIATYYCLQYGNLWTFGGGTKVEIKGSEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECLEKPTENNEDFNIVAVASNFATTDLDADRGKLPGKKLPLEVLKEMEANARKAGCTRGCLICLSHIKCTPKMKKFIPGRCHTYEGDKESAQGGIGEAIVDIPEIPGFKDLEPMEQFIAQVDLCVDCTTGCLKGLANVQCSDLLKKWLPQRCATFASKIQGQVDKIKGAGGD

SEQ ID NO: 20 - protein, 496 aa, artificial sequence
  pDualyx-112 LC
MNFGFSLIFLVLVLKGVQCEVKLVPRQLDYKDDDDKELDIVMTQSHKFMSTSVGDRVSITCKASQDVDTAVAWYQQKPGQSPKLLIYWASTRHTGVPDRFTGSGSGTDYTLTISSVQAEDLARYYCQQYYSVPPTFGGGTKLGSEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECLEQIQLVQSGPELKKPGETVKISCKASGYTFTTAGMQWVQKMPGKGFKWIGWINTHSGEPKYAEDFKGRFAFSLETSASTAYLQISNLKNEDTATYFCARWDGTGYWGQGTTLTVSSRSSTKGPKLEEGEFSEAQLQIVLTQSPAIMSASPGEKVTITCSASSSVSYMHWFQQKPGTSPKLWIYSTSNLASGVPARFSGSGSGTSYSLTISRMEAEDAATYYCQQRSSYPPTFGGGTKLEIK

SEQ ID NO: 21 - protein, 503 aa, artificial sequence
  pDualyx-113 LC
MNFGFSLIFLVLVLKGVQCEVKLVPRQLDYKDDDDKELDIVMTQSHKFMSTSVGDRVSITCKASQDVDTAVAWYQQKPGQSPKLLIYWASTRHTGVPDRFTGSGSGTDYTLTISSVQAEDLARYYCQQYYSVPPTFGGGTKLGSEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECLEEVKLLQSGGGLVQPGGSLKLSCEASGIDFSRYWMSWVRRAPGKGLEWIGESDPDSSTINYAPSLKDKFIISRDSARNTLYLQMSKVRSEDTALYYCAREGYYDNEGYFDVWGTGTTVTVSSRSSTKGPKLEEGEFSEAQLNIVMTQSPKSLSMSVGERVTLNCKASENVKTYVSWYQQKPEQSPKLLIYGASNRYTGVPDRFTGSGSATDFTLTISSVQAEDLADYHCGQSYSYPFTFGAGTKLERK

SEQ ID NO: 22 - protein, 500 aa, artificial sequence
  pDualyx-114 LC
MNFGFSLIFLVLVLKGVQCEVKLVPRQLDYKDDDDKELDIVMTQSHKFMSTSVGDRVSITCKASQDVDTAVAWYQQKPGQSPKLLIYWASTRHTGVPDRFTGSGSGTDYTLTISSVQAEDLARYYCQQYYSVPPTFGGGTKLGSEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECLEEIQLQQSGTVLARPGASVKMSCKTSGYTFTNYWMHWLKQRPGQGLEWIGTIYPGNSETNYNPNFKGKAKLTAVSSASTAYMELSSLTNEDSAVYYCSRGEDYWGQGTSVTVSSRSSTKGPKLEEGEFSEAQLDVVMTQTPLSLPVSLGDQASISCRASQSLVHSNGNTYLQWYLQKPGQSPKLLIYKVSNRFSGVPDRFSGSGSGTDFTLKISRVEAEDLGVFFCSQSTHVPFTFGSGTKLEIK

SEQ ID NO: 23 - protein, 506 aa, Artificial Sequence
  pDualyx-115 LC
MNFGFSLIFLVLVLKGVQCEVKLVPRQLDYKDDDDKELDIVMTQSHKFMSTSVGDRVSITCKASQDVDTAVAWYQQKPGQSPKLLIYWASTRHTGVPDRFTGSGSGTDYTLTISSVQAEDLARYYCQQYYSVPPTFGGGTKLGSEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECLEDVQLQESGPGLVKPSQSLSLTCSVTGSSITSGYYWNWIRQFPGNKLEWMGYITNDGSNNYNPSLKNRISITRDTSENQFFLKLNSVTTEDTATYYCARDGGWFHYFDYWGQGTTLTVSSRSSTKGPKLEEGEFSEAQLDVVMTQTPLSLPVSLGDQASISCRASQSLVHSNGNTYLQWYLQKPGQSPKLLIYKVSNRFSGVPDRFSGSGSGTDFTLKISRVEAEDLGVFFCSQSTHVPFTFGSGTKLEIK

SEQ ID NO: 24 - protein, 500 aa, artificial sequence
  pDualyx-116 LC
MNFGFSLIFLVLVLKGVQCEVKLVPRQLDYKDDDDKELDIVMTQSHKFMSTSVGDRVSITCKASQDVDTAVAWYQQKPGQSPKLLIYWASTRHTGVPDRFTGSGSGTDYTLTISSVQAEDLARYYCQQYYSVPPTFGGGTKLGSEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECLEQVTLKESGPGILQPSQTLSLTCSFSGFSLSTFGMGVGWIRQPSGKGLEWLAHIWWDDDKYYNPALKSRLTISKDTSENQVFLKIANVDTADTATYYCVRIAGTRYFDVWGTGTTVTVSSRSSTKGPKLEEGEFSEAQLDIQMTQSPSSLSASLGGKVTITCKASQDINKFIAWYQHKPGKGPRLLIHYTSTLQPGIPSRFSGSGSGRDYSFSISNLEPEDIATYYCLQYDNLYTFGGGTKLEIK

SEQ ID NO: 25 - protein, 396 aa, artificial sequence
  pDualyx-151 LC
MNFGFSLIFLVLVLKGVQCEVKLVPRQLDYKDDDDKEFQIQLVQSGPEVKKPGETVKISCKASGYTFTIHGMSWVKQAPGKGLKWMGWINTYSGVPTYANDFKGRFAFSLETSASTAYLQINNLKNEDTATYFCARDEVRRGFGFAYWGQGTLVTVSARSSTKGPKLEEGEFSEAQLDIVMAQSQKFMSVSVGDRVSVTCKASQNVGTNVAWYQQRPGQSPKALIYSASYRYSGVPDRFTGSGSGTDFTLTISNVQSEDLAEYFCQQFDSHPLTFGAGTKLELKGSEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 397 aa, artificial sequence
  pDualyx-156 LC
MNFGFSLIFLVLVLKGVQCEVKLVPRQLDYKDDDDKEFEVKLLQSGGGLVQPGGSLKLSCEASGIDFSRYWMSWVRRAPGKGLEWIGESDPDSSTINYAPSLKDKFIISRDSARNTLYLQMSKVRSEDTALYYCAREGYYDNEGYFDVWGTGTTVTVSSRSSTKGPKLEEGEFSEAQLNIVMTQSPKSLSMSVGERVTLNCKASENVKTYVSWYQQKPEQSPKLLIYGASNRYTGVPDRFTGSGSATDFTLTISSVQAEDLADYHCGQSYSYPFTFGAGTKLERKGSEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 394 aa, artificial sequence
  pDualyx-161 LC
MNFGFSLIFLVLVLKGVQCEVKLVPRQLDYKDDDDKEFQVTLKESGPGILQPSQTLSLTCSFSGFSLSTFGMGVGWIRQPSGKGLEWLAHIWWDDDKYYNPALKSRLTISKDTSENQVFLKIANVDTADTATYYCVRIAGTRYFDVWGTGTTVTVSSRSSTKGPKLEEGEFSEAQLDIQMTQSPSSLSASLGGKVTITCKASQDINKFIAWYQHKPGKGPRLLIHYTSTLQPGIPSRFSGSGSGRDYSFSISNLEPEDIATYYCLQYDNLYTFGGGTKLEIKGSEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 481 aa, artificial sequence
  pDualyx-011pre HC
MNFGFSLIFLVLVLKGVQCEVKLVPRQLQVQLLQSGPELVKPGASVKLSCKASGYSFTSYDINWVKQRPGQGLEWVGWIYPRDGDTKYNEKFKGKAILTVDTSSNTAYMNLHSLTSEDSAVYFCARLTGPYWYFDVWGTGTTVTVSSGSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 481 aa, artificial sequence
  pDualyx-029pre HC
MNFGFSLIFLVLVLKGVQCEVKLVPRQLQVQLLQSGPELVKPGASVKLSCKASGYSFTSYDINWVKQRPGQGLEWVGWIYPRDGDTKYNEKFKGKAILTVDTSSNTAYMNLHSLTSEDSAVYFCARLTGPYWYFDVWGTGTTVTVSSGSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 728 aa, artificial sequence
  pDualyx-012pre HC
MNFGFSLIFLVLVLKGVQCEVKLVPRQLQVQLLQSGPELVKPGASVKLSCKASGYSFTSYDINWVKQRPGQGLEWVGWIYPRDGDTKYNEKFKGKAILTVDTSSNTAYMNLHSLTSEDSAVYFCARLTGPYWYFDVWGTGTTVTVSSGSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKLEQVQLLQSGPELVKPGASVKLSCKASGYSFTSYDINWVKQRPGQGLEWVGWIYPRDGDTKYNEKFKGKAILTVDTSSNTAYMNLHSLTSEDSAVYFCARLTGPYWYFDVWGTGTTVTVSSRSSTKGPKLEEGEFSEAQLDIVMTQSHKFMSTSVGDRVSITCKASQDVDTAVAWYQQKPGQSPKLLIYWASTRHTGVPDRFTGSGSGTDYTLTISSVQAEDLARYYCQQYYSVPPTFGGGTKLEIK

SEQ ID NO: 31 - protein, 269 aa, artificial sequence
  pDualyx-019pre HC
MNFGFSLIFLVLVLKGVQCEVKLVPRQLDYKDDDDKELQVQLLQSGPELVKPGASVKLSCKASGYSFTSYDINWVKQRPGQGLEWVGWIYPRDGDTKYNEKFKGKAILTVDTSSNTAYMNLHSLTSEDSAVYFCARLTGPYWYFDVWGTGTTVTVSSGSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 32 - protein, 265 aa, artificial sequence
  pDualyx-020pre HC
MNFGFSLIFLVLVLKGVQCEVKLVPRQLQVQLLQSGPELVKPGASVKLSCKASGYSFTSYDINWVKQRPGQGLEWVGWIYPRDGDTKYNEKFKGKAILTVDTSSNTAYMNLHSLTSEDSAVYFCARLTGPYWYFDVWGTGTTVTVSSGSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPA

SEQ ID NO: 33 - protein, 530 aa, artificial sequence
  pDualyx-044 HC
MNFGFSLIFLVLVLKGVQCEVKLVPRQLDYKDDDDKELQVQLLQSGPELVKPGASVKLSCKASGYSFTSYDINWVKQRPGQGLEWVGWIYPRDGDTKYNEKFKGKAILTVDTSSNTAYMNLHSLTSEDSAVYFCARLTGPYWYFDVWGTGTTVTVSSGSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKEFLEDIACGCAAAPDIKDLLSRLEELEGLVSSLREQGTG

SEQ ID NO: 34 - protein, 491 aa, artificial sequence
  pDualyx-118 HC
MNFGFSLIFLVLVLKGVQCEVKLVPRQLDYKDDDDKELQVQLLQSGPELVKPGASVKLSCKASGYSFTSYDINWVKQRPGQGLEWVGWIYPRDGDTKYNEKFKGKAILTVDTSSNTAYMNLHSLTSEDSAVYFCARLTGPYWYFDVWGTGTTVTVSSGSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPRRPQVYTLPPSRDELTKNQVSLTCLVKGFYPSDIAVEWESNGQPENNYKTTPPVLDSDGSFFLYSKLTVDKSRWQQGNVFSCSVMHGALHNHYTQKYLSLSPGK

SEQ ID NO: 35 - protein, 780 aa, artificial sequence
  pDualyx-119 HC
MNFGFSLIFLVLVLKGVQCEVKLVPRQLDYKDDDDKELQVQLLQSGPELVKPGASVKLSCKASGYSFTSYDINWVKQRPGQGLEWVGWIYPRDGDTKYNEKFKGKAILTVDTSSNTAYMNLHSLTSEDSAVYFCARLTGPYWYFDVWGTGTTVTVSSGSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKEFLEDIACGCAAAPDIKDLLSRLEELEGLVSSLREQGTGGGSVEQVQLLQSGPELVKPGASVKLSCKASGYSFTSYDINWVKQRPGQGLEWVGWIYPRDGDTKYNEKFKGKAILTVDTSSNTAYMNLHSLTSEDSAVYFCARLTGPYWYFDVWGTGTTVTVSSRSSTKGPKLEEGEFSEAQLDIVMTQSHKFMSTSVGDRVSITCKASQDVDTAVAWYQQKPGQSPKLLIYWASTRHTGVPDRFTGSGSGTDYTLTISSVQAEDLARYYCQQYYSVPPTFGGGTKLEIK

SEQ ID NO: 36 - protein, 617 aa, artificial sequence
  pDualyx-021 HC
MNFGFSLIFLVLVLKGVQCEVKLVPRQLDYKDDDDKEFQVQLLQSGPELVKPGASVKLSCKASGYSFTSYDINWVKQRPGQGLEWVGWIYPRDGDTKYNEKFKGKAILTVDTSSNTAYMNLHSLTSEDSAVYFCARLTGPYWYFDVWGTGTTVTVSSRSSTKGPKLEEGEFSEAQLDIVMTQSHKFMSTSVGDRVSITCKASQDVDTAVAWYQQKPGQSPKLLIYWASTRHTGVPDRFTGSGSGTDYTLTISSVQAEDLARYYCQQYYSVPPTFGGGTKLEIKGSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - protein, 864 aa, artificial sequence
  pDualyx-024 HC
MNFGFSLIFLVLVLKGVQCEVKLVPRQLDYKDDDDKEFQVQLLQSGPELVKPGASVKLSCKASGYSFTSYDINWVKQRPGQGLEWVGWIYPRDGDTKYNEKFKGKAILTVDTSSNTAYMNLHSLTSEDSAVYFCARLTGPYWYFDVWGTGTTVTVSSRSSTKGPKLEEGEFSEAQLDIVMTQSHKFMSTSVGDRVSITCKASQDVDTAVAWYQQKPGQSPKLLIYWASTRHTGVPDRFTGSGSGTDYTLTISSVQAEDLARYYCQQYYSVPPTFGGGTKLEIKGSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKLEQVQLLQSGPELVKPGASVKLSCKASGYSFTSYDINWVKQRPGQGLEWVGWIYPRDGDTKYNEKFKGKAILTVDTSSNTAYMNLHSLTSEDSAVYFCARLTGPYWYFDVWGTGTTVTVSSRSSTKGPKLEEGEFSEAQLDIVMTQSHKFMSTSVGDRVSITCKASQDVDTAVAWYQQKPGQSPKLLIYWASTRHTGVPDRFTGSGSGTDYTLTISSVQAEDLARYYCQQYYSVPPTFGGGTKLEIK

SEQ ID NO: 38 - protein, 654 aa, artificial sequence
  pDualyx-051 HC
MNFGFSLIFLVLVLKGVQCEVKLVPRQLDYKDDDDKEFQVQLLQSGPELVKPGASVKLSCKASGYSFTSYDINWVKQRPGQGLEWVGWIYPRDGDTKYNEKFKGKAILTVDTSSNTAYMNLHSLTSEDSAVYFCARLTGPYWYFDVWGTGTTVTVSSRSSTKGPKLEEGEFSEAQLDIVMTQSHKFMSTSVGDRVSITCKASQDVDTAVAWYQQKPGQSPKLLIYWASTRHTGVPDRFTGSGSGTDYTLTISSVQAEDLARYYCQQYYSVPPTFGGGTKLEIKGSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKLEDIACGCAAAPDIKDLLSRLEELEGLVSSLREQGTG

SEQ ID NO: 39 - protein, 741 aa, artificial sequence
  pDualyx-080 HC
MNFGFSLIFLVLVLKGVQCEVKLVPRQLDYKDDDDKELQVQLLQSGPELVKPGASVKLSCKASGYSFTSYDINWVKQRPGQGLEWVGWIYPRDGDTKYNEKFKGKAILTVDTSSNTAYMNLHSLTSEDSAVYFCARLTGPYWYFDVWGTGTTVTVSSGSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKEFLEQIQLVQSGPEVKKPGETVKISCKASGYTFTIHGMSWVKQAPGKGLKWMGWINTYSGVPTYANDFKGRFAFSLETSASTAYLQINNLKNEDTATYFCARDEVRRGFGFAYWGQGTLVTVSARSSTKGPKLEEGEFSEAQLDIVMAQSQKFMSVSVGDRVSVTCKASQNVGTNVAWYQQRPGQSPKALIYSASYRYSGVPDRFTGSGSGTDFTLTISNVQSEDLAEYFCQQFDSHPLTFGAGTKLELK

SEQ ID NO: 40 - protein, 742 aa, artificial sequence
  pDualyx-082 HC
MNFGFSLIFLVLVLKGVQCEVKLVPRQLDYKDDDDKELQVQLLQSGPELVKPGASVKLSCKASGYSFTSYDINWVKQRPGQGLEWVGWIYPRDGDTKYNEKFKGKAILTVDTSSNTAYMNLHSLTSEDSAVYFCARLTGPYWYFDVWGTGTTVTVSSGSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKEFLEEVKLLQSGGGLVQPGGSLKLSCEASGIDFSRYWMSWVRRAPGKGLEWIGESDPDSSTINYAPSLKDKFIISRDSARNTLYLQMSKVRSEDTALYYCAREGYYDNEGYFDVWGTGTTVTVSSRSSTKGPKLEEGEFSEAQLNIVMTQSPKSLSMSVGERVTLNCKASENVKTYVSWYQQKPEQSPKLLIYGASNRYTGVPDRFTGSGSATDFTLTISSVQAEDLADYHCGQSYSYPFTFGAGTKLERK

SEQ ID NO: 41 - protein, 739 aa, artificial sequence
  pDualyx-063 HC
MNFGFSLIFLVLVLKGVQCEVKLVPRQLDYKDDDDKELQVQLLQSGPELVKPGASVKLSCKASGYSFTSYDINWVKQRPGQGLEWVGWIYPRDGDTKYNEKFKGKAILTVDTSSNTAYMNLHSLTSEDSAVYFCARLTGPYWYFDVWGTGTTVTVSSGSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKEFLEQVTLKESGPGILQPSQTLSLTCSFSGFSLSTFGMGVGWIRQPSGKGLEWLAHIWWDDDKYYNPALKSRLTISKDTSENQVFLKIANVDTADTATYYCVRIAGTRYFDVWGTGTTVTVSSRSSTKGPKLEEGEFSEAQLDIQMTQSPSSLSASLGGKVTITCKASQDINKFIAWYQHKPGKGPRLLIHYTSTLQPGIPSRFSGSGSGRDYSFSISNLEPEDIATYYCLQYDNLYTFGGGTKLEIK

SEQ ID NO: 42 - protein, 486 aa, artificial sequence
  pDualyx-006 HC
MNFGFSLIFLVLVLKGVQCEVKLVPRQLDYKDDDDKEFQIQLVQSGPEVKKPGETVKISCKASGYTFTIHGMSWVKQAPGKGLKWMGWINTYSGVPTYANDFKGRFAFSLETSASTAYLQINNLKNEDTATYFCARDEVRRGFGFAYWGQGTLVTVSARSSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFPAPIEKTISKTKGRPKAPQVYTIPPPKEQMAKDKVSLTCMITNFFPEDITVEWQWNGQPAENYKNTQPIMDTDGSYFVYSKLNVQKSNWEAGNTFTCSVLHEGLHNHHTEKSLSHSPGK

SEQ ID NO: 43 - protein, 482 aa, artificial sequence
  pDualyx-031 HC
MNFGFSLIFLVLVLKGVQCEVKLVPRQLQIQLVQSGPEVKKPGETVKISCKASGYTFTIHGMSWVKQAPGKGLKWMGWINTYSGVPTYANDFKGRFAFSLETSASTAYLQINNLKNEDTATYFCARDEVRRGFGFAYWGQGTLVTVSAGSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - protein, 493 aa, artificial sequence
  pDualyx-091 HC
MNFGFSLIFLVLVLKGVQCEVKLVPRQLDYKDDDDKEFEVKLLQSGGGLVQPGGSLKLSCEASGIDFSRYWMSWVRRAPGKGLEWIGESDPDSSTINYAPSLKDKFIISRDSARNTLYLQMSKVRSEDTALYYCAREGYYDNEGYFDVWGTGTTVTVSSRSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - protein, 491 aa, artificial sequence
  pDualyx-075 HC
MNFGFSLIFLVLVLKGVQCEVKLVPRQLDYKDDDDKEFQVTLKESGPGILQPSQTLSLTCSFSGFSLSTFGMGVGWIRQPSGKGLEWLAHIWWDDDKYYNPALKSRLTISKDTSENQVFLKIANVDTADTATYYCVRIAGTRYFDVWGTGTTVTVSSRSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 730 aa, artificial sequence
  pDualyx-017pre HC
MNFGFSLIFLVLVLKGVQCEVKLVPRQLQIQLVQSGPEVKKPGETVKISCKASGYTFTIHGMSWVKQAPGKGLKWMGWINTYSGVPTYANDFKGRFAFSLETSASTAYLQINNLKNEDTATYFCARDEVRRGFGFAYWGQGTLVTVSAGSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKEFQIQLVQSGPEVKKPGETVKISCKASGYTFTIHGMSWVKQAPGKGLKWMGWINTYSGVPTYANDFKGRFAFSLETSASTAYLQINNLKNEDTATYFCARDEVRRGFGFAYWGQGTLVTVSARSSTKGPKLEEGEFSEAQLDIVMAQSQKFMSVSVGDRVSVTCKASQNVGTNVAWYQQRPGQSPKALIYSASYRYSGVPDRFTGSGSGTDFTLTISNVQSEDLAEYFCQQFDSHPLTFGAGTKLELK

SEQ ID NO: 47 - protein, 742 aa, artificial sequence
  pDualyx-108 HC
MNFGFSLIFLVLVLKGVQCEVKLVPRQLDYKDDDDKEFEVKLLQSGGGLVQPGGSLKLSCEASGIDFSRYWMSWVRRAPGKGLEWIGESDPDSSTINYAPSLKDKFIISRDSARNTLYLQMSKVRSEDTALYYCAREGYYDNEGYFDVWGTGTTVTVSSRSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKLEEVKLLQSGGGLVQPGGSLKLSCEASGIDFSRYWMSWVRRAPGKGLEWIGESDPDSSTINYAPSLKDKFIISRDSARNTLYLQMSKVRSEDTALYYCAREGYYDNEGYFDVWGTGTTVTVSSRSSTKGPKLEEGEFSEAQLNIVMTQSPKSLSMSVGERVTLNCKASENVKTYVSWYQQKPEQSPKLLIYGASNRYTGVPDRFTGSGSATDFTLTISSVQAEDLADYHCGQSYSYPFTFGAGTKLERK

SEQ ID NO: 48 - protein, 737 aa, artificial sequence
  pDualyx-111 HC
MNFGFSLIFLVLVLKGVQCEVKLVPRQLDYKDDDDKEFQVTLKESGPGILQPSQTLSLTCSFSGFSLSTFGMGVGWIRQPSGKGLEWLAHIWWDDDKYYNPALKSRLTISKDTSENQVFLKIANVDTADTATYYCVRIAGTRYFDVWGTGTTVTVSSRSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKLEQVTLKESGPGILQPSQTLSLTCSFSGFSLSTFGMGVGWIRQPSGKGLEWLAHIWWDDDKYYNPALKSRLTISKDTSENQVFLKIANVDTADTATYYCVRIAGTRYFDVWGTGTTVTVSSRSSTKGPKLEEGEFSEAQLDIQMTQSPSSLSASLGGKVTITCKASQDINKFIAWYQHKPGKGPRLLIHYTSTLQPGIPSRFSGSGSGRDYSFSISNLEPEDIATYYCLQYDNLYTFGGGTKLEIK

SEQ ID NO: 49 - protein, 481 aa, artificial sequence
  pDualyx-027 HC
MNFGFSLIFLVLVLKGVQCEVKLVPRQLEVQLQQSGAELVRPGASVKLSCTASGFNIKDDYIHWVKQRPEQGLEWIGWIDPENSDTEYASKFQGKATITADTSSNTAYLQLSSLTSEDTAVYYCTTGLWLRTAIDYWGQGTSITVSSGSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 487 aa, artificial sequence
  pDualyx-085 HC
MNFGFSLIFLVLVLKGVQCEVKLVPRQLDYKDDDDKEFQIQLVQSGPELKKPGETVKISCKASGYTFTTAGMQWVQKMPGKGFKWIGWINTHSGEPKYAEDFKGRFAFSLETSASTAYLQISNLKNEDTATYFCARWDGTGYWGQGTTLTVSSRSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 485 aa, artificial sequence
  pDualyx-088 HC
MNFGFSLIFLVLVLKGVQCEVKLVPRQLDYKDDDDKEFEIQLQQSGTVLARPGASVKMSCKTSGYTFTNYWMHWLKQRPGQGLEWIGTIYPGNSETNYNPNFKGKAKLTAVSSASTAYMELSSLTNEDSAVYYCSRGEDYWGQGTSVTVSSRSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 491 aa, artificial sequence
  pDualyx-069 HC
MNFGFSLIFLVLVLKGVQCEVKLVPRQLDYKDDDDKEFDVQLQESGPGLVKPSQSLSLTCSVTGSSITSGYYWNWIRQFPGNKLEWMGYITNDGSNNYNPSLKNRISITRDTSENQFFLKLNSVTTEDTATYYCARDGGWFHYFDYWGQGTTLTVSSRSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 491 aa, artificial sequence
  pDualyx-041pre HC
MNFGFSLIFLVLVLKGVQCEVKLVPRQLDYKDDDDKEFQVTLKESGPALVKPTQTLTLTCTFSGFSLSTSGMGVGWIRQPPGKALEWLAHIWWDDDKFYNPSLKSRLTISKDTSKNQVVLTMTNMDPVDTATYYCARLTGTRYFDYWGQGTTVTVSSRSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 529 aa, artificial sequence
  pDualyx-149 HC
MNFGFSLIFLVLVLKGVQCEVKLVPRQLDYKDDDDKEFQIQLVQSGPEVKKPGETVKISCKASGYTFTIHGMSWVKQAPGKGLKWMGWINTYSGVPTYANDFKGRFAFSLETSASTAYLQINNLKNEDTATYFCARDEVRRGFGFAYWGQGTLVTVSARSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKLEDIACGCAAAPDIKDLLSRLEELEGLVSSLREQGTG

SEQ ID NO: 55 - protein, 618 aa, artificial sequence
  pDualyx-150 HC
MNFGFSLIFLVLVLKGVQCEVKLVPRQLDYKDDDDKEFQIQLVQSGPEVKKPGETVKISCKASGYTFTIHGMSWVKQAPGKGLKWMGWINTYSGVPTYANDFKGRFAFSLETSASTAYLQINNLKNEDTATYFCARDEVRRGFGFAYWGQGTLVTVSARSSTKGPKLEEGEFSEAQLDIVMAQSQKFMSVSVGDRVSVTCKASQNVGTNVAWYQQRPGQSPKALIYSASYRYSGVPDRFTGSGSGTDFTLTISNVQSEDLAEYFCQQFDSHPLTFGAGTKLELKGSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - protein, 655 aa, artificial sequence
  pDualyx-152 HC
MNFGFSLIFLVLVLKGVQCEVKLVPRQLDYKDDDDKEFQIQLVQSGPEVKKPGETVKISCKASGYTFTIHGMSWVKQAPGKGLKWMGWINTYSGVPTYANDFKGRFAFSLETSASTAYLQINNLKNEDTATYFCARDEVRRGFGFAYWGQGTLVTVSARSSTKGPKLEEGEFSEAQLDIVMAQSQKFMSVSVGDRVSVTCKASQNVGTNVAWYQQRPGQSPKALIYSASYRYSGVPDRFTGSGSGTDFTLTISNVQSEDLAEYFCQQFDSHPLTFGAGTKLELKGSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKLEDIACGCAAAPDIKDLLSRLEELEGLVSSLREQGTG

SEQ ID NO: 57 - protein, 439 aa, artificial sequence
  pDualyx1-153 HC
MNFGFSLIFLVLVLKGVQCEVKLVPRQLDYKDDDDKEFQIQLVQSGPEVKKPGETVKISCKASGYTFTIHGMSWVKQAPGKGLKWMGWINTYSGVPTYANDFKGRFAFSLETSASTAYLQINNLKNEDTATYFCARDEVRRGFGFAYWGQGTLVTVSARSSTKGPKLEEGEFSEAQLDIVMAQSQKFMSVSVGDRVSVTCKASQNVGTNVAWYQQRPGQSPKALIYSASYRYSGVPDRFTGSGSGTDFTLTISNVQSEDLAEYFCQQFDSHPLTFGAGTKLELKGSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPALEDIACGCAAAPDIKDLLSRLEELEGLVSSLREQGTG

SEQ ID NO: 58 - protein, 530 aa, artificial sequence
  pDualyx-154 HC
MNFGFSLIFLVLVLKGVQCEVKLVPRQLDYKDDDDKEFEVKLLQSGGGLVQPGGSLKLSCEASGIDFSRYWMSWVRRAPGKGLEWIGESDPDSSTINYAPSLKDKFIISRDSARNTLYLQMSKVRSEDTALYYCAREGYYDNEGYFDVWGTGTTVTVSSRSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKLEDIACGCAAAPDIKDLLSRLEELEGLVSSLREQGTG

SEQ ID NO: 59 - protein, 619 aa, artificial sequence
  pDualyx-155 HC
MNFGFSLIFLVLVLKGVQCEVKLVPRQLDYKDDDDKEFEVKLLQSGGGLVQPGGSLKLSCEASGIDFSRYWMSWVRRAPGKGLEWIGESDPDSSTINYAPSLKDKFIISRDSARNTLYLQMSKVRSEDTALYYCAREGYYDNEGYFDVWGTGTTVTVSSRSSTKGPKLEEGEFSEAQLNIVMTQSPKSLSMSVGERVTLNCKASENVKTYVSWYQQKPEQSPKLLIYGASNRYTGVPDRFTGSGSATDFTLTISSVQAEDLADYHCGQSYSYPFTFGAGTKLERKGSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - protein, 656 aa, artificial sequence
  pDualyx-157 HC
MNFGFSLIFLVLVLKGVQCEVKLVPRQLDYKDDDDKEFEVKLLQSGGGLVQPGGSLKLSCEASGIDFSRYWMSWVRRAPGKGLEWIGESDPDSSTINYAPSLKDKFIISRDSARNTLYLQMSKVRSEDTALYYCAREGYYDNEGYFDVWGTGTTVTVSSRSSTKGPKLEEGEFSEAQLNIVMTQSPKSLSMSVGERVTLNCKASENVKTYVSWYQQKPEQSPKLLIYGASNRYTGVPDRFTGSGSATDFTLTISSVQAEDLADYHCGQSYSYPFTFGAGTKLERKGSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKLEDIACGCAAAPDIKDLLSRLEELEGLVSSLREQGTG

SEQ ID NO: 61 - protein, 440 aa, artificial sequence
  pDualyx-158 HC
MNFGFSLIFLVLVLKGVQCEVKLVPRQLDYKDDDDKEFEVKLLQSGGGLVQPGGSLKLSCEASGIDFSRYWMSWVRRAPGKGLEWIGESDPDSSTINYAPSLKDKFIISRDSARNTLYLQMSKVRSEDTALYYCAREGYYDNEGYFDVWGTGTTVTVSSRSSTKGPKLEEGEFSEAQLNIVMTQSPKSLSMSVGERVTLNCKASENVKTYVSWYQQKPEQSPKLLIYGASNRYTGVPDRFTGSGSATDFTLTISSVQAEDLADYHCGQSYSYPFTFGAGTKLERKGSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPALEDIACGCAAAPDIKDLLSRLEELEGLVSSLREQGTG

SEQ ID NO: 62 - protein, 528 aa, artificial sequence
  pDualyx-159 HC
MNFGFSLIFLVLVLKGVQCEVKLVPRQLDYKDDDDKEFQVTLKESGPGILQPSQTLSLTCSFSGFSLSTFGMGVGWIRQPSGKGLEWLAHIWWDDDKYYNPALKSRLTISKDTSENQVFLKIANVDTADTATYYCVRIAGTRYFDVWGTGTTVTVSSRSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKLEDIACGCAAAPDIKDLLSRLEELEGLVSSLREQGTG

SEQ ID NO: 63 - protein, 616 aa, artificial sequence
  pDualyx-160 HC
MNFGFSLIFLVLVLKGVQCEVKLVPRQLDYKDDDDKEFQVTLKESGPGILQPSQTLSLTCSFSGFSLSTFGMGVGWIRQPSGKGLEWLAHIWWDDDKYYNPALKSRLTISKDTSENQVFLKIANVDTADTATYYCVRIAGTRYFDVWGTGTTVTVSSRSSTKGPKLEEGEFSEAQLDIQMTQSPSSLSASLGGKVTITCKASQDINKFIAWYQHKPGKGPRLLIHYTSTLQPGIPSRFSGSGSGRDYSFSISNLEPEDIATYYCLQYDNLYTFGGGTKLEIKGSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 64 - protein, 653 aa, artificial sequence
  pDualyx-162 HC
MNFGFSLIFLVLVLKGVQCEVKLVPRQLDYKDDDDKEFQVTLKESGPGILQPSQTLSLTCSFSGFSLSTFGMGVGWIRQPSGKGLEWLAHIWWDDDKYYNPALKSRLTISKDTSENQVFLKIANVDTADTATYYCVRIAGTRYFDVWGTGTTVTVSSRSSTKGPKLEEGEFSEAQLDIQMTQSPSSLSASLGGKVTITCKASQDINKFIAWYQHKPGKGPRLLIHYTSTLQPGIPSRFSGSGSGRDYSFSISNLEPEDIATYYCLQYDNLYTFGGGTKLEIKGSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKLEDIACGCAAAPDIKDLLSRLEELEGLVSSLREQGTG

SEQ ID NO: 65 - protein, 437 aa, artificial sequence
  pDualyx-163 HC
MNFGFSLIFLVLVLKGVQCEVKLVPRQLDYKDDDDKEFQVTLKESGPGILQPSQTLSLTCSFSGFSLSTFGMGVGWIRQPSGKGLEWLAHIWWDDDKYYNPALKSRLTISKDTSENQVFLKIANVDTADTATYYCVRIAGTRYFDVWGTGTTVTVSSRSSTKGPKLEEGEFSEAQLDIQMTQSPSSLSASLGGKVTITCKASQDINKFIAWYQHKPGKGPRLLIHYTSTLQPGIPSRFSGSGSGRDYSFSISNLEPEDIATYYCLQYDNLYTFGGGTKLEIKGSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPALEDIACGCAAAPDIKDLLSRLEELEGLVSSLREQGTG

SEQ ID NO: 66 - protein, 21 aa, artificial sequence
  C1 VL CDR1
RSSTGAVTTSNYANGTNNRTP

SEQ ID NO: 67 - protein, 7 aa, artificial sequence
  C1 VL CDR2
GTNNRTP

SEQ ID NO: 68 - protein, 9 aa, artificial sequence
  C1 VL CDR3
ALWYSNHWV

SEQ ID NO: 69 - protein, 11 aa, artificial sequence
  C4 VL CDR1
KASQDVDTAVA

SEQ ID NO: 70 - protein, 7 aa, artificial sequence
  C4 VL CDR2
WASTRHT

SEQ ID NO: 71 - protein, 9 aa, artificial sequence
  C4 VL CDR3
QQYYSVPPT

SEQ ID NO: 72 - protein, 10 aa, artificial sequence
  C8 VL CDR1
KASQDVDTAV

SEQ ID NO: 73 - protein, 7 aa, artificial sequence
  C8 VL CDR2
WASTRHT

SEQ ID NO: 74 - protein, 9 aa, artificial sequence
  C8 VL CDR3
QQYYSVPPT

SEQ ID NO: 75 - protein, 10 aa, artificial sequence
  C9 VL CDR1
SASSSVSYMH

SEQ ID NO: 76 - protein, 7 aa, artificial sequence
  C9 VL CDR2
STSNLAS

SEQ ID NO: 77 - protein, 9 aa, artificial sequence
  C9 VL CDR3
QQRSSYPPT

SEQ ID NO: 78 - protein, 15 aa, artificial sequence
  C15 VL CDR1
RASENIDNYGISFMY

SEQ ID NO: 79 - protein, 7 aa, artificial sequence
  C15 VL CDR2
AASNQGS

SEQ ID NO: 80 - protein, 9 aa, artificial sequence
  C15 VL CDR3
QQSKEVPYT

SEQ ID NO: 81 - protein, 11 aa, artificial sequence
  C16 VL CDR1
KASQNVGTNVA

SEQ ID NO: 82 - protein, 7 aa, artificial sequence
  C16 VL CDR2
SASFRYS

SEQ ID NO: 83 - protein, 9 aa, artificial sequence
  C16 VL CDR3
QHFNSFPLT

SEQ ID NO: 84 - protein, 11 aa, artificial sequence
  C19 VL CDR1
KASQNVGTNVA

SEQ ID NO: 85 - protein, 7 aa, artificial sequence
  C19 VL CDR2
SASYRYS

SEQ ID NO: 86 - protein, 9 aa, artificial sequence
  C19 VL CDR3
QQFDSHPLT

SEQ ID NO: 87 - protein, 11 aa, artificial sequence
  C25 VL CDR1
KASQNVGSNVD

SEQ ID NO: 88 - protein, 7 aa, artificial sequence
  C25 VL CDR2
SASYRYS

SEQ ID NO: 89 - protein, 9 aa, artificial sequence
  C25 VL CDR3
QQYNSHPLT

SEQ ID NO: 90 - protein, 16 aa, artificial sequence
  C31-1 VL CDR1
RASQSLVHSNGNTYLQ

SEQ ID NO: 91 - protein, 7 aa, artificial sequence
  C31-1 VL CDR2
KVSNRFS

SEQ ID NO: 92 - protein, 9 aa, artificial sequence
  C31-1 VL CDR3
SQSTHVPFT

SEQ ID NO: 93 - protein, 14 aa, artificial sequence
  C31-2 VL CDR1
RSSTGAVTTSNYAN

SEQ ID NO: 94 - protein, 6 aa, artificial sequence
  C31-2 VL CDR2
TNNRAP

SEQ ID NO: 95 - protein, 9 aa, artificial sequence
  C31-2 VL CDR3
ALWYSNHWV

SEQ ID NO: 96 - protein, 11 aa, artificial sequence
  C40 VL CDR1
KASQDINKFIA

SEQ ID NO: 97 - protein, 7 aa, artificial sequence
  C40 VL CDR2
YTSTLQP

SEQ ID NO: 98 - protein, 8 aa, artificial sequence
  C40 VL CDR3
LQYDNLYT

SEQ ID NO: 99 - protein, 5 aa, artificial sequence
  C1 VH CDR1
DDYIH

SEQ ID NO: 100 - protein, 20 aa, artificial sequence
  C1 VH CDR2
WIDPENSDTEYASKFQGKAT

SEQ ID NO: 101 - protein, 12 aa, artificial sequence
  C1 VH CDR3
TTGLWLRTAIDY

SEQ ID NO: 102 - protein, 5 aa, artificial sequence
  C4 VH CDR1
SYDIN

SEQ ID NO: 103 - protein, 20 aa, artificial sequence
  C4 VH CDR2
WIYPRDGDTKYNEKFKGKAI

SEQ ID NO: 104 - protein, 10 aa, artificial sequence
  C4 VH CDR3
LTGPYWYFDV

SEQ ID NO: 105 - protein, 5 aa, artificial sequence
  C8 VH CDR1
DDYIH

SEQ ID NO: 106 - protein, 20 aa, artificial sequence
  C8 VH CDR2
WIDPENSDTEYASKFQGKAT

SEQ ID NO: 107 - protein, 10 aa, artificial sequence
  C8 VH CDR3
GLWLRTAIDY

SEQ ID NO: 108 - protein, 5 aa, artificial sequence
  C9 VH CDR1
TAGMQ

SEQ ID NO: 109 - protein, 20 aa, artificial sequence
  C9 VH CDR2
WINTHSGEPKYAEDFKGRFA

SEQ ID NO: 110 - protein, 6 aa, artificial sequence
  C9 VH CDR3
WDGTGY

SEQ ID NO: 111 - protein, 5 aa, artificial sequence
  C15 VH CDR1
DYEMH

SEQ ID NO: 112 - protein, 17 aa, artificial sequence
  C15 VH CDR2
VVDPGTGDTASNQKFKG

SEQ ID NO: 113 - protein, 11 aa, artificial sequence
  C15 VH CDR3
RYYGSRESMDY

SEQ ID NO: 114 - protein, 5 aa, artificial sequence
  C16 VH CDR1
RYWMS

SEQ ID NO: 115 - protein, 20 aa, artificial sequence
  C16 VH CDR2
ESDPDSSTINYAPSLKDKFI

SEQ ID NO: 116 - protein, 12 aa, artificial sequence
  C16 VH CDR3
EGYYDNEGYFDV

SEQ ID NO: 117 - protein, 5 aa, artificial sequence
  C19 VH CDR1
IHGMS

SEQ ID NO: 118 - protein, 20 aa, artificial sequence
  C19 VH CDR2
WINTYSGVPTYANDFKGRFA

SEQ ID NO: 119 - protein, 13 aa, artificial sequence
  C19 VH CDR3
ARDEVRRGFGFAY

SEQ ID NO: 120 - protein, 5 aa, artificial sequence
  C25 VH CDR1
DLYIH

SEQ ID NO: 121 - protein, 20 aa, artificial sequence
  C25 VH CDR2
WIDPETDNTIYDPKFQGKAS

SEQ ID NO: 122 - protein, 11 aa, artificial sequence
  C25 VH CDR3
STGLLQWYFDV

SEQ ID NO: 123 - protein, 5 aa, artificial sequence
  C31 VH CDR1
GYYWN

SEQ ID NO: 124 - protein, 19 aa, artificial sequence
  C31 VH CDR2
YITNDGSNNYNPSLKNRIS

SEQ ID NO: 125 - protein, 10 aa, artificial sequence
  C31 VH CDR3
DGGWFHYFDY

SEQ ID NO: 126 - protein, 5 aa, artificial sequence
  C31 VH CDR1
GYYWN

SEQ ID NO: 127 - protein, 19 aa, artificial sequence
  C31 VH CDR2
YITNDGSNNYNPSLKNRIS

SEQ ID NO: 128 - protein, 10 aa, artificial sequence
  C31 VH CDR3
DGGWFHYFDY

SEQ ID NO: 129 - protein, 5 aa, artificial sequence
  C40 VH CDR1
GMGVG

SEQ ID NO: 130 - protein, 19 aa, artificial sequence
  C40 VH CDR2
HIWWDDDKYYNPALKSRLT

SEQ ID NO: 131 - protein, 9 aa, Artificial Sequence
  C40 VH CDR3
IAGTRYFDV

SEQ ID NO: 132 - protein, 233 aa, Homo sapiens
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 133 - protein, 205 aa, Homo sapiens
MTPPERLFLPRVCGTTLHLLLLGLLLVLLPGAQGLPGVGLTPSAAQTARQHPKMHLAHSTLKPAAHLIGDPSKQNSLLWRANTDRAFLQDGFSLSNNSLLVPTSGIYFVYSQVVFSGKAYSPKATSSPLYLAHEVQLFSSQYPFHVPLLSSQKMVYPGLQEPWLHSMYHGAAFQLTQGDQLSTHTDGIPHLVLSPSTVFFGAFAL

SEQ ID NO: 134 - protein, 559 aa, artificial sequence
  TNC-sc(mu)TNF(143N/145R)
MNFGFSLIFLVLVLKGVQCEVKLVPRGSGSDYKDDDDKDIACGCAAAPDIKDLLSRLEELEGLVSSLREQGTGGGSGGRGEFEFTRDKPVAHVVANHQVEEQLEWLSQRANALLANGMDLKDNQLVVPADGLYLVYSQVLFKGQGCPDYVLLTHTVSRFAISYQEKVNLLSAVKSPCPKDTPEGAELKPWYEPIYLGGVFQLEKGDQLSAEVNLPKYLNFRESGQVYFGVIALGGGSGGGSGGGSGGGSDKPVAHVVANHQVEEQLEWLSQRANALLANGMDLKDNQLVVPADGLYLVYSQVLFKGQGCPDYVLLTHTVSRFAISYQEKVNLLSAVKSPCPKDTPEGAELKPWYEPIYLGGVFQLEKGDQLSAEVNLPKYLNFRESGQVYFGVIALGGGSGGGSGGGSGGGSDKPVAHVVANHQVEEQLEWLSQRANALLANGMDLKDNQLVVPADGLYLVYSQVLFKGQGCPDYVLLTHTVSRFAISYQEKVNLLSAVKSPCPKDTPEGAELKPWYEPIYLGGVFQLEKGDQLSAEVNLPKYLNFRESGQVYFGVIAL

SEQ ID NO: 135 - protein, 330 aa, artificial sequence
  full Durv heavy chain constant region sequences with CH1
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 136 - protein, 107 aa, artificial sequence [sequence omitted]

SEQ ID NO: 137 - protein, 474 aa, artificial sequence
  TNFR2(mu)-pCMV-SPORT6 (Tnfrsf1b) (Origene) cDNA clone
MAPAALWVALVFELQLWATGHTVPAQVVLTPYKPEPGYECQISQEYYDRKAQMCCAKCPPGQYVKHFCNKTSDTVCADCEASMYTQVWNQFRTCLSCSSSCTTDQVEIRACTKQQNRVCACEAGRYCALKTHSGSCRQCMRLSKCGPGFGVASSRAPNGNVLCKACAPGTFSDTTSSTDVCRPHRICSILAIPGNASTDAVCAPESPTLSAIPRTLYVSQPEPTRSQPLDQEPGPSQTPSILTSLGSTPIIEQSTKGGISLPIGLIVGVTSLGLLMLGLVNCIILVQRKKKPSCLQRDAKVPHVPDEKSQDAVGLEQQHLLTTAPSSSSSSLESSASAGDRRAPPGGHPQARVMAEAQGFQEARASSRISDSSHGSHGTHVNVTCIVNVCSSSDHSSQCSSQASATVGDPDAKPSASPKDEQVPFSQEECPSQSPCETTETLQSHEKPLPLGVPDMGMKPSQAGWFDQIAVKVA

SEQ ID NO: 138 - protein, 445 aa, Macaca fascicularis
MAVFPSSFQVAFTPYAPEPGGTCRLREYYDQTAQMCCSKCPPGQHAKVFCTKTSDTVCDSCEDSTYTQLWNWVPECLSCGSRCSSDQVETQACTREQNRICTCRPGWYCALSKQEGCRLCAQLRKCRPGFGVARPGTETSDVVCKPCAPGTFSNTTSSTDICRPHQICHVVAIPGNASMDAVCTSTSPTRSMAPGAVHLPQPVSTRSQHTQPTPAPSTAPGTSFLLPVGPSPPAEGSTGDIVLPVGLIVGVTALGLLIIGVVNCVIMTQVKKKPLCLQRETKVPHLPADKARGAQGPEQQHLLTTVPSSSSSSLESSASALDRRAPTRNQPQAPGAEKASGAGEARASTGSSDSSPGGHGTQVNVTCIVNVCSSSDHSSQCSSQASSTMGDTDASPSGSPKDEQVPFSKEESAFRSQLETPETLLGSTEEKPLPLGVPDAGMKPS

SEQ ID NO: 139 - protein, 30 aa, Homo sapiens
ACGCAAAPDVKELLSRLEELENLVSSLREQ

SEQ ID NO: 140 - protein, 127 aa, artificial sequence
  Clone 188
DVQLVESGGGSVQTGGSLTLSCAISGSTSERYCLGWFRQAPGREREGVAATSLTGRGAQFYADSVKGRFTISLDDAKNTLYLQMDSLRPDDTAVYYCAEDVGFLCGYDSDNDPFYDWGQGTQVTVSS

SEQ ID NO: 141 - protein, 127 aa, artificial sequence
  Clone 8
HVQLVESGGGSVQAGGSLRLSCAASGYTASRHCFAWFRQAPGKEREGVARLDSGDSNLAYAVFVEGRFAISQDNALNTVYLQMNNLKPDDTAMYYCAADPAPCHRAPTFRPVDFRSWGQGTQVTVSS

SEQ ID NO: 142 - protein, 117 aa, artificial sequence
  Clone 12
DVQLVESGGGLVQPGGSLRLSCGGSGFTFSSYGMSWVRQAPGKGLEWVSGIYDDGSVTDYADSVKGRFTISRDDAKNTVYLQMNSLKSEDTALYYCATGEDGELNYWGQGTQVTVSS

SEQ ID NO: 143 - protein, 127 aa, artificial sequence
  Clone 18
DVQLVESGGGSVQTGGSLTLSCAISGSTSERYCMGWFRQAPGREREGVAATSLTGRGAQFYADSVKGRFTISLDDAKNTLYLQMDSLRPDDTAVYYCAADVGFLCGYDSDNDPFYDWGQGTQVTVSS

SEQ ID NO: 144 - protein, 118 aa, artificial sequence
  Clone 36
HVQLVESGGGSVQAGGSLRLSCAASGFTFSSYYMSWVRQAPGKGLEWVSAIYSDDSDNYSDSVKGRFTISKDNAKNTLYLQMNSLKPEDTAMYYCAAIRIGTDQYKFWGQGTQVTVSS

SEQ ID NO: 145 - protein, 120 aa, artificial sequence
  Clone 74
DVQLVESGGGLVQSGGSLRLSCAASGFPFSSSDMTWVRQTSGSVFEWVSDINPDGQNTRYADSVKGRFIISRDNAKSTLYLQMDSLKTDDTAVYYCAADPGSSGGSLSYWGKGTLVTVSS

SEQ ID NO: 146 - protein, 127 aa, artificial sequence
  Clone 79
HVQLVESGGGSVQAGGSLRLSCAASVYTYSSTCMGWFRQAPGKEREGVARIDTGGGNTYYLSSVKGRFTISQDNAENTVYLQMNSLKPEDTAMYFCAADPAPCHQFLGLRGVDFRYWGQGTQVTVSS

SEQ ID NO: 147 - protein, 129 aa, artificial sequence
  Clone 82
EVQLVESGGGSVQAGETLRLSCTASGFTFLDSDMGWYRQAPGNECELVSAISSDGSTYYADSVKGRFAISQDNAKNTLYLQMNSLKTEDTAMYYCAATFYCGTTQTSRTPEVVDFATHVGQGTQVTVSS

SEQ ID NO: 148 - protein, 127 aa, artificial sequence
  Clone 90
EVQLVESGGGSVQAGGSLTLSCTASGFTFDDTDMGWYRQAPGNECELVTVISTDGDIYYTDSVKGRFTISQDNAKNTLYLQMNRLKPEDTAMYYCAARDSYRLYSACGLSTADFDDWGQGTQVTVSS

SEQ ID NO: 149 - protein, 126 aa, artificial sequence
  Clone 118
QVQLVESGGGSVQAGGSLRLSCAASGHTYRSNCMGWFRQAPGKEREGVAAMYTGTDWKYYADSVKGRFTISQDKAKNTLYLVMNNLKPDDTGVYYCAADFGFICGYDSDDDPYDAWGQGTQVTVSS

SEQ ID NO: 150 - protein, 124 aa, artificial sequence
  Clone 1248
HVQLVESGGGLVQPGGSLSLSCVGSGFTFGDYGMMWVRQAPGKGLEWVAGIYSDGSYTDYRDSVKGRFTISQDSAKNTVYLQMNSLKPEDTAVYYCAVRNYYSSYCQVNDFGYWGQGTQVTVSS

SEQ ID NO: 151 - protein, 124 aa, artificial sequence
  Clone 178
DVQLVESGGGLVHPGGSLRLSCAASGFTFSADDATWARQPPGKGLEWISTLYFDTEEKTYADSVKGRFIISRDIAKNTVYLQMNNLKPEDTGMYVCTATWAGGSLLRPASFGVWGQGTQVTVSS

SEQ ID NO: 152 - protein, 127 aa, artificial sequence
  Clone 210
HVQLVESGGGSVQAGGSLRLSCAASGYTASRHCFVWFRQAPGKEREGVARLDSGDSNLAYAVFVEGRFAISQDNALNTVYLQMNNLKPDDTAMYYCAADPAPCHRAPTFRPVDFRSWGQGTQVTVSS

SEQ ID NO: 153 - protein, 125 aa, artificial sequence
  Clone 238
QVQLVESGGGSVQPGGSLTLSCVDSGSAYLSYCMAWFRQAPGKEREGVAAIYYGGDSYYADSVKGRFTISQDHAKNTMYLQMNSLKPEDTAVYYCAADVGFICGYDSDNDPPNDWGQGTQVTVSS

SEQ ID NO: 154 - protein, 5 aa, artificial sequence
  Clone 188
RYCLG

SEQ ID NO: 155 - protein, 18 aa, artificial sequence
  Clone 188
ATSLTGRGAQFYADSVKG

SEQ ID NO: 156 - protein, 17 aa, artificial sequence
  Clone 188
DVGFLCGYDSDNDPFYD

SEQ ID NO: 157 - protein, 5 aa, artificial sequence
  Clone 8
RHCFA

SEQ ID NO: 158 - protein, 17 aa, artificial sequence
  Clone 8
RLDSGDSNLAYAVFVEG

SEQ ID NO: 159 - protein, 18 aa, artificial sequence
  Clone 8
DPAPCHRAPTFRPVDFRS

SEQ ID NO: 160 - protein, 5 aa, artificial sequence
  Clone 12
SYGMS

SEQ ID NO: 161 - protein, 17 aa, artificial sequence
  Clone 12
GIYDDGSVTDYADSVKG

SEQ ID NO: 162 - protein, 8 aa, artificial sequence
  Clone 12
GEDGELNY

SEQ ID NO: 163 - protein, 5 aa, artificial sequence
  Clone 18
RYCMG

SEQ ID NO: 164 - protein, 18 aa, artificial sequence
  Clone 18
ATSLTGRGAQFYADSVKG

SEQ ID NO: 165 - protein, 17 aa, artificial sequence
  Clone 18
DVGFLCGYDSDNDPFYD

SEQ ID NO: 166 - protein, 5 aa, artificial sequence
  Clone 36
SYYMS

SEQ ID NO: 167 - protein, 16 aa, artificial sequence
  Clone 36
AIYSDDSDNYSDSVKG

SEQ ID NO: 168 - protein, 10 aa, artificial sequence
  Clone 36
IRIGTDQYKF

SEQ ID NO: 169 - protein, 5 aa, artificial sequence
  Clone 74
SSDMT

SEQ ID NO: 170 - protein, 17 aa, artificial sequence
  Clone 74
DINPDGQNTRYADSVKG

SEQ ID NO: 171 - protein, 11 aa, artificial sequence
  Clone 74
DPGSSGGSLSY

SEQ ID NO: 172 - protein, 5 aa, artificial sequence
  Clone 79
STCMG

SEQ ID NO: 173 - protein, 17 aa, artificial sequence
  Clone 79
RIDTGGGNTYYLSSVKG

SEQ ID NO: 174 - protein, 18 aa, artificial sequence
  Clone 79
DPAPCHQFLGLRGVDFRY

SEQ ID NO: 175 - protein, 5 aa, artificial sequence
  Clone 82
DSDMG

SEQ ID NO: 176 - protein, 16 aa, artificial sequence
  Clone 82
AISSDGSTYYADSVKG

SEQ ID NO: 177 - protein, 21 aa, artificial sequence
  Clone 82
TFYCGTTQTSRTPEVVDFATH

SEQ ID NO: 178 - protein, 5 aa, artificial sequence
  Clone 90
DTDMG

SEQ ID NO: 179 - protein, 16 aa, artificial sequence
  Clone 90
VISTDGDIYYTDSVKG

SEQ ID NO: 180 - protein, 19 aa, artificial sequence
  Clone 90
RDSYRLYSACGLSTADFDD

SEQ ID NO: 181 - protein, 5 aa, artificial sequence
  Clone 118
SNCMG

SEQ ID NO: 182 - protein, 17 aa, artificial sequence
  Clone 118
AMYTGTDWKYYADSVKG

SEQ ID NO: 183 - protein, 17 aa, artificial sequence
  Clone 118
DFGFICGYDSDDDPYDA

SEQ ID NO: 184 - protein, 5 aa, artificial sequence
  Clone 1248
DYGMM

SEQ ID NO: 185 - protein, 17 aa, artificial sequence
  Clone 1248
GIYSDGSYTDYRDSVKG

SEQ ID NO: 186 - protein, 15 aa, artificial sequence
  Clone 1248
RNYYSSYCQVNDFGY

SEQ ID NO: 187 - protein, 5 aa, artificial sequence
  Clone 178
ADDAT

SEQ ID NO: 188 - protein, 17 aa, artificial sequence
  Clone 178
TLYFDTEEKTYADSVKG

SEQ ID NO: 189 - protein, 15 aa, artificial sequence
  Clone 178
TWAGGSLLRPASFGV

SEQ ID NO: 190 - protein, 5 aa, artificial sequence
  Clone 210
RHCFV

SEQ ID NO: 191 - protein, 17 aa, artificial sequence
  Clone 210
RLDSGDSNLAYAVFVEG

SEQ ID NO: 192 - protein, 18 aa, artificial sequence
  Clone 210
DPAPCHRAPTFRPVDFRS

SEQ ID NO: 193 - protein, 5 aa, artificial sequence
  Clone 238
SYCMA

SEQ ID NO: 194 - protein, 16 aa, artificial sequence
  Clone 238
AIYYGGDSYYADSVKG

SEQ ID NO: 195 - protein, 17 aa, artificial sequence
  Clone 238
DVGFICGYDSDNDPPND

SEQ ID NO: 196 - protein, 644 aa, artificial sequence
  3XVHHC188(G4S)1-Fc(Durv)(mini)
MDMRVPAQLLGLLLLWLRGARCDVQLVESGGGSVQTGGSLTLSCAISGSTSERYCLGWFRQAPGREREGVAATSLTGRGAQFYADSVKGRFTISLDDAKNTLYLQMDSLRPDDTAVYYCAEDVGFLCGYDSDNDPFYDWGQGTQVTVSSGGGGSDVQLVESGGGSVQTGGSLTLSCAISGSTSERYCLGWFRQAPGREREGVAATSLTGRGAQFYADSVKGRFTISLDDAKNTLYLQMDSLRPDDTAVYYCAEDVGFLCGYDSDNDPFYDWGQGTQVTVSSGGGGSDVQLVESGGGSVQTGGSLTLSCAISGSTSERYCLGWFRQAPGREREGVAATSLTGRGAQFYADSVKGRFTISLDDAKNTLYLQMDSLRPDDTAVYYCAEDVGFLCGYDSDNDPFYDWGQGTQVTVSSEPKSDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 197 - protein, 622 aa, artificial sequence
  3XVHHC188(G4S)1-Fc(Durv)(mini)
DVQLVESGGGSVQTGGSLTLSCAISGSTSERYCLGWFRQAPGREREGVAATSLTGRGAQFYADSVKGRFTISLDDAKNTLYLQMDSLRPDDTAVYYCAEDVGFLCGYDSDNDPFYDWGQGTQVTVSSGGGGSDVQLVESGGGSVQTGGSLTLSCAISGSTSERYCLGWFRQAPGREREGVAATSLTGRGAQFYADSVKGRFTISLDDAKNTLYLQMDSLRPDDTAVYYCAEDVGFLCGYDSDNDPFYDWGQGTQVTVSSGGGGSDVQLVESGGGSVQTGGSLTLSCAISGSTSERYCLGWFRQAPGREREGVAATSLTGRGAQFYADSVKGRFTISLDDAKNTLYLQMDSLRPDDTAVYYCAEDVGFLCGYDSDNDPFYDWGQGTQVTVSSEPKSDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 198 - protein, 127 aa, artificial sequence
  C188-VHH_1
DVQLVESGGGLVQPGGSLRLSCAISGSTSERYCLGWFRQAPGKGREGVAATSLTGRGAQFYADSVKGRFTISLDDAKNTLYLQMNSLRPEDTAVYYCAEDVGFLCGYDSDNDPFYDWGQGTLVTVSS

SEQ ID NO: 199 - protein, 127 aa, artificial sequence
  C188-VHH_2
EVQLVESGGGLVQPGGSLRLSCAISGSTSERYCLGWFRQAPGKGREGVAATSLTGRGAQFYADSVKGRFTISLDDAKNTLYLQMNSLRPEDTAVYYCAEDVGFLCGYDSDNDPFYDWGQGTLVTVSS

SEQ ID NO: 200 - protein, 127 aa, artificial sequence
  C188-VHH_3
DVQLVESGGGLVQPGGSLRLSCKISGSTSERYCLGWFRQAPGKGREGVAATSLTGRGAQFYADSVKGRFTISLDDAKNTLYLQMNSLRAEDTAVYYCAEDVGFLCGYDSDNDPFYDWGQGTLVTVSS

SEQ ID NO: 201 - protein, 127 aa, artificial sequence
  C188-VHH_4
EVQLVESGGGLVQPGGSLRLSCKISGSTSERYCLGWFRQAPGKGREGVAATSLTGRGAQFYADSVKGRFTISLDDAKNTLYLQMNSLRAEDTAVYYCAEDVGFLCGYDSDNDPFYDWGQGTLVTVSS

SEQ ID NO: 202 - protein, 127 aa, artificial sequence
  C188-VHH_5
DVQLVESGGGLVQPGGSLRLSCAISGSTSERYCLGWFRQAPGKGREGVAATSLTGRGAQFYADSVKGRFTISLDDAKNTLYLQMNSLRPEDTAVYYCAEDVGFLCGYDTDNDPFYDWGQGTLVTVSS

SEQ ID NO: 203 - protein, 127 aa, artificial sequence
  C188-VHH_6
EVQLVESGGGLVQPGGSLRLSCAISGSTSERYCLGWFRQAPGKGREGVAATSLTGRGAQFYADSVKGRFTISLDDAKNTLYLQMNSLRPEDTAVYYCAEDVGFLCGYDTDNDPFYDWGQGTLVTVSS

SEQ ID NO: 204 - protein, 127 aa, artificial sequence
  C188-VHH_7
DVQLVESGGGLVQPGGSLRLSCAISGSTSERYCLGWFRQAPGKGREGVAATSLTGRGAQFYADSVKGRFTISLDDAKNTLYLQMNSLRPEDTAVYYCAEDVGFLCGYDSDNEPFYDWGQGTLVTVSS

SEQ ID NO: 205 - protein, 127 aa, artificial sequence
  C188-VHH_8
EVQLVESGGGLVQPGGSLRLSCAISGSTSERYCLGWFRQAPGKGREGVAATSLTGRGAQFYADSVKGRFTISLDDAKNTLYLQMNSLRPEDTAVYYCAEDVGFLCGYDSDNEPFYDWGQGTLVTVSS

SEQ ID NO: 206 - protein, 127 aa, artificial sequence
  C188-VHH_9
DVQLVESGGGLVQPGGSLRLSCAISGSTSERYCLGWFRQAPGKGREGVAATSLTGRGAQFYADSVKGRFTISLDDAKNTLYLQMNSLRPEDTAVYYCAEDVGFLCGYDTDNEPFYDWGQGTLVTVSS

SEQ ID NO: 207 - protein, 127 aa, artificial sequence
  C188-VHH_10
EVQLVESGGGLVQPGGSLRLSCAISGSTSERYCLGWFRQAPGKGREGVAATSLTGRGAQFYADSVKGRFTISLDDAKNTLYLQMNSLRPEDTAVYYCAEDVGFLCGYDTDNEPFYDWGQGTLVTVSS

SEQ ID NO: 208 - protein, 127 aa, artificial sequence
  C188-VHH_11
DVQLVESGGGLVQPGGSLRLSCKISGSTSERYCLGWFRQAPGKGREGVAATSLTGRGAQFYADSVKGRFTISLDDAKNTLYLQMNSLRAEDTAVYYCAEDVGFLCGYDTDNEPFYDWGQGTLVTVSS

SEQ ID NO: 209 - protein, 127 aa, artificial sequence
  C188-VHH_12
EVQLVESGGGLVQPGGSLRLSCKISGSTSERYCLGWFRQAPGKGREGVAATSLTGRGAQFYADSVKGRFTISLDDAKNTLYLQMNSLRAEDTAVYYCAEDVGFLCGYDTDNEPFYDWGQGTLVTVSS

SEQ ID NO: 210 - protein, 127 aa, artificial sequence
  C188-VHH_13
DVQLVESGGGLVQPGGSLRLSCAISGSTSERYCLGWFRQAPGKGREGVAATSLTGRGAQFYADSVKGRFTISLDDAKNTLYLQMNSLRAEDTAVYYCAEDVGFLCGYDTDNEPFYDWGQGTLVTVSS

SEQ ID NO: 211 - protein, 127 aa, artificial sequence
  C188-VHH_14
EVQLVESGGGLVQPGGSLRLSCAISGSTSERYCLGWFRQAPGKGREGVAATSLTGRGAQFYADSVKGRFTISLDDAKNTLYLQMNSLRAEDTAVYYCAEDVGFLCGYDTDNEPFYDWGQGTLVTVSS

SEQ ID NO: 212 - protein, 127 aa, artificial sequence
  C188-VHH 15
DVQLVESGGGLVQPGGSLRLSCAISGSTSERYALGWFRQAPGKGREGVAATSLTGRGAQFYADSVKGRFTISLDDAKNTLYLQMNSLRPEDTAVYYCAEDVGFLAGYDSDNDPFYDWGQGTLVTVSS

SEQ ID NO: 213 - protein, 4 aa, artificial sequence
  C188-VHH_1
RYCL

SEQ ID NO: 214 - protein, 18 aa, artificial sequence
  C188-VHH_1
ATSLTGRGAQFYADSVKG

SEQ ID NO: 215 - protein, 17 aa, artificial sequence
  C188-VHH_1
DVGFLCGYDSDNDPFYD

SEQ ID NO: 216 - protein, 4 aa, artificial sequence
  C188-VHH_2
RYCL

SEQ ID NO: 217 - protein, 18 aa, artificial sequence
  C188-VHH_2
ATSLTGRGAQFYADSVKG

SEQ ID NO: 218 - protein, 17 aa, artificial sequence
  C188-VHH_2
DVGFLCGYDSDNDPFYD

SEQ ID NO: 219 - protein, 4 aa, artificial sequence
  C188-VHH_3
RYCL

SEQ ID NO: 220 - protein, 18 aa, artificial sequence
  C188-VHH_3
ATSLTGRGAQFYADSVKG

SEQ ID NO: 221 - protein, 17 aa, artificial sequence
  C188-VHH_3
DVGFLCGYDSDNDPFYD

SEQ ID NO: 222 - protein, 4 aa, artificial sequence
  C188-VHH_4
RYCL

SEQ ID NO: 223 - protein, 18 aa, artificial sequence
  C188-VHH_4
ATSLTGRGAQFYADSVKG

SEQ ID NO: 224 - protein, 17 aa, artificial sequence
  C188-VHH_4
DVGFLCGYDSDNDPFYD

SEQ ID NO: 225 - protein, 4 aa, artificial sequence
  C188-VHH_5
RYCL

SEQ ID NO: 226 - protein, 18 aa, artificial sequence
  C188-VHH_5
ATSLTGRGAQFYADSVKG

SEQ ID NO: 227 - protein, 17 aa, artificial sequence
  C188-VHH_5
DVGFLCGYDTDNDPFYD

SEQ ID NO: 228 - protein, 4 aa, artificial sequence
  C188-VHH_6
RYCL

SEQ ID NO: 229 - protein, 18 aa, artificial sequence
  C188-VHH_6
ATSLTGRGAQFYADSVKG

SEQ ID NO: 230 - protein, 17 aa, artificial sequence
  C188-VHH_6
DVGFLCGYDTDNDPFYD

SEQ ID NO: 231 - protein, 4 aa, artificial sequence
  C188-VHH_7
RYCL

SEQ ID NO: 232 - protein, 18 aa, artificial sequence
  C188-VHH_7
ATSLTGRGAQFYADSVKG

SEQ ID NO: 233 - protein, 17 aa, artificial sequence
  C188-VHH_7
DVGFLCGYDSDNEPFYD

SEQ ID NO: 234 - protein, 4 aa, artificial sequence
  C188-VHH_8
RYCL

SEQ ID NO: 235 - protein, 18 aa, artificial sequence
  C188-VHH_8
ATSLTGRGAQFYADSVKG

SEQ ID NO: 236 - protein, 17 aa, artificial sequence
  C188-VHH_8
DVGFLCGYDSDNEPFYD

SEQ ID NO: 237 - protein, 4 aa, artificial sequence
  C188-VHH_9
RYCL

SEQ ID NO: 238 - protein, 18 aa, artificial sequence
  C188-VHH_9
ATSLTGRGAQFYADSVKG

SEQ ID NO: 239 - protein, 17 aa, artificial sequence
  C188-VHH_9
DVGFLCGYDTDNEPFYD

SEQ ID NO: 240 - protein, 4 aa, artificial sequence
  C188-VHH_10
RYCL

SEQ ID NO: 241 - protein, 18 aa, artificial sequence
  C188-VHH_10
ATSLTGRGAQFYADSVKG

SEQ ID NO: 242 - protein, 17 aa, artificial sequence
  C188-VHH_10
DVGFLCGYDTDNEPFYD

SEQ ID NO: 243 - protein, 4 aa, artificial sequence
  C188-VHH_11
RYCL

SEQ ID NO: 244 - protein, 18 aa, artificial sequence
  C188-VHH_11
ATSLTGRGAQFYADSVKG

SEQ ID NO: 245 - protein, 17 aa, artificial sequence
  C188-VHH_11
DVGFLCGYDTDNEPFYD

SEQ ID NO: 246 - protein, 4 aa, artificial sequence
  C188-VHH_12
RYCL

SEQ ID NO: 247 - protein, 18 aa, artificial sequence
  C188-VHH_12
ATSLTGRGAQFYADSVKG

SEQ ID NO: 248 - protein, 17 aa, artificial sequence
  C188-VHH_12
DVGFLCGYDTDNEPFYD

SEQ ID NO: 249 - protein, 4 aa, artificial sequence
  C188-VHH_13
RYCL

SEQ ID NO: 250 - protein, 18 aa, artificial sequence
  C188-VHH_13
ATSLTGRGAQFYADSVKG

SEQ ID NO: 251 - protein, 17 aa, artificial sequence
  C188-VHH_13
DVGFLCGYDTDNEPFYD

SEQ ID NO: 252 - protein, 4 aa, artificial sequence
  C188-VHH_14
RYCL

SEQ ID NO: 253 - protein, 18 aa, artificial sequence
  C188-VHH_14
ATSLTGRGAQFYADSVKG

SEQ ID NO: 254 - protein, 17 aa, artificial sequence
  C188-VHH_14
DVGFLCGYDTDNEPFYD

SEQ ID NO: 255 - protein, 4 aa, artificial sequence
  C188-VHH 15
RYAL

SEQ ID NO: 256 - protein, 18 aa, artificial sequence
  C188-VHH 15
ATSLTGRGAQFYADSVKG

SEQ ID NO: 257 - protein, 17 aa, artificial sequence
  C188-VHH 15
DVGFLAGYDSDNDPFYD

SEQ ID NO: 258 - protein, 714 aa, artificial sequence
  Heavy chain (signal peptide + C4-VH + DURVA[CH1 + Hinge + CH2 + CH3] + scFvC4(C4-Vh-linker-C4-VL)
MDWTWRILFLVAAATGAHSQVQLLQSGPELVKPGASVKLSCKASGYSFTSYDINWVKQRPGQGLEWVGWIYPRDGDTKYNEKFKGKAILTVDTSSNTAYMNLHSLTSEDSAVYFCARLTGPYWYFDVWGTGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKQVQLLQSGPELVKPGASVKLSCKASGYSFTSYDINWVKQRPGQGLEWVGWIYPRDGDTKYNEKFKGKAILTVDTSSNTAYMNLHSLTSEDSAVYFCARLTGPYWYFDVWGTGTTVTVSSGGGGSGGGGSGGGGSGGGGSDIVMTQSHKFMSTSVGDRVSITCKASQDVDTAVAWYQQKPGQSPKLLIYWASTRHTGVPDRFTGSGSGTDYTLTISSVQAEDLARYYCQQYYSVPWTFGQGTKVEIK

SEQ ID NO: 259 - protein, 695 aa, artificial sequence
  Heavy chain (C4-VH + DURVA[CH1 + Hinge + CH2 + CH3] + scFvC4(C4-Vh-linker-C4-VL)
QVQLLQSGPELVKPGASVKLSCKASGYSFTSYDINWVKQRPGQGLEWVGWIYPRDGDTKYNEKFKGKAILTVDTSSNTAYMNLHSLTSEDSAVYFCARLTGPYWYFDVWGTGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKQVQLLQSGPELVKPGASVKLSCKASGYSFTSYDINWVKQRPGQGLEWVGWIYPRDGDTKYNEKFKGKAILTVDTSSNTAYMNLHSLTSEDSAVYFCARLTGPYWYFDVWGTGTTVTVSSGGGGSGGGGSGGGGSGGGGSDIVMTQSHKFMSTSVGDRVSITCKASQDVDTAVAWYQQKPGQSPKLLIYWASTRHTGVPDRFTGSGSGTDYTLTISSVQAEDLARYYCQQYYSVPWTFGQGTKVEIK

SEQ ID NO: 260 - protein, 236 aa, artificial sequence
  Light chain (signal peptide + C4-VL + durvaCL)
MDMRVPAQLLGLLLLWLRGARCDIVMTQSHKFMSTSVGDRVSITCKASQDVDTAVAWYQQKPGQSPKLLIYWASTRHTGVPDRFTGSGSGTDYTLTISSVQAEDLARYYCQQYYSVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 261 - protein, 214 aa, artificial sequence
  Light chain (C4-VL + durvaCL)
DIVMTQSHKFMSTSVGDRVSITCKASQDVDTAVAWYQQKPGQSPKLLIYWASTRHTGVPDRFTGSGSGTDYTLTISSVQAEDLARYYCQQYYSVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 262 - protein, 107 aa, artificial sequence
  Light chain sequences
DIVMTQSPSSLSTSVGDRVTITCKASQDVDTAVAWYQQKPGQSPKLLIYWASTRHTGVPSRFSGSGSGTDYTLTISSLQPEDVATYYCQQYYSVPPTFGGGTKLEIK

SEQ ID NO: 263 - protein, 107 aa, artificial sequence
  Light chain sequences
DIVMTQSPSSLSASVGDRVTITCKASQDVDTAVAWYQQKPGQSPKLLIYWASTRHTGVPSRFSGSGSGTDYTLTISSLQPEDVATYYCQQYYSVPPTFGGGTKLEIK

SEQ ID NO: 264 - protein, 107 aa, artificial sequence
  Light chain sequences
DIQMTQSPSSLSTSVGDRVTITCKASQDVDTAVAWYQQKPGQSPKLLIYWASTRHTGVPSRFSGSGSGTDYTLTISSLQPEDVATYYCQQYYSVPPTFGGGTKLEIK

SEQ ID NO: 265 - protein, 107 aa, artificial sequence
  Light chain sequences
DIQMTQSPSSLSASVGDRVTITCKASQDVDTAVAWYQQKPGQSPKLLIYWASTRHTGVPSRFSGSGSGTDYTLTISSLQPEDVATYYCQQYYSVPPTFGGGTKLEIK

SEQ ID NO: 266 - protein, 11 aa, artificial sequence
  Light chain sequences
KASQDVDTAVA

SEQ ID NO: 267 - protein, 7 aa, artificial sequence
  Light chain sequences
WASTRHT

SEQ ID NO: 268 - protein, 9 aa, artificial sequence
  Light chain sequences
QQYYSVPPT

SEQ ID NO: 269 - protein, 11 aa, artificial sequence
  Light chain sequences
KASQDVDTAVA

SEQ ID NO: 270 - protein, 7 aa, artificial sequence
  Light chain sequences
WASTRHT

SEQ ID NO: 271 - protein, 9 aa, artificial sequence
  Light chain sequences
QQYYSVPPT

SEQ ID NO: 272 - protein, 11 aa, artificial sequence
  Light chain sequences
KASQDVDTAVA

SEQ ID NO: 273 - protein, 7 aa, artificial sequence
  Light chain sequences
WASTRHT

SEQ ID NO: 274 - protein, 9 aa, artificial sequence
  Light chain sequences
QQYYSVPPT

SEQ ID NO: 275 - protein, 11 aa, artificial sequence
  Light chain sequences
KASQDVDTAVA

SEQ ID NO: 276 - protein, 7 aa, artificial sequence
  Light chain sequences
WASTRHT

SEQ ID NO: 277 - protein, 9 aa, artificial sequence
  Light chain sequences
QQYYSVPPT

SEQ ID NO: 278 - protein, 119 aa, artificial sequence
  Heavy chain sequences
QVQLVQSGPEVKKPGASVKVSCKASGYSFTSYDINWVRQAPGQGLEWMGWIYPRDGDTKYAEKFQGRVTLTVDTSISTAYMELSSLRSEDTAVYFCARLTGPYWYFDVWGQGTLVTVSS

SEQ ID NO: 279 - protein, 119 aa, artificial sequence
  Heavy chain sequences
QVQLVQSGPEVKKPGASVKVSCKASGYSFTSYDINWVRQAPGQGLEWMGWIYPRDGDTKYAEKFQGRVTLTVDTSSSTAYMELSSLRSEDTATYFCARLTGPYWYFDVWGQGTLVTVSS

SEQ ID NO: 280 - protein, 119 aa, artificial sequence
  Heavy chain sequences
QVQLVQSGPEVKKPGASVKVSCKASGYSFTSYDINWVRQAPGQGLEWMGWIYPREGDTKYAEKFQGRVTLTVDTSISTAYMELSSLRSEDTAVYFCARLTGPYWYFDVWGQGTLVTVSS

SEQ ID NO: 281 - protein, 119 aa, artificial sequence
  Heavy chain sequences
QVQLVQSGPEVKKPGASVKVSCKASGYSFTSYDINWVRQAPGQGLEWMGWIYPREGDTKYAEKFQGRVTLTVDTSSSTAYMELSSLRSEDTATYFCARLTGPYWYFDVWGQGTLVTVSS

SEQ ID NO: 282 - protein, 5 aa, artificial sequence
  Heavy chain sequences
SYDIN

SEQ ID NO: 283 - protein, 20 aa, artificial sequence
  Heavy chain sequences
WIYPRDGDTKYAEKFQGRVT

SEQ ID NO: 284 - protein, 10 aa, artificial sequence
  Heavy chain sequences
LTGPYWYFDV

SEQ ID NO: 285 - protein, 5 aa, artificial sequence
  Heavy chain sequences
SYDIN

SEQ ID NO: 286 - protein, 20 aa, artificial sequence
  Heavy chain sequences
WIYPRDGDTKYAEKFQGRVT

SEQ ID NO: 287 - protein, 10 aa, artificial sequence
  Heavy chain sequences
LTGPYWYFDV

SEQ ID NO: 288 - protein, 5 aa, artificial sequence
  Heavy chain sequences
SYDIN

SEQ ID NO: 289 - protein, 20 aa, artificial sequence
  Heavy chain sequences
WIYPREGDTKYAEKFQGRVT

SEQ ID NO: 290 - protein, 10 aa, artificial sequence
  Heavy chain sequences
LTGPYWYFDV

SEQ ID NO: 291 - protein, 5 aa, artificial sequence
  Heavy chain sequences
SYDIN

SEQ ID NO: 292 - protein, 20 aa, artificial sequence
  Heavy chain sequences
WIYPREGDTKYAEKFQGRVT

SEQ ID NO: 293 - protein, 10 aa, artificial sequence
  Heavy chain sequences
LTGPYWYFDV

SEQ ID NO: 294 - protein, 135 aa, artificial sequence
  C1 VL
QAVVTQESALTTSPGETVTLTCRSSTGAVTTSNYANWIQEKPDHLFTGLIGGTNNRTPGVPARFSGSLIGDKAALTVTGAQTEDEAVYFCALWYSNHWVFGGGTKLTVLGQPKSSPSVTLFPPSSEELETNKATL

SEQ ID NO: 295 - protein, 109 aa, artificial sequence
  C4 VL
DIVMTQSHKFMSTSVGDRVSITCKASQDVDTAVAWYQQKPGQSPKLLIYWASTRHTGVPDRFTGSGSGTDYTLTISSVQAEDLARYYCQQYYSVPPTFGGGTKLGSEIK

SEQ ID NO: 296 - protein, 107 aa, artificial sequence
  C8 VL
DIVMTQSHKFMSTSVGDRVSITCKASQDVDTAVAWYQQKPGQSPKLLIYWASTRHTGVPDRFTGSGSGTDYTLTISSVQAEDLARYYCQQYYSVPPTFGGGTKLEIK

SEQ ID NO: 297 - protein, 106 aa, artificial sequence
  C9 VL
QIVLTQSPAIMSASPGEKVTITCSASSSVSYMHWFQQKPGTSPKLWIYSTSNLASGVPARFSGSGSGTSYSLTISRMEAEDAATYYCQQRSSYPPTFGGGTKLEIK

SEQ ID NO: 298 - protein, 111 aa, artificial sequence
  C15 VL
DIVLTQSPASLAVSLGQRATISCRASENIDNYGISFMYWFQQKPGQPPKLLIYAASNQGSGVPARFSGSGSGTDFSLNIHPMEEDDTAMYFCQQSKEVPYTFGGGTKLEIK

SEQ ID NO: 299 - protein, 107 aa, artificial sequence
  C16 VL
YIVMTQSQKFMSTSVGDRVSVTCKASQNVGTNVAWYQQKPGQTPKVLIYSASFRYSGVPDRFTGSGSGTNFTLTISNVQSEDLAEYFCQHFNSFPLTFGAGTKLELK

SEQ ID NO: 300 - protein, 107 aa, artificial sequence
  C19 VL
DIVMAQSQKFMSVSVGDRVSVTCKASQNVGTNVAWYQQRPGQSPKALIYSASYRYSGVPDRFTGSGSGTDFTLTISNVQSEDLAEYFCQQFDSHPLTFGAGTKLELK

SEQ ID NO: 301 - protein, 107 aa, artificial sequence
  C25 VL
DIVMTQAQKFMSTSVGDRVSVTCKASQNVGSNVDWYQQKPGQSPKALLYSASYRYSGVPDRFTGSGSGTDFTLTISNVQSEDLAEYFCQQYNSHPLTFGAGTKLELK

SEQ ID NO: 302 - protein, 112 aa, artificial sequence
  C31-1 VL
DVVMTQTPLSLPVSLGDQASISCRASQSLVHSNGNTYLQWYLQKPGQSPKLLIYKVSNRFSGVPDRFSGSGSGTDFTLKISRVEAEDLGVFFCSQSTHVPFTFGSGTKLEIK

SEQ ID NO: 303 - protein, 110 aa, artificial sequence
  C31-2 VL
QAVVTQESALTTSPGETVTLTCRSSTGAVTTSNYANWVQEKPDHLFTGLIGGTNNRAPGVPARFSGSVIGDKAALTITGAQTEDEAIYFCALWYSNHWVFGGGTKLTVLG

SEQ ID NO: 304 - protein, 106 aa, artificial sequence
  C40 VL
DIQMTQSPSSLSASLGGKVTITCKASQDINKFIAWYQHKPGKGPRLLIHYTSTLQPGIPSRFSGSGSGRDYSFSISNLEPEDIATYYCLQYDNLYTFGGGTKLEIK

SEQ ID NO: 305 - protein, 119 aa, artificial sequence
  C1 Vh
EVQLQQSGAELVRPGASVKLSCTASGFNIKDDYIHWVKQRPEQGLEWIGWIDPENSDTEYASKFQGKATITADTSSNTAYLQLSSLTSEDTAVYYCTTGLWLRTAIDYWGQGTSITVSS

SEQ ID NO: 306 - protein, 119 aa, artificial sequence
  C4 VH
QVQLLQSGPELVKPGASVKLSCKASGYSFTSYDINWVKQRPGQGLEWVGWIYPRDGDTKYNEKFKGKAILTVDTSSNTAYMNLHSLTSEDSAVYFCARLTGPYWYFDVWGTGTTVTVSS

SEQ ID NO: 307 - protein, 119 aa, artificial sequence
  C8 VH
EVQLEESGAELVRPGASVKLSCTASGFNIKDDYIHWVKQRPEQGLEWIGWIDPENSDTEYASKFQGKATITADTSSNTAYLQLSSLTSEDTAVYYCTTGLWLRTAIDYWGQGTSITVSS

SEQ ID NO: 308 - protein, 115 aa, artificial sequence
  C9 VH
QIQLVQSGPELKKPGETVKISCKASGYTFTTAGMQWVQKMPGKGFKWIGWINTHSGEPKYAEDFKGRFAFSLETSASTAYLQISNLKNEDTATYFCARWDGTGYWGQGTTLTVSS

SEQ ID NO: 309 - protein, 120 aa, artificial sequence
  C15 VH
QVQLQQSGAELVRPGASVTLSCTASGYIFTDYEMHWVKQTPVHGLEWIGVVDPGTGDTASNQKFKGKAMLTADKSSRTAYMELRSLTSEDSAVYYCTRRYYGSRESMDYWGQGTSVTVSS

SEQ ID NO: 310 - protein, 121 aa, artificial sequence
  C16 VH
EVKLLQSGGGLVQPGGSLKLSCEASGIDFSRYWMSWVRRAPGKGLEWIGESDPDSSTINYAPSLKDKFIISRDSARNTLYLQMSKVRSEDTALYYCAREGYYDNEGYFDVWGTGTTVTVSS

SEQ ID NO: 311 - protein, 120 aa, artificial sequence
  C19 VH
QIQLVQSGPEVKKPGETVKISCKASGYTFTIHGMSWVKQAPGKGLKWMGWINTYSGVPTYANDFKGRFAFSLETSASTAYLQINNLKNEDTATYFCARDEVRRGFGFAYWGQGTLVTVSA

SEQ ID NO: 312 - protein, 118 aa, artificial sequence
  C25 VH
EVLLQQSGTELVRPGALVKLSCKASGFNIQDLYIHWVKQRPEQGLEWIGWIDPETDNTIYDPKFQGKASITADTSSNTAYLQLSSLTSEDTAMYYCSTGLLQWYFDVWGAGTAVTVSS

SEQ ID NO: 313 - protein, 119 aa, artificial sequence
  C31 VH
DVQLQESGPGLVKPSQSLSLTCSVTGSSITSGYYWNWIRQFPGNKLEWMGYITNDGSNNYNPSLKNRISITRDTSENQFFLKLNSVTTEDTATYYCARDGGWFHYFDYWGQGTTLTVSS

SEQ ID NO: 314 - protein, 119 aa, artificial sequence; identical to SEQ ID NO: 313
  C31 VH

SEQ ID NO: 315 - protein, 119 aa, artificial sequence
  C40 VH
QVTLKESGPGILQPSQTLSLTCSFSGFSLSTFGMGVGWIRQPSGKGLEWLAHIWWDDDKYYNPALKSRLTISKDTSENQVFLKIANVDTADTATYYCVRIAGTRYFDVWGTGTTVTVSS

SEQ ID NO: 316 - protein, 217 aa, artificial sequence
  Durv heavy chain constant region sequences with CH1 deletion
APEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 317 - protein, 714 aa, artificial sequence
  C4_var7
MEFGLSWVFLVALFRGVQCQVQLVQSGPEVKKPGASVKVSCKASGYSFTSYDINWVRQAPGQGLEWMGWIYPRDGDTKYAEKFQGRVTLTVDTSSSTAYMELSSLRSEDTATYFCARLTGPYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKQVQLVQSGPEVKKPGASVKVSCKASGYSFTSYDINWVRQAPGQGLEWMGWIYPRDGDTKYAEKFQGRVTLTVDTSSSTAYMELSSLRSEDTATYFCARLTGPYWYFDVWGQGTLVTVSSGGGGSGGGGSGGGGSGGGGSDIQMTQSPSSLSTSVGDRVTITCKASQDVDTAVAWYQQKPGQSPKLLIYWASTRHTGVPSRFSGSGSGTDYTLTISSLQPEDVATYYCQQYYSVPPTFGGGTKLEIK

SEQ ID NO: 318 - protein, 468 aa, artificial sequence
  C4_var7
MEFGLSWVFLVALFRGVQCQVQLVQSGPEVKKPGASVKVSCKASGYSFTSYDINWVRQAPGQGLEWMGWIYPRDGDTKYAEKFQGRVTLTVDTSSSTAYMELSSLRSEDTATYFCARLTGPYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 319 - protein, 714 aa, artificial sequence
  C4_var16
MEFGLSWVFLVALFRGVQCQVQLVQSGPEVKKPGASVKVSCKASGYSFTSYDINWVRQAPGQGLEWMGWIYPREGDTKYAEKFQGRVTLTVDTSSSTAYMELSSLRSEDTATYFCARLTGPYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKQVQLVQSGPEVKKPGASVKVSCKASGYSFTSYDINWVRQAPGQGLEWMGWIYPREGDTKYAEKFQGRVTLTVDTSSSTAYMELSSLRSEDTATYFCARLTGPYWYFDVWGQGTLVTVSSGGGGSGGGGSGGGGSGGGGSDIQMTQSPSSLSASVGDRVTITCKASQDVDTAVAWYQQKPGQSPKLLIYWASTRHTGVPSRFSGSGSGTDYTLTISSLQPEDVATYYCQQYYSVPPTFGGGTKLEIK

SEQ ID NO: 320 - protein, 468 aa, artificial sequence
  C4_var16
MEFGLSWVFLVALFRGVQCQVQLVQSGPEVKKPGASVKVSCKASGYSFTSYDINWVRQAPGQGLEWMGWIYPREGDTKYAEKFQGRVTLTVDTSSSTAYMELSSLRSEDTATYFCARLTGPYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 321 - protein, 236 aa, artificial sequence
  C4_var7
MDMRVPAQLLGLLLLWLRGARCDIQMTQSPSSLSTSVGDRVTITCKASQDVDTAVAWYQQKPGQSPKLLIYWASTRHTGVPSRFSGSGSGTDYTLTISSLQPEDVATYYCQQYYSVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 322 - protein, 482 aa, artificial sequence
  C4_var7
MDMRVPAQLLGLLLLWLRGARCDIQMTQSPSSLSTSVGDRVTITCKASQDVDTAVAWYQQKPGQSPKLLIYWASTRHTGVPSRFSGSGSGTDYTLTISSLQPEDVATYYCQQYYSVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECQVQLVQSGPEVKKPGASVKVSCKASGYSFTSYDINWVRQAPGQGLEWMGWIYPRDGDTKYAEKFQGRVTLTVDTSSSTAYMELSSLRSEDTATYFCARLTGPYWYFDVWGQGTLVTVSSGGGGSGGGGSGGGGSGGGGSDIQMTQSPSSLSTSVGDRVTITCKASQDVDTAVAWYQQKPGQSPKLLIYWASTRHTGVPSRFSGSGSGTDYTLTISSLQPEDVATYYCQQYYSVPPTFGGGTKLEIK

SEQ ID NO: 323 - protein, 236 aa, artificial sequence
  C4_var16
MDMRVPAQLLGLLLLWLRGARCDIQMTQSPSSLSASVGDRVTITCKASQDVDTAVAWYQQKPGQSPKLLIYWASTRHTGVPSRFSGSGSGTDYTLTISSLQPEDVATYYCQQYYSVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 324 - protein, 482 aa, artificial sequence
  C4_var16
MDMRVPAQLLGLLLLWLRGARCDIQMTQSPSSLSASVGDRVTITCKASQDVDTAVAWYQQKPGQSPKLLIYWASTRHTGVPSRFSGSGSGTDYTLTISSLQPEDVATYYCQQYYSVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECQVQLVQSGPEVKKPGASVKVSCKASGYSFTSYDINWVRQAPGQGLEWMGWIYPREGDTKYAEKFQGRVTLTVDTSSSTAYMELSSLRSEDTATYFCARLTGPYWYFDVWGQGTLVTVSSGGGGSGGGGSGGGGSGGGGSDIQMTQSPSSLSASVGDRVTITCKASQDVDTAVAWYQQKPGQSPKLLIYWASTRHTGVPSRFSGSGSGTDYTLTISSLQPEDVATYYCQQYYSVPPTFGGGTKLEIK

SEQ ID NO: 325 - protein, 107 aa, artificial sequence; identical to SEQ ID NO: 296
  C4′_VL

SEQ ID NO: 326 - protein, 119 aa, artificial sequence; identical to SEQ ID NO: 306
  C4′_HL

SEQ ID NO: 327 - protein, 707 aa, artificial sequence
  3xVHHC188(G4S)5-Fc(DANA)-Flag
MNFGFSLIFLVLVLKGVQCEVKLVPRGTDVQLVESGGGSVQTGGSLTLSCAISGSTSERYCLGWFRQAPGREREGVAATSLTGRGAQFYADSVKGRFTISLDDAKNTLYLQMDSLRPDDTAVYYCAEDVGFLCGYDSDNDPFYDWGQGTQVTVSSRSGGGGSGGGGSGGGGSGGGGSGGGGSDVQLVESGGGSVQTGGSLTLSCAISGSTSERYCLGWFRQAPGREREGVAATSLTGRGAQFYADSVKGRFTISLDDAKNTLYLQMDSLRPDDTAVYYCAEDVGFLCGYDSDNDPFYDWGQGTQVTVSSGGGGSGGGGSGGGGSGGGGSGGGGSQLDVQLVESGGGSVQTGGSLTLSCAISGSTSERYCLGWFRQAPGREREGVAATSLTGRGAQFYADSVKGRFTISLDDAKNTLYLQMDSLRPDDTAVYYCAEDVGFLCGYDSDNDPFYDWGQGTQVTVSSGASAGSKTHTCPPCPAPELLGGPSVFLFPPKPKDTLMISRTPEVTCVVVAVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKEFDYKDDDDKLE

SEQ ID NO: 328 - protein, 15 aa, Artificial Sequence
  Hinge with 3 cysteines
EPKSCDKTHTCPPCP

SEQ ID NO: 329 - protein, 14 aa, Artificial Sequence
  Hinge with 2 cysteines
EPKSDKTHTCPPCP

SEQ ID NO: 330 - protein, 622 aa, Artificial Sequence; identical to SEQ ID NO: 197
  3XVHHC188(G4S)1-Fc(Durv)

SEQ ID NO: 331 - protein, 622 aa, Artificial Sequence
  3XVHHC188-var14(G4S)1-Fc(Durv)
EVQLVESGGGLVQPGGSLRLSCAISGSTSERYCLGWFRQAPGKGREGVAATSLTGRGAQFYADSVKGRFTISLDDAKNTLYLQMNSLRAEDTAVYYCAEDVGFLCGYDTDNEPFYDWGQGTLVTVSSGGGGSEVQLVESGGGLVQPGGSLRLSCAISGSTSERYCLGWFRQAPGKGREGVAATSLTGRGAQFYADSVKGRFTISLDDAKNTLYLQMNSLRAEDTAVYYCAEDVGFLCGYDTDNEPFYDWGQGTLVTVSSGGGGSEVQLVESGGGLVQPGGSLRLSCAISGSTSERYCLGWFRQAPGKGREGVAATSLTGRGAQFYADSVKGRFTISLDDAKNTLYLQMNSLRAEDTAVYYCAEDVGFLCGYDTDNEPFYDWGQGTLVTVSSEPKSDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 332 - protein, 214 aa, Artificial Sequence; identical to SEQ ID NO: 261
  C4-IgG1(Durv)-HCscFvC4(G4S)4 - Light chain (C4-VL + durva-CL)

SEQ ID NO: 333 - protein, 695 aa, Artificial Sequence; identical to SEQ ID NO: 259
  C4-IgG1(Durv)-HCscFvC4(G4S)4 - Heavy chain (C4-VH + DURVA[CH1 + Hinge + CH2 + CH3] + scFvC4(C4-Vh-linker-C4-VL)

SEQ ID NO: 334 - protein, 214 aa, Artificial Sequence
  C4var7-IgG1(Durv)-HCscFvC4var7(G4S)4 - Light chain ˜= C4var7-VL + durva-CL)
DIQMTQSPSSLSTSVGDRVTITCKASQDVDTAVAWYQQKPGQSPKLLIYWASTRHTGVPSRFSGSGSGTDYTLTISSLQPEDVATYYCQQYYSVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 335 - protein, 695 aa, Artificial Sequence
  C4var7-IgG1(Durv)-HCscFvC4var7(G4S)4 - Heavy chain ˜= C4var7-VH + DURVA[CH1 + Hinge + CH2 + CH3] + scFvC4var7[C4-Vh_2-linker-C4-VL_3]
QVQLVQSGPEVKKPGASVKVSCKASGYSFTSYDINWVRQAPGQGLEWMGWIYPRDGDTKYAEKFQGRVTLTVDTSSSTAYMELSSLRSEDTATYFCARLTGPYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKQVQLVQSGPEVKKPGASVKVSCKASGYSFTSYDINWVRQAPGQGLEWMGWIYPRDGDTKYAEKFQGRVTLTVDTSSSTAYMELSSLRSEDTATYFCARLTGPYWYFDVWGQGTLVTVSSGGGGSGGGGSGGGGSGGGGSDIQMTQSPSSLSTSVGDRVTITCKASQDVDTAVAWYQQKPGQSPKLLIYWASTRHTGVPSRFSGSGSGTDYTLTISSLQPEDVATYYCQQYYSVPPTFGGGTKLEIK

SEQ ID NO: 336 - protein, 460 aa, Artificial Sequence
  C4var16-IgG1(Durv)-LCscFvC4var16(G4S)4 - Light chain ˜= C4var16-VL + durva-CL + scFvC4var16[C4-Vh_4-linker-C4-VL_4]
DIQMTQSPSSLSASVGDRVTITCKASQDVDTAVAWYQQKPGQSPKLLIYWASTRHTGVPSRFSGSGSGTDYTLTISSLQPEDVATYYCQQYYSVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECQVQLVQSGPEVKKPGASVKVSCKASGYSFTSYDINWVRQAPGQGLEWMGWIYPREGDTKYAEKFQGRVTLTVDTSSSTAYMELSSLRSEDTATYFCARLTGPYWYFDVWGQGTLVTVSSGGGGSGGGGSGGGGSGGGGSDIQMTQSPSSLSASVGDRVTITCKASQDVDTAVAWYQQKPGQSPKLLIYWASTRHTGVPSRFSGSGSGTDYTLTISSLQPEDVATYYCQQYYSVPPTFGGGTKLEIK

SEQ ID NO: 337 - protein, 449 aa, Artificial Sequence
  C4var16-IgG1(Durv)-LCscFvC4var16(G4S)4 - Heavy chain ˜= C4var16-VH + DURVA[CH1 + Hinge + CH2 + CH3]
QVQLVQSGPEVKKPGASVKVSCKASGYSFTSYDINWVRQAPGQGLEWMGWIYPREGDTKYAEKFQGRVTLTVDTSSSTAYMELSSLRSEDTATYFCARLTGPYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 338 - protein, 644 aa, Artificial Sequence
  3XVHHC188-var14(G4S)1-Fc(Durv) - (signal peptide + C188variant14 + G4S + C188variant14 + G4S + C188variant14 + hinge + Fc(Durvalumab)
MDMRVPAQLLGLLLLWLRGARCEVQLVESGGGLVQPGGSLRLSCAISGSTSERYCLGWFRQAPGKGREGVAATSLTGRGAQFYADSVKGRFTISLDDAKNTLYLQMNSLRAEDTAVYYCAEDVGFLCGYDTDNEPFYDWGQGTLVTVSSGGGGSEVQLVESGGGLVQPGGSLRLSCAISGSTSERYCLGWFRQAPGKGREGVAATSLTGRGAQFYADSVKGRFTISLDDAKNTLYLQMNSLRAEDTAVYYCAEDVGFLCGYDTDNEPFYDWGQGTLVTVSSGGGGSEVQLVESGGGLVQPGGSLRLSCAISGSTSERYCLGWFRQAPGKGREGVAATSLTGRGAQFYADSVKGRFTISLDDAKNTLYLQMNSLRAEDTAVYYCAEDVGFLCGYDTDNEPFYDWGQGTLVTVSSEPKSDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK